(19) United States
(12) Patent Application Publication
(10) Pub. No.: US 2023/0027361 A1
(43) Pub. Date: Jan. 26, 2023

(54) METTL3 MODULATORS

(71) Applicant: Accent Therapeutics, Inc., Lexington, MA (US)

(72) Inventors: Thomas Andrew Wynn, Lexington, MA (US); Brian Lewis Hodous, Arlington, MA (US); Paula Ann Boriack-Sjodin, Lexington, MA (US); Ernest Allen Sickmier, Needham, MA (US); James Edward John Mills, Sheffield (GB); Robert A. Copeland, Lexington, MA (US); Andrew Stewart Tasker, Simi Valley, CA (US); Matthew H. Daniels, Somerville, MA (US); Kenneth W. Duncan, Westwood, MA (US); Brian Andrew Sparling, Melrose, MA (US)

(73) Assignee: Accent Therapeutics, Inc., Lexington, MA (US)

(21) Appl. No.: 17/770,708

(22) PCT Filed: Oct. 20, 2020
(86) PCT No.: PCT/IB2020/001135

§ 371 Date: Apr. 21, 2022

Related U.S. Application Data
(60) Provisional application No. 62/923,936, filed on Oct. 21, 2019.

(51) Int. Cl. C07D 487/04; C07D 215/04; C07D 221/02; C07D 307/02; A61P 35/02

(52) U.S. Cl. CPC C07D 487/04; C07D 215/04; C07D 221/02; C07D 307/02; A61P 35/02

(57) ABSTRACT

Provided are compounds of Formula (I′) or (II′), or pharmaceutically acceptable salts thereof, and methods for their use and production.

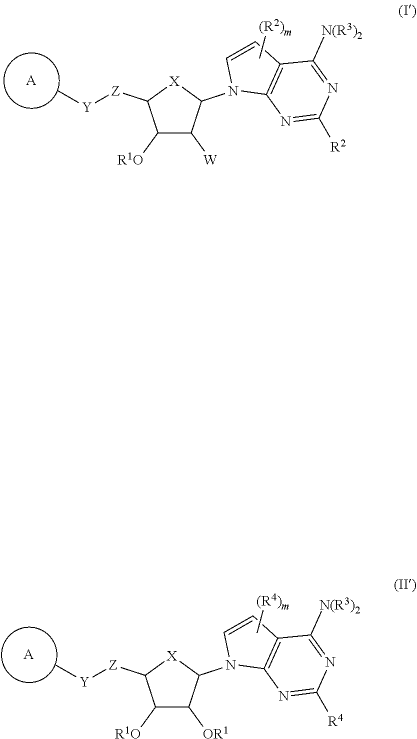

### RELATED APPLICATION

This application claims the benefit of the filing date, under 35 U.S.C. § 119(e), of U.S. Provisional Application No. 62/923,936, filed on Oct. 21, 2019, the entire contents of which are incorporated herein by reference

### TECHNICAL FIELD

This invention relates to compounds that are METTL3 modulating agents, and methods of making and using such compounds.

### BACKGROUND

Among all RNA modifications, N⁶-methyladenosine (m⁶A) is the most abundant mRNA internal modification. It plays important roles in the biogenesis and functions of RNA. m⁶A deposition on mRNA is regulated by the dynamic interplay between RNA specific methylase (“writers”), binding proteins (“readers”), and demethylases (“erasers”) (Ying Yang, Cell Research volume 28, pages 616-624, 2018). m⁶A methylation is controlled by a large RNA methyltransferase complex (MTase), composed of the methyltransferase-like 3 and 14 (METTL3 and METTL14) proteins and their cofactor, Wilms' tumor 1-associated protein (WTAP). METTL3 is the catalytic component that forms a heterodimer with METTL14, which facilitates the interactions with its target mRNA.

METTL3 has been demonstrated to modulate embryonic development, cell reprogramming, spermatogenesis, regulation of T cell homeostasis and endothelial-to-hematopoietic transition via methylation of specific target transcripts. Aberrant METTL3 expression has been associated with various pathophysiology, such as cancer, obesity, infection, inflammation and immune response (Sibbritt et al., 2013). AML is one of the cancers with the highest expression of both METTL3 and METTL14. Both genes were found upregulated in all subtypes of AML compared to normal hematopoietic cells.

Despite recent advances in METTL3 research, there is still a great need for small molecule METTL3 inhibitors as potential therapeutic agent for treating diseases that are responsive to modulation of METTL3 activities.

### SUMMARY

In accordance with the purpose(s) of the invention, as embodied and broadly described herein, the invention, in an aspect, relates to compounds useful as METTL3 modulators, pharmaceutical compositions, methods of making and methods of treating disorders using the same. In some embodiments, the compounds of the invention are METTL3 inhibitors.

In one aspect, the present invention provides a compound of formula (I′) or (II′):

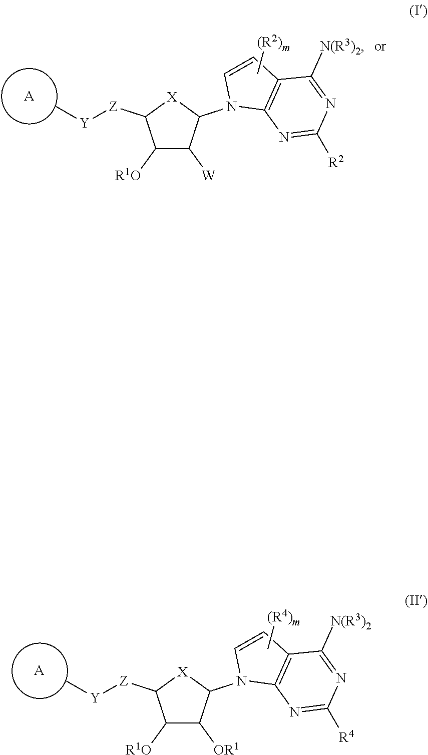

or a pharmaceutically acceptable salt thereof, wherein:

X is selected from O and CH₂;

R¹ is selected from H, C₁₋₆alkyl and —C(═O)—C₁₋₆alkyl;

W is selected from H, halo, C₁₋₆alkyl and —NH₂;

Y is selected from O, S, C(R^{a})₂ and NR^{b};

R^{a}, for each occurrence, is independently selected from H, C₁₋₆alkyl and halo;

R^{b} is H or C₁₋₆alkyl;

Z is selected from C₁₋₆alkyl, C₂₋₆alkenyl and C₂₋₆ alkynyl, each of which is optionally substituted with 1 to 3 halo;

Ring A is selected from benzene, naphthalene, 4 to 7-membered monocyclic heterocycloalkyl, 5 to 6-membered monocyclic heteroaromatic ring, and 8- to 10-membered bicyclic heteroaromatic ring, each of which is optionally substituted with 1 to 4 independently selected R₅;

R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —C(═O)OR^{2a}, —C(═O)R^{2a}, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂.

R^{2a}, for each occurrence, is independently selected from H, C₁₋₆alkyl, C₃₋₈cycloalkyl, and 4 to 6-membered heterocycloalkyl, wherein the C₁₋₆alkyl is optionally substituted with C₁₋₆alkoxy;

R³, for each occurrence, is H or C₁₋₆alkyl optionally substituted with 1 to 3 substituents independently selected from C₃₋₆cycloalkyl, phenyl and halo;

R⁴, for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{4a}, —C(═O)N(R^{4a})₂, and —N(R^{4a})₂.

R^{4a}, for each occurrence, is independently selected from H, C₁₋₆alkyl, C₃₋₈cycloalkyl, and 4 to 6-membered heterocycloalkyl;

R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂, —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, phenyl, 5- to 6-membered heteroaryl, halo, —CN, —OR^{5a}—N(R^{5a})₂, —C(O)N(R^{5a})₂, —C(O)R^{5a}, and —C(O)OR^{5a};

R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, —NH₂, —SO₂C₁₋₆alkyl, —OC₁₋₆alkyl, C₁₋₆alkyl, C₁₋₆ haloalkyl, C₃₋₆cycloalkyl, phenyl, and 4 to 7-membered heterocycloalkyl;

or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, —OH, —NH₂, C₁₋₄alkyl and C₁₋₄haloalkyl; and
m is 1 or 2,

provided that the compound is not any one of the following, or a pharmaceutically acceptable salt thereof:

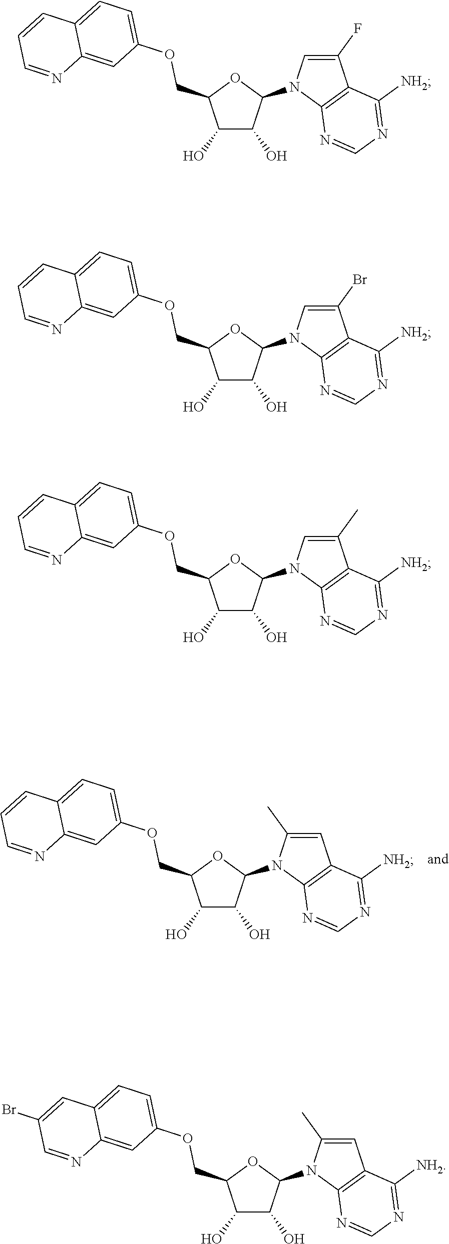 [SMILES: Nc1ncnc2c1c(F)cn2[C@@H]1O[C@H](COc2ccc3cccnc3c2)[C@@H](O)[C@H]1O ; Nc1ncnc2c1c(Br)cn2[C@@H]1O[C@H](COc2ccc3cccnc3c2)[C@@H](O)[C@H]1O ; Cc1cn([C@@H]2O[C@H](COc3ccc4cccnc4c3)[C@@H](O)[C@H]2O)c2ncnc(N)c12 ; Cc1cc2c(N)ncnc2n1[C@@H]1O[C@H](COc2ccc3cccnc3c2)[C@@H](O)[C@H]1O ; Cc1cc2c(N)ncnc2n1[C@@H]1O[C@H](COc2ccc3cc(Br)cnc3c2)[C@@H](O)[C@H]1O]

In another aspect, the present invention provides a compound of formula (I) or (II):

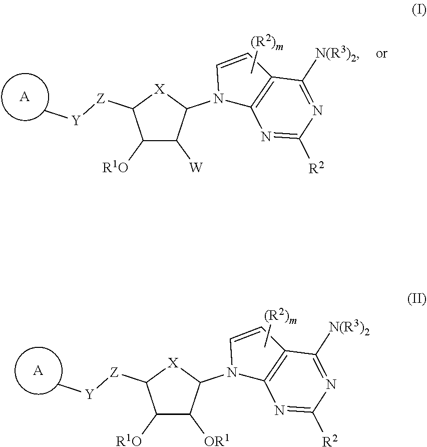

or a pharmaceutically acceptable salt thereof, wherein:

X is selected from O and CH₂;

R¹ is selected from H, C₁₋₆alkyl and —C(═O)—C₁₋₆alkyl;

W is selected from H, halo, C₁₋₆alkyl and —NH₂;

Y is selected from O, S, C(R^{a})₂ and NR^{b};

R^{a}, for each occurrence, is independently selected from H, C₁₋₆alkyl and halo;

R^{b} is H or C₁₋₆alkyl;

Z is selected from C₁₋₆alkyl, C₂₋₆alkenyl and C₂₋₆ alkynyl, each of which is optionally substituted with 1 to 3 halo;

Ring A is selected from benzene, naphthalene, 5 to 6-membered monocyclic heteroaromatic ring, and 8- to 10-membered bicyclic heteroaromatic ring, each of which is optionally substituted with 1 to 4 independently selected R⁵;

R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl, halo, —CN, —OR^{2a}, —N(R^{2a})₂, —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂.

R^{2a}, for each occurrence, is independently selected from H, C₁₋₆alkyl, C₃₋₈cycloalkyl, and 4 to 6-membered heterocycloalkyl;

R³, for each occurrence, is H or C₁₋₆alkyl optionally substituted with 1 to 3 substituents independently selected from C₃₋₆cycloalkyl, phenyl and halo;

R⁴, for each occurrence, is independently selected from C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl, halo, —CN, —OR², —N(R^{2a})₂, —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{4a}, —C(═O)N(R^{4a})₂, and —N(R^{4a})₂; R^{4a}, for each occurrence, is independently selected from H, C₁₋₆alkyl, C₃₋₈cycloalkyl, and 4 to 6-membered heterocycloalkyl;

R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂, —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{5a}—N(R^{5a})₂, —C(O)N(R^{5a})₂, —C(O)R^{5a}, and —C(O)OR^{5a};

R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl, and 4 to 7-membered heterocycloalkyl; or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl; and

m is 1 or 2,

provided that the compound is not any one of the following, or a pharmaceutically acceptable salt thereof:

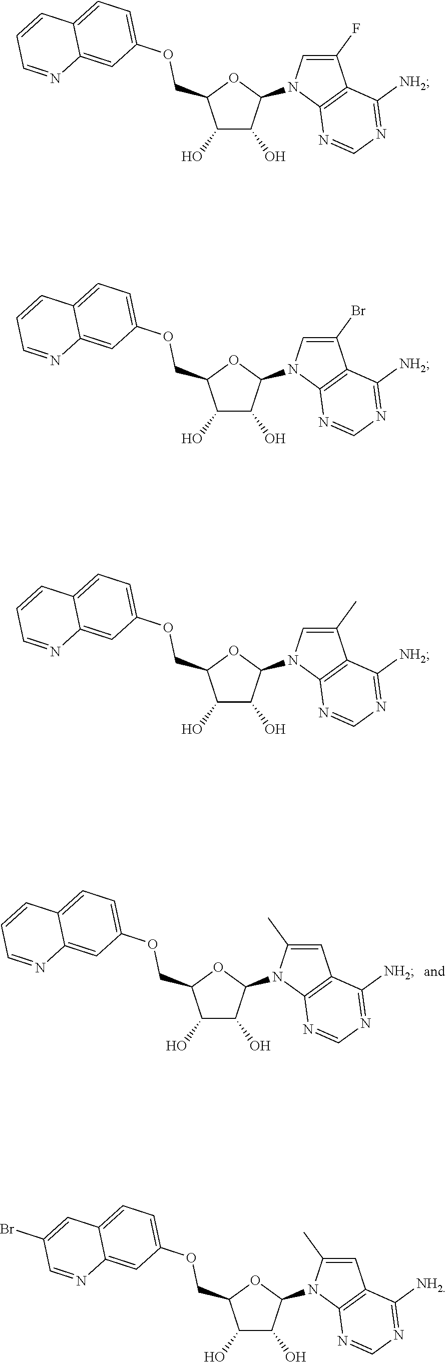 [SMILES: Nc1ncnc2c1c(F)cn2[C@@H]1O[C@H](COc2ccc3cccnc3c2)[C@@H](O)[C@H]1O ; Nc1ncnc2c1c(Br)cn2[C@@H]1O[C@H](COc2ccc3cccnc3c2)[C@@H](O)[C@H]1O ; Cc1cn([C@@H]2O[C@H](COc3ccc4cccnc4c3)[C@@H](O)[C@H]2O)c2ncnc(N)c12 ; Cc1cc2c(N)ncnc2n1[C@@H]1O[C@H](COc2ccc3cccnc3c2)[C@@H](O)[C@H]1O ; Cc1cc2c(N)ncnc2n1[C@@H]1O[C@H](COc2ccc3cc(Br)cnc3c2)[C@@H](O)[C@H]1O]

The present invention also provides a pharmaceutical composition comprising at least one compound described herein, or a pharmaceutically acceptable salt thereof, and at least one pharmaceutically acceptable excipient.

In one embodiment, the invention is a method of treating a disorder responsive to inhibition of METTL3 activity in a subject comprising administering to said subject an effective amount of at least one compound described herein, or a pharmaceutically acceptable salt thereof.

The present invention also includes the use of at least one compound described herein, or a pharmaceutically acceptable salt thereof, for the manufacture of a medicament for the treatment of a disorder responsive to inhibition of METTL3 activity. Also provided is a compound described herein, or a pharmaceutically acceptable salt thereof for use in treating a disorder responsive to inhibition of METTL3 activity.

Other features or advantages will be apparent from the following detailed description of several embodiments, and also from the appended claims.

### DETAILED DESCRIPTION

It has been found that the compounds of the present invention are useful as METTL3 inhibitors. The compounds according to the invention and compositions thereof, may be useful for the treatment of autoimmune diseases, cancer, inflammatory diseases, and infectious diseases, such as viral infections.

In a first embodiment of the present invention, the compound is represented by formula (I′) or (II′), or a pharmaceutically acceptable salt thereof, wherein the definitions for the variables are as defined above.

In a second embodiment of the present invention, the compound is represented by formula (I) or (II), or a pharmaceutically acceptable salt thereof, wherein the definitions for the variables are as defined above.

In a third embodiment of the present invention, the compound is represented by the following formula:

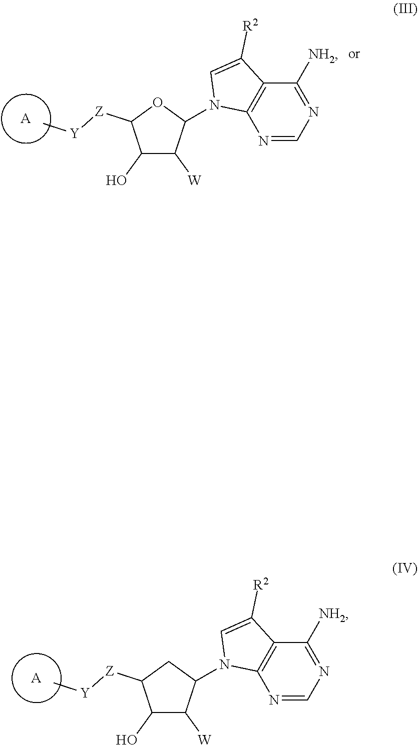

or a pharmaceutically acceptable salt thereof; and the definitions for the variables are as defined in the first embodiment.

In a fourth embodiment of the present invention, the compound is represented by the following formula:

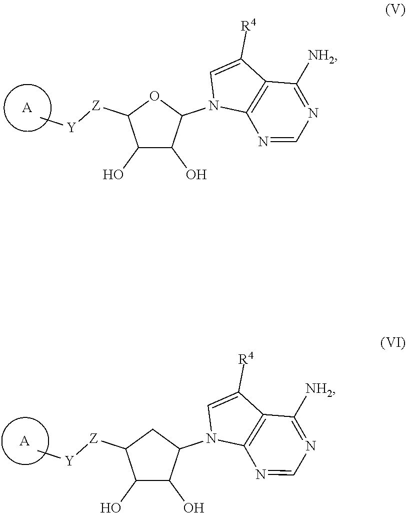

or a pharmaceutically acceptable salt thereof; and the definitions for the variables are as defined in the first embodiment.

In a fifth embodiment of the present invention, the compound is represented by formula (I′), (II′), (I), (II), (III), (IV), (V) or (VI), or a pharmaceutically acceptable salt thereof, wherein Y is O or C(R^{a})₂ and R^{a}, for each occurrence, is independently H or halo; and the definitions for the other variables are as defined in the first, second, third, or fourth embodiment.

In a sixth embodiment of the present invention, the compound is represented by formula (I′), (II′), (I), (II), (III), (IV), (V) or (VI), or a pharmaceutically acceptable salt thereof, wherein Y is O; and the definitions for the other variables are as defined in the first, second, third, fourth or fifth embodiment.

In a seventh embodiment of the present invention, the compound is represented by formula (I′), (II′), (I), (II), (III), (IV), (V) or (VI), or a pharmaceutically acceptable salt thereof, wherein Y is CH₂; and the definitions for the other variables are as defined in the first, second, third, fourth or fifth embodiment.

In an eighth embodiment of the present invention, the compound is represented by formula (I′), (II′), (I), (II), (III), (IV), (V) or (VI), or a pharmaceutically acceptable salt thereof, wherein Z is selected from C₁₋₄alkyl and C₂₋₄ alkenyl, each of which is optionally substituted with 1 to 3 halo; and the definitions for the other variables are as defined in the first, second, third, fourth, fifth, sixth or seventh embodiment.

In a ninth embodiment of the present invention, the compound is represented by formula (I′), (II′), (I), (II), (III), (IV), (V) or (VI), or a pharmaceutically acceptable salt thereof, wherein Z is CH₂; and the definitions for the other variables are as defined in the first, second, third, fourth, fifth, sixth, seventh, or eighth embodiment.

In a tenth embodiment of the present invention, the compound is represented by the following formula:

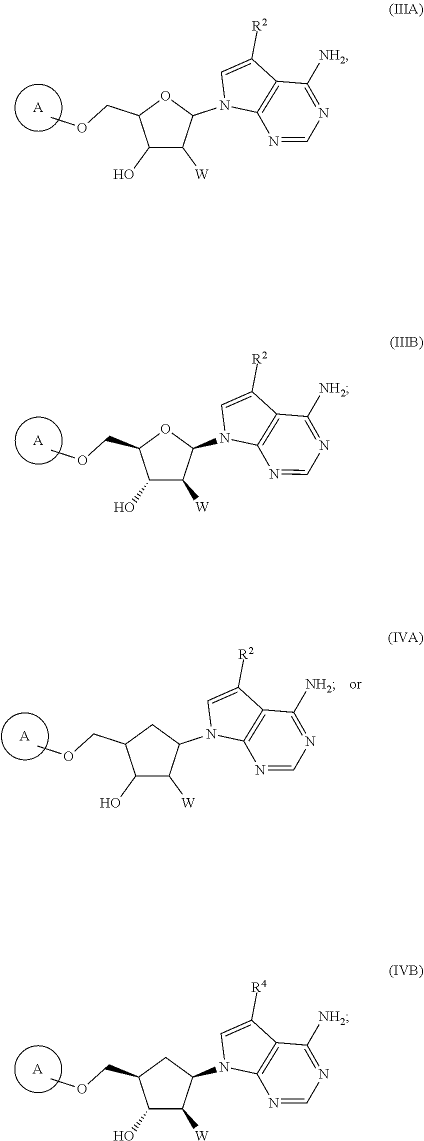

or a pharmaceutically acceptable salt thereof; and the definitions for the other variables are as defined in the first or second embodiment.

In an eleventh embodiment of the present invention, the compound is represented by the following formula:

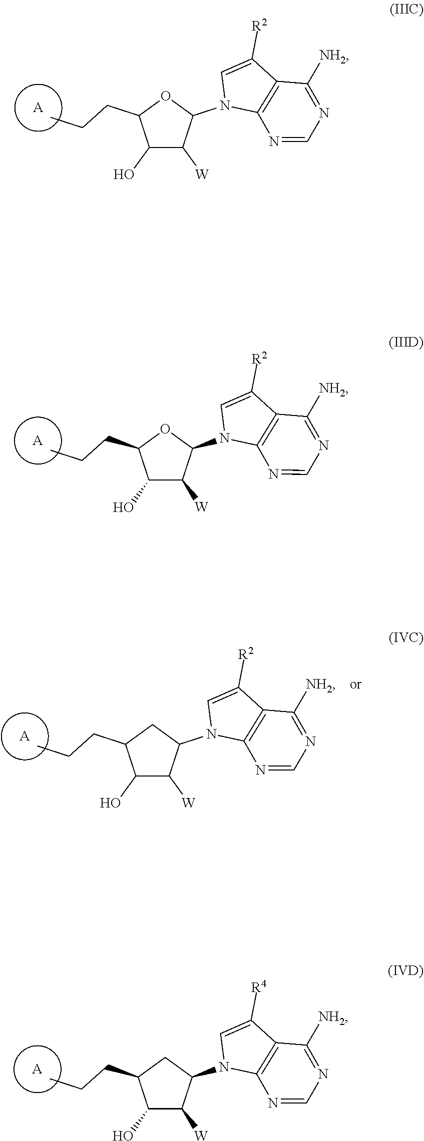

or a pharmaceutically acceptable salt thereof; and the definitions for the other variables are as defined in the first or second embodiment.

In a twelfth embodiment of the present invention, the compound is represented by the following formula:

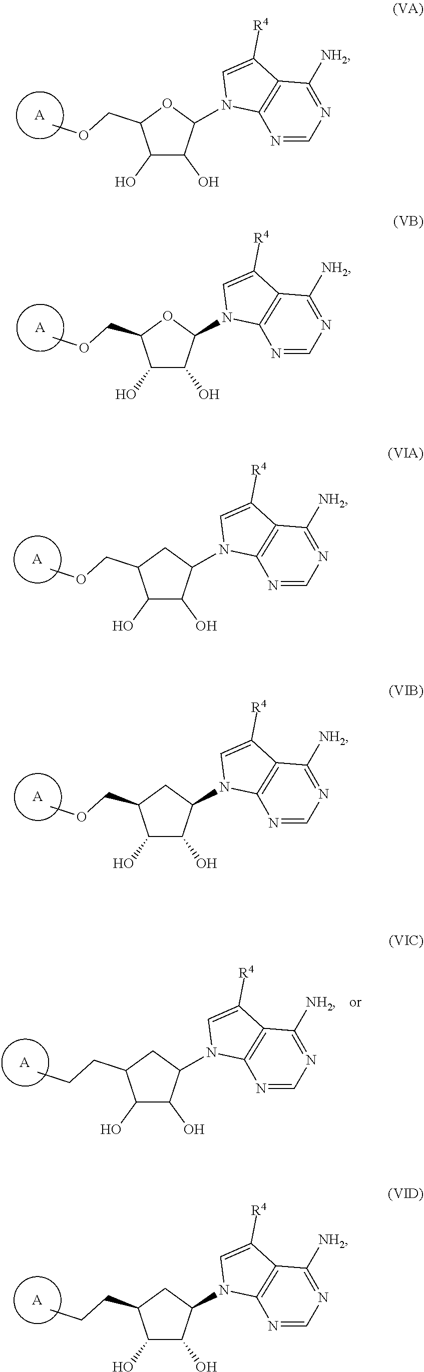

or a pharmaceutically acceptable salt thereof, wherein the definitions for the variables are as defined in the first or second embodiment.

In a thirteenth embodiment of the present invention, the compound is represented by the following formula:

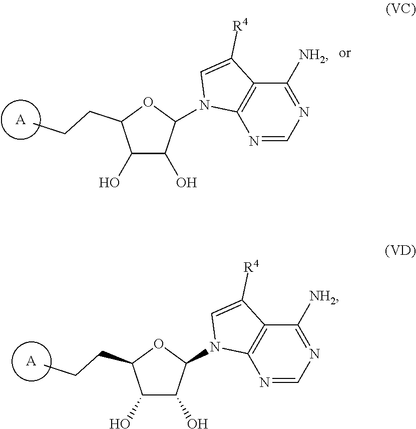

or a pharmaceutically acceptable salt thereof; and the definitions for the other variables are as defined in the first or second embodiment.

In a fourteenth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is a 9- to 10-membered bicyclic heteroaromatic ring optionally substituted with 1 to 4 independently selected R⁵ groups; and the definitions for the other variables are as defined in the first, second, third, fourth, fifth, sixth, seventh, eighth, ninth, tenth, eleventh, twelfth, or thirteenth embodiment.

In a fifteenth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is selected from quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, isothiazolopyridine, thienopyridine, pyridine, piperidine, and benzene, each of which is optionally substituted with 1 to 3 independently selected R⁵; and the definitions for the other variables are as defined in the first, second, third, fourth, fifth, sixth,seventh, eighth, ninth, tenth, eleventh, twelfth, or thirteenth embodiment.

In a sixteenth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is selected from quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, and isothiazolopyridine, each of which is optionally substituted with 1 to 3 independently selected R⁵; and the definitions for the other variables are as defined in the first, second, third, fourth, fifth, sixth, seventh, eighth, ninth, tenth, eleventh, twelfth, or thirteenth embodiment.

In a seventeenth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is selected from quinoline, quinazoline, quinoxaline, benzoimidiazole, benzothiazole, napththyridine, indole, pyrrolopyrimidine and indazole, each of which is optionally substituted with 1 to 3 independently selected R⁵; and the definitions for the other variables are as defined in the sixteenth embodiment.

In an eighteenth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is selected from benzene, naphthalene and pyridine; and the definitions for the other variables are as defined in the first, second, third, fourth, fifth, sixth, seventh, eighth, ninth, tenth, eleventh, twelfth, or thirteenth embodiment.

In a nineteenth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is quinoline optionally substituted with 1 to 3 independently selected R⁵; and the definitions for the other variablese are as defined in the seventeenth embodiment.

In a twentieth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is represented by the following formula:

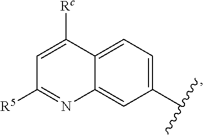

wherein R^{c} is selected from H, halo, C₁₋₄alkyl, 4 to 6-membered heterocycloalkyl, —OR^{c1} and —N(R^{c1})₂, wherein R^{c1}, for each occurrence, is independently H, C₁₋₄alkyl, or C₃₋₆ cycloalkyl, wherein the C₁₋₄alkyl is optionally substituted with C₃₋₆cycloalkyl or phenyl; and the definitions for the other variables are as defined in the first, second, third, fourth, fifth, sixth, seventh, eighth, ninth, tenth, eleventh, twelfth, or thirteenth embodiment.

In a twenty-first embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein ring A is represented by the following formula:

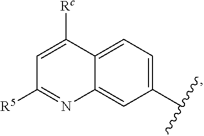

wherein R^{c} is selected from H, halo, C₁₋₄alkyl, —OR^{c1} and —N(R^{c1})₂, and R^{c1}, for each occurrence, is independently H or C₁₋₄alkyl optionally substituted with C₃₋₆cycloalkyl or phenyl; and the definitions for the other variables are as defined in the first, second, third, fourth, fifth, sixth, seventh, eighth, ninth, tenth, eleventh, twelfth, or thirteenth embodiment.

In a twenty-second embodiment of the present invention, the compound is as defined in the twentieth or twenty-first embodiment, or a pharmaceutically acceptable salt thereof, wherein R^{c} is H.

In a twenty-third embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein R⁵, for each occurrence, is independently selected from H, C₁₋₆alkyl, C₂₋₆alkynyl, C₃₋₆cycloalkyl, 4 to 6-membered heterocycloalkyl, 5 to 6-membered heteroaryl, halo, —OR^{5a}, —C(═O)N(R^{5a})₂, —N(R^{5a})₂—NR^{5a}C(═O)R^{5a}, and —NR^{5a}C(═O)N(R^{5a})₂, wherein the C₁₋₆alkyl, C₂₋₆alkynyl, C₃₋₆cycloalkyl, 4 to 6-membered heterocycloalkyl, and 5 to 6-membered heteroaryl are each optionally substituted with 1 to 3 substituents independently selected from C₁₋₆alkyl, C₁₋₆ haloalkyl, C₃₋₆cycloalkyl, —N(R^{5a})₂, phenyl, halo, —OH, —NH₂, and —CN; and

R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OH, —NH₂, phenyl, —SO₂C₁₋₃alkyl, —OC₁₋₃alkyl, C₁₋₃alkyl and C₃₋₈cycloalkyl, or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, —OH, —NH₂, C₁₋₄alkyl and C₁₋₄ haloalkyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-fourth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein R⁵, for each occurrence, is independently selected from H, C₁₋₆alkyl, C₂₋₆alkynyl, 4 to 6-membered heterocycloalkyl, 5 to 6-membered heteroaryl, halo, —OR^{5a}, —N(R^{5a})₂, —NR^{5a}C(═O)R^{5a}, and —NR^{5a}C(═O)N(R^{5a})₂, wherein the C₁₋₆alkyl, C₂₋₆alkynyl, 4 to 6-membered heterocycloalkyl, 5 to 6-membered heteroaryl are each optionally substituted with 1 to 3 substituents independently selected from C₁₋₆alkyl, C₁₋₆haloalkyl, C₃₋₆ cycloalkyl, phenyl, halo and —CN; and

R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OH, C₁₋₃alkyl and C₃₋₈cycloalkyl, or two R^{5a} together with the N atom from which they are attached form a 4 to 6-membered heterocycloalkyl optionally containing an additional heteroatom selected from O, N and S; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-fifth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein R⁵, for each occurrence, is independently selected from H, C₁₋₆alkyl and —N(R^{a})₂; and R^{5a}, for each occurrence, is independently H or C₁₋₆alkyl, or two R^{5a} together with the N atom from which they are attached form a 4 to 6-membered heterocycloalkyl optionally containing an additional heteroatom selected from O, N and S; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, or twenty-fourth embodiment.

In a twenty-sixth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein R⁵, for each occurrence, is independently selected from H, Br, F, —CH₃, —CH₂CH₃, —CH₂N(CH₃)₂, —OH, —OCH3, —NH₂, —NHCH₃, —NHCH₂CH₃, —N(CH₃)₂, —NHCH(CH₃)₂, —NHCH₂CH₂CH₃, —NHCH₂CH₂OH, —NHCH₂-cyclopropyl, —NH-cyclobutyl, —NHCH₂Ph, —N(CH₃)CH₂Ph, —NHPh, —NHC(O)NH₂, —NH—C(═O)-cyclopropyl, —NHC(═O)NHCH₃, —C≡C-Ph, imidazolyl, pyrrolidinyl, morpholinyl and azetidinyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-seventh embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein R⁵, for each occurrence, is independently selected from H, Br, F, —CH₃, —CH₂CH₃, —CH₂N(CH₃)₂, —OH, —OCH₃, —NH₂, —NHCH₃, —NHCH₂CH₃, —N(CH₃)₂, —NHCH(CH₃)₂, —NHCH₂CH₂CH₃, —NHCH₂CH₂OH, —NHCH₂-cyclopropyl, —NHCH₂Ph, —N(CH₃)CH₂Ph, —NHPh, —NHC(O)NH₂, —NH—C(═O)-cyclopropyl, —NHC(═O)NHCH₃, —C≡C-Ph, imidazolyl, pyrrolidinyl and morpholinyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-eighth embodiment of the present invention, the compound is represented by formula (I′), (II′), (I), (II), (III), (IV), (V), (VI), (IIIA), (IIIB), (IIIC), (IIID), (IVA), (IVB), (IVC), (IVD), (VA), (VB), (VC), (VD), (VIA), (VIB), (VIC) or (VID), or a pharmaceutically acceptable salt thereof, wherein R⁵, for each occurrence, is independently selected from —NHCH₃, —NHCH₂CH₃, —NH-cyclobutyl, and azetidinyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-ninth embodiment, the compound is represented by formula (I′), (II′), (I), (II), (III), (IV), (IIIA), (IIIB), (IIIC), (IIID), (IVA), (IVB), (IVC), or (IVD), or a pharmaceutically acceptable salt thereof, wherein W is selected from H and halo; and the definitions for the other variables are as defined in the first, second, third, fifth, sixth, seventh, eighth, ninth, tenth, eleventh, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, or twenty-eighth embodiment.

In a thirtieth embodiment of the present invention, the compound is as defined in the twenty-ninth embodiment, or a pharmaceutically acceptable salt thereof, wherein W is selected from H and F.

In a thirty-first embodiment of the present invention, the compound is represented by formula (I′), (II′), (I), (II), (III), (IV), (IIIA), (IIIB), (IIIC), (IIID), (IVA), (IVB), (IVC), or (IVD), or a pharmaceutically acceptable salt thereof, wherein R² is H, halo, C₁₋₆alkyl, C₃₋₈cycloalkyl, C₅₋₈cycloalkenyl, 4 to 6-membered heterocycloalkyl, 4 to 6-membered heterocycloalkenyl, phenyl, 5 to 6-membered heteroaryl, wherein the C₁₋₆alkyl, C₃₋₈cycloalkyl, C₅₋₈cycloalkenyl, 4 to 6-membered heterocycloalkyl, 4 to 6-membered heterocycloalkenyl and 5 to 6-membered heteroaryl are each optionally substituted with 1 to 3 substituents independently selected from halo, C₁₋₄alkyl, C₁₋₄haloalkyl and C₃₋₆cycloalkyl; and the definitions for the other variables are as defined in the first, second, third, fifth, sixth, seventh, eighth, ninth, tenth, eleventh, fourteen, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, or thirtieth embodiment.

In a thirty-second embodiment of the present invention, the compound is as defined in the thirty-first embodiment, or a pharmaceutically acceptable salt thereof, wherein R² is selected from halo, C₃₋₆cycloalkyl, 4 to 6-membered heterocycloalkyl, and 5 to 6-membered heteroaryl, wherein the C₃₋₆cycloalkyl, 4 to 6-membered heterocycloalkyl, and 5 to 6-membered heteroaryl are each optionally substituted with halo, C₁₋₄alkyl and C₁₋₄haloalkyl.

In a thirty-third embodiment of the present invention, the compound is as defined in the thirty-first embodiment, or a pharmaceutically acceptable salt thereof, wherein R² is selected from halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

In a thirty-fourth embodiment of the present invention, the compound is as defined in the thirty-first embodiment, or a pharmaceutically acceptable salt thereof, wherein R² is selected from H, Br, Cl, —CH₃, —CF3, —CH₂-cyclopropyl, cyclopropyl, cyclopentyl, 1-methylimidazolyl, dihydropyrrolyl, 1-methyl-1,2,3, 6-tetrahydropyridinyl, tetrahydrofuranyl, tetrahydro-2H-pyranyl, 5-methylfuranyl, 1-methylpyrazolyl, 1-ethylpyrazolyl, 1-isopropylpyrazolyl, methyltetrahydropyridinyl, pyridinyl, 1-methylpyrrolidinyl, 1-methylpiperidinyl, and diflurophenyl.

In a thirty-fifth embodiment of the present invention, the compound is as defined in the thirty-first embodiment, or a pharmaceutically acceptable salt thereof, wherein R² is selected from H, Br, Cl, —CH₃, —CF3, —CH₂-cyclopropyl, cyclopropyl, cyclopentyl, 1-methylimidazolyl, dihydropyrrolyl, 1-methyl-1,2,3, 6-tetrahydropyridinyl, tetrahydro-2H-pyranyl, 1-methylpyrazolyl, 1-ethylpyrazolyl, 1-isopropylpyrazolyl, methyltetrahydropyridinyl, pyridinyl, 1-methylpyrrolidinyl, 1-methylpiperidinyl, and diflurophenyl.

In a thirty-sixth embodiment of the present invention, the compound is as defined in the thirty-first embodiment, or a pharmaceutically acceptable salt thereof, wherein R² is selected from cyclopentyl, tetrahydrofuranyl, 5-methylfuranyl, and 1-methylpyrazolyl.

In a thirty-seventh embodiment of the present invention, the compound is represented by formula (I′), (II′), (I), (II), (V), (VI), (VA), (VB), (VC), (VD), (VIA), (VIB), (VIC) or (VID), or a pharmaceutically acceptable salt thereof, wherein R⁴ is selected from C₃₋₈cycloalkyl, C₅₋₈cycloalkenyl, 4 to 6-membered heterocycloalkyl, 4 to 6-membered heterocycloalkenyl, phenyl, 5 to 6-membered heteroaryl, —CN, —OR^{2a}, —N(R^{2a})₂, —C(O)N(R^{2a})₂, wherein the C₃₋₈cycloalkyl, C₅₋₈cycloalkenyl, 4 to 6-membered heterocycloalkyl, 4 to 6-membered heterocycloalkenyl, phenyl, 5 to 6-membered heteroaryl are each optionally substituted with 1 to 3 substituents independently selected from C₁₋₆alkyl, C₁₋₆ haloalkyl, C₃₋₈cycloalkyl, 4 to 7-membered heterocycloalkyl, phenyl, 5- to 6-membered heteroaryl, halo, —CN, —OR^{4a}, —C(O)N(R^{4a})₂, and —N(R^{4a})₂; and

R^{4a}, for each occurrence, is independently selected from H, C₁₋₆alkyl, C₃₋₈cycloalkyl, and 4 to 6-membered heterocycloalkyl; and the definitions for the other variables are as defined in the first, second, fourth, fifth, sixth, seventh, eighth, ninth, twelfth, thirteenth, fourteen, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, or thirtieth embodiment.

In a thirty-eighth embodiment of the present invention, the compound is as defined in the thirty-seventh embodiment, or a pharmaceutically acceptable salt thereof, wherein R⁴ is selected from halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

In a thirty-ninth embodiment of the present invention, the compound is as defined in the thirty-seventh embodiment, wherein R⁴ is selected from C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

In a fortieth embodiment of the present invention, the compound is as defined in the thirty-seventh embodiment, or a pharmaceutically acceptable salt thereof, wherein R⁴ is selected from H, Cl, Br, —CH₃, —CH₂CH₂CH₃, propyl, —CH₂-cyclopentyl, —CH₂—OH, cyclopentyl, cyclohexyl, difluorocyclohexyl, tetrahydrofuranyl, tetrahydropyranyl, and methylpyrazolyl.

In a forty-first embodiment of the present invention, the compound is as defined in the thirty-seventh embodiment, or a pharmaceutically acceptable salt thereof, wherein R⁴ is selected from cyclopentyl and 1-methylpyrazolyl.

In a forty-second embodiment of the present invention, the compound is represented by the following formula:

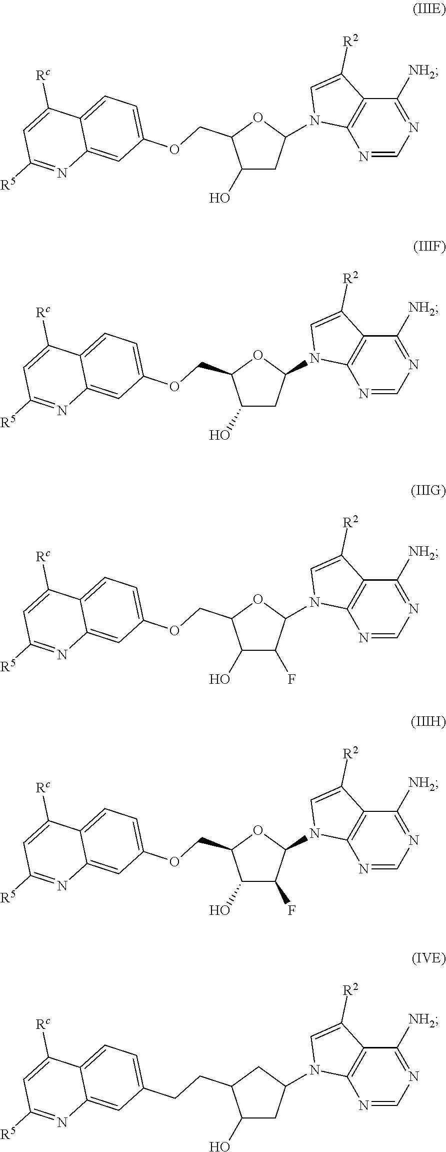

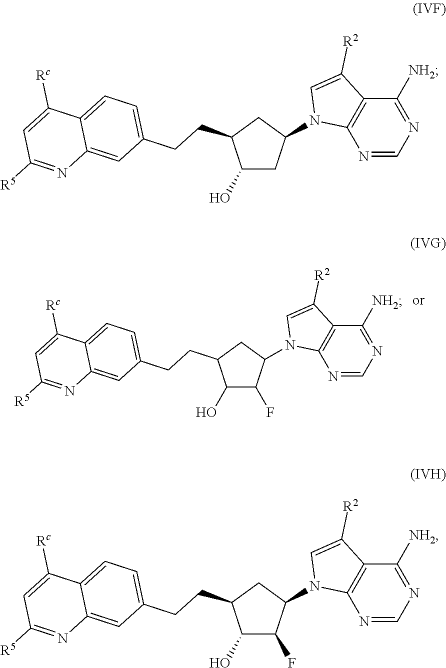

or a pharmaceutically acceptable salt thereof, wherein:

W is H or F;

R² is selected from H, halo, C₃₋₆cycloalkyl, 4 to 6-membered heteroccloalkyl, and 5 to 6-membered heteroaryl, wherein the C₃₋₆cycloalkyl, 4 to 6-membered heteroccloalkyl, and 5 to 6-membered heteroaryl are each optionally substituted with halo, C₁₋₄alkyl and C₁₋₄haloalkyl; and

R⁵ is —N(R^{5a})₂;

R^{5a}, for each occurrence, is independently selected from H, C₁₋₆alkyl, and C₃₋₆cycloalkyl, or two R^{5a} together with the N atom from which they are attached form a 4 to 6-membered heterocycloalkyl optionally containing an additional heteroatom selected from 0 and N;

R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂, —NR^{c1}(═O)R^{c1}, —NR^{c1}(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and

R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl and 4 to 7-membered heterocycloalkyl, or two R^{c1} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl; and the definitions for the other variables are as defined in the first embodiment.

In a forty-third embodiment of the present invention, the compound is represented by the following formula:

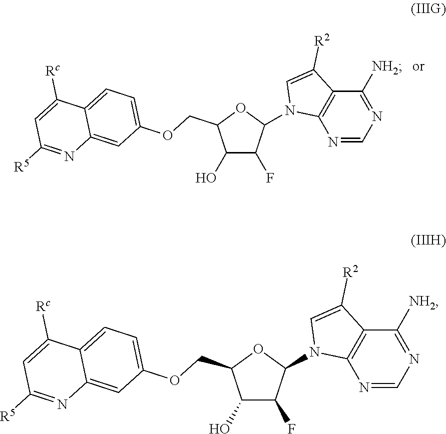

or a pharmaceutically acceptable salt thereof, wherein:

R² is selected from H, halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl; and

R⁵ is —N(R^{5a})₂;

R^{5a}, for each occurrence, is independently selected from H and C₁₋₆alkyl, or two R^{5a} together with the N atom from which they are attached form a 4 to 6-membered heterocycloalkyl optionally containing an additional heteroatom selected from O and N;

R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂, —NR^{c1}(═O)R^{c1}, —NR^{c1}(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and

R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl and 4 to 7-membered heterocycloalkyl, or two R^{c1} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl; and the definitions for the other variables are as defined in the first or second embodiment.

In a forty-fourth embodiment of the present invention, the compound is represented by the following formula:

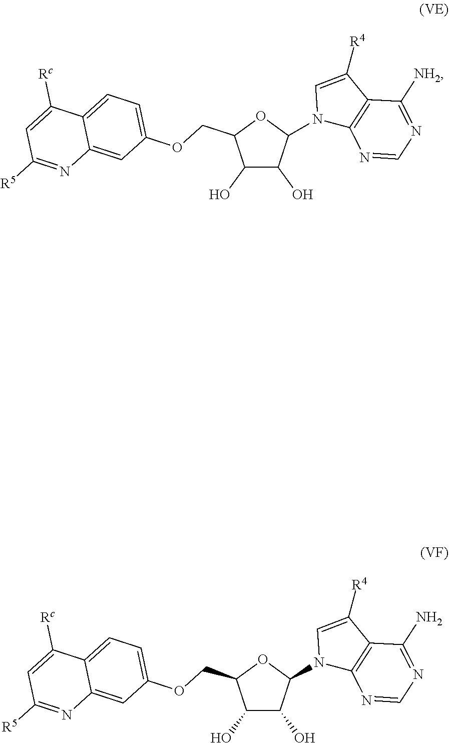

or a pharmaceutically acceptable salt thereof, wherein:

R⁴ is selected from H, halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl;

R⁵ is —N(R^{5a})₂;

R^{5a}, for each occurrence, is independently selected from H and C₁₋₆alkyl, or two R^{5a} together with the N atom from which they are attached form a 4 to 6-membered heterocycloalkyl optionally containing an additional heteroatom selected from O and N;

R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂—NR^{c1}C(═O)R^{c1}, —NR^{c1}C(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and

R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl and 4 to 7-membered heterocycloalkyl, or two R^{c} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl; and the definitions for the other variables are as defined in the first or second embodiment.

In a forty-fifth embodiment of the present invention, the compound is represented by formula (IIIE), (IIIF), (IIIG), (IIIH), (IVE), (IVF), (IVG), (IVH), (VE), or (VF), or a pharmaceutically acceptable salt thereof, wherein R^{c} is selected from H, halo, C₁₋₄alkyl and —N(R^{c1})₂, and R^{c1}, for each occurrence, is independently H or C₁₋₄alkyl; and the definitions for the other variables are as defined in the forty-second, forty-third, or forty-fourth embodiment.

In a forty-sixth embodiment of the present invention, the compound is represented by formula (IIIE), (IIIF), (IIIG), (IIIH), (IVE), (IVF), (IVG), (IVH), (VE), or (VF), or a pharmaceutically acceptable salt thereof, wherein R is H; and the definitions for the other variables are as defined in the forty-second, forty-third, or forty-fourth embodiment.

In a forty-seventh embodiment of the present invention, the compound is as defined in the forty-second, forty-third, forty-fourth, forty-fifth, or forty-sixth embodiment, or a pharmaceutically acceptable salt thereof, wherein for formula (IIIE), (IIIF), (IIIG), (IIIH), (IVE), (IVF), (IVG) or (IVH), R² is cyclopentyl, 5-membered heterocycloalkyl or 5-membered heteroaryl, each of which is optionally substituted with C₁₋₄alkyl; and for formula (VE) or (VF), R⁴ is cyclopentyl, 5-membered heterocycloalkyl or 5-membered heteroaryl, each of which is optionally substituted with 1 to 2 substituents independently selected from C₁₋₄alkyl.

In a forty-eighth embodiment of the present invention, the compound is as defined in the forty-seventh embodiment, or a pharmaceutically acceptable salt thereof, wherein R² is cyclopentyl, tetrahydrofuranyl, furanyl or pyrazolyl, each of which is optionally substituted with 1 or 2 independently selected from C₁₋₄alkyl; and R⁴ is cyclopentyl, tetrahydrofuranyl, furanyl or pyrazolyl, each of which is optionally substituted with 1 or 2 independently selected from C₁₋₄alkyl.

In a forty-ninth embodiment of the present invention, the compound of the present invention is selected from the compounds of Table 1 or a pharmaceutically acceptable salt thereof.

### Definitions

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 6 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, or n-decyl.

The number of carbon atoms in a group is specified herein by the prefix “Cₓ₋ₓₓ”, wherein x and xx are integers. For example, “C₁₋₄alkyl” is an alkyl group which has from 1 to 4 carbon atoms; and C₁₋₄haloalkyl is a haloalkyl group which has from 1 to 4 carbon atoms.

As used herein, the term “alkenyl” refers to an olefinically unsaturated branched or linear group having at least one double bond. Preferably the alkenyl comprises 2 to 20 carbon atoms, more preferably 2 to 16 carbon atoms, 2 to 10 carbon atoms, 2 to 6 carbon atoms, or 2 to 4 carbon atoms. Alkenyl groups include, but are not limited to, propenyl, 1,3-butadienyl, 1-butenyl, hexenyl, pentenyl, heptenyl, octenyl and the like.

As used herein, the term “alkynyl” refers to an unsaturated branched or linear group having at least one triple bond. Preferably the alkynyl comprises 2 to 20 carbon atoms, more preferably 2 to 16 carbon atoms, 2 to 10 carbon atoms, 2 to 6 carbon atoms, or 2 to 4 carbon atoms. Alkynyl groups include, but are not limited to, propynyl, 1-butynyl, hexynyl, pentynyl, hexynyl, heptynyl, octynyl and the like.

As used herein, the term “carbocyclyl” refers to saturated or partially unsaturated (but not aromatic) monocyclic, bicyclic or tricyclic hydrocarbon groups of 3-14 carbon atoms, preferably 3-9, or more preferably 3-8 carbon atoms. Carbocyclyls include fused, bridged, or spiro ring systems. The term “carbocyclyl” encompasses cycloalkyl groups. The term “cycloalkyl” refers to completely saturated monocyclic, bicyclic or tricyclic hydrocarbon groups of 3-12 carbon atoms, preferably 3-9, or more preferably 3-8 carbon atoms. Exemplary monocyclic carbocyclyl groups include, but are not limited to, cyclopropyl, cyclobutyl, cyclopentyl, cyclopentenyl, cyclohexyl or cyclohexenyl. Exemplary bicyclic carbocyclyl groups include bornyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[1.1.1]pentane, or bicyclo[2.2.2]octyl. Exemplary tricyclic carbocyclyl groups include adamantyl.

As used herein, the term “halocycloalkyl” refers to a cycloalkyl, as defined herein, that is substituted by one or more halo groups as defined herein. Preferably the halocycloalkyl can be monohalocycloalkyl, dihalocycloalkyl or polyhalocycloalkyl including perhalocycloalkyl. A monohalocycloalkyl can have one iodo, bromo, chloro or fluoro substituent. Dihalocycloalkyl and polyhalocycloalkyl groups can be substituted with two or more of the same halo groups or a combination of different halo groups.

As used herein, the term “cycloalkenyl” refers to a partially unsaturated monocyclic, bicyclic or tricyclic hydrocarbon groups having 3-12 ring carbon atoms, preferably 3-9, or more preferably 3-8 carbon atoms, and having one or more double bonds. Exemplary monocyclic cycloalkenyl groups include, but are not limited to, cyclopentenyl, cyclopentadienyl, cyclohexenyl, and the like. Exemplary bicyclic cycloalkenyl groups include, but are not limited to, bicyclo[2.2.1]hept-5-enyl and bicycle[2.2.2]oct-2-enyl.

As used herein, the term “haloalkyl” refers to an alkyl, as defined herein, that is substituted by one or more halo groups as defined herein. Preferably, the haloalkyl can be monohaloalkyl, dihaloalkyl or polyhaloalkyl including perhaloalkyl. A monohaloalkyl can have one iodo, bromo, chloro or fluoro substituent. Dihaloalkyl and polyhaloalkyl groups can be substituted with two or more of the same halo groups or a combination of different halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms. Preferred haloalkyl groups are trifluoromethyl and difluoromethyl.

“Halogen” or “halo” may be fluoro, chloro, bromo or iodo.

The term “aryl” refers to monocyclic, bicyclic or tricyclic aromatic hydrocarbon groups having from 6 to 14 ring carbon atoms. In one embodiment, the term aryl refers to monocyclic or bicyclic aromatic hydrocarbon groups having from 6 to 10 carbon atoms.

Representative examples of aryl groups include phenyl (Ph), naphthyl, fluorenyl, and anthracenyl.

The term “aryl” also refers to a bicyclic or tricyclic group in which at least one ring is aromatic and is fused to one or two non-aromatic hydrocarbon ring(s). Nonlimiting examples include tetrahydronaphthalene, dihydronaphthalenyl and indanyl.

As used herein, the term “heterocyclyl” refers to a saturated or unsaturated, non-aromatic monocyclic, bicyclic or tricyclic ring system which has from 3- to 15-ring members at least one of which is a heteroatom, and up to 10 of which may be heteroatoms, wherein the heteroatoms are independently selected from O, S and N, and wherein N and S can be optionally oxidized to various oxidation states. In one embodiment, a heterocyclyl is a 3-8-membered monocyclic. In another embodiment, a heterocyclyl is a 6-12-membered bicyclic.

In yet another embodiment, a heterocyclyl is a 10-15-membered tricyclic ring system. The heterocyclyl group can be attached at a heteroatom or a carbon atom. Heterocyclyls include fused or bridged ring systems. The term “heterocyclyl” encompasses heterocycloalkyl and heterocycloalkenyl groups. The term “heterocycloalkyl” refers to completely saturated monocyclic, bicyclic or tricyclic heterocyclyl comprising 3-15 ring members, at least one of which is a heteroatom, and up to 10 of which may be heteroatoms, wherein the heteroatoms are independently selected from O, S and N, and wherein N and S can be optionally oxidized to various oxidation states. In one embodiment, a heterocyclyl is a 4 to 7-membered heterocycloalkyl. Examples of heterocyclyls include dihydrofuranyl, [1,3]dioxolane, 1,4-dioxane, 1,4-dithiane, piperazinyl, 1,3-dioxolane, imidazolidinyl, imidazolinyl, pyrrolidine, dihydropyran, oxathiolane, dithiolane, I,3-dioxane, 1,3-dithianyl, oxathianyl, thiomorpholinyl, oxiranyl, aziridinyl, oxetanyl, azetidinyl, tetrahydrofuranyl, pyrrolidinyl, tetrahydropyranyl, piperidinyl, morpholinyl, piperazinyl, azepinyl, oxapinyl, oxazepinyl and diazepinyl. The term “heterocycloalkenyl” refers to partially unsaturated monocyclic, bicyclic or tricyclic heterocyclyl comprising 3-15 ring members, with at least one double bond and at least one of the ring members is a heteroatom, and up to 10 of which may be heteroatoms, wherein the heteroatoms are independently selected from O, S and N, and wherein N and S can be optionally oxidized to various oxidation states. In one embodiment, a heterocyclyl is a 4 to 7-membered heterocycloalkenyl. Examples of heterocycloalkenyl include 1,2,3,4-tetrahydropyridinyl, 1,2-dihydropyridinyl, 1,4-dihydropyridinyl, 1,2, 3, 6-tetrahydro-pyridinyl, 1,4,5,6-tetrahydro-pyrimidinyl, 2-pyrrolinyl, 3-pyrrolinyl, 2-imidazolinyl, 2-pyrazolinyl, 3,4-dihydro-2H-pyran, dihydrofuranyl, fluoro-dihydro-furyl group, dihydro-thienyl and dihydro-thiopyran-yl.

As used herein, the term “heteroaryl” refers to a 5-14 membered monocyclic-, bicyclic-, or tricyclic-ring system, having 1 to 10 heteroatoms independently selected from N, O or S, wherein N and S can be optionally oxidized to various oxidation states, and wherein at least one ring in the ring system is aromatic. In one embodiment, the heteroaryl is a 5 to 6-membered monocyclic heteroaromatic ring. Examples of monocyclic heteroaryl groups include pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. In another embodiment, the heteroaryl is an 8- to 10-membered bicyclic heteroaromatic ring. Examples of bicyclic heteroaryl groups include quinolinyl, quinozalinyl, phthalazinyl, quinoxalinyl, cinnolinyl, naphthyridinyl, pyridoprimidinyl, pyridopyrazinyl, pteridinyl, indolyl, isoindolyl, indolizinyl, indazolyl, benzoimidazolyl, benzotriazolyl, benzooxazolyl, benzoisoxazolyl, benzothiazolyl, benzofuranyl, isobenzofuranyl, benzothiophenyl, benzothiadiazolyl, azaindolyl, purine, imidazopyridinyl, pyrrolopyrimidinyl, imidazopyridazinyl, imidazopyrazinyl, pyrazolopyrimidinyl, pyrazolopyridinyl, pyrazolotriazinyl, oxazolopyridinyl, isoxazolopyridinyl, thiazolopyridinyl, isothiazolopyridinyl, indolyl, benzofuranyl, quinolyl, isoquinolyl indazolyl, indolinyl, isoindolyl, indolizinyl, benzamidazolyl and quinolinyl.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy, cyclohexyloxy and the like. Preferably, alkoxy groups have about 1-6 carbon atoms, more preferably about 1-4 carbon atoms.

The term “bicyclic” or “bicyclic ring system,” as used herein, can include a fused ring system, a bridged ring system, or a spiro ring system.

The term “fused ring system,” as used herein, is a ring system that has two or three rings (preferably two rings) independently selected from carbocyclyl, heterocyclyl, aryl or heteroaryl rings that share one side A fused ring system may have from 4-15 ring members, preferably form 5-10 ring members. Examples of fused ring systems include octahydroisoquinolin-2(1H)-yl, 2,3-dihydro-1H-indenyl, octahydro-1H-pyrido[1,2-a]pyrazinyl, and decahydroisoquinolinyl).

The term “bridged ring system,” as used herein, is a ring system that has a carbocyclyl or heterocyclyl ring wherein two non-adjacent atoms of the ring are connected (bridged) by one or more (preferably from one to three) atoms. A bridged ring system can have more than one bridge within the ring system (e.g., adamantyl). A bridged ring system may have from 6-10 ring members, preferably from 7-10 ring members. Examples of bridged ring systems include adamantly, 9-azabicyclo[3.3.1]nonan-9-yl, 8-azabicyclo[3.2.1]octanyl, bicyclo[2.2.2]octanyl, 3-azabicyclo[3.1.1]heptanyl, bicycle[1.1.1]pentane, bicyclo[2.2.1]heptanyl, (1R,5S)-bicyclo[3.2.1]octanyl, 3-azabicyclo[3.3.1]nonanyl, and bicyclo[2.2.1]heptanyl. More preferably, the bridged ring system is selected from the group consisting of 9-azabicyclo[3.3.1]nonan-9-yl, 8-azabicyclo[3.2.1]octanyl, and bicyclo[2.2.2]octanyl.

The term “spiro ring system,” as used herein, is a ring system that has two rings each of which are independently selected from a carbocyclyl or a heterocyclyl, wherein the two ring structures having one atom in common. Spiro ring systems have from 5 to 14 ring members. Example of spiro ring systems include 2-azaspiro[3.3]heptanyl, spiropentanyl, 2-oxa-6-azaspiro[3.3]heptanyl, 2,7-diazaspiro[3.5]nonanyl, 2-oxa-7-azaspiro[3.5]nonanyl, 6-oxa-9-azaspiro[4.5]decanyl, 6-oxa-2-azaspiro[3.4]octanyl, 5-azaspiro[2.3]hexanyl and 2,8-diazaspiro[4.5]decanyl.

The term “spiroheterocycloalkyl” as used herein, is a heterocycloalkyl that has one ring atom in common with the group to which it is attached. Spiroheterocycloalkyl groups may have from 3 to 15 ring members. In a preferred embodiment, the spiroheterocycloalkyl has from 3 to 8 ring atoms selected from carbon, nitrogen, sulfur and oxygen and is monocyclic.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase).

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers at least 20%, 30%, 40%, 50%, 60%, 70%, 80%, 90%, 95%, 97%, 99%, 99.5% or 99.9%. The stereoisomeric purity the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. In addition, some compounds may exhibit polymorphism.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

In one embodiment, the present invention provides deuterated compounds described herein or a pharmaceutically acceptable salt thereof.

Another embodiment is a pharmaceutical composition comprising at least one compound described herein, or a pharmaceutically acceptable salt thereof, and at least one pharmaceutically acceptable carrier.

The compounds described herein have METTL3 modulating activity. In one embodiment, the compounds described herein have METTL3 inhibitory activity. In one embodiment, the compounds described herein are selective METTL3 inhibitors. In one embodiment, the compounds described herein have inhibitory activities against METTL3 that are higher than inhibitory activities against other protein targets, such as protein arginine N-methyltransferase 5 (PRMT5). In one embodiment, the compounds described herein have METTL3 inhibitory activities that are at least 2, 3, 5, 10, 15, 20, 30, 40, 50, 75, 100, 200, 400 or 1000 times greater than their inhibitory activities towards PRMT5.

In some embodiments, the METTL3 inhibitors described herein have an IC₅₀ value of less than 1 μM, less than 750 nM, less than 500 nM, less than 250 nM or less than 100 nM.

As used herein, “METTL3 modulating activity” refers to the ability of a compound or composition to induce a detectable change in METTL3 activity in vivo or in vitro (e.g., at least 10% increase or decrease in METTL3 activity as measured by a given assay such as the bioassay described in the examples and known in the art). A decrease in METLL3 activity is METTL3 inhibitory activity.

### Methods of Use

In one aspect, the present invention discloses a method of treating a disease or disorder responsive to inhibition of METTL3 activity in a subject comprising administering to the subject an effective amount of the compound described herein, or a pharmaceutically acceptable salt thereof.

In one embodiment, the disease or disorder is an infection, such as, a viral infection. In a specific embodiment, the viral infection is caused by RNA virus or retrovirus. Examples of viral infections include, but are not limited to, Dengue, Yellow Fever, Japanese encephalitis, Zika virus, Ebola virus, severe acute respiratory syndrome (SARS), rabies, HIV, influenza, hepatitis C, hepatitis E, West Nile fever, polio, measles, COVID-19, and Middle East respiratory syndrome (MERS-CoV).

In one embodiment, the disease or disorder is a cancer.

The term “cancer” includes diseases or disorders involving abnormal cell growth and/or proliferation.

In some embodiments, the cancer is selected from glioblastoma, leukemia, stomach cancer, prostate cancer, colorectal cancer, endometrial cancer, breast cancer, pancreatic cancer, kidney cancer, lung cancer, bladder cancer, ovarian cancer, esophageal/upper aerodigestive cancer, liver cancer, bone cancer, acute lymphocytic leukemia, non-Hodgkin's lymphoma (NHL), multiple myeloma, mesothelioma, and sarcoma.

In a specific embodiment, the cancer is acute myeloid leukemia.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or ‘treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The compounds of the invention may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

Compounds or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### Exemplifications

### Instrument Details (and Conditions)

1. ¹H NMR or ¹⁹F NMR, NOESY spectra were recorded on Bruker AVβ 400.

2. LCMS measurement was run on Agilent 1200 HPLC/6100 SQ System using the follow conditions:

Method A: Mobile Phase: A: Water (0.01% TFA) B: Acetonitrile (0.01% TFA); Gradient Phase: 5% B to 95% B within 1.4 min, 95% B with 1.6 min (total runtime: 3 min); Flow Rate: 2.0 mL/min; Column: SunFire C18, 4.6*50 mm, 3.5 μm; Column Temperature: 40° C. Detectors: ADC ELSD, DAD(214 nm and 254 nm), ES-API.

Method B: Mobile Phase: A: Water (10 mM NH₄HCO₃) B: Acetonitrile; Gradient Phase: 5% to 95% B within 1.4 min, 95% B with 1.6 min (total runtime:3 min); Flow Rate: 2.0 mL/min; Column: XBridge C18, 4.6*50 mm, 3.5 um; Column Temperature: 40° C. Detectors: ADC ELSD, DAD(214 nm and 254 nm), MSD (ES-API).

3. HPLC was taken on Agilent LC 1200 series.

Method A: Mobile Phase: A: Water (0.01% TFA) B: Acetonitrile (0.01% TFA); Gradient Phase: 5% B to 95% B within 9.5 min, 95% B with 5 min (total runtime: 14.5 min); Flow Rate: 1.0 mL/min; Column: SunFire C18, 4.6*100 mm, 3.5 μm; Column Temperature: 40° C. Detectors: ADC ELSD, DAD(214 nm and 254 nm), ES-API.

4. Prep-HPLC:

Instrument: Gilson 281 (PHG-009)

Column: Xtimate Prep C18 10 μm 21.2×250 mm

Method A: Mobile Phase: A: water (0.01% FA) B:acetonitrile

Method B: Mobile Phase: A: Water (10 mmol NH₄HCO₃); B:acetonitrile

Flow Rate(ml/min): 30.00

Detective Wavelength (nm): 214/254

### Compound Synthesis

### General Method A

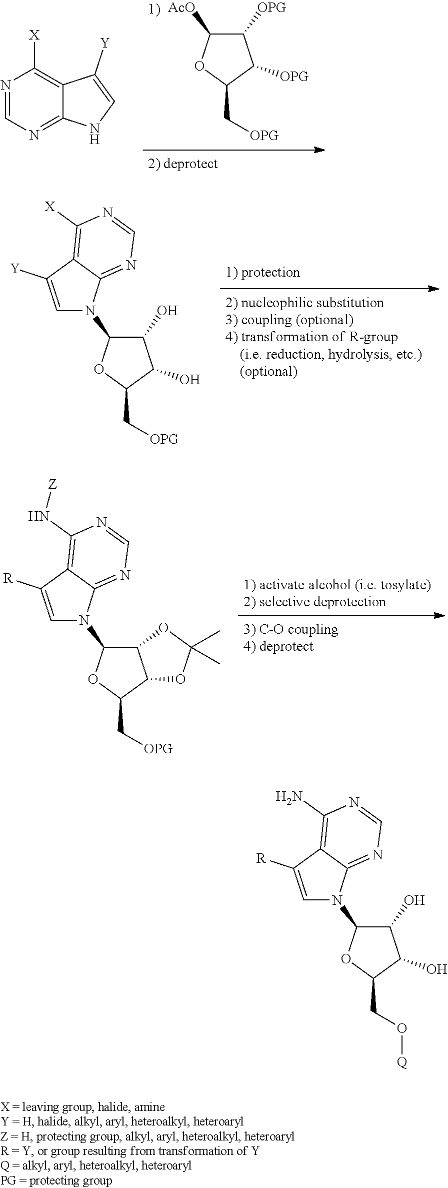

### Example 1: Synthesis of (2R,3R,4S,5R)-2-(4-amino-5-cyclopentyl-7H-pyrrolo[2,3-d]pyrimidin-7-yl)-5-(((2-(methylamino)quinolin-7-yl)oxy)methyl)tetrahydrofuran-3,4-diol (compound I-33)

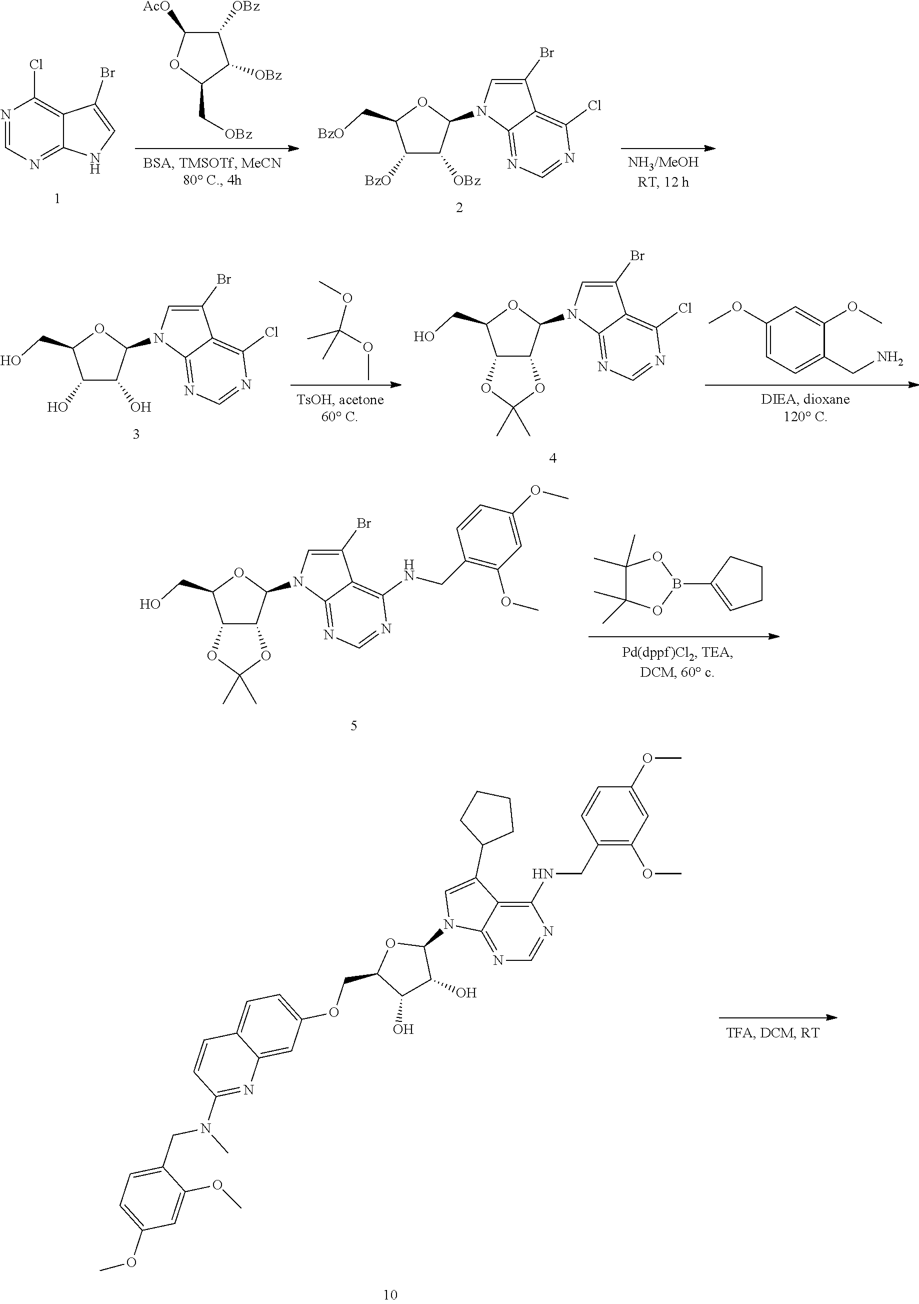

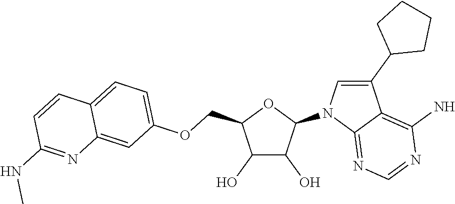 [SMILES: CNc1ccc2ccc(OC[C@H]3O[C@@H](n4cc(C5CCCC5)c5c(N)ncnc54)C(O)C3O)cc2n1]

### Synthesis of (2R,3R,4R,5R)-2-((benzoyloxy)methyl)-5-(5-bromo-4-chloro-7H-pyrrolo[2,3-d]pyrimidin-7-yl)tetrahydrofuran-3,4-diyl dibenzoate (2)

BSA (15.6 g, 77.3 mmol) was added to a stirred suspension of 5-bromo-4-chloro-7H-pyrrolo[2,3-d]pyrimidine (15.0 g, 64.5 mmol) in anhydrous MeCN (35 mL). The solution became clear and [(2R,3R,4R,5S)-5-(acetyloxy)-3,4-bis(benzoyloxy)oxolan-2-yl]methyl benzoate (48.7 g, 96.7 mmol) was added followed by the addition of TMSOTf (17.1 g, 77.3 mmol). The reaction mixture was stirred at 80° C. for 16 hrs. Cooled to room temperature and diluted with DCM. The organic phase was washed with saturated NaHCO₃/H2O, and dried with Na2SO₄. The crude product was purified by silica column (DCM/MeOH=0˜1%) to get the target compound (25 g, yield 57%) as a yellow solid. ES LC-MS m/z=676.1 [M+H]⁺.

### Synthesis of (2R,3R,4S,5R)-2-(5-bromo-4-chloro-7H-pyrrolo[2,3-d]pyrimidin-7-yl)-5-(hydroxymethyl)tetrahydrofuran-3,4-diol (3)

2R,3R,4R,5R)-2-((benzoyloxy)methyl)-5-(5-bromo-4-chloro-7H-pyrrolo[2,3-d]pyrimidin-7-yl)tetrahydrofuran-3,4-diyl dibenzoate (8.00 g, 11.8 mmol) was added to DCM (10 mL. then NH3/MeOH (50 mL) was added to the solution. The mixture was stirred at room temperature for 12 h. LCMS show SM was reacted completed. The reaction solution was concentrated and the crude product was purified by flash column (DCM/MeOH=0˜20%) to get the target compound (2.3 g, yield 53%) as a white solid. ES LC-MS m/z=364.0 [M+H]⁺.

### Synthesis of ((3aR,4R,6R,6aR)-6-(5-bromo-4-chloro-7H-pyrrolo[2,3-d]pyrimidin-7-yl)-2,2-dimethyltetrahydrofuro[3,4-d][1,3]dioxol-4-yl)methanol (4)

(2R,3R,4S,5R)-2-(5-bromo-4-chloro-7H-pyrrolo[2,3-d]pyrimidin-7-yl)-5-(hydroxymethyl)tetrahydrofuran-3,4-diol (2.30 g, 6.30 mmol), 2,2-dimethoxypropane (3.28 g, 31.5 mmol), TsOH (130 mg, 0.6 mmol) were mixed in Acetone (50 mL). The mixture was stirred at 60° C. for 4 h under N2 atmosphere. LCMS show SM was reacted completely. The reaction solution was concentrated under reduced pressure after it was neutralized with NaHCO3. The crude product was extracted with DCM and washed with water to get the target compound (2.0 g, yield: 79%) as a solid. ES LC-MS m/z=404.0 [M+H]⁺.

### Synthesis of ((3aR,4R,6R,6aR)-6-(5-bromo-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methanol (5)

((3aR,4R,6R,6aR)-6-(5-bromo-4-chloro-7H-pyrrolo[2,3-d]pyrimidin-7-yl)-2,2-dimethyltetrahydrofuro[3,4-d][1,3]dioxol-4-yl)methanol (2.00 g, 4.94 mmol), 1-(2,4-dimethoxyphenyl)methanamine (2.47 g, 14.8 mmol), DIEA (1.90 g, 14.8 mmol) were mixed in dioxane (4 mL). The mixture was sealed at 120° C. for 12 h. The reaction solution was concentrated. The crude product was washed with water and directly used to next step without further purification. ES LC-MS m/z=534.8 [M+H]⁺.

### Synthesis of ((3aR,4R,6R,6aR)-6-(5-(cyclopent-1-en-1-yl)-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methanol (6)

((3aR,4R,6R,6aR)-6-(5-bromo-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methanol (2.30 g, 4.29 mmol), 2-(cyclopent-1-en-1-yl)-4,4,5,5-tetramethyl-1,3,2-dioxaborolane (4.15 g, 21.4 mmol), Pd(dppf)C12 (350 mg, 429 μmol), K3PO4 (2.71 g, 12.8 mmol) were mixed in THE (20 mL) and H₂O (5 mL). The mixture was stirred at 70° C. for 12 h. LCMS show SM was reacted completely. The reaction solution was concentrated without other workup. The crude was purified by silica column to get the target compound (1.5 g, yield 67%) as a solid. ES LC-MS m/z=523.3[M+H]⁺.

### Synthesis of ((3aR,4R,6R,6aR)-6-(5-cyclopentyl-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methanol (7)

((3aR,4R,6R,6aR)-6-(5-(cyclopent-1-en-1-yl)-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methanol (1.50 g, 2.87 mmol), Pd/C (10% wet, 333 mg, 3.15 mmol) were mixed in MeOH (20 mL) under N2. The suspension was degassed under vacuum and purged with H2 three times and stirred at room temperature for 2 h. The reaction solution was concentrated under vacuum after filtration to get the target compound (1.4 g, yield 93%) as a solid. ES LC-MS m/z=525.3[M+H]⁺.

### Synthesis of ((3aR,4R,6R,6aR)-6-(5-cyclopentyl-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methyl 4-methylbenzenesulfonate (8)

((3aR,4R,6R,6aR)-6-(5-cyclopentyl-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methanol (500 mg, 953 μmol), 4-methylbenzene-1-sulfonyl chloride (726 mg, 3.81 mmol), DMAP (117 mg, 953 μmol), TEA (542 mg, 4.76 mmol) were mixed in DCM (10 mL). The mixture was stirred at room temperature for 4 h. The crude product was purified by flash column (PE/EA=0-100%) after concentrated to get the target compound (440 mg, yield 68%) as a solid. ES LC-MS m/z=679.2[M+H]⁺.

### Synthesis of ((2R,3S,4R,5R)-5-(5-cyclopentyl-4-((2,4-dimethoxybenzyl)amino)-7H-pyrrolo[2,3-d]pyrimidin-7-yl)-3,4-dihydroxytetrahydrofuran-2-yl)methyl 4-methylbenzenesulfonate (9)

((3aR,4R,6R,6aR)-6-(5-cyclopentyl-4-((2,4-dimethoxybenzyl)amino)-7H-pyrrolo[2,3-d]pyrimidin-7-yl)-2,2-dimethyltetrahydrofuro[3,4-d][1,3]dioxol-4-yl)methyl 4-methylbenzenesulfonate (440 mg, 648 μmol) was stirred in HCl/dioxane (10 mL) for 4 h. LCMS show SM was reacted completely. The crude product (450 mg, yield 108%) was directly used to next step after concentration under reduced pressure without further purification. ES LC-MS m/z=639.3 [M+H]⁺.

### Synthesis of (2R,3R,4S,5R)-2-(5-cyclopentyl-4-((2,4-dimethoxybenzyl)amino)-7H-pyrrolo[2,3-d]pyrimidin-7-yl)-5-(((2-((2,4-dimethoxybenzyl)(methyl)amino)quinolin-7-yl)oxy)methyl)tetrahydrofuran-3,4-diol (10)

((2R,3 S,4R,5R)-5-(5-cyclopentyl-4-((2,4-dimethoxybenzyl)amino)-7H-pyrrolo[2,3-d]pyrimidin-7-yl)-3,4-dihydroxytetrahydrofuran-2-yl)methyl 4-methylbenzenesulfonate (380 mg, 594 μmol), 2-{[(2,4-dimethoxyphenyl)methyl](methyl)amino}quinolin-7-ol (192 mg, 594 μmol), Cs2CO3 (959 mg, 2.96 mmol) were mixed in DMF (4 mL). The mixture was stirred at 60° C. for 2 h. LCMS show SM was reacted completely. The crude product was purified by prep TLC after concentration to get the target compound (200 mg, yield 43%) as a solid. ES LC-MS m/z=791.3 [M+H]⁺.

### Synthesis of (2R,3R,4S,5R)-2-(4-amino-5-cyclopentyl-7H-pyrrolo[2,3-d]pyrimidin-7-yl)-5-(((2-(methylamino)quinolin-7-yl)oxy)methyl)tetrahydrofuran-3,4-diol (compound I-34)

(2R,3R,4S,5R)-2-(5-cyclopentyl-4-((2,4-dimethoxybenzyl)amino)-7H-pyrrolo[2,3-d]pyrimidin-7-yl)-5-(((2-((2,4-dimethoxybenzyl)(methyl)amino)quinolin-7-yl)oxy)methyl)tetrahydrofuran-3,4-diol (200 mg, 240 μmol) was added to TFA (10 mL). The mixture was stirred at room temperature for 4 h. The solution was concentrated and neutralized by NH3/MeOH(7M). Then concentrated again and purified by prep HPLC (NH4HCO3). The desired product was isolated as a white solid (30 mg, yield 24%). ES LC-MS m/z=491.1 [M+H]⁺. 1H NMR (400 MHz, DMSO) δ 8.04 (s, 1H), 7.74 (d, J=8.9 Hz, 1H), 7.52 (d, J=8.4 Hz, 1H), 7.09 (s, 1H), 7.01 (s, 1H), 6.95 (s, 1H), 6.82 (d, J=7.3 Hz, 1H), 6.65-6.44 (m, 3H), 6.17 (d, J=5.7 Hz, 1H), 5.37 (d, J=6.3 Hz, 1H), 5.32 (d, J=4.5 Hz, 1H), 4.45-4.40 (m, 1H), 4.39-4.28 (m, 1H), 4.27-4.16 (m, 3H), 2.88 (d, J=4.5 Hz, 3H), 2.10-1.75 (m, 3H), 1.73-1.62 (m, 4H), 1.54-1.33 (m, 3H).

### General Method B

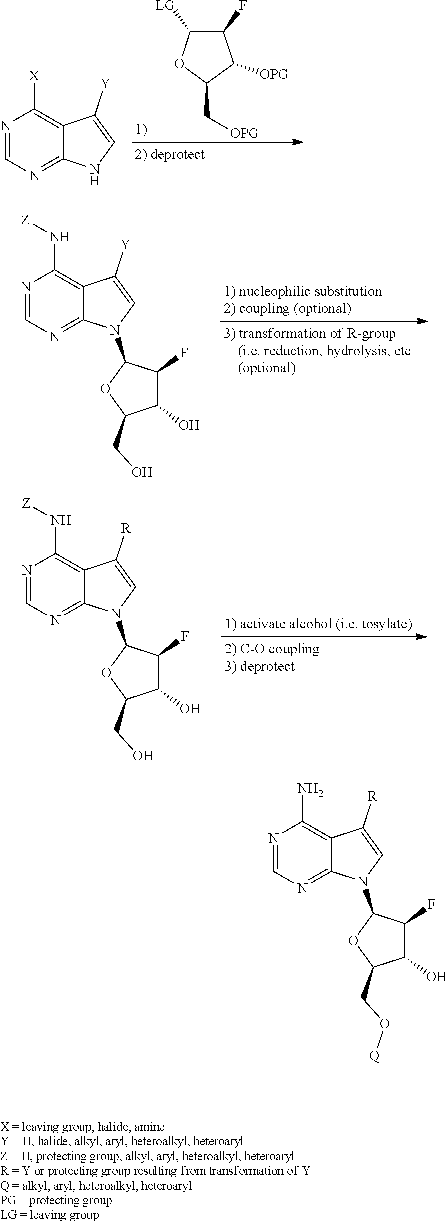

### Example 2: Synthesis of (2R,3R,4S,5R)-5-(4-amino-5-chloro-7H-pyrrolo[2,3-d]pyrimidin-7-yl)-4-fluoro-2-(((2-(methylamino)quinolin-7-yl)oxy)methyl)tetrahydrofuran-3-ol (compound I-45)

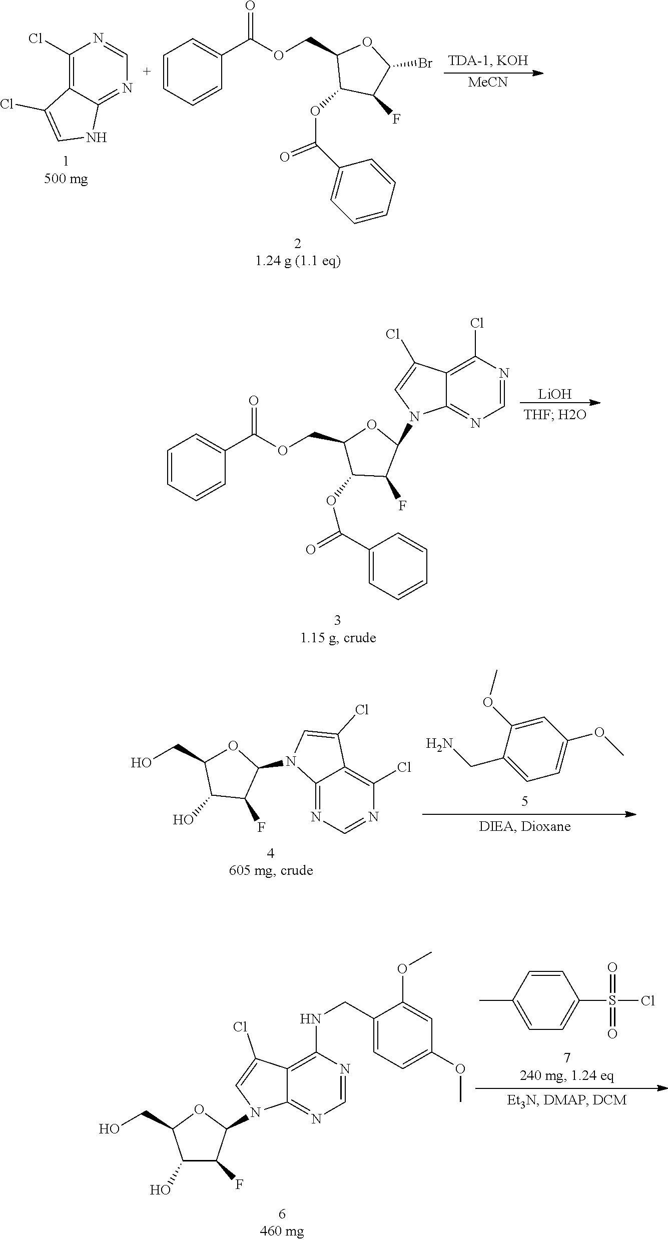

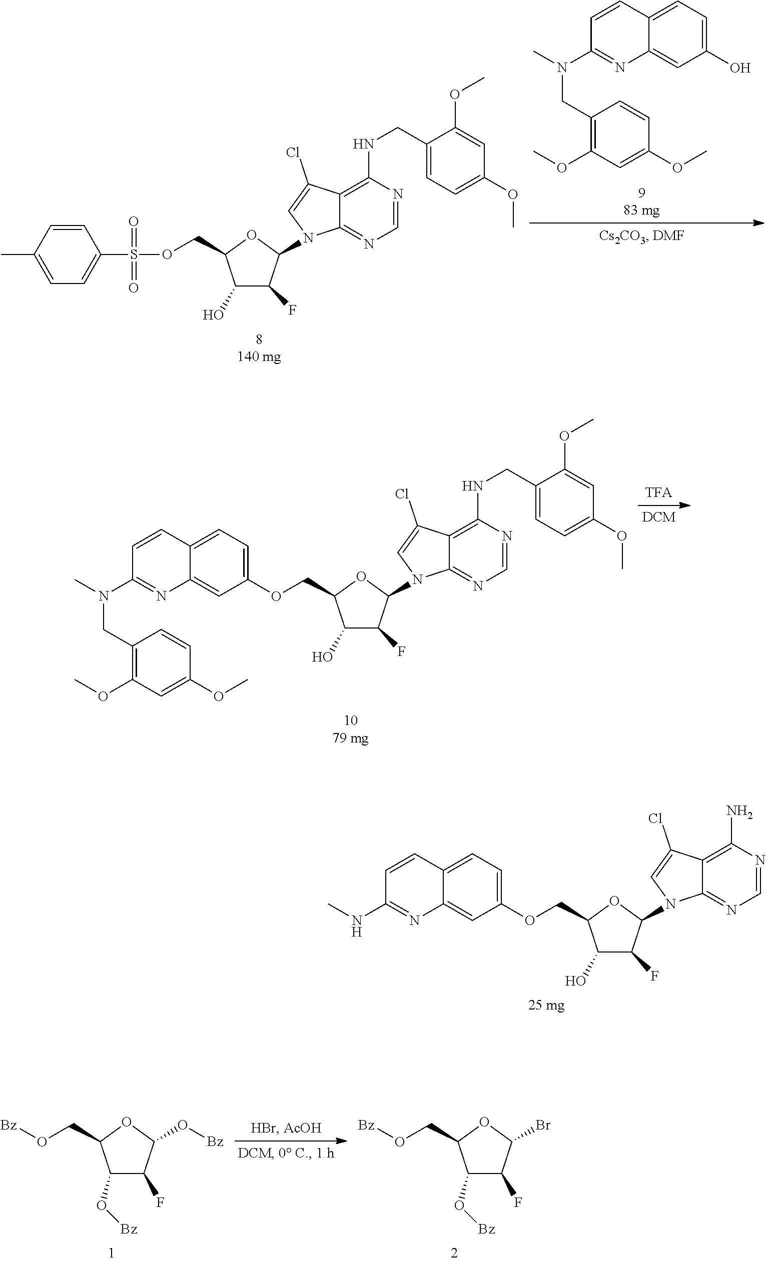

### Synthesis of (2R,3R,4S,5R)-2-[(benzoyloxy)methyl]-5-bromo-4-fluorooxolan-3-yl-benzoate (2)

(2R,3S,4R,5R)-4-(benzoyloxy)-5-[(benzoyloxy)methyl]-3-fluorooxolan-2-yl benzoate (30 g, 64.5 mmol) was dissolved in DCM (50 mL) and HBr/AcOH (60 mL, 30% w/w) was added at 0° C. It was then stirred at room temperature for 1-2 h, and monitored by TLC (PE/EA=5/1). The mixture was extracted with EA (200 mL×3) and the combined organic layers were washed with water, NaHCO₃ aq and brine, then dried (Na₂SO₄). After the solvent was removed, the residue was purified by Silica Gel Column (PE:EA=5:1) to get (2R,3R,4S,5R)-2-[(benzoyloxy)methyl]-5-bromo-4-fluorooxolan-3-yl benzoate (18.0 g, 42.5 mmol) as a colorless oil. CAUTION: Keep it at −18° C. It's not stable at room temperature.

### Synthesis of [(2R,3R,4S,5R)-3-(benzoyloxy)-5-{4,5-dichloro-7H-pyrrolo[2,3-d]pyrimidin-7-yl}-4-fluorooxolan-2-yl]methyl benzoate (3)

To a solution of potassium hydroxide (451 mg, 8.03 mmol) in MeCN (35 ml) was added 8-[2-(2-methoxyethoxy)ethyl]-2,5,11,14-tetraoxa-8-azapentadecane (TDA-1) (120 mg, 371 μmol) at rt. After stirring for 20 min under N₂, 4,5-dichloro-7H-pyrrolo[2,3-d]pyrimidine (500 mg, 2.65 mmol) was added at room temperature and stirred for 20 min under N2. Then a solution of (2R,3R,4S,5R)-2-[(benzoyloxy)methyl]-5-bromo-4-fluorooxolan-3-yl-benzoate (1.24 g, 2.92 mmol) in 6 ml MeCN was added at room temperature and stirred for 2.5 hrs under N₂. LCMS showed the reaction was completed. The mixture was diluted with water (120 ml) and extracted with EA (120 ml×2). Combined organic layers were washed with H₂O (100 ml×2) and brine (60 ml), and dried (Na₂SO₄). The solvent was removed under reduced pressure to get crude [(2R,3R,4S,5R)-3-(benzoyloxy)-5-{4,5-dichloro-7H-pyrrolo[2,3-d]pyrimidin-7-yl}-4-fluorooxolan-2-yl]methyl benzoate (1.15 g, 2.16 mmol), yield 82%.

### Synthesis of (2R,3R,4S,5R)-5-{4,5-dichloro-7H-pyrrolo[2,3-d]pyrimidin-7-yl}-4-fluoro-2-(hydroxymethyl)oxolan-3-ol (4)

[(2R,3R,4S,5R)-3-(benzoyloxy)-5-{4,5-dichloro-7H-pyrrolo[2,3-d]pyrimidin-7-yl}-4-fluorooxolan-2-yl]methyl benzoate (1.15 g, 2.16 mmol) and LiGH (370 mg, 8.81 mmol) were mixed in THE (100 ml) and H2O (25 ml), stirred at 24° C. for 3.5 hrs. LCMS showed the reaction was complete. The mixture was diluted with water (80 ml) and extracted with EA (80 ml×2). Combined organic layers were washed with H₂O (100 ml×2) and brine (60 ml), and dried (Na₂SO₄). The solvent was removed under reduced pressure to get crude (2R,3R,4S,5R)-5-{4,5-dichloro-7H-pyrrolo[2,3-d]pyrimidin-7-yl}-4-fluoro-2-(hydroxymethyl)oxolan-3-ol (605 mg, 1.87 mmol), yield 87%.

### Synthesis of (2R,3R,4S,5R)-5-(5-chloro-4-{[(2,4-dimethoxyphenyl)methyl]amino}-7H-pyrrolo[2,3-d]pyrimidin-7-yl)-4-fluoro-2-(hydroxymethyl)oxolan-3-ol (6)

A mixture of (2R,3R,4S,5R)-5-{4,5-dichloro-7H-pyrrolo[2,3-d]pyrimidin-7-yl}-4-fluoro-2-(hydroxymethyl)oxolan-3-ol (605 mg, 1.87 mmol) in dioxane (2.5 ml) was added 1-(2,4-dimethoxyphenyl)methanamine (595 mg, 3.55 mmol) and N,N-diisopropylethylamine (489 mg, 3.78 mmol). The mixture was stirred at 120° C. for 16 hrs. The solvent was removed under reduced pressure and the residue was purified by TLC (PE:EA=1:10) to give (2R,3R,4S,5R)-5-(5-chloro-4-{[(2,4-dimethoxyphenyl)methyl]amino}-7H-pyrrolo[2,3-d]pyrimidin-7-yl)-4-fluoro-2-(hydroxymethyl)oxolan-3-ol (460 mg, 1.01 mmol), yield 54%. ES LC-MS m/z=453 [M+H]⁺.

### Synthesis of [(2R,3R,4S,5R)-5-(5-chloro-4-{[(2,4-dimethoxyphenyl)methyl]amino}-7H-pyrrolo[2,3-d]pyrimidin-7-yl)-4-fluoro-3-hydroxyoxolan-2-yl]methyl 4-methylbenzene-1-sulfonate (8)

To a solution of (2R,3R,4S,5R)-5-(5-chloro-4-{[(2,4-dimethoxyphenyl)methyl]amino}-7H-pyrrolo[2,3-d]pyrimidin-7-yl)-4-fluoro-2-(hydroxymethyl)oxolan-3-ol (460 mg, 1.01 mmol) and triethylamine (236 mg, 2.33 mmol) in DCM (10 ml) was added 4-dimethylaminopyridine (5.00 mg, 40.9 μmol) and 4-methylbenzene-1-sulfonyl chloride (240 mg, 1.25 mmol) and stirred at room temperature for 16 hrs. LC-MS analysis indicated that reaction was complete. The mixture was purified by TLC (PE:EA=1:4) to give [(2R,3R,4S,5R)-5-(5-chloro-4-{[(2,4-dimethoxyphenyl)methyl]amino}-7H-pyrrolo[2,3-d]pyrimidin-7-yl)-4-fluoro-3-hydroxyoxolan-2-yl]methyl 4-methylbenzene-1-sulfonate (140 mg, 230 μmol), yield 23%. ES LC-MS m/z=607 [M+H]⁺.

### Synthesis of (2R,3R,4S,5R)-5-(5-chloro-4-{[(2,4-dimethoxyphenyl)methyl]amino}-7H-pyrrolo[2,3-d]pyrimidin-7-yl)-2-{[(2-{[(2,4-dimethoxyphenyl)methyl](methyl)amino}quinolin-7-yl)oxy]methyl}-4-fluorooxolan-3-ol (10)

A mixture of [(2R,3R,4S,5R)-5-(5-chloro-4-{[(2,4-dimethoxyphenyl)methyl]amino}-7H-pyrrolo[2,3-d]pyrimidin-7-yl)-4-fluoro-3-hydroxyoxolan-2-yl]methyl 4-methylbenzene-1-sulfonate (140 mg, 230 μmol) in DMF (2 ml) was added 2-{[(2,4-dimethoxyphenyl)methyl](methyl)amino}quinolin-7-ol(83.0 mg, 255 μmol) and cesium carbonate (550 mg, 1.68 mmol). The mixture was stirred at 30° C. for 16 hrs. LC-MS analysis indicated that reaction was complete. The mixture was diluted with water (30 ml) and extracted with EA (30 ml×2). Combined organic layers were washed with H₂O (50 ml×2) and brine (30 ml), and dried (Na₂SO₄). The solvent was removed under reduced pressure and the residue was purified by prep. TLC (PE:EA=1:5) to give (2R,3R,4S,5R)-5-(5-chloro-4-{[(2,4-dimethoxyphenyl)methyl]amino}-7H-pyrrolo[2,3-d]pyrimidin-7-yl)-2-{[(2-{[(2,4-dimethoxyphenyl)methyl](methyl)amino}quinolin-7-yl)oxy]methyl}-4-fluorooxolan-3-ol (79.0 mg, 104 μmol), yield 45%. ES LC-MS m/z=759 [M+H]⁺.

### Synthesis of (2R,3R,4S,5R)-5-(4-amino-5-chloro-7H-pyrrolo[2,3-d]pyrimidin-7-yl)-4-fluoro-2-(((2-(methylamino)quinolin-7-yl)oxy)methyl)tetrahydrofuran-3-ol (compound I-45)

To a solution of (2R,3R,4S,5R)-5-(5-chloro-4-{[(2,4-dimethoxyphenyl)methyl]amino}-7H-pyrrolo[2,3-d]pyrimidin-7-yl)-2-{[(2-{[(2,4-dimethoxyphenyl)methyl](methyl)amino}quinolin-7-yl)oxy]methyl}-4-fluorooxolan-3-ol (79.0 mg, 104 μmol) in DCM (1 ml) was added TFA(6 ml). The mixture was stirred at room temperature for 5 hrs. LCMS showed the reaction was complete. Then the mixture was concentrated and then, neutralized by 4 ml 7M NH₃ in MeOH. The crude material was purified by Prep-HPLC and concentrated to afford 25 mg, yield 52%. MS(ESI): 459.7 [M+H]⁺. ¹H NMR (400 MHz, DMSO-d6) δ ppm 8.14 (s, 1H), 7.74 (d, J=9.2 Hz, 1H), 7.53 (d, J=8.8 Hz, 1H), 7.50 (d, J=2.0 Hz, 1H), 7.05 (d, J=2.4 Hz, 1H), 6.95 (brd, J=4.4 Hz, 2H), 6.81 (dd, J=8.8, 2.4 Hz, 1H), 6.67 (dd, J=14.0, 4.8 Hz, 1H), 6.58 (d, J=9.2 Hz, 1H), 6.14 (d, J=4.8 Hz, 1H), 5.23 (dt, J=52.8, 4.4 Hz, 1H), 4.58-4.50 (m, 1H), 4.40-4.31 (m, 2H), 4.19-4.16 (m, 1H), 2.89 (d, J=5.2 Hz, 3H).

General Method C

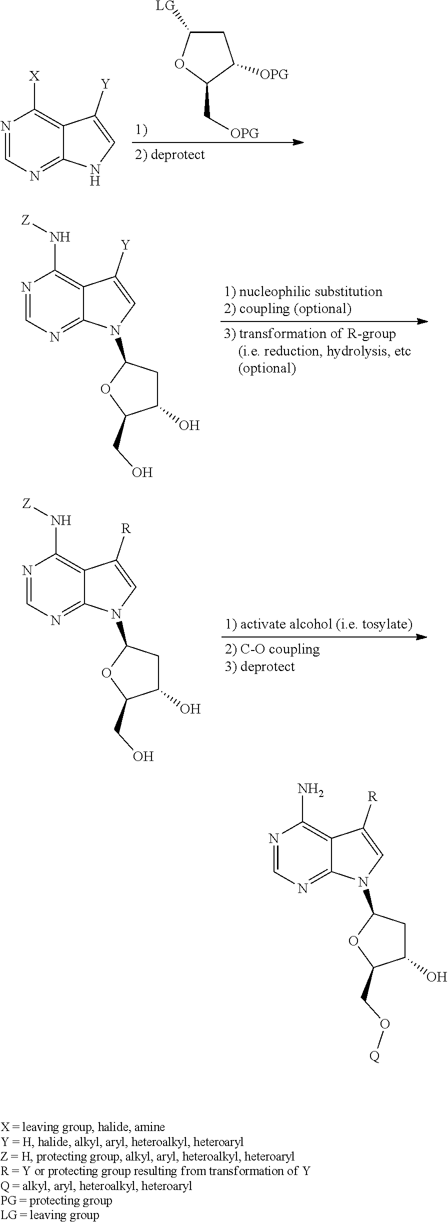

### Example 3: Synthesis of (2R,3S,5R)-5-(4-amino-5-cyclopentyl-7H-pyrrolo[2,3-d]pyrimid in-7-yl)-2-(((2-(methylamino)quinolin-7-yl)oxy)methyl)tetrahydrofuran-3-ol (compound 1-30)

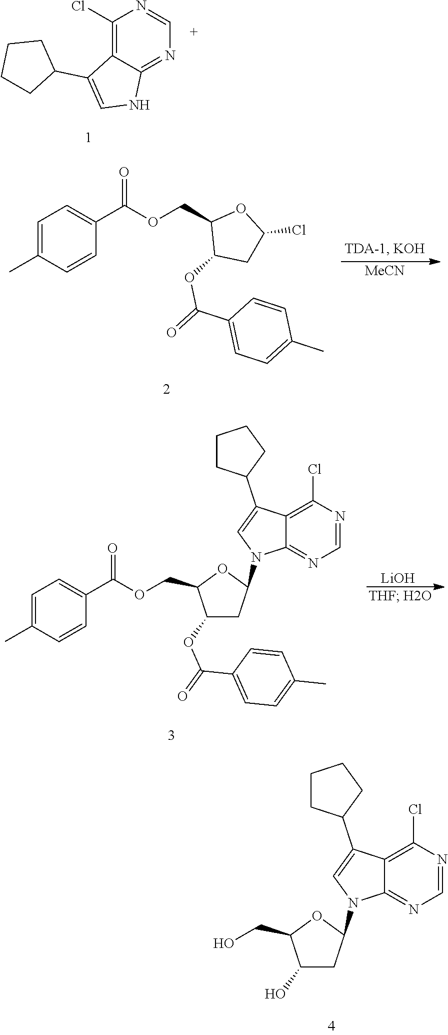

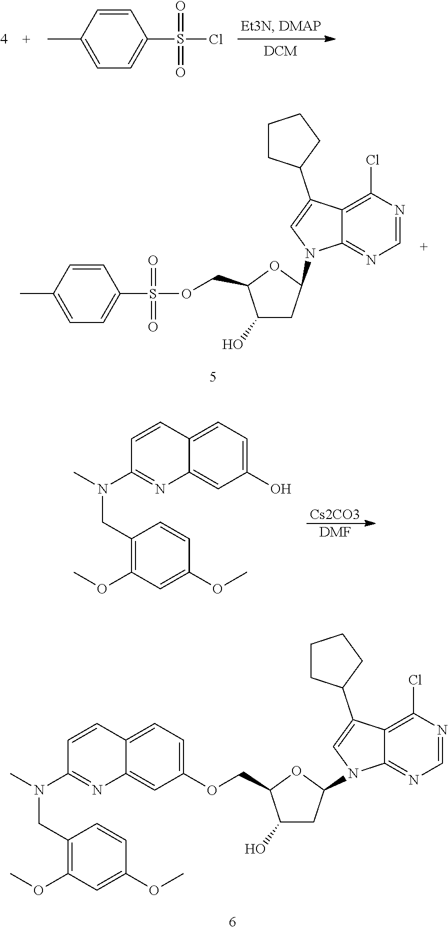

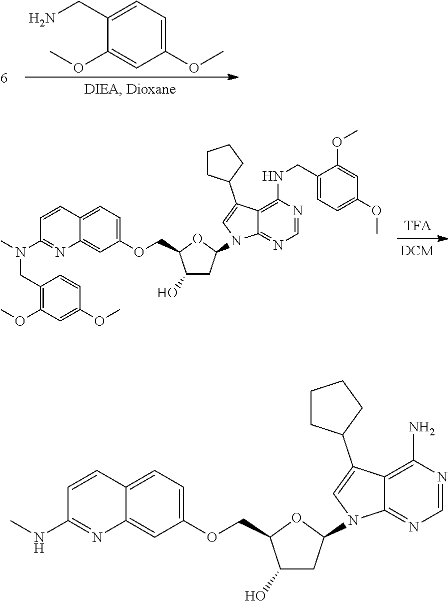

### Synthesis of [(2R,3S,5R)-5-{4-chloro-5-cyclopentyl-7H-pyrrolo[2,3-d]pyrimidin-7-yl}-3-(4-methylbenzoyloxy)oxolan-2-yl]methyl 4-methylbenzoate (3)

A solution of potassium hydroxide (56 mg, 998 μmol) in MeCN (5 mL) was added 8-[2-(2-methoxyethoxy)ethyl]-2,5,11,14-tetraoxa-8-azapentadecane (8 μL) at room temperature and stirred for 5 min under N2. Then 4-chloro-5-cyclopentyl-7H-pyrrolo[2,3-d]pyrimidine (100 mg, 451 μmol) was added at room temperature and stirred for 5 mins under N2. Then (2R,3S,5R)-5-chloro-2-[(4-methylbenzoyloxy)methyl]oxolan-3-yl 4-methylbenzoate(179 mg, 460 μmol) was added at room temperature and stirred for another 25 mins under N2. TLC(PE/EA=5/1) showed the starting material was consumed completely. The mixture was concentrated without any work up, the residue was purified by silica gel column chromatography (silica, 10 g, EA/PE: 0˜10%)) to yield [(2R,3S,5R)-5-{4-chloro-5-cyclopentyl-7H-pyrrolo[2,3-d]pyrimidin-7-yl}-3-(4-methylbenzoyloxy)oxolan-2-yl]methyl 4-methylbenzoate (180 mg, 313 μmol) as a yellow solid, ESI LC-MS m/z=575 [M+H]⁺.

### Synthesis of (2R,3S,5R)-5-{4-chloro-5-cyclopentyl-7H-pyrrolo[2,3-d]pyrimidin-7-yl}-2-(hydroxymethyl)oxolan-3-ol (4)

[(2R,3 S,5R)-5-{4-chloro-5-cyclopentyl-7H-pyrrolo[2,3-d]pyrimidin-7-yl}-3-(4-methylbenzoyloxy)oxolan-2-yl]methyl 4-methylbenzoate (153 mg, 266 μmol) and LiOH. H₂O (48.0 mg, 1.14 mmol) were mixed in THF (4 mL) and H₂O (1 mL), the mixture was stirred at 50° C. for 6 hrs. TLC(PE/EA=5/1) showed the start material was consumed completely. The mixture was diluted with water (15 ml) and extracted with EA (20 mL×2). The organic layer was separated, washed with H₂O (20 mL) and brine (20 mL), and dried with anhydrous Na₂SO₄. The mixture was concentrated under reduced pressure to get (2R,3S,5R)-5-{4-chloro-5-cyclopentyl-7H-pyrrolo[2,3-d]pyrimidin-7-yl}-2-(hydroxymethyl)oxolan-3-ol (80.0 mg, 236 μmol) as a yellow solid, ESI LC-MS m/z=338.1 [M+H]⁺.

### Synthesis of [(2R,3S,5R)-5-{4-chloro-5-cyclopentyl-7H-pyrrolo[2,3-d]pyrimidin-7-yl}-3-hydroxyoxolan-2-yl]methyl 4-methylbenzene-1-sulfonate (5)

To a solution of (2R,3S,5R)-5-{4-chloro-5-cyclopentyl-7H-pyrrolo[2,3-d]pyrimidin-7-yl}-2-(hydroxymethyl)oxolan-3-ol (71.0 mg, 210 μmol) and triethylamine (63.0 mg, 622 μmol) in DCM (3 mL) was added 4-dimethylaminopyridine (6.00 mg, 49.1 μmol) and TsC1 (58.0 mg, 304 μmol). The mixture was stirred at room temperature for 16 hrs. The mixture was purified by Prep-TLC (PE:EA=1:1) directly to yield [(2R,3S,5R)-5-{4-chloro-5-cyclopentyl-7H-pyrrolo[2,3-d]pyrimidin-7-yl}-3-hydroxyoxolan-2-yl]methyl 4-methylbenzene-1-sulfonate (36.0 mg, 73.1 μmol) as a yellow solid, ESI LC-MS m/z=491.9 [M+H]⁺.

### Synthesis of (2R,3S,5R)-5-{4-chloro-5-cyclopentyl-7H-pyrrolo[2,3-d]pyrimidin-7-yl}-2-{[(2-{[(2,4-dimethoxyphenyl)methyl](methyl)amino}quinolin-7-yl)oxy]methyl}oxolan-3-ol (6)

To a solution of [(2R,3S,5R)-5-{4-chloro-5-cyclopentyl-7H-pyrrolo[2,3-d]pyrimidin-7-yl}-3-hydroxyoxolan-2-yl]methyl 4-methylbenzene-1-sulfonate (36.0 mg, 73.1 μmol) in DMF (2 mL) was added 2-{[(2,4-dimethoxyphenyl)methyl](methyl)amino}quinolin-7-ol (31.0 mg, 95.5 μmol) and caesium carbonate (35.0 mg, 107 μmol), then the mixture was stirred at 30° C. for 16 hrs. The mixture was concentrated under reduced pressure. The residue was purified by Prep-TLC (PE:EA=1:3) to yield (2R,3S,5R)-5-{4-chloro-5-cyclopentyl-7H-pyrrolo[2,3-d]pyrimidin-7-yl}-2-{[(2-{[(2,4-dimethoxyphenyl)methyl](methyl)amino}quinolin-7-yl)oxy]methyl}oxolan-3-ol (25.0 mg, 38.8 μmol) as a white solid, ESI LC-MS m/z=644.3 [M+H]⁺.

### Synthesis of (2R,3S,5R)-5-(5-cyclopentyl-4-{[(2,4-dimethoxyphenyl)methyl]amino}-7H-p yrrolo[2,3-d]pyrimidin-7-yl)-2-{[(2-{[(2,4-dimethoxyphenyl)methyl](methyl)amino}quin olin-7-yl)oxy]methyl}oxolan-3-ol (7)

A mixture of (2R,3S,5R)-5-{4-chloro-5-cyclopentyl-7H-pyrrolo[2,3-d]pyrimidin-7-yl}-2-{[(2-{[(2,4-dimethoxyphenyl)methyl](methyl)amino}quinolin-7-yl)oxy]methyl}oxolan-3-ol (25.0 mg, 38.8 μmol) in Dioxane (2 mL) was added 1-(2,4-dimethoxyphenyl)methanamine (80.0 mg, 478 μmol) and N,N-diisopropylethylamine (75.0 mg, 580 μmol) and the mixture stirred at 120° C. for 16 hrs. The mixture was concentrated under reduced pressure and the residue was purified by TLC (PE:EA=1:10) to yield (2R,3S,5R)-5-(5-cyclopentyl-4-{[(2,4-dimet hoxyphenyl)methyl]amino}-7H-pyrrolo[2,3-d]pyrimidin-7-yl)-2-{[(2-{[(2,4-dimethoxyphenyl)methyl](methyl)amino}quinolin-7-yl)oxy]methyl}oxolan-3-ol (20.0 mg, 25.8 μmol), as a ye llow solid, ESI LC-MS m/z=775.3 [M+H]⁺.

### Synthesis of (2R,3S,5R)-5-(4-amino-5-cyclopentyl-7H-pyrrolo[2,3-d]pyrimidin-7-yl)-2-(((2-(methylamino)quinolin-7-yl)oxy)methyl)tetrahydrofuran-3-ol (compound I-30)

To a solution of (2R,3S,5R)-5-(5-cyclopentyl-4-{[(2,4-dimethoxyphenyl)methyl]amino}-7H-pyrrolo[2,3-d]pyrimidin-7-yl)-2-{[(2-{[(2,4-dimethoxyphenyl)methyl](methyl)amino}quinolin-7-yl)oxy]methyl}oxolan-3-ol (20.0 mg, 25.8 μmol) in DCM (0.2 mL) was added TFA (3 mL). The mixture was stirred at room temperature for 1 hrs. Then the mixture was concentrated under reduced pressure, the residue was neutralized by 3 ml 7M NH₃ in MeOH to PH=8, and filtered. The filtrate was purified by Prep-HPLC to yield (2R,3S,5R)-5-(4-amino-5-cyclopentyl-7H-pyrrolo[2,3-d]pyrimidin-7-yl)-2-(((2-(methylamino)quinolin-7-yl)oxy)methyl)tetrahydrofuran-3-ol (2.4 mg, 4.2 μmol) as a white solid, ES LC-MS m/z=475.7 [M+H]⁺. ¹H NMR (400 MHz, DMSO-d6) δ ppm 8.03 (s, 1H), 7.74 (d, J=8.8 Hz, 1H), 7.51 (d, J=8.8 Hz, 1H), 7.11 (s, 1H), 7.01 (d, J=2.4 Hz, 1H), 6.95-6.93 (m, 1H), 6.81 (dd, J=8.8, 2.0 Hz, 1H), 6.64-6.61 (m, 1H), 6.57 (d, J=9.0 Hz, 1H), 6.52 (s, 2H), 5.45 (d, J=4.0 Hz, 1H), 4.49 (s, 1H), 4.29-4.26 (m, 1H), 4.20-4.10 (m, 2H), 3.30-3.28 (m, 1H), 2.88 (d, J=4.8 Hz, 3H), 2.68-2.61 (m, 1H), 2.24-2.19 (m, 1H), 1.99-1.89 (m, 2H), 1.63-1.55 (m, 4H), 1.47-1.35 (m, 2H).

General Method D

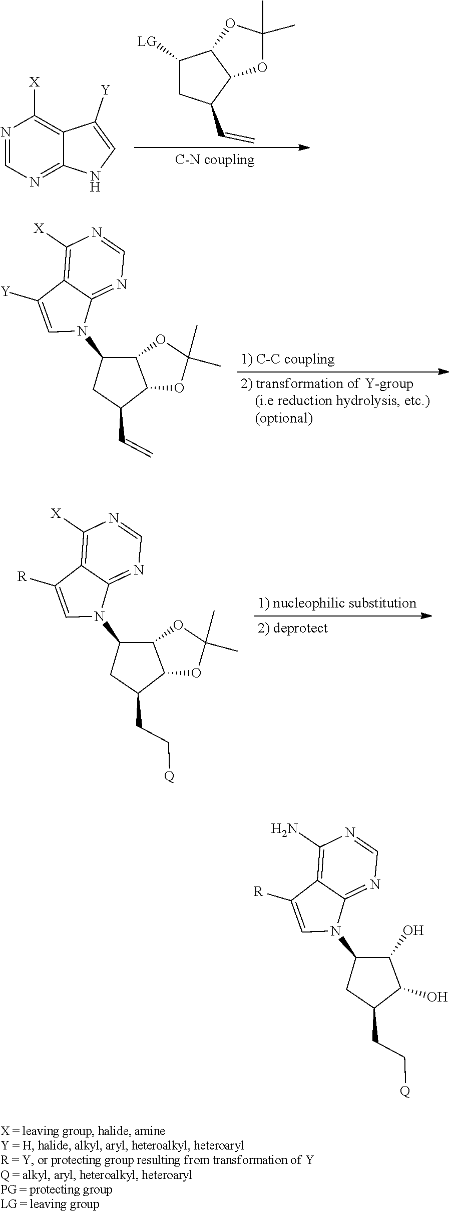

### Example 4: Synthesis of (1R,2S,3R,5S)-3-[4-amino-5-(hydroxymethyl)-7H-pyrrolo[2,3-d]pyrimidin-7-yl]-5-{2-[2-(methylamino)quinolin-7-yl]ethyl}cyclopentane-1,2-diol (compound I-52)

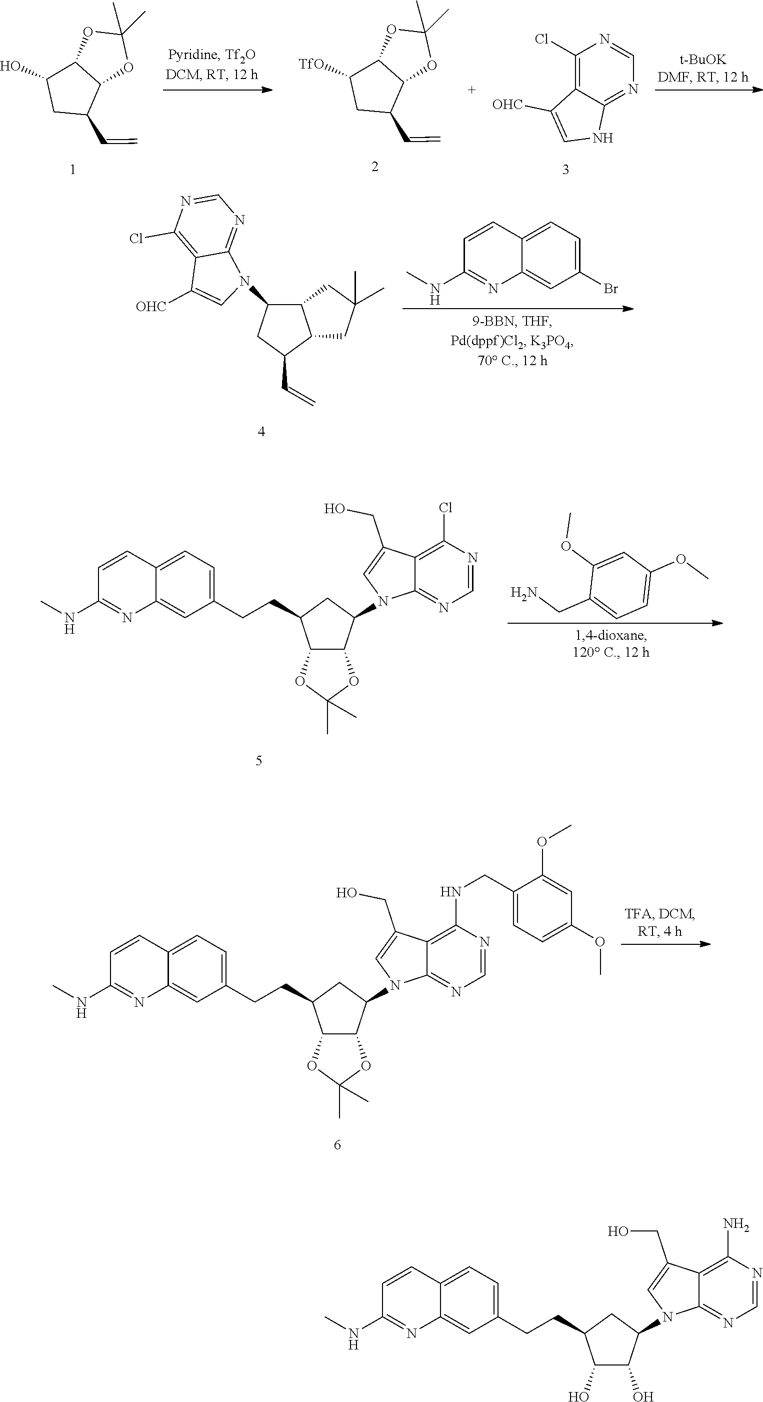

### Synthesis of (3aR,4S,6R,6aR)-6-ethenyl-2,2-dimethyl-hexahydrocyclopenta[d][1,3]dioxol-4-yl trifluoromethanesulfonate (2)

A mixture of (3aS,4S,6R,6aR)-6-ethenyl-2,2-dimethyl-hexahydrocyclopenta[d][1,3]dioxol-4-ol (600 mg, 3.25 mmol) and pyridine (1.28 g, 16.2 mmol) in DCM (20 mL) was added Tf₂O (920 mg, 3.25 mmol). The mixture was stirred at room temperature for 12 hrs. TLC showed the starting material was consumed completely. Then the mixture was diluted with DCM (50 mL), then washed with H₂O (20 mL×3) and brine. The organic layer was dried with Na₂SO₄, filtered and concentrated under reduced pressure, The crude was purified by flash chromatography (silica, 10 g, EA/PE: 0˜30%) to get (3aR,4S,6R,6aR)-6-ethenyl-2,2-dimethyl-hexahydrocyclopenta[d][1,3]dioxol-4-yl trifluoromethanesulfonate (630 mg, 1.99 mmol yield 61%) as a yellow oil.

### Synthesis of 7-[(3aS,4S,6R,6aR)-6-ethenyl-2,2-dimethyl-hexahydrocyclopenta[d][1,3]dioxol-4-yl]-4-chloro-7H-pyrrolo[2,3-d]pyrimidine-5-carbaldehyde (4)

4-chloro-7-potassio-7H-pyrrolo[2,3-d]pyrimidine-5-carbaldehyde (300 mg, 1.36 mmol) was dissolved in THE (4 mL) then t-BuOK (152 mg, 1.36 mmol) was added into the above reaction mixture, stirred at room temperature for 1 hr. The solvent was removed under reduced pressure to get the crude reaction mixture. The residue was dissolved in 2 mL of DMF, and (3aR,4S,6R,6aR)-6-ethenyl-2,2-dimethyl-hexahydrocyclopenta[d][1,3]dioxol-4-yl trifluoromethanesulfonate (430 mg, 1.36 mmol) in 1 mL of DMF was added into the above reaction mixture, stirred at room temperature for 12 h. Then the mixture was washed with water (15 mL), extracted with EA (100 mL), the organic layer was dried with Na2SO4, filtered and concentrated under reduced pressure, the residue was purified by pre-TLC (silica, EA/PE=1/1) to get 7-[(3aS,4S,6R,6aR)-6-ethenyl-2,2-dimethyl-hexahydrocyclopenta[d][1,3]dioxol-4-yl]-4-chloro-7H-pyrrolo[2,3-d]pyrimidine-5-carbaldehyde (108 mg, 310 μmol, yield 23%) as a yellow solid. ESI LC-MS m/z=348.1 [M+H]⁺.

### Synthesis of (4-chloro-7-((3aS,4R,6S,6aR)-2,2-dimethyl-6-(2-(2-(methylamino)quinolin-7-yl)ethyl)tetrahydro-3aH-cyclopenta[d][1,3]dioxol-4-yl)-7H-pyrrolo[2,3-d]pyrimidin-5-yl)methanol (5)

A mixture of 7-[(3aS,4R,6R,6aR)-6-ethenyl-2,2-dimethyl-hexahydrocyclopenta[d][1,3]dioxol-4-yl]-4-chloro-7H-pyrrolo[2,3-d]pyrimidine-5-carbaldehyde (180 mg, 517 μmol) and 9-borabicyclo[3.3.1]nonane/THF (5 mL, 0.5 M, 2.58 mmol) was heated to 50° C. for 1 h. Then the mixture was cooled to 0° C., tripotassium phosphate (329 mg, 1.55 mmol) in water (0.5 mL) was added into the above reaction mixture, stirred at room temperature for 5 min. Then 7-bromo-N-methylquinolin-2-amine (147 mg, 620 μmol) in THE (6 mL) was added into the above reaction mixture. The mixture was heated to reflux for 12 hrs, diluted with water (10 mL), extracted with EA (100 mL), the organic layer was dried with Na2SO4, filtered and concentrated under reduced pressure, the residue was purified by pre-TLC(silica, PE\EA=1\2) to get (4-chloro-7-((3aS,4R,6S,6aR)-2,2-dimethyl-6-(2-(2-(methylamino)quinolin-7-yl)ethyl)tetrahydro-3aH-cyclopenta[d][1,3]dioxol-4-yl)-7H-pyrrolo[2,3-d]pyrimidin-5-yl)methanol (98.0 mg, 193 μmol, yield: 38%). ESI LC-MS m/z=505.9 [M+H]⁺.

### Synthesis of {7-[(3aS,4R,6S,6aR)-2,2-dimethyl-6-{2-[2-(methylamino)quinolin-7-yl]ethyl}-hexahydrocyclopenta[d][1,3]dioxol-4-yl]-4-{[(2,4-dimethoxyphenyl)methyl]amino}-7H-pyrrolo[2,3-d]pyrimidin-5-yl}methanol (6)

A mixture of {7-[(3aS,4R,6S,6aR)-2,2-dimethyl-6-{2-[2-(methylamino)quinolin-7-yl]ethyl}-hexahydrocyclopenta[d][1,3]dioxol-4-yl]-4-chloro-7H-pyrrolo[2,3-d]pyrimidin-5-yl}methanol (140 mg, 275 μmol) and 1-(2,4-dimethoxyphenyl)methanamine (137 mg, 824 μmol) in 1,4-dioxane (4 mL) was heated to 120° C. for 12 h under N2. Then the mixture was concentrated, and the residue was purified by prep. TLC (silica, PE\EA=1\2) to get {7-[(3aS,4R,6S,6aR)-2,2-dimethyl-6-{2-[2-(methylamino)quinolin-7-yl]ethyl}-hexahydrocyclopenta[d][1,3]dioxol-4-yl]-4-{[(2,4-dimethoxyphenyl)methyl]amino}-7H-pyrrolo[2,3-d]pyrimidin-5-yl}methanol (60.0 mg, 93.9 μmol, yield: 34%), ESI LC-MS m/z=639.3 [M+H]⁺.

### Synthesis of (1R,2S,3R,5S)-3-[4-amino-5-(hydroxymethyl)-7H-pyrrolo[2,3-d]pyrimidin-7-yl]-5-{2-[2-(methylamino)quinolin-7-yl]ethyl}cyclopentane-1,2-diol (compound I-52)

A mixture of {7-[(3aS,4R,6S,6aR)-2,2-dimethyl-6-{2-[2-(methylamino)quinolin-7-yl]ethyl}-hexahydrocyclopenta[d][1,3]dioxol-4-yl]-4-{[(2,4-dimethoxyphenyl)methyl]amino}-7H-pyrrolo[2,3-d]pyrimidin-5-yl}methanol (30 mg, 46.9 μmol) and TFA (3 mL) in DCM (3 mL) was stirred at room temperature for 3 h. Then the mixture was concentrated, the residue was neutralized with 7M NH3 in methanol solution. The mixture was concentrated, the residue was purified by pre-HPLC to get (1R,2S,3R,5S)-3-[4-amino-5-(hydroxymethyl)-7H-pyrrolo[2,3-d]pyrimidin-7-yl]-5-{2-[2-(methylamino)quinolin-7-yl]ethyl}cyclopentane-1,2-diol (5.0 mg, 11 μmol, yield 24%) as a white solid. ESI LC-MS m/z=449.2 [M+H]⁺. ¹H NMR (400 MHz, DMSO-d6) δ ppm 8.05 (br, 1H), 7.77 (d, J=5.2 Hz, 1H), 7.51 (d, J=8 Hz, 1H), 7.35 (s, 1H), 7.02 (dd, J=8, 1.2 Hz, 1H), 7.94-7.92 (m, 1H), 6.85 (br, 1H), 6.66 (d, J=9.2 Hz, 1H), 4.81-4.76 (m, 1H), 4.59 (s, 2H), 4.17-4.14 (m, 1H), 3.74-3.71 (m, 1H), 2.88 (d, J=3.6 Hz, 3H), 2.75-2.67 (m, 2H), 2.23-2.21 (m, 1H), 1.96-1.85 (m, 2H), 1.70-1.69 (m, 1H), 1.47-1.44 (m, 1H)

General Method E

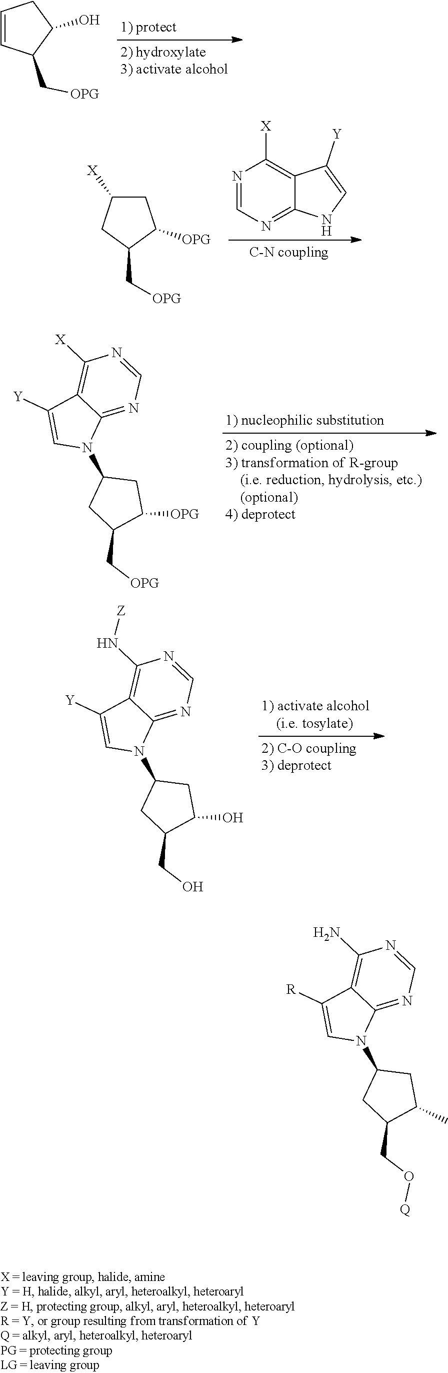

### Example 5: Synthesis of Compound I-169

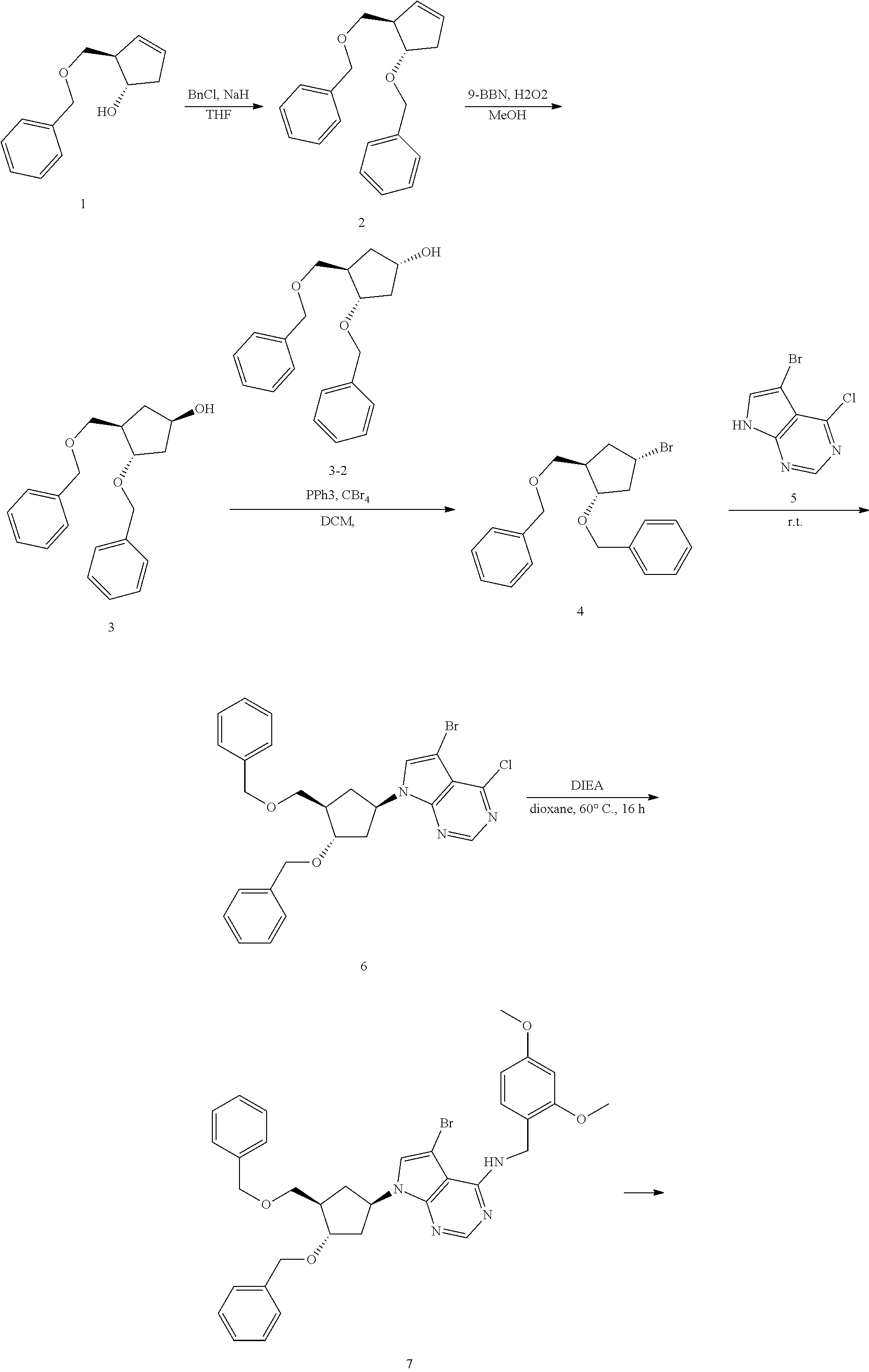

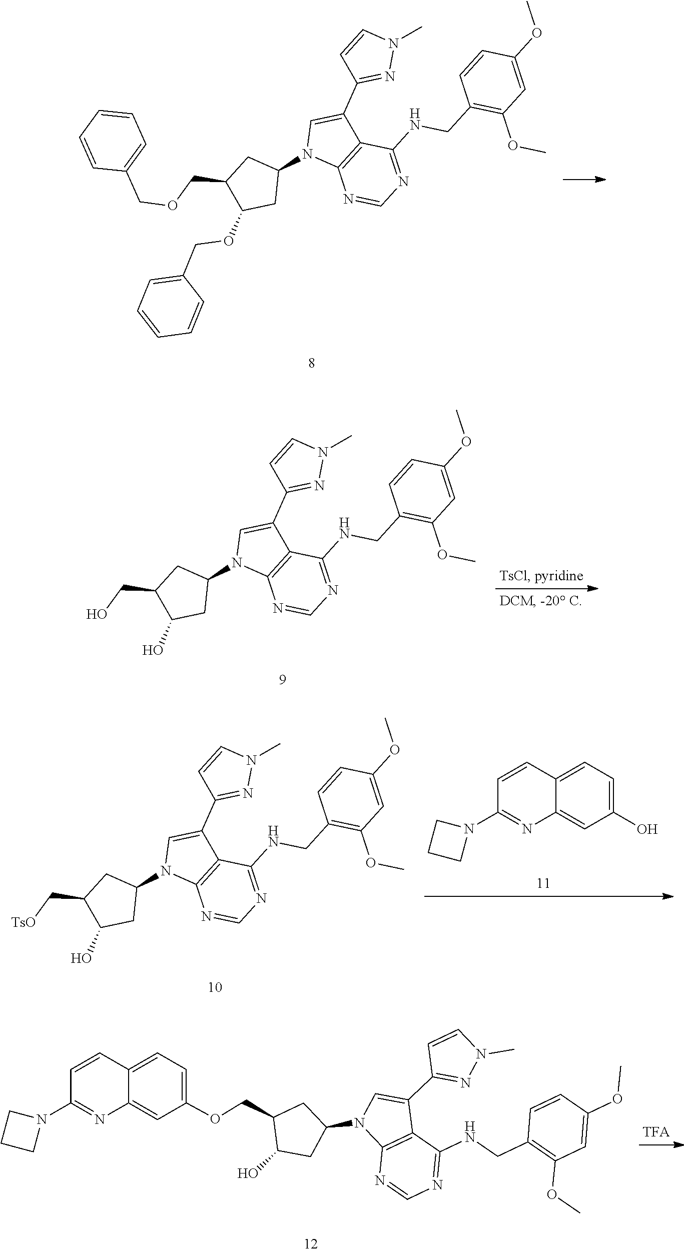

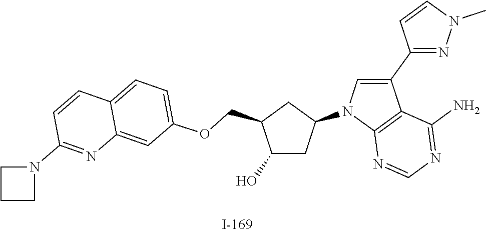 [SMILES: Cn1ccc(-c2cn([C@@H]3C[C@H](COc4ccc5ccc(N6CCC6)nc5c4)[C@@H](O)C3)c3ncnc(N)c23)n1]

### Synthesis of Compound 2

(1S,2R)-2-[(benzyloxy)methyl]cyclopent-3-en-1-ol (45 g, 220 mmol) was added dropwise to a stirred suspension of sodium hydride (6.33 g, 264 mmol)

in tetrahydrofuran (500 mL) at 0° C. under nitrogen. After 1 h at
RT, (bromomethyl)benzene (48.9 g, 286 mmol) was added. The reaction mixture was kept overnight at RT. Crushed ice was added, the mixture was stirred for 0.5 h. Extracted with ethyl acetate (200 mL*3), washed with brine, dried over Na2SO4 and concentrated in vacuo to get the expected product ({[(1R,5S)-5-(benzyloxy)cyclopent-2-en-1-yl]methoxy}methyl)benzene (82.5 g, 280 mmol), as brown oil. Used for next step without further purification.

### Synthesis of Compound 3

A 0.5 M solution of 9-borabicyclo[3.3.1]nonane (880 mL, 440 mmol) was added dropwise to a solution of ({[(1R,5S)-5-(benzyloxy)cyclopent-2-en-1-yl]methoxy}methyl)benzene (65 g, 220 mmol) in anhyd THF (10 mL) at 0° C. under nitrogen. The reaction was slowly warmed to r.t. overnight. The reaction was cooled to 0° C. and treated sequentially with EtOH (70 mL), 3 N NaOH solution (200 mL), and H2O2 (33%, 200 mL). The resulting mixture was stirred at r.t. overnight. The resulting residue was filtered and washed with EtOAc (200 mL). To this suspension, water was added (300 mL) and after separation of the phases, the aq layer was extracted with EtOAc (3×150 mL). The combined organic layers were dried (Na2SO4) and concentrated to dryness. The crude product was purified on silica gel (PE-EtOAc, 1:1 to yield (1R,3S,4R)-3-(benzyloxy)-4-[(benzyloxy)methyl]cyclopentan-1-ol (39.4 g, 126 mmol), as light yellow oils. LC-MS m/z=313.2[M+Na]⁺.

### Synthesis of Compound 4

To a solution of (1R,3S,4R)-3-(benzyloxy)-4-[(benzyloxy)methyl]cyclopentan-1-ol (1.9 g, 6.08 mmol) and tetrabromomethane (6.03 g, 18.2 mmol) in methylene chloride (50 mL) stirred at −60° C. for 0.5 h, Then added triphenylphosphane (4.77 g, 18.2 mmol) over 10 min. The mixture was stirred at r.t. for 2 h an aliquot checked by LC-MS analysis indicates that the reaction is complete. The residue was purified by TLC (PE:EA=8:1) to afford ({[(1R,2S,4S)-2-(benzyloxy)-4-bromocyclopentyl]methoxy}methyl)benzene (1.20 g, 3.19 mmol), as colorless oil. LC-MS m/z=397.1[M+Na]*.

### Synthesis of Compound 6

To a solution of 4-chloro-5-cyclopentyl-7H-pyrrolo[2,3-d]pyrimidine (500 mg, 2.15 mmol) and potassium 2-methylpropan-2-olate (241.3 mg, 2.15 mmol) in tetrahydrofuran (10 mL) was stirred at r.t. for 1 h. The mixture was concentrated under reduced pressure. The residue was dissolved in dimethylformamide (20 mL) and added ({[(1R,2S,4S)-2-(benzyloxy)-4-bromocyclopentyl]methoxy}methyl)benzene (1.2 g, 3.19 mmol) stirred at r.t. for 16 h. an aliquot checked by LC-MS analysis indicates that the reaction is complete. The mixture was partitioned between EA (20 mL) and saturated aqueous H2O (10 mL). The layers were separated and the aqueous phase was extracted with ethyl acetate (20 mL×3). The combined organic layers were washed with brine (30 mL), dried over Na2SO4, filtered and concentrated under reduced pressure. The residue was purified by TLC(PE:EA=3:1) to afford 7-[(1R,3S,4R)-3-(benzyloxy)-4-[(benzyloxy)methyl]cyclopentyl]-5-bromo-4-chloro-7H-pyrrolo[2,3-d]pyrimidine (530 mg, 1.00 mmol), as colorless oil. LC-MS m/z=525.9[M+H]⁺.

### Synthesis of Compound 7

To a solution of 7-[(1R,3S,4R)-3-(benzyloxy)-4-[(benzyloxy)methyl]cyclopentyl]-5-bromo-4-chloro-7H-pyrrolo[2,3-d]pyrimidine (530 mg, 1.00 mmol) in dioxane (4 mL) was added 2,4-dimethoxyaniline (306 mg, 2.00 mmol) and tris(propan-2-yl)amine (429 mg, 3.00 mmol). The reaction mixture was heated to 60° C. overnight. Cooled to room temperature and then the solvent was removed in vacuo. The crude was purified by TLC (EA:PE=2:1) to get the target compound7-[(1R,3 S,4R)-3-(benzyloxy)-4-[(benzyloxy)methyl]cyclopentyl]-5-bromo-N-[(2,4-dimethoxyphenyl)methyl]-7H-pyrrolo[2,3-d]pyrimidin-4-amine (532 mg, 0.8089 mmol), as colorless oil. LC-MS m/z=657.2[M+H]⁺.

### Synthesis of Compound 8

To a solution of 7-[(1R,3S,4R)-3-(benzyloxy)-4-[(benzyloxy)methyl]cyclopentyl]-5-bromo-N-[(2,4-dimethoxyphenyl)methyl]-7H-pyrrolo[2,3-d]pyrimidin-4-amine (500 mg, 0.7603 mmol) in dioxane (6 mL) was added [3-(4,4,5,5-tetramethyl-1,3,2-dioxaborolan-2-yl)-1H-pyrazol-1-yl]methylium (173 mg, 0.8363 mmol), sodium methaneperoxoate sodium hydride (246 mg, 2.28 mmol), lambda2-iron(2+) palladium(2+) bis(2-(diphenylphosphanyl)cyclopenta-2,4-dien-1-ide) dichloride (27.8 mg, 0.03801 mmol) and water (1.5 mL). The reaction mixture was flushed with nitrogen for 15 min and then heated to 90° C. for 3 hunder N2 atmosphere. Diluted with water (50 mL), extracted with ethyl acetate (60 mL*4), dried over Na2SO4, filtered and concentrated in vacuo. The crude was purified by flash chromatography (silica, 12 g, ethyl acetate:petroleum ether:2:1) to get the expected product 7-[(1R,3S,4R)-3-(benzyloxy)-4-[(benzyloxy)methyl]cyclopentyl]-N-[(2,4-dimethoxyphenyl)methyl]-5-(1-methyl-1H-pyrazol-3-yl)-7H-pyrrolo[2,3-d]pyrimidin-4-amine (423 mg, 0.6420 mmol), as colorless oil. LC-MS m/z=659.2[M+H]⁺.

### Synthesis of Compound 9

To a solution of 7-[(1R,3S,4R)-3-(benzyloxy)-4-[(benzyloxy)methyl]cyclopentyl]-N-[(2,4-dimethoxyphenyl)methyl]-5-(1-methyl-1H-pyrazol-3-yl)-7H-pyrrolo[2,3-d]pyrimidin-4-amine (423 mg, 0.6420 mmol) and Pd/C(200 mg) in methanol (100 mL) was stirred at 40° C. for 3 days. an aliquot checked by LC-MS analysis indicates that the reaction is complete. Filtered via celite, concentrated in vacuo to get the expected product (1 S,2R,4R)-4-(4-{[(2,4-dimethoxyphenyl)methyl]amino}-5-(1-methyl-1H-pyrazol-3-yl)-7H-pyrrolo[2,3-d]pyrimidin-7-yl)-2-(hydroxymethyl)cyclopentan-1-ol (300 mg, 0.6268 mmol), as colorless oil. LC-MS m/z=479.0[M+H]⁺.

### Synthesis of Compound 10

(1S,2R,4R)-4-(4-{[(2,4-dimethoxyphenyl)methyl]amino}-5-(1-methyl-1H-pyrazol-3-yl)-7H-pyrrolo[2,3-d]pyrimidin-7-yl)-2-(hydroxymethyl)cyclopentan-1-ol (300 mg, 0.6268 mmol) was dissolved in methylene chloride (10 mL), the solution cooled at −25° C., the pyridine (988 mg, 12.5 mmol) was added. 4-methylbenzene-1-sulfonyl chloride (1.55 g, 8.14 mmol) was added in portions at −25° C. The reaction warmed to −20° C. and stirred at −20° C. overnight. LCMS showed the reaction completed. Quenched by adding MeOH (4 mL), followed by the addition NaOH (1 mol/L, 15 mL) at −20° C. and stirred 10 min at −20° C. Then warmed to RT, diluted with methylene chloride and water. Extracted with DCM 3 times. The organic phase was washed with brine, dried over Na₂SO₄, concentrated in vacuo. Thbe crude was purified by flash chromatography (silica, 12 g, methanol˜dicholormethane:0 20%) to get the expected product [(1R,2S,4R)-4-(4-{[(2,4-dimethoxyphenyl)methyl]amino}-5-(1-methyl-1H-pyrazol-3-yl)-7H-pyrrolo[2,3-d]pyrimidin-7-yl)-2-hydroxycyclopentyl]methyl 4-methylbenzene-1-sulfonate (240 mg, 0.3793 mmol), as white solid. LC-MS m/z=633.2[M+H]⁺.

### Synthesis of Compound 12

To a solution of [(1R,2S,4R)-4-(4-{[(2,4-dimethoxyphenyl)methyl]amino}-5-(1-methyl-1H-pyrazol-3-yl)-7H-pyrrolo[2,3-d]pyrimidin-7-yl)-2-hydroxycyclopentyl]methyl 4-methylbenzene-1-sulfonate (120 mg, 0.1896 mmol) in dimethylformamide (5 mL) was added 2-(azetidin-1-yl)quinolin-7-ol (37.9 mg, 0.1896 mmol) and Cesium carbonate (186 mg, 0.5688 mmol). The reaction was heated to 80° C. for 3 h. Filtered, the solvent was removed under reduced pressure. The crude was purified by flash chromatography(silica, 12 g, methanol˜ dicholormethane: 0˜15%) to get the expected product (1S,2R,4R)-2-({[2-(azetidin-1-yl)quinolin-7-yl]oxy}methyl)-4-(4-{[(2,4-dimethoxyphenyl)methyl]amino}-5-(1-methyl-1H-pyrazol-3-yl)-7H-pyrrolo[2,3-d]pyrimidin-7-yl)cyclopentan-1-ol (51.0 mg, 0.07718 mmol), as white solid. LC-MS m/z=661.2[M+H]⁺.

### Synthesis of Compound I-169

The (1S,2R,4R)-2-({[2-(azetidin-1-yl)quinolin-7-yl]oxy}methyl)-4-(4-{[(2,4-dimethoxyphenyl)methyl]amino}-5-(1-methyl-1H-pyrazol-3-yl)-7H-pyrrolo[2,3-d]pyrimidin-7-yl)cyclopentan-1-ol (59 mg, 0.08928 mmol) was dissolved in trifluoroacetic acid (4 mL). The reaction mixture was stirred at 40° C. for 2 h. The solvent was removed under reduced pressure. Neutralized with NH₃ in methanol (7 mol/L) to pH>7. The crude was purified by Prep-HPLC to get the target compound(1S,2R,4R)-4-[4-amino-5-(1-methyl-1H-pyrazol-3-yl)-7H-pyrrolo[2,3-d]pyrimidin-7-yl]-2-({[2-(azetidin-1-yl)quinolin-7-yl]oxy}methyl)cyclopentan-1-ol (10.1 mg, 0.01978 mmol), as white solid. LC-MS m/z=511.1[M+H]⁺. ¹H NMR (400 MHz, DMSO-d₆) δ 8.95 (s, 1H), 8.04 (s, 1H), 7.91 (d, J=8.8 Hz, 1H), 7.82 (s, 1H), 7.71 (d, J=2.2 Hz, 1H), 7.58 (d, J=8.8 Hz, 1H), 7.13-7.06 (m, 2H), 6.87 (dd, J=8.7, 2.5 Hz, 1H), 6.63 (d, J=2.3 Hz, 1H), 6.51 (d, J=8.8 Hz, 1H), 5.32 (d, J=9.6 Hz, 1H), 5.01 (s, 1H), 4.27-4.21 (m, 2H), 4.12-4.02 (m, 5H), 3.87 (s, 3H), 3.32 (s, 1H), 2.39-2.30 (m, 3H), 2.26-2.20 (m, 1H), 2.08-2.03 (m, 1H), 1.80-1.74 (m, 1H).

General Method F

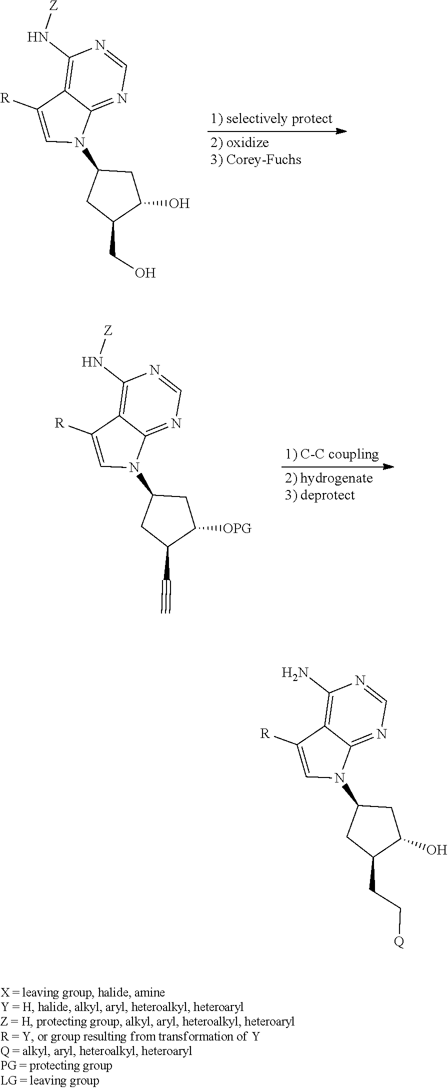

### Example 6: Synthesis of Compound I-206

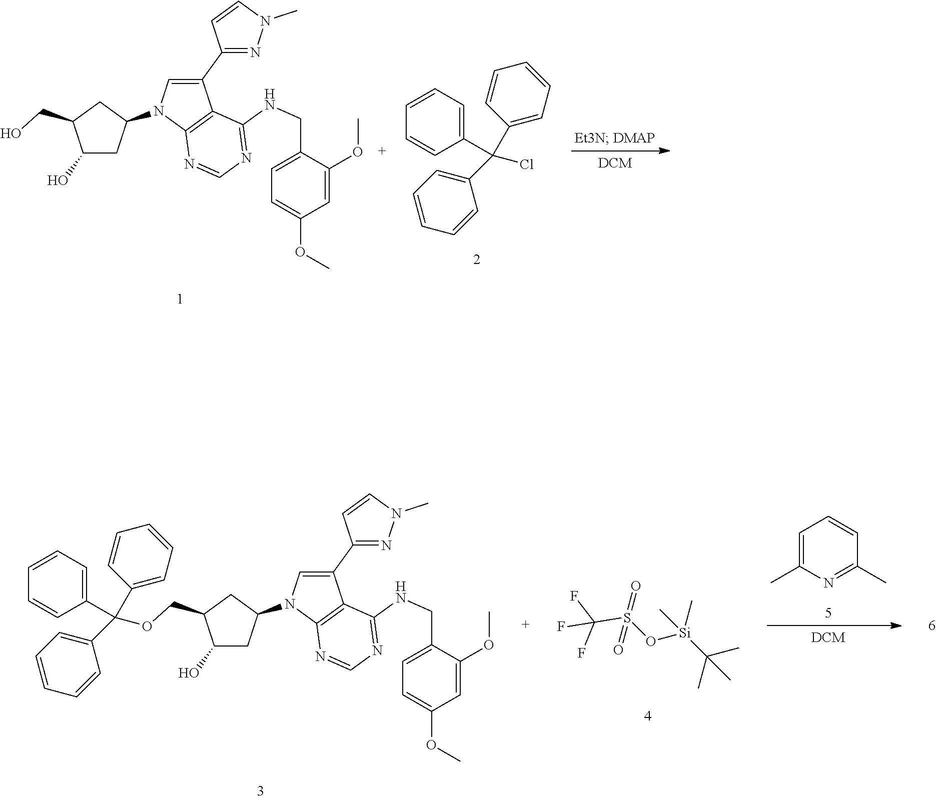

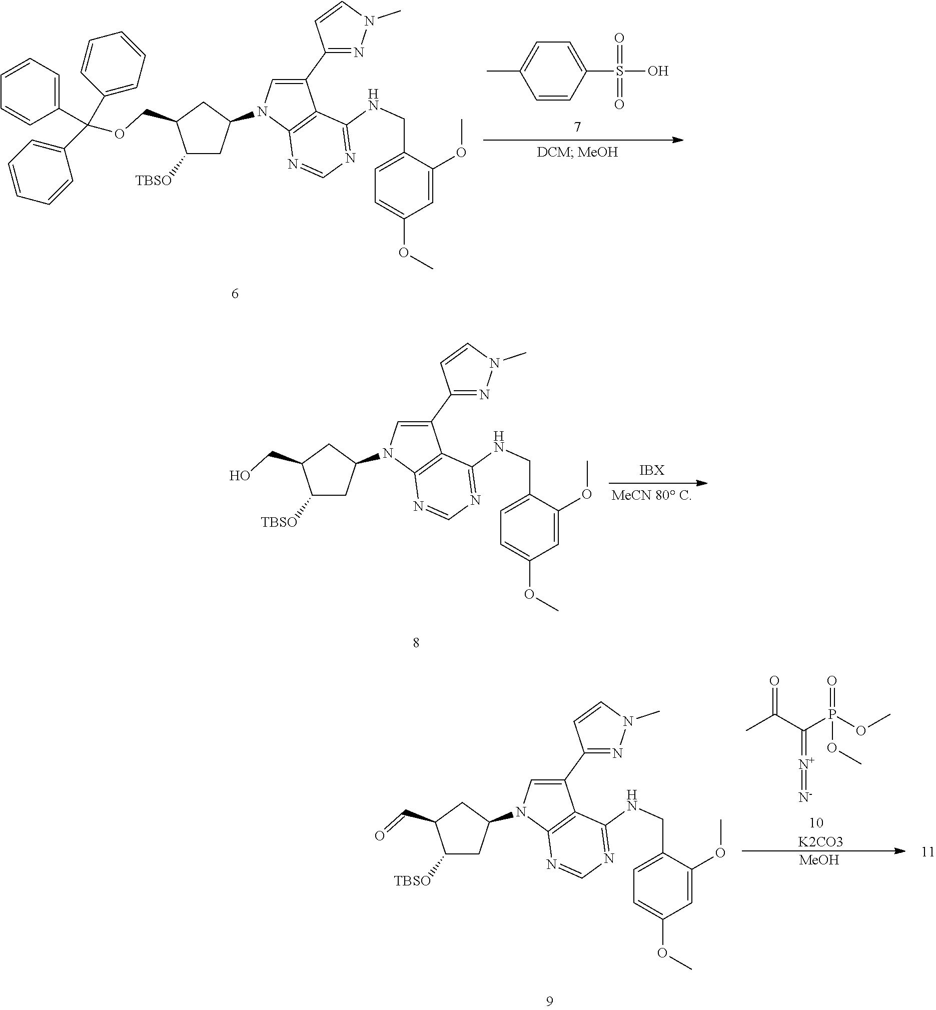

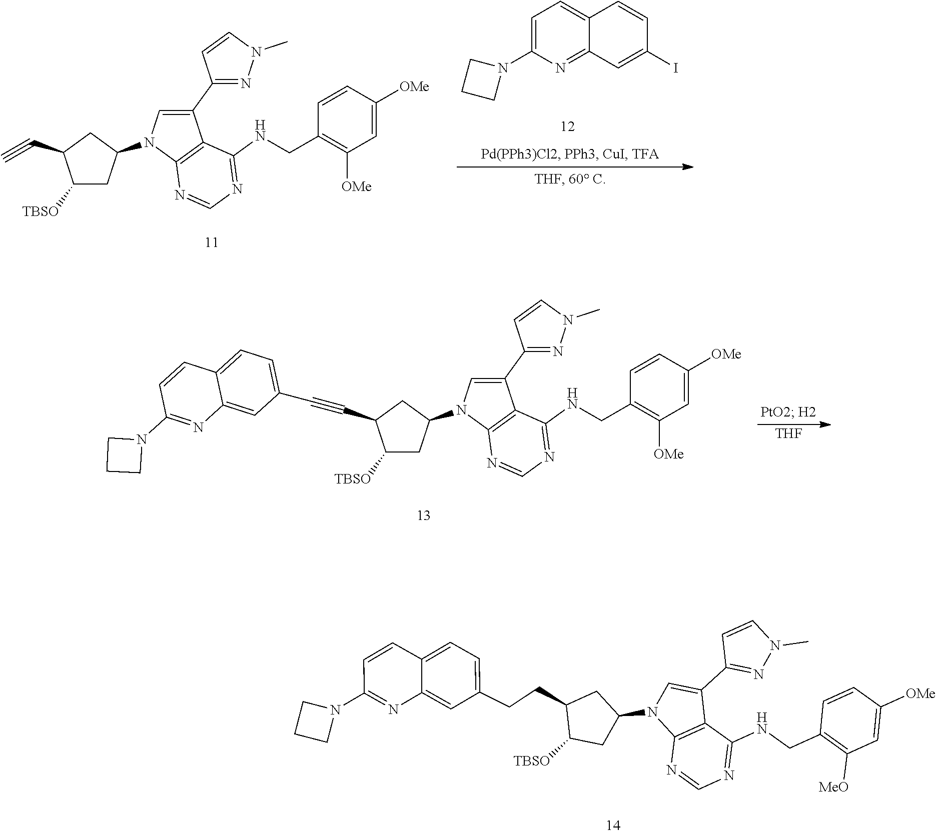 [SMILES: C#C[C@H]1C[C@@H](n2cc(-c3ccn(C)n3)c3c(NCc4ccc(OC)cc4OC)ncnc32)C[C@@H]1O[Si](C)(C)C(C)(C)C ; Ic1ccc2ccc(N3CCC3)nc2c1 ; COc1ccc(CNc2ncnc3c2c(-c2ccn(C)n2)cn3[C@H]2C[C@H](O[Si](C)(C)C(C)(C)C)[C@@H](C#Cc3ccc4ccc(N5CCC5)nc4c3)C2)c(OC)c1 ; "THF" ; COc1ccc(CNc2ncnc3c2c(-c2ccn(C)n2)cn3[C@@H]2C[C@H](CCc3ccc4ccc(N5CCC5)nc4c3)[C@@H](O[Si](C)(C)C(C)(C)C)C2)c(OC)c1]

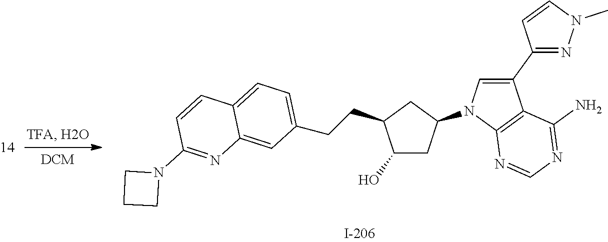 [SMILES: Cn1ccc(-c2cn([C@@H]3C[C@H](CCc4ccc5ccc(N6CCC6)nc5c4)[C@@H](O)C3)c3ncnc(N)c23)n1]

### Synthesis of Compound 3

To a solution of (1S,2R,4R)-4-(4-{[(2,4-dimethoxyphenyl)methyl]amino}-5-(1-methyl-1H-pyrazol-3-yl)-7H-pyrrolo[2,3-d]pyrimidin-7-yl)-2-(hydroxymethyl)cyclopentan-1-ol (1 g, 2.08 mmol), and triethylamine (2.1 g, 20.7 mmol) in DCM (30 mL), was added 4-dimethylaminopyridine (12 mg, 98.2 μmol) and (chlorodiphenylmethyl)benzene (1.15 g, 4.16 mmol) and stirred at 50° C. for 1 h. TLC (PE/EA=1/1) showed the reaction was completed. The solvent was removed under reduced pressure and the residue purified by silica gel column chromatography (EA 80%) to give (1S,2R,4R)-4-(4-{[(2,4-dimethoxyphenyl)methyl]amino}-5-(1-methyl-1H-pyrazol-3-yl)-7H-pyrrolol[2,3-d]pyrimidin-7-yl)-2-[(triphenylmethoxy)methyl]cyclopentan-1-ol (1.20 g, 1.66 mmol).

### Synthesis of Compound 6

To a solution of (1S,2R,4R)-4-(4-{[(2,4-dimethoxyphenyl)methyl]amino}-5-(1-methyl-1H-pyrazol-3-yl)-7H1-pyrrolo[2,3-d]pyrimidin-7-yl)-2-[(triphenylmethoxy)methyl]cyclopentan-1-ol (1.2 g, 1.66 mmol) in DCM (20 mL), was added lutidine (1.17 g, 10.9 mmol) and tert-butyldimethylsilyl trifluoromethanesulfonate (1.7 g, 6.43 mmol) at rt and stirred at rt for 10 mins. TLC (PE/EA=1/1) showed the reaction was completed. The solvent was removed under reduced pressure and the residue purified by silica gel column chromatography (EA 80%) to give 7-[(1R,3S,4R)-3-[(tert-

[symbol]

### Synthesis of Compound 6

To a solution of (1S,2R,4R)-4-(4-{[(2,4-dimethoxyphenyl)methyl]amino}-5-(1-methyl-1H-pyrazol-3-yl)-7H-pyrrolo[2,3-d]pyrimidin-7-yl)-2-[(triphenylmethoxy)methyl]cyclopentan-1-ol (1.2 g, 1.66 mmol) in DCM (20 mL), was added lutidine (1.17 g, 10.9 mmol) and tert-butyldimethylsilyl trifluoromethanesulfonate (1.7 g, 6.43 mmol) at rt and stirred at rt for 10 mins. TLC (PE/EA=1/1) showed the reaction was completed. The solvent was removed under reduced pressure and the residue purified by silica gel column chromatography (EA 80%) to give 7-[(1R,3S,4R)-3-[(tert-butyldimethylsilyl)oxy]-4-[(triphenylmethoxy)methyl]cyclopentyl]-N-[(2,4-dimethoxyphenyl)methyl]-5-(1-methyl-1H-pyrazol-3-yl)-7H-pyrrolo[2,3-d]pyrimidin-4-amine (1.24 g, 1.48 mmol).

### Synthesis of Compound 8

To a solution of 7-[(1R,3S,4R)-3-[(tert-butyldimethylsilyl)oxy]-4-[(triphenylmethoxy)methyl]cyclopentyl]-N-[(2,4-dimethoxyphenyl)methyl]-5-(1-methyl-1H-pyrazol-3-yl)-7H-pyrrolo[2,3-d]pyrimidin-4-amine (1.24 g, 1.48 mmol) in DCM (36 mL), was added MeOH (3 mL) and para-toluene sulfonate (130 mg, 754 μmol) at 20° C. and stirred at 20° C. for 1 h. TLC (PE/EA=1/1) indicated that reaction was well. The mixture was neutralized with NaHCO₃ aq at 10° C. The organic layer was separated, washed with H₂O (50 mL×2) and brine (50 mL), and dried (Na₂SO₄). The solvent was removed under reduced pressure and the residue purified by silica gel column chromatography (80% EA) to give [(1R,2S,4R)-2-[(tert-butyldimethylsilyl)oxy]-4-(4-{[(2,4-dimethoxyphenyl)methyl]amino}-5-(1-methyl-1H-pyrazol-3-yl)-7H-pyrrolo[2,3-d]pyrimidin-7-yl)cyclopentyl]methanol (495 mg, 834 μmol). ESI LCMS m/z=593.3[M+1]⁺.

### Synthesis of Compound 9

[(1R,2S,4R)-2-[(tert-butyldimethylsilyl)oxy]-4-(4-{[(2,4-dimethoxyphenyl)methyl]amino}-5-(1-methyl-1H-pyrazol-3-yl)-7H-pyrrolo[2,3-d]pyrimidin-7-yl)cyclopentyl]methanol (20.0 mg, 33.7 μmol), 2-iodylbenzoic acid (47.0 mg, 168 μmol) were mixed in MeCN (2 mL). The mixture was heated to 80° C. and stirred for 10 min. LCMS show reaction completed. Reaction solution was filtrated and concentrated under reduced pressure to get the crude(1S,2S,4R)-2-[(tert-butyldimethylsilyl)oxy]-4-(4-{[(2,4-dimethoxyphenyl)methyl]amino}-5-(1-methyl-1H-pyrazol-3-yl)-7H-pyrrolo[2,3-d]pyrimidin-7-yl)cyclopentane-1-carbaldehyde (17.6 mg, 29.7 μmol). The crude was directly used to next step without further purification. ESI LCMS m/z=567.3[M+1]⁺.

### Synthesis of Compound 11

(1S,2S,4R)-2-[(tert-butyldimethylsilyl)oxy]-4-(4-{[(2,4-dimethoxyphenyl)methyl]amino}-5-(1-methyl-1H-pyrazol-3-yl)-7H-pyrrolo[2,3-d]pyrimidin-7-yl)cyclopentane-1-carbaldehyde (600 mg, 1.01 mol), dimethyl (1-diazo-2-oxopropyl)phosphonate (582 mg, 50.6 μmol), K2CO3 (974 mg, 7.06 mol) were mixed in MeOH (10 mL). The mixture was stirred for 1 h at rt. LCMS show reaction completed. reaction solution was concentrated under reduced pressure to get the crude. The crude was purified by fish chromatography (MeOH˜ DCM: 0˜10%) to get 7-[(1R,3S,4R)-3-[(tert-butyldimethylsilyl)oxy]-4-ethynylcyclopentyl]-N-[(2,4-dimethoxyphenyl)methyl]-5-(1-methyl-1H-pyrazol-3-yl)-7H-pyrrolo[2,3-d]pyrimidin-4-amine (330, 562 μmol) as a liquid. ESI LCMS m/z=563.3[M+1]⁺.

### Synthesis of Compound 13

To a solution of 7-[(1R,3S,4R)-3-[(tert-butyldimethylsilyl)oxy]-4-ethynylcyclopentyl]-N-[(2,4-dimethoxyphenyl)methyl]-5-(1-methyl-1H-pyrazol-3-yl)-7H-pyrrolo[2,3-d]pyrimidin-4-amine (30 mg, 51.1 μmol), 2-(azetidin-1-yl)-7-iodoquinoline (23.7 mg, 76.6 μmol), palladium(2+) bis(triphenylphosphine) dichloride (1.78 mg, 2.55 μmol), triethylamine (20.6 mg, 204 μmol), triphenylphosphine (2.67 mg, 10.2 μmol) and lambda1-copper(1+) iodide (485 μg, 2.55 μmol) in THF (5 mL) was stirred under N2 at 60° C. for 16 h. LCMS showed the reaction was completed. The mixture was purified by prep-HPLC to get 7-[(1R,3R,4S)-3-{2-[2-(azetidin-1-yl)quinolin-7-yl]ethynyl}-4-[(tert-butyldimethylsilyl)oxy]cyclopentyl]-N-[(2,4-dimethoxyphenyl)methyl]-5-(1-methyl-1H-pyrazol-3-yl)-7H-pyrrolo[2,3-d]pyrimidin-4-amine (20.0 mg, 26.0 μmol) as yellow soild. ESI LCMS m/z=769.3[M+1]⁺.

### Synthesis of Compound 14

7-[(1R,3R,4S)-3-{2-[2-(azetidin-1-yl)quinolin-7-yl]ethynyl}-4-[(tert-butyldimethylsilyl)oxy]cyclopentyl]-N-[(2,4-dimethoxyphenyl)methyl]-5-(1-methyl-1H-pyrazol-3-yl)-7H-pyrrolo[2,3-d]pyrimidin-4-amine (20 mg, 26.0 μmol) and PtO₂ (5.90 mg, 26.0 μmol) were mixed with THE (2 mL) and attached to a hydrogenation apparatus. The system was evacuated and then refilled with hydrogen. The mixture was stirred at r.t for 10 min. LCMS showed the reaction was completed. Then filter off PtO₂ and the solvent was removed under reduced pressure to get 7-[(1R,3S,4S)-3-{2-[2-(azetidin-1-yl)quinolin-7-yl]ethyl}-4-[(tert-butyldimethylsilyl)oxy]cyclopentyl]-N-[(2,4-dimethoxyphenyl)methyl]-5-(1-methyl-1H-pyrazol-3-yl)-7H-pyrrolo[2,3-d]pyrimidin-4-amine (17.0 mg, 21.9 μmol) as yellow soild. ESI LCMS m/z=773.2[M+1]⁺.

### Synthesis of Compound I-206

To a solution of 7-[(1R,3S,4S)-3-{2-[2-(azetidin-1-yl)quinolin-7-yl]ethyl}-4-[(tert-butyldimethylsilyl)oxy]cyclopentyl]-N-[(2,4-dimethoxyphenyl)methyl]-5-(1-methyl-1H-pyrazol-3-yl)-7H-pyrrolo[2,3-d]pyrimidin-4-amine (17 mg, 21.9 μmol) in DCM (1 mL) was added TFA (2 mL). The mixture was stirred at r.t for 0.5 h. LCMS showed the reaction was completed. Then the mixture was concentrated to get crude, neutralized by 4 mL 7M NH3 in MeOH. The MeOH was removed under reduced pressure to get crude. Then crude is dissolved using THE and filter off solids. The product was purified by Prep-HPLC to afford (1S,2S,4R)-4-[4-amino-5-(1-methyl-1H-pyrazol-3-yl)-7H-pyrrolo[2,3-d]pyrimidin-7-yl]-2-{2-[2-(azetidin-1-yl)quinolin-7-yl]ethyl}cyclopentan-1-ol (3.10 mg, 6.09 μmol) as white soild. ESI LCMS m/z=509.0[M+1]⁺1H NMR (400 MHz, DMSO-d6) δ 8.96 (s, 1H), 8.03 (s, 1H), 7.95 (d, J=8.8 Hz, 1H), 7.80 (s, 1H), 7.71 (d, J=2.2 Hz, 1H), 7.60 (d, J=8.2 Hz, 1H), 7.42 (s, 1H), 7.10 (dd, J=8.1, 1.6 Hz, 2H), 6.64 (dd, J=16.3, 5.5 Hz, 2H), 5.21 (dt, J=17.3, 8.6 Hz, 1H), 4.86 (d, J=4.7 Hz, 1H), 4.04 (dt, J=11.9, 7.2 Hz, 5H), 3.87 (s, 3H), 2.86-2.65 (m, 2H), 2.35 (dt, J=14.9, 7.3 Hz, 3H), 2.25-2.12 (m, 1H), 2.10-1.92 (m, 2H), 1.83 (d, J=5.1 Hz, 1H), 1.75-1.55 (m, 2H).

General Method G

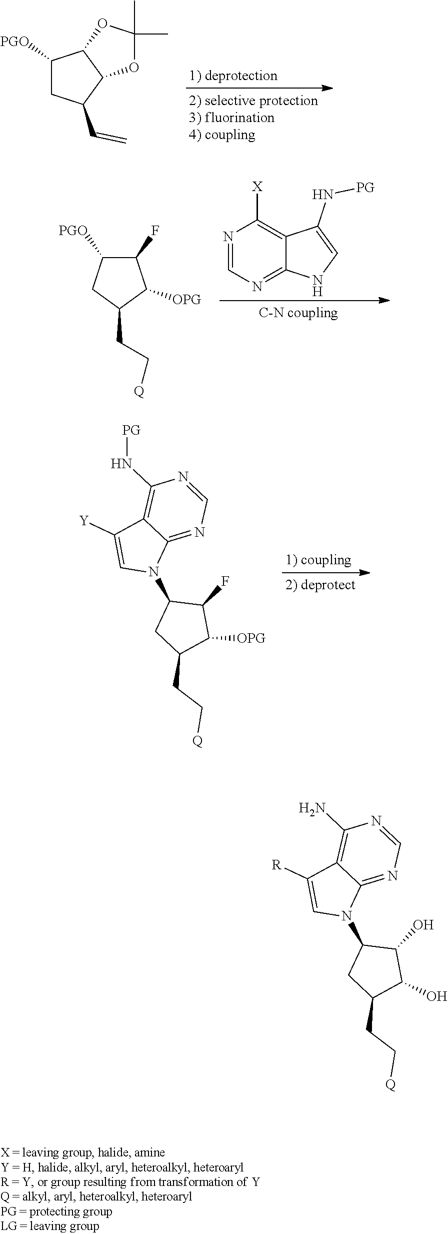

### Example 7: Synthesis of Compound I-212

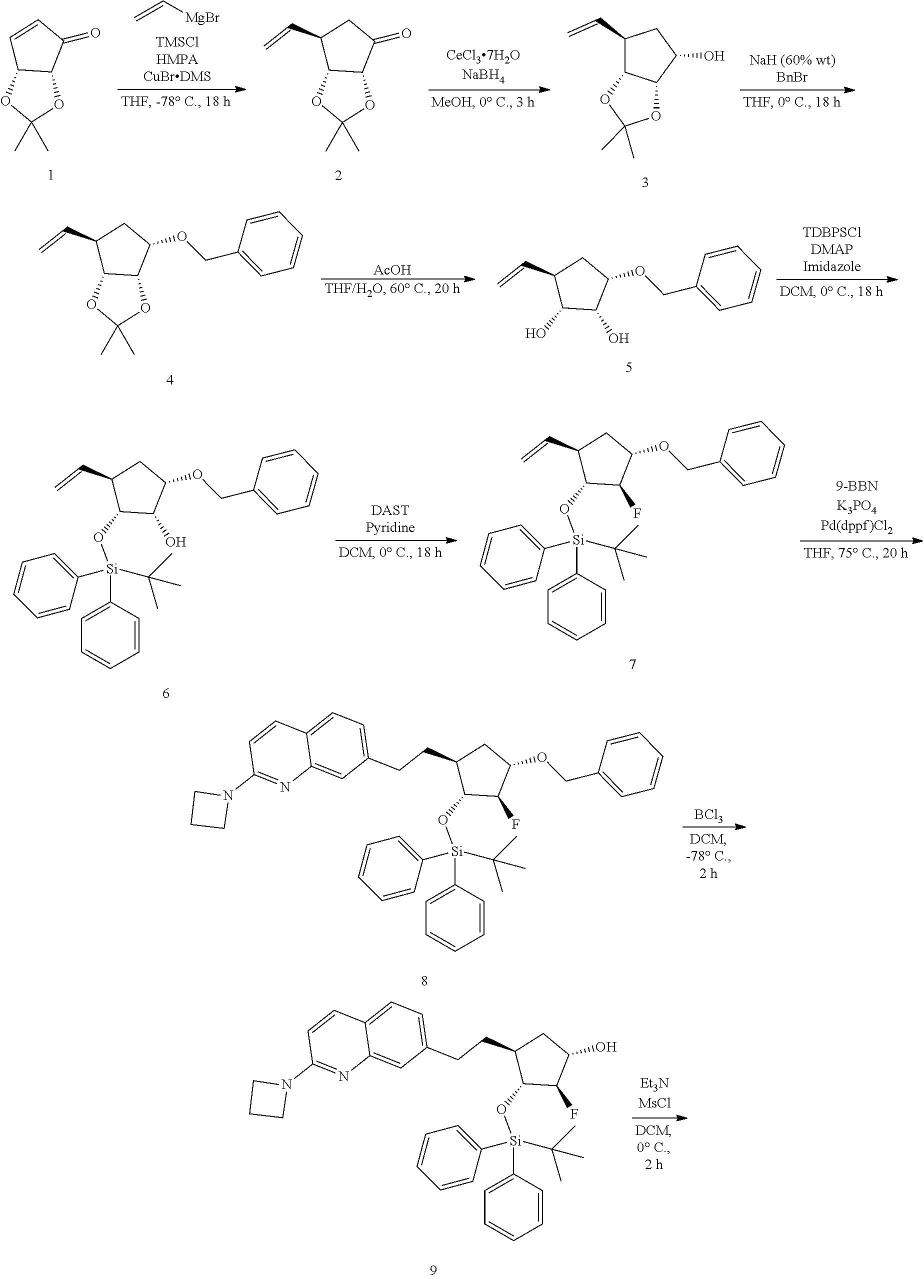

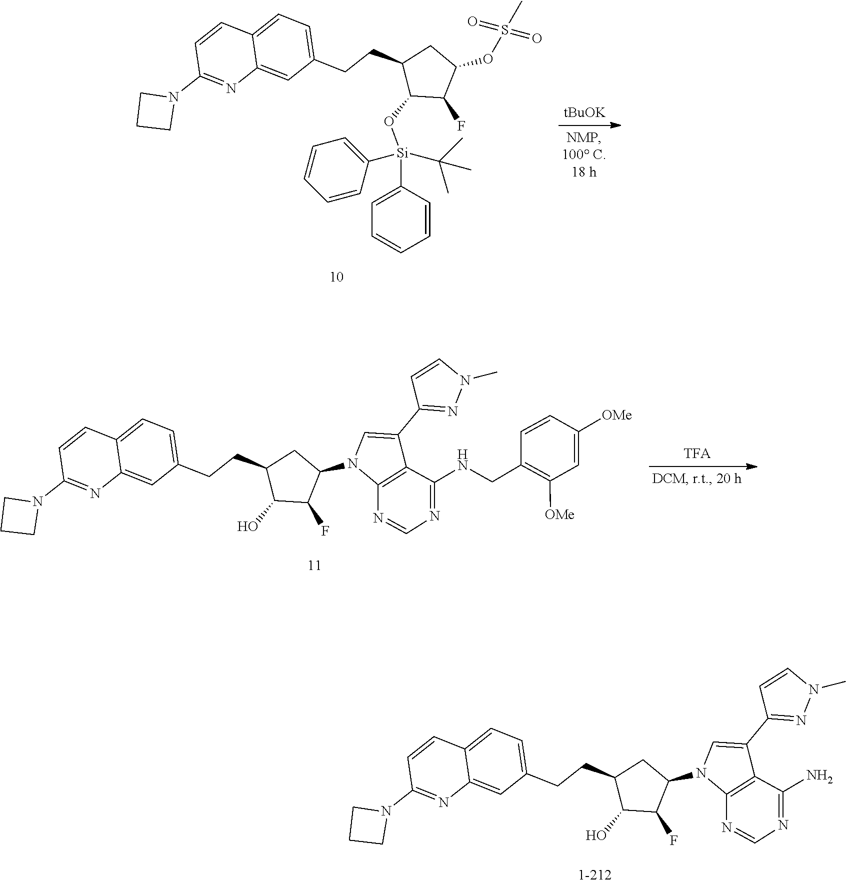 [SMILES: "tBuOK" ; CC(C)(C)[Si](O[C@@H]1[C@@H](CCc2ccc3ccc(N4CCC4)nc3c2)C[C@H](OS(C)(=O)=O)[C@H]1F)(c1ccccc1)c1ccccc1 ; COc1ccc(CNc2ncnc3c2c(-c2ccn(C)n2)cn3[C@@H]2C[C@H](CCc3ccc4ccc(N5CCC5)nc4c3)[C@@H](O)[C@@H]2F)c(OC)c1 ; Cn1ccc(-c2cn([C@@H]3C[C@H](CCc4ccc5ccc(N6CCC6)nc5c4)[C@@H](O)[C@@H]3F)c3ncnc(N)c23)n1]

### Synthesis of (3aR,6R,6aR)-6-Ethenyl-2,2-dimethyl-hexahydrocyclopenta[d][1,3]dioxol-4-one (2)

To a suspension of copper bromide dimethyl complex (571 mg, 2.78 mmol) in THF (60 mL) cooled at −78° C. with a dry-ice/acetone bath was added dropwise magnesium vinyl bromide (1M in THF) (36 mL, 36 mmol) over 10 mins. After 15 mins stirring at this temperature, a solution of(3aR,6aR)-2,2-dimethyl-2H,3aH,4H,6aH-cyclopenta[d][1,3]dioxol-4-one (4.3 g, 27.8 mmol), hexamethylphosphoramide (12.5 mL, 72.2 mmol) and chlorotrimethylsilane (7.05 mL, 55.6 mmol) in THF (15 mL) was added slowly over 35 mins while maintaining the temperature of the reaction mixture below −70° C. The reaction mixture was then allowed to reach room temperature over 18 h. After cooling with an ice-bath, sat. NH₄Cl (50 mL) was added and the phase separated. The aqueous layer was extracted with EtOAc (3×50 mL) and the combined organic layers were washed with water (50 mL) and brine (2×100 mL). The organic layer was dried over Na₂SO₄ and concentrated under vacuum. The crude product was purified by silica gel column chromatography (Heptane/EtOAc=0-40%) to afford (3aR,6R,6aR)-6-ethenyl-2,2-dimethyl-hexahydrocyclopenta[d][1,3]dioxol-4-one (4.2 g, 83% yield), as a light brown oil. ¹H NMR (400 MHz, CDCl₃) δ ppm 1.26 (s, 3H), 1.35 (s, 3H), 2.19 (d, J=18 Hz, 1H), 2.74 (dd, J=18.1, 8.7 Hz, 1H), 3.01 (t, J=7.1 Hz, 1H), 4.10 (d, J=5.3 Hz, 1H), 4.54 (d, J=5.3 Hz, 1H), 4.97-5.09 (m, 2H), 5.68-5.79 (m, 1H).

### Synthesis of (3aS,4S,6R,6aR)-6-Ethenyl-2,2-dimethyl-hexahydrocyclopenta[d][1,3]dioxol-4-ol (3)

To a solution of (3aR,6R,6aR)-6-ethenyl-2,2-dimethyl-hexahydrocyclopenta[d][1,3]dioxol-4-one (5.3 g, 29.0 mmol) in MeOH (80 mL) cooled with an ice-bath, was added cerium(III) chloride heptahydrate (11.8 g, 31.8 mmol) in 2 portions. After 15 min stirring, sodium borohydride (2.19 g, 58.0 mmol) was added portionwise. The reaction mixture was stirred at this temperature for 1.5 hour then allowed to reach room temperature, stirring was maintained for an extra 1.5 hour. After cooling with an ice-bath, the reaction mixture was quenched with slow addition of 2 N HCl (15 mL, pH=4-5). Solvent was partially removed under vacuum to reach 40 mL of aqueous MeOH. The later was extracted with Et₂O (3×150 mL). The combined organic layers were dried over Na₂SO₄ and concentrated under vacuum afford (3aS,4S,6R,6aR)-6-ethenyl-2,2-dimethyl-hexahydrocyclopenta[d][1,3]dioxol-4-ol (4.9 g, 91% yield) as a black oil. ¹H NMR (400 MHz, CDCl₃) δ ppm 1.35 (s, 3H), 1.51 (s, 3H), 1.86-1.91 (m, 1H), 2.70-2.77 (m, 1H), 4.02-4.11 (m, 1H), 4.47 (d, J=3.1 Hz, 2H), 5.03-5.11 (m, 2H), 5.69-5.80 (m, 1H).

### Synthesis of (3aS,4S,6R,6aR)-4-(Benzyloxy)-6-ethenyl-2,2-dimethyl-hexahydrocyclopenta[d][1,3]dioxol (4)

To a suspension of sodium hydride (60% wt) (766 mg, 19.2 mmol) in THF (92.1 mL) was added (3aS,4S,6R,6aR)-6-ethenyl-2,2-dimethyl-hexahydrocyclopenta[d][1,3]dioxol-4-ol (3.23 g, 17.5 mmol) dropwise at 0° C. After stirring at this temperature for 45 min, (bromomethyl)benzene (2.16 mL, 18.3 mmol) and sodium iodide (34.0 mg, 0.227 mmol) were added, and the reaction mixture was stirred at room temperature during 18 hours. The

[symbol]

### Synthesis of (1R,2R,3S,5R)-3-(benzyloxy)-2-[(tert-butyldiphenylsilyl)oxy]-5-ethenylcyclopentan-1-ol (6)

To a solution of (1R,2R,3S,5R)-3-(benzyloxy)-5-ethenylcyclopentane-1,2-diol (636.3 mg, 2.71 mmol), N,N-dimethylpyridin-4-amine (16.4 mg, 0.135 mmol), in dry DCM (5.4 mL) was added 1H-imidazole (184 mg, 2.71 mmol) and tert-butyl(chloro)diphenylsilane (819 mg, 2.98 mmol) at 0° C. The mixture was warmed to room temperature and stirred at this temperature for 18 hours. Water was added and the product was extracted with EtOAc (×3). The combined organic layers dried on Na₂SO₄, filtered and concentrated under vacuum. The residue was purified was purified by C₁₈ column chromatography (H2O/MeOH=0-100%) to afford (1R,2R,3S,5R)-3-(benzyloxy)-2-[(tert-butyldiphenylsilyl)oxy]-5-ethenylcyclopentan-1-ol (1.12 g, 87% yield, 8:2 mixture of regioisomers) as a colourless oil. ¹H NMR (400 MHz, CDCl₃) δ ppm 1.09 (s, 7.2H), 1.13 (s, 1.8H), 1.35 (ddd, J=13.8, 8.4, 5.1 Hz, 0.2H), 1.63 (dt, J=14.1, 7.2 Hz, 0.8H), 2.07-2.17 (m, 1H), 2.83 (s, 1.6H), 3.03-3.09 (m, 0.2H), 3.54-3.62 (m, 0.2H), 3.68-3.73 (m, 0.2H), 3.76-3.87 (m, 2.6H), 3.96 (dd, J=5.4, 3.9 Hz, 0.2H), 4.52-4.61 (m, 2H), 4.78-4.99 (m, 2H), 5.39 (ddd, J=17.4, 10.0, 7.8 Hz, 0.8H), 5.68 (ddd, J=17.4, 10.3, 7.3 Hz, 0.2H), 7.28-7.49 (m, 11H), 7.68-7.75 (m, 4H).

### Synthesis of {[(1R,2R,3S,5R)-3-(Benzyloxy)-5-ethenyl-2-fluorocyclopentyl]oxy}(tert-butyl)diphenylsilane (7)

To a solution of (1S,2R,3R,5S)-5-(benzyloxy)-2-[(tert-butyldiphenylsilyl)oxy]-3-ethenylcyclopentan-1-ol (385.6 mg, 0.814 mmol) in dry DCM (4.0 mL) was added pyridine (245 μL, 3.04 mmol) and (diethylamino)sulfur trifluoride (200 PL, 1.52 mmol) at 0° C. The resulting solution was warmed to room temperature under N₂ for 18 hours. The reaction was quenched with saturated aqueous Na₂CO₃ solution. The product was extracted with EtOAc (×3) and the combined organic layers dried over Na₂SO₄, filtered and concentrated under vacuum. The crude product was purified by silica gel column chromatography (Heptane/EtOAc=0-10%) to afford {[(1S,2S,3R,5S)-5-(benzyloxy)-3-ethenyl-2-fluorocyclopentyl]oxy}(tert-butyl)diphenylsilane (243 mg, 51% yield, 9:1 mixture of regioisomers) as a colourless oil. ESI LC-MS m/z=475.5 [M+H]⁺.

### Synthesis of 2-(Azetidin-1-yl)-7-{2-[(1S,2S,3S,4S)-4-(benzyloxy)-3-[(tert-butyldiphenylsilyl)oxy]-2-fluorocyclopentyl]ethyl}quinoline (8)

A solution of {[(1R,2R,3S,5R)-3-(benzyloxy)-5-ethenyl-2-fluorocyclopentyl]oxy}(tert-butyl)diphenylsilane (320 mg, 0.589 mmol, mixture of regioisomers) and 9-borabicyclo[3.3.1]nonane (0.5M in THF) (3.96 mL, 1.98 mmol) was stirred at 75° C. for 1 hour. Conversion was monitored by HPLC. After cooling to room temperature, to this solution were added tripotassium phosphate (430 mg, 2.02 mmol), 2-(azetidin-1-yl)-7-bromoquinoline (355 mg, 1.34 mmol), water (0.825 mL), THF (3.9 mL) and [1,1′-bis(diphenylphosphino)ferrocene]dichloropalladium(II) (49 mg, 66.9 μmol). The tube was sealed and mixture was stirred at 75° C. for 20 hours. Water was added and the product was extracted with AcOEt (×3). Combined organic layers were washed with saturated NaCl, dried with Na₂SO₄, filtered and reduced under vacuum. The crude product was purified by silica gel column chromatography (Heptane/EtOAc=0-40%) then by C₁₈ column chromatography (H₂O/MeOH=0-100%) to afford 2-(azetidin-1-yl)-7-{2-[(1S,2S,3S,4S)-4-(benzyloxy)-3-[(tert-butyldiphenylsilyl)oxy]-2-fluorocyclopentyl]ethyl}quinoline (333 mg, 77% yield) as a white solid. ESI LC-MS m/z=659.7 [M+H]⁺.

### Synthesis of (1S,2R,3R,4S)-4-{2-[2-(Azetidin-1-yl)quinolin-7-yl]ethyl}-3-[(tert-butyldiphenylsilyl)oxy]-2-fluorocyclopentan-1-ol (9)

To a solution of 2-(azetidin-1-yl)-7-{2-[(1S,2R,3R,4S)-4-(benzyloxy)-2-[(tert-butyldiphenylsilyl)oxy]-3-fluorocyclopentyl]ethyl}quinoline (218.8 mg, 0.330 mmol) in DCM (4.7 mL) cooled at −78° C. with a dry-ice/acetone bath was added slowly trichloroborane (1.65 mL, 1.65 mmol). The reaction mixture was stirred at this temperature for 2 hours. Then trichloroborane (0.8 mL, 800 μmol) was added and the mixture was warmed to −50° C. and stirred at this temperature for 45 min. Saturated aqueous NaHCO₃ was added and the product was extracted with DCM (×3). Combined organics layers were dried with Na₂SO₄, filtered and concentrated under reduced pressure. The crude product was purified by C₁₈ column chromatography (H2O/MeOH=0-100%) to afford (1S,2R,3R,4S)-4-{2-[2-(azetidin-1-yl)quinolin-7-yl]ethyl}-3-[(tert-butyldiphenylsilyl)oxy]-2-fluorocyclopentan-1-ol (152 mg, 71% yield) as a white solid. ESI LC-MS m/z=569.3 [M+H]⁺.

### Synthesis of (1S,2S,3R,4S)-4-{2-[2-(azetidin-1-yl)quinolin-7-yl]ethyl}-3-[(tert-butyldiphenylsilyl)oxy]-2-fluorocyclopentyl methanesulfonate (10)

At 0° C., triethylamine (57.4 mg, 0.568 mmol) and methanesulfonyl chloride (57.8 mg, 0.505 mmol) were added to a solution of (1S,2R,3R,4S)-4-{2-[2-(azetidin-1-yl)quinolin-7-yl]ethyl}-3-[(tert-butyldiphenylsilyl)oxy]-2-fluorocyclopentan-1-ol (180 mg, 0.316 mmol) in DCM (3.2 mL). The resulting mixture was stirred at 0° C. for 2 hours then at room temperature overnight. Saturated aqueous solution of NaHCO₃ was added and product was extracted with DCM (×3). Organic layers were combined, dried with Na₂SO₄, filtered and concentrated under vacuum to afford (1S,2S,3R,4S)-4-{2-[2-(azetidin-1-yl)quinolin-7-yl]ethyl}-3-[(tert-butyldiphenylsilyl)oxy]-2-fluorocyclopentyl methanesulfonate (205 mg, quantitative yield) as a white solid. ESI LC-MS m/z=647.3 [M+H]⁺.

### Synthesis of (1R,2R,3R,5S)-5-{2-[2-(Azetidin-1-yl)quinolin-7-yl]ethyl}-3-(4-{[(2,4-dimethoxyphenyl)methyl]amino}-5-(1-methyl-1H-pyrazol-3-yl)-7H-pyrrolo[2,3-d]pyrimidin-7-yl)-2-fluorocyclopentan-1-ol (11)

To a solution of (1S,2S,3R,4S)-4-{2-[2-(azetidin-1-yl)quinolin-7-yl]ethyl}-3-[(tert-butyldiphenylsilyl)oxy]-2-fluorocyclopentyl methanesulfonate (159 mg, 0.245 mmol), N-[(2,4-dimethoxyphenyl)methyl]-5-(1-methyl-1H-pyrazol3-yl)-7H-pyrrolo[2,3-d]pyrimidin-4-amine (178 mg, 0.490 mmol) in NMP (2.4 mL) was added potassium 2-methylpropan-2-olate (54.9 mg, 0.490 mmol) at room temperature. The reaction mixture was stirred at 100° C. for 18 hours. Water was added and the product was extracted with AcOEt (×4). The combined organic layers were dried on Na₂SO₄, filtered and concentrated under vacuum. The crude product was purified by C₁₈ column chromatography (H₂O+10 mM Ammonium bicarbonate/MeCN=0-100%) to afford (1R,2R,3R,5S)-5-{2-[2-(azetidin-1-yl)quinolin-7-yl]ethyl}-3-(4-{[(2,4-dimethoxyphenyl)methyl]amino}-5-(1-methyl-1H-pyrazol-3-yl)-7H-pyrrolo[2,3-d]pyrimidin-7-yl)-2-fluorocyclopentan-1-ol (71 mg, 43% yield) as a white solid. ESI LC-MS m/z=677.2 [M+H]⁺.

### Synthesis of (1R,2R,3R,5S)-3-[4-amino-5-(1-methyl-1H-pyrazol-3-yl)-7H-pyrrolo[2,3-d]pyrimidin-7-yl]-5-{2-[2-(azetidin-1-yl)quinolin-7-yl]ethyl}-2-fluorocyclopentan-1-ol (I-212)

To a solution of (1R,2R,3R,5S)-5-{2-[2-(azetidin-1-yl)quinolin-7-yl]ethyl}-3-(4-{[(2,4-dimethoxyphenyl)methyl]amino}-5-(1-methyl-1H-pyrazol-3-yl)-7H-pyrrolo[2,3-d]pyrimidin-7-yl)-2-fluorocyclopentan-1-ol (69 mg, 0.101 mmol) in DCE (3 mL) at room temperature was added the TFA (1.5 mL). The mixture was stirred at room temperature for 20 hours and solvents were removed under vacuum. The crude mixture was diluted with DCM and washed with aqueous solution of NaHCO₃. The product was extracted three times with DCM and one time with AcOEt. Combined organic layers were dried on Na₂SO₄, filtered and reduced under vacuum. The crude product was purified by C₁₈ column chromatography (H₂O+10 mM Ammonium bicarbonate/MeCN=0-100%). The pure fractions were combined and evaporated to dryness, coevaporated with H₂O (2×15 mL) and the residue was lyophilized in 1 mL of MeCN and 6 mL of H₂O to afford 1R,2R,3R,5S)-3-[4-amino-5-(1-methyl-1H-pyrazol-3-yl)-7H-pyrrolo[2,3-d]pyrimidin-7-yl]-5-{2-[2-(azetidin-1-yl)quinolin-7-yl]ethyl}-2-fluorocyclopentan-1-ol (45 mg, 84% yield) as a white solid. ¹H NMR (400 MHz, DMSO-d₆) δ ppm 1.73-1.94 (m, 2H), 1.99-2.21 (m, 2H), 2.30-2.43 (m, 3H), 2.71-2.92 (m, 2H), 3.84-3.95 (m, 4H), 4.08 (t, J=7.3 Hz, 4H), 4.68-4.92 (m, 1H), 5.05-5.29 (m, 1H), 5.45 (d, J=5.4 Hz, 1H), 6.63 (d, J=8.8 Hz, 1H), 6.69 (d, J=2.2 Hz, 1H), 7.14 (d, J=7.1 Hz, 2H), 7.45 (s, 1H), 7.62 (d, J=8.3 Hz, 1H), 7.73 (dd, J=14.2, 1.7 Hz, 2H), 7.97 (d, J=9.0 Hz, 1H), 8.06 (s, 1H), 9.01 (br. s, 1H). ¹⁹F NMR (377 MHz, DMSO-d₆) δ ppm-192.48 (s, 1 F). ESI-MS m/z calc. 526.26, found 527.3 [M+H]*, 264.2 [M+2H]2+H), 5.05-5.29 (m, 1H), 5.45 (d, J=5.4 Hz, 1H), 6.63 (d, J=8.8 Hz, 1H), 6.69 (d, J=2.2 Hz, 1H), 7.14 (d, J=7.1 Hz, 2H), 7.45 (s, 1H), 7.62 (d, J=8.3 Hz, 1H), 7.73 (dd, J=14.2, 1.7 Hz, 2H), 7.97 (d, J=9.0 Hz, 1H), 8.06 (s, 1H), 9.01 (br. s, 1H). ¹⁹F NMR (377 MHz, DMSO-d₆) δ ppm-192.48 (s, 1 F). ESI-MS m/z calc. 526.26, found 527.3 [M+H]*, 264.2 [M+2H]²+.

General Method H

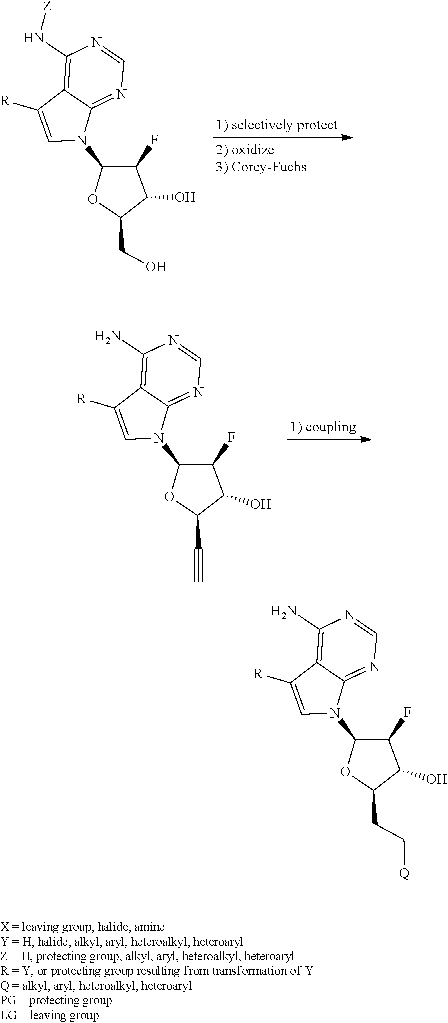

### Example 8: Synthesis of Compound I-191

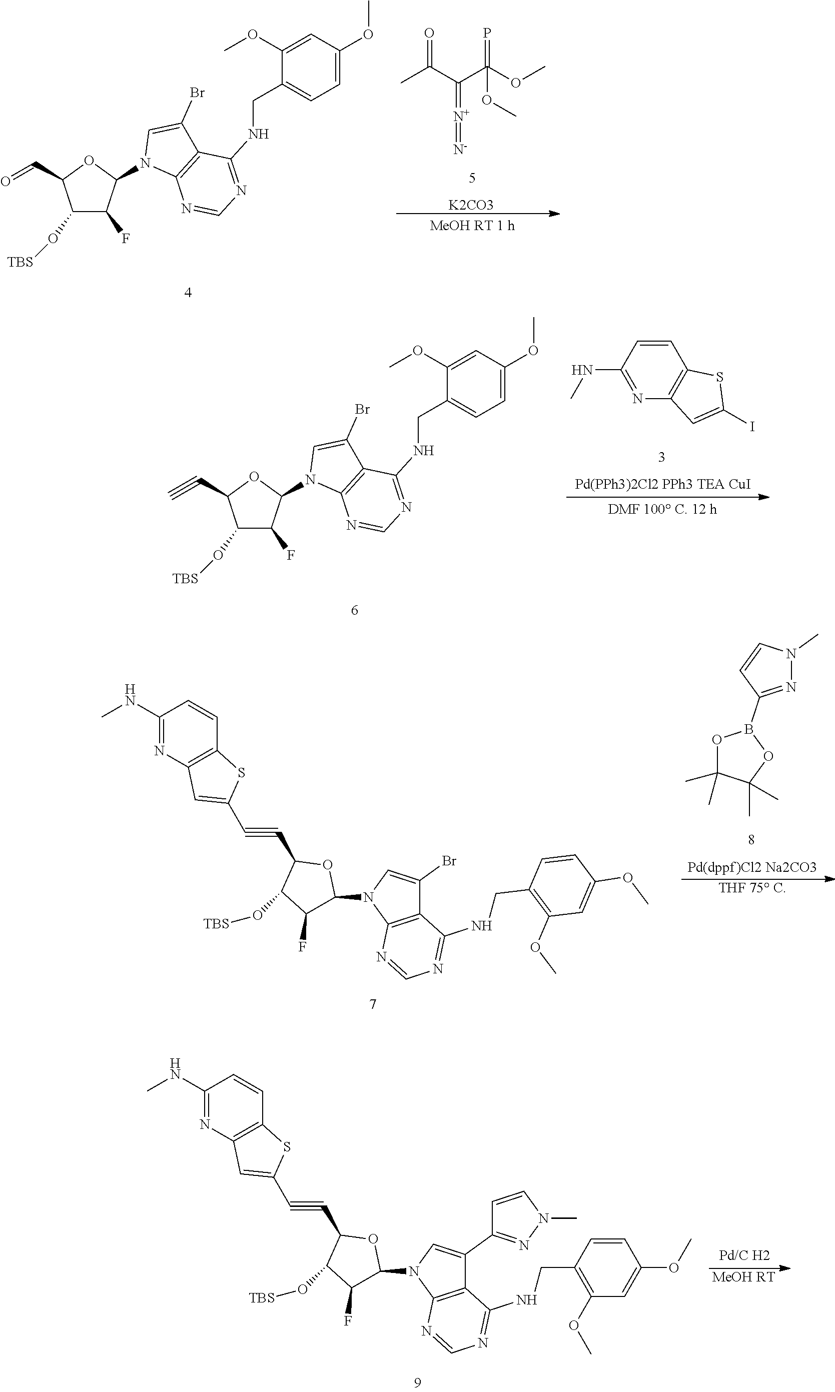

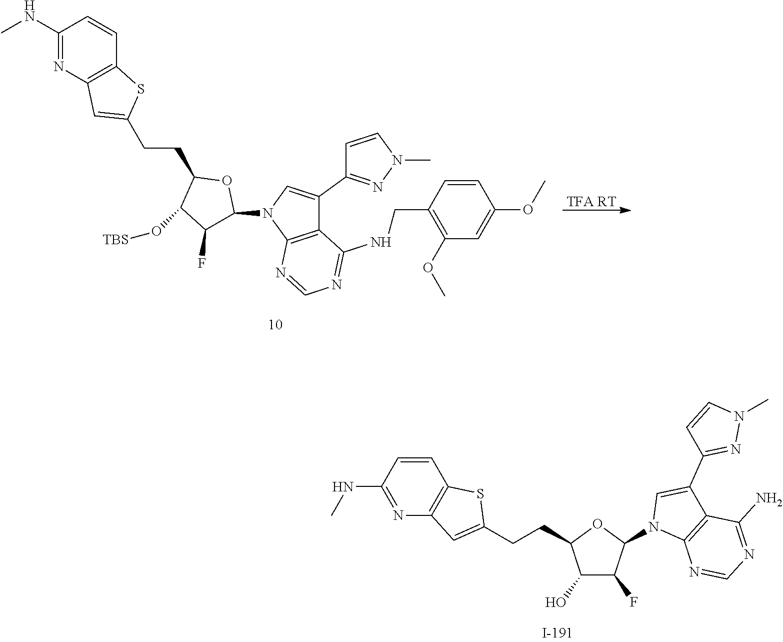

### Synthesis of Compound 6

2S,3R,4S,5R)-5-(5-bromo-4-{[(2,4-dimethoxyphenyl)methyl]amino}-7H-pyrrolo[2,3-d]pyrimidin-7-yl)-3-[(tert-butyldimethylsilyl)oxy]-4-fluorooxolane-2-carbaldehyde (400 mg, 656 μmol), dimethyl (1-diazo-2-oxopropyl)phosphonate (376 mg, 1.96 mmol), K2CO3 (270 mg, 1.96 mmol) were mixed in MeOH (10 mL). The mixture was stirred at rt for 1 h. LCMS showed the reaction was completed. Reaction solution was filtrated and concentrated under reduced pressure to get the crude. The crude was purified by SGC (EA/PE=0-40) to get 5-bromo-7-[(2R,3S,4R,5R)-4-[(tert-butyldimethylsilyl)oxy]-5-ethynyl-3-fluorooxolan-2-yl]-N-[(2,4-dimethoxyphenyl)methyl]-7H-pyrrolo[2,3-d]pyrimidin-4-amine (240 mg, 396 μmol) as a solid. ESI LCMS m/z=605.1[M+1]⁺

### Synthesis of Compound 7

5-bromo-7-[(2R,3S,4R,5R)-4-[(tert-butyldimethylsilyl)oxy]-5-ethynyl-3-fluorooxolan-2-yl]-N-[(2,4-dimethoxyphenyl)methyl]-7H-pyrrolo[2,3-d]pyrimidin-4-amine (200 mg, 330 μmol), triphenylphosphane (17.3 mg, 66.0 μmol), iodocopper (6.28 mg, 33.0 μmol), 2-iodo-N-methylthieno[3,2-b]pyridin-5-amine (114 mg, 396 μmol) TEA (133 mg, 1.32 mmol), palladium(2+) bis(triphenylphosphane) dichloride (23.1 mg, 33.0 μmol) were mixed in DMF (5 mL). The mixture was heated to 65° C. and stirred for 12 h under N2 atmosphere. LCMS showed the reaction was completed. The mixture was diluted with water (80 mL) and extracted with EA (80 mL×2). Combined organic layers were washed with H₂O (100 mL×2) and brine (60 mL), and dried (Na2SO4). The solvent was removed under reduced pressure to get crude. the crude was purified by flash chromatography (EA/PE: 0˜50%) to get 5-bromo-7-[(2R,3S,4R,5R)-4-[(tert-butyldimethylsilyl)oxy]-3-fluoro-5-{2-[5-(methylamino)thieno[3,2-b]pyridin-2-yl]ethynyl}oxolan-2-yl]-N-[(2,4-dimethoxyphenyl)methyl]-7H-pyrrolo[2,3-d]pyrimidin-4-amine (60.0 mg, 78.1 μmol) as a solid. ESI LCMS m/z=767.2[M+1]⁺

### Synthesis of Compound 9

1-methyl-3-(4,4,5,5-tetramethyl-1,3,2-dioxaborolan-2-yl)-1H-pyrazole (64.7 mg, 311 μmol), 5-bromo-7-[(2R,3S,4R,5R)-4-[(tert-butyldimethylsilyl)oxy]-3-fluoro-5-{2-[5-(methylamino)thieno[3,2-b]pyridin-2-yl]ethynyl}oxolan-2-yl]-N-[(2,4-dimethoxyphenyl)methyl]-7H-pyrrolo[2,3-d]pyrimidin-4-amine (200 mg, 260 μmol), palladium(2+) bis(triphenylphosphane) dichloride (18.2 mg, 26.0 μmol), Na₂CO3 (82.5 mg, 779 μmol) were mixed in THE (10 mL) and H₂O (2 mL). The mixture was heated to 70° C. and stirred for 12 h under N2 atmosphere. LCMS showed the reaction was completed. The mixture was diluted with water (80 ml) and extracted with EA (80 mL×2). Combined organic layers were washed with H₂O (100 mL×2) and brine (60 mL), and dried (Na₂SO₄). The solvent was removed under reduced pressure to get crude. the crude was purified by flash chromatography (EA˜ PE: 0˜100%) to get 7-[(2R,3S,4R,5R)-4-[(tert-butyldimethylsilyl)oxy]-3-fluoro-5-{2-[5-(methylamino)thieno[3,2-b]pyridin-2-yl]ethynyl}oxolan-2-yl]-N-[(2,4-dimethoxyphenyl)methyl]-5-(1-methyl-1H-pyrazol-3-yl)-7H-pyrrolo[2,3-d]pyrimidin-4-amine (68.0 mg, 88.4 μmol) as a solid. ESI LCMS m/z=769.3[M+1]⁺

### Synthesis of Compound 10

7-[(2R,3S,4R,5R)-4-[(tert-butyldimethylsilyl)oxy]-3-fluoro-5-{2-[5-(methylamino)thieno[3,2-b]pyridin-2-yl]ethynyl}oxolan-2-yl]-N-[(2,4-dimethoxyphenyl)methyl]-5-(1-methyl-1H-pyrazol-3-yl)-7H-pyrrolo[2,3-d]pyrimidin-4-amine (58.0 mg, 75.4 μmol), Pd/C (6.15 mg, 7.54 μmol) were mixed in MeOH (10 mL). the mixture was stirred at rt for 1 h under H2 atmosphere. LCMS showed the reaction was completed. Reaction solution was filtrated and concentrated under reduced pressure to get the crude 7-[(2R,3S,4R,5R)-4-[(tert-butyldimethylsilyl)oxy]-3-fluoro-5-{2-[5-(methylamino)thieno[3,2-b]pyridin-2-yl]ethyl}oxolan-2-yl]-N-[(2,4-dimethoxyphenyl)methyl]-5-(1-methyl-1H-pyrazol-3-yl)-7H-pyrrolo[2,3-d]pyrimidin-4-amine (55.0 mg, 71.1 μmol). The crude was directly used to next step without further purification. ESI LCMS m/z=773.3[M+1]⁺

### Synthesis of Compound I-191

7-[(2R,3S,4R,5R)-4-[(tert-butyldimethylsilyl)oxy]-3-fluoro-5-{2-[5-(methylamino)thieno[3,2-b]pyridin-2-yl]ethyl}oxolan-2-yl]-N-[(2,4-dimethoxyphenyl)methyl]-5-(1-methyl-1H-pyrazol-3-yl)-7H-pyrrolo[2,3-d]pyrimidin-4-amine (55.0 mg, 71.1 μmol) was added to TFA (5 mL) and H₂O (1 mL). The mixture was stirred at rt for 4 h. LCMS showed the reaction was completed. Then the mixture was concentrated to get crude, neutralized by 4 mL 7M NH₃ in MeOH. The product was purified by Prep-HPLC to get (2R,3R,4S,5R)-5-[4-amino-5-(1-methyl-1H-pyrazol-3-yl)-7H-pyrrolo[2,3-d]pyrimidin-7-yl]-4-fluoro-2-{2-[5-(methylamino)thieno[3,2-b]pyridin-2-yl]ethyl}oxolan-3-ol (2.30 mg, 4.52 μmol) as a white solid. ESILC-MS m/z=508.9[M+H]⁺. 1H NMR (400 MHz, CD30D) δ 8.10 (s, 1H), 7.80 (d, J=8.8 Hz, 1H), 7.62-7.53 (m, 2H), 7.08 (s, 1H), 6.65-6.57 (m, 1H), 6.56 (d, J=2.3 Hz, 1H), 6.50 (d, J=8.9 Hz, 1H), 5.12-4.94 (m, 1H), 4.36-4.24 (m, 1H), 4.02-3.96 (m, 1H), 3.95 (s, 3H), 3.25-3.05 (m, 2H), 2.93 (s, 3H), 2.35-2.20 (m, 2H).

### Building Block Synthesis

### Example 9: General Procedure for Preparation of Building Block 1

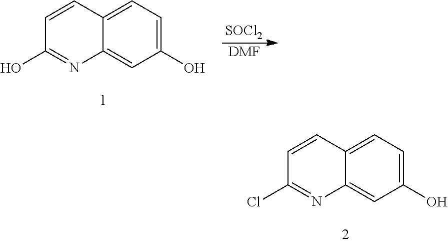

To a solution of compound 1 (120 g, 745 mmol, 1.00 eq) in DMF (1.20 L) was added SOCl₂ (354 g, 2.98 mol, 216 mL, 4.00 eq) at 0° C. The mixture was stirred at 25° C. for 0.5 hr and then stirred at 70° C. for 2 hrs. TLC(Petroleum ether:Ethyl acetate=2:1) showed compound 1 (Rf=0.3) was consumed and new spot(Rf=0.5) was detected. The mixture was poured into the water (5.00 L), adjusted to pH=8 with Na₂CO₃(sat), filtered and the yellow solid was collected. Compound 2 (120 g, 664.8 mmol, 89.2% yield, 99.5% purity) was obtained as yellow solid which was checked by LCMS; LCMS: RT=0.604 min, MS+1=179.8.

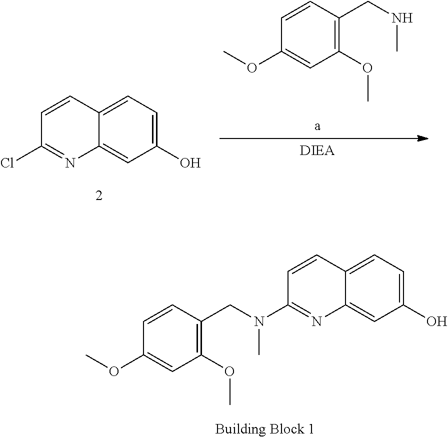

A mixture of compound 2 (150 g, 835 mmol, 1.00 eq), compound a (151 g, 835 mmol, 1.00 eq) and DIEA (324 g, 2.51 mol, 436 mL, 3.00 eq) was stirred at 120° C. for 12 hrs. LCMS (EW20099-8-P1A) showed compound 2 was consumed and desired mass was detected. The mixture was dissolved with MeOH (200 mL), then water (100 mL) and EtOAc (100 mL) were added to the mixture and stirred at 25° C. for 0.5 hrs, then filtered and off-white solid was collected. The crude product was triturated with EtOAc (100 mL) at 25° C. for 1 hr. Building Block 1 (105 g, 311 mmol, 37.2% yield, and 96.0% purity) was obtained as white solid. LCMS: RT=0.785, MS+1=325.0.

Compounds in Table 1 were prepared according to general methods A-H shown above using experimental procedures similar to those described in the above Examples MS and ¹H NMR data are shown below.

TABLE 1

| | Syn- | | |
|---|---|---|---|
| | the- | MS | |
| | tic | (ESI) | |
| | Me- | [M + | |
| Compound No. | thod | H]⁺ | ¹H NMR |
| 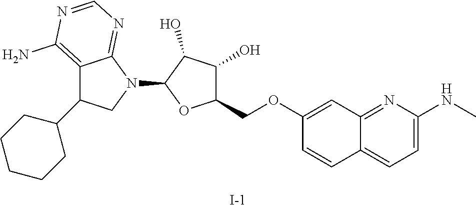 [SMILES: CNc1ccc2ccc(OC[C@H]3O[C@@H](N4CC(C5CCCCC5)c5c(N)ncnc54)[C@H](O)[C@@H]3O)cc2n1 ; [I-]] | A | 505.3 | ¹H NMR (400 MHz, DMSO- d6) δ 8.04 (s, 1H), 7.74 (d, J = 8.9 Hz, 1H), 7.52 (d, J = 8.7 Hz, 1H), 7.19-6.99 (m, 2H), 6.95-6.87 (m, 1H), 6.83 (dd, J = 8.7, 2.5 Hz, 1H), 6.57 (d, J = 8.8 Hz, 1H), 6.44 (s, 2H), 6.17 (d, J = 5.8 Hz, 1H), 5.35 (d, J = 6.3 Hz, 1H), 5.30 (d, J = 5.0 Hz, 1H), 4.52-4.42 (m 1H), 4.39-4.28 (m, 1H), 4.27- 4.16 (m, 3H), 2.88 (d, J = 8.0 Hz, 4H), 1.96 (d, J = 12.6 Hz, 1H), 1.84 (d, J = 13.1 Hz, 1H), |
| | | | 1.76-1.56 (m, 3H), 1.54-1.36 |
| | | | (m, 2H), 1.30-1.02 (m, 3H). |
| 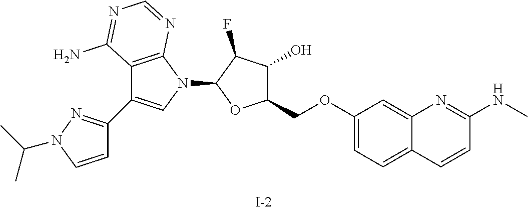 | B | 533.0 | ¹H NMR (400 MHz, DMSO- d₆) δ ppm 9.16 (s, 1H), 8.09 (s, 1H), 7.79 (d, J = 1.6 Hz, 1H), 7.74 (d, J = 8.8 Hz, 1H), 7.84 (d, J = 2.4 Hz, 1H), 7.52 (d, J = 8 Hz, 1H), 7.28 (s, 1H), 7.06 (d, J = 2.4 Hz, 1H), 6.95-6.91 (m, 1H), 6.84-6.81 (m, 1H), 6.72-6.67 (m, 1H), 6.61-6.57 (m, 1H), 6.13 (d, J = 4.4 Hz, 1H), 5.30-5.14 (m, 1H), 4.60- 4.49 (m, 1H), 4.46-4.42 (m, 1H), 4.38-4.34 (m, 1H), 4.22- 4.18 (m, 1H), 2.88 (d, J = 4.8 |
| | | | Hz, 3H), 1.45 (d, J = 6 Hz, 6H) |
| 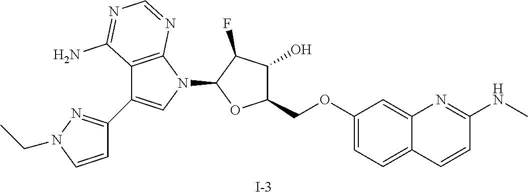 | B | 520.2 | ¹H NMR (400 MHz, DMSO- d₆) δ ppm 9.11 (s, 1H), 8.09 (s, 1H), 7.76 (d, J = 2.4 Hz, 1H), 7.74 (d, J = 8.8 Hz, 1H), 7.85 (d, J = 2.4 Hz 1H), 7.52 (d, J = 8 Hz, 1H), 7.26 (s, 1H), 7.06 (d, J = 2.4 Hz, 1H), 6.95-6.91 (m, 1H), 6.84-6.81 (m, 1H), 6.72-6.67 (m, 1H), 6.61 (d, J = 4 Hz, 1H), 6.58 (d, J = 8.8 Hz, 2H), 6.13 (d, J = 5.2 Hz, 1H), 5.30-5.15 (m, 1H), 4.46-4.51 (m, 1H), 4.46-4.33 (m, 2H), 4.21-4.17 (m, 2H), 4.15 (d, J = |
| | | | 8.8 Hz, 1H), 2.88 (d, J = 8.8 |
| | | | Hz, 3H), 1.05 (t, J = 7.2 Hz, |
| | | | 3H) |
| 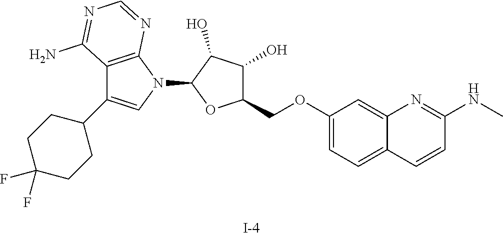 | A | 541.2 | ¹H NMR (400 MHz, DMSO- d₆) δ ppm 8.06 (s, 1H), 7.74 (d, J = 8.8 Hz, 1H), 7.51 (d, J = 8.8 Hz, 1H), 7.15 (s, 1H), 7.01 (d, J = 2.4 Hz, 1H), 6.92 (d, J = 4.8 Hz, 1H), 6.81 (dd, J = 8.7, 2.5 Hz, 1H), 6.63 (s, 2H), 6.57 (d, J = 8.8 Hz, 1H), 6.17 (d, J = 5.8 Hz, 1H), 5.37 (d, J = 6.3 Hz, 1H), 5.31 (d, J = 5.0 Hz, 1H), 4.50-4.45 (m, 1H), 4.34-4.31 (m, 3H), 3.16-3.10 (m, 1H), 2.88 (d, J = 4.7 Hz, 3H), 2.14-2.00 (m, 6H), 1.56- 1.46 (m, 2H) |
| 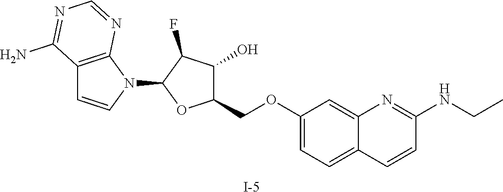 | B | 439.1 | ¹H NMR (400 MHz, DMSO- d₆) δ 4.50-4.56 (m, 1H), 4.34- 4.39 (m, 1H), 4.27-4.31 (m, 1H), 4.17 (dd, J = 9.2, 5.6 Hz, 1H), 3.35-3.43 (m, 2H), 1.17 (t, J = 7.2 Hz, 3H) |
| 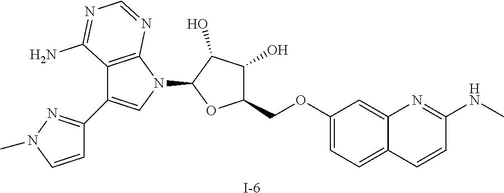 | A | 503.1 | ¹H NMR (400 MHz, DMSO- d₆) δ 9.00 (s, 1H), 8.08 (s, 1H), 7.81 (s, 1H), 7.79-7.69 (m, 2H), 7.52 (d, J = 8.5 Hz, 1H), 7.40- 7.10 (m, 1H), 7.10-7.00 (m, 1H), 7.00-6.90 (m, 1H), 6.90- 6.80 (m, 1H), 6.75-6.50 (m, 2H), 6.22 (d, J = 5.9 Hz, 1H), 5.48 (d, J = 6.3 Hz, 1H), 5.39 (d, J = 5.1 Hz, 1H), 4.66-4.50 (m, 1H), 4.42-4.22 (m, 4H), 3.85 (s, 3H), 2.89 (d, J = 4.6 Hz, 3H). |
| 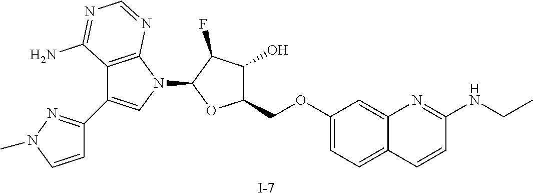 | B | 519.1 | ¹H NMR (400 MHz, DMSO- d₆) δ ppm 9.04 (s, 1H), 8.09 (s, 1H), 7.73 (d, J = 8.9 Hz, 1H), 7.71 (d, J = 2.3 Hz, 1H), 7.65 (d, J = 2.2 Hz, 1H), 7.50 (d, J = 8.8 Hz, 1H), 7.27 (s, 1H), 7.03 (d, J = 2.4 Hz, 1H), 6.93 (t, J = 5.3 Hz, 1H), 6.82 (dd, J = 8.7, 2.5 Hz, 1H), 6.70 (dd, J = 15.8. 4.5 Hz, 1H), 6.62 (d, J = 2.3 Hz, 1H), 6.58 (d, J = 8.9 Hz, 1H), 6.14 (d, J = 4.9 Hz, 1H), 5.32-5.14 (m, 1H), 4.63-4.52 (m, 1H), 4.45-4.41 (m, 1H), |
| | | | 4.37-4.33 (m, 1H), 4.21-4.18 |
| | | | (m, 1H), 3.88 (s, 3H), 3.43-3.36 |
| | | | (m, 2H), 1.18 (t, J = 7.2 Hz, |
| | | | 1H). |
| 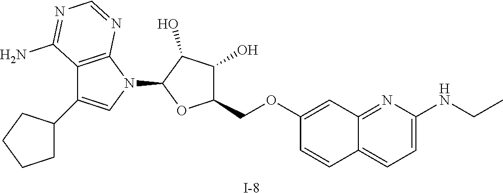 | A | 505.3 | ¹H NMR (400 MHz, DMSO- d₆) δ ppm ¹H 8.04 (s, 1H), 7.73 (d, J = 8.9 Hz, 1H), 7.51 (d, J = 8.7 Hz, 1H), 7.09 (s, 1H), 6.98 (d, J = 2.4 Hz, 1H), 6.91 (t, J = 5.2 Hz, 1H), 6.81 (dd, J = 8.7, 2.5 Hz, 1H), 6.57 (d, J = 8.9 Hz, 1H), 6.51 (s, 2H), 6.17 (d, J = 6.0 Hz, 1H), 5.36 (d, J = 6.4 Hz, 1H), 5.30 (d, J = 4.9 Hz, 1H), 4.50-4.45 (m, 1H), 4.32-4.31 (m, 1H), 4.27-4.16 (m, 3H), 3.43-3.36 (m, 3H), 2.02-1.90 (m, 2H), 1.66-1.55 |
| | | | (m, 4H), 1.51-1.36 (m, 2H), |
| | | | 1.20-1.16 (m, 3H). |
| 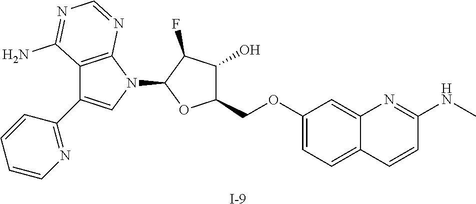 | B | 502.1 | ¹H NMR (400 MHz, CD3OD) δ ppm 8.49-8.48 (M, 1H), 8.09 (s, 1H), 7.83 (d, J = 9.0 Hz, 1H), 7.80 (d, J = 2.0 Hz, 1H), 7.60-7.56 (m, 2H), 7.48 (d, J = 8.2 Hz, 1H), 7.30 (d, J = 2.4 Hz, 1H), 7.17-7.14 (m, 1H), 7.03-7.00 (m, 1H), 6.74-6.69 (m, 1H), 6.64 (d, J = 9.0 Hz, 1H), 5.28-5.09 (m, 1H), 4.75- 4.66 (m, 1H), 4.57-4.54 (m, 1H), 4.47-4.43 (m, 1H), 4.39- 4.36 (m, 1H), 3.00 (s, 3H). |
| 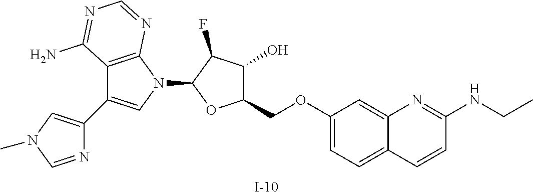 | B | 519.3 | ¹H NMR (400 MHz, DMSO- d₆) δ 9.84 (s, 1H), 8.06 (s, 1H), 7.81-7.67 (m, 2H), 7.52 (d, J = 8.7 Hz, 1H), 7.49 (d, J = 2.2 Hz, 1H), 7.42 (s, 1H), 7.16 (s, 1H), 7.03 (d, J = 2.3 Hz, 1H), 6.93 (t, J = 5.3 Hz, 1H), 6.83 (dd, J = 8.7, 2.4 Hz, 1H), 6.69 (dd, J = 16.2, 4.4 Hz, 1H), 6.58 (d, J = 8.8 Hz, 1H), 6.13 (d, J = 4.7 Hz, 1H), 5.31-5.08 (m, 1H), 4.58-4.53 (m, 1H), 4.44-4.41 (m, 1H), 4.34-4.30 (m, 1H), 4.22-4.18 (m, 1H), 3.66 (s, 3H), |
| | | | 3.49-3.35 (m, 2H), 1.18 (t, J = |
| | | | 7.2 Hz, 3H). |
| 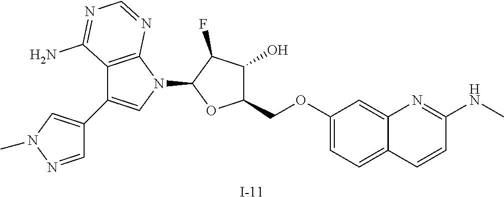 | B | 505.1 | ¹H NMR (400 MHz, DMSO- d₆) δ ppm 8.14 (s, 1H), 7.81 (s, 1H), 7.73 (d, J = 8.8 Hz, 1H), 7.53 (s, 1H), 7.49 (d, J = 8.8 Hz, 1H), 7.28 (d, J = 2.0 Hz, 1H), 7.02 (s, 1H), 6.92-6.91 (m, 1H), 6.77 (dd, J = 8.8, 2.8 Hz, 1H), 6.70 (dd, J = 14.8, 4.4 Hz, 1H), 6.56 (d, J = 8.8 Hz, 1H), 6.25 (br, 1H), 6.11 (d, J = 4.8 Hz, 1H), 5.30-5.17 (m, 1H), 4.57-4.53 (m, 1H), 4.38-4.31 (m, 2H), 4.18-4.17 (m, 1H), 3.87 (s, 3H), 2.87 (d, J = 8.8 |
| | | | Hz, 3H) |
| 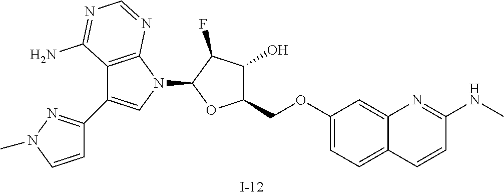 | B | 505.0 | ¹H NMR (400 MHz, DMSO- d₆) δ ppm 9.03 (s, 1H), 8.09 (s, 1H), 7.76-7.71 (m, 2H), 7.65 (d, J = 2.2 Hz, 1H), 7.52 (d, J = 8.4 Hz, 1H), 7.23 (s, 1H), 7.06 (d, J = 2.3 Hz, 1H), 6.94 (s, 1H), 6.83 (dd, J = 8.7, 2.5 Hz, 1H), 6.70 (dd, J = 15.9, 4.5 Hz, 1H), 6.61 (d, J = 2.3 Hz, 1H), 6.58 (d, J = 8.9 Hz, 1H), 6.13 (d, J = 4.9 Hz, 1H), 5.30-5.25 (m, 1H), 5.17-5.12 (m, 1H), 4.64-4.51 (m, 1H), 4.46-4.42 (m, 1H), 4.38-4.34 (m, 1H), |
| | | | 4.22-4.18 (m, 1H), 3.88 (s, |
| | | | 3H), 2.88 (d, J = 4.7 Hz, 3H). |
| 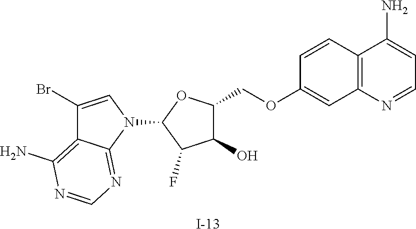 | B | 488.9 | ¹H NMR (400 MHz, DMSO- d₆) δ ppm 8.29-8.21 (m, 2H), 8.17-8.05 (m, 2H), 7.54 (d, J = 2.1 Hz, 1H), 7.26 (d, J = 2.5 Hz, 1H), 7.11 (dd, J = 9.2, 2.6 Hz, 1H), 7.00 (s, 2H), 6.68 (dd, J = 13.9, 4.8 Hz, 1H), 6.46 (d, J = 5.5 Hz, 1H), 5.34-5.30 (m, 1H), 5.20-5.17 (m, 1H), 4.60- 4.52 (m, 1H), 4.44-4.35 (m, 2H), 4.21-4.17 (m, 1H). |
| 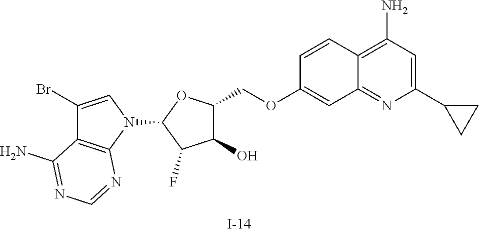 | B | 529.1 | ¹H NMR (400 MHz, DMSO) δ 8.14 (s, 1H), 7.99 (d, J = 9.0 Hz, 1H), 7.52 (d, J = 1.9 Hz, 1H), 6.98 (d, J = 2.5 Hz, 1H), 6.92-6.89 (m, 1H), 6.69-6.64 (m, 1H), 6.28 (s, 2H), 6.15 (d, J = 4.9 Hz, 1H), 5.24 (dt, J = 52.8, 4.4 Hz, 1H), 4.58-4.52 (m, 1H), 4.39-4.30 (m, 2H), 4.22-4.14 (m, 1H), 2.36-2.26 (m, 1H), 1.09-1.00 (m, 2H), 0.72-0.59 (m, 2H). |
| 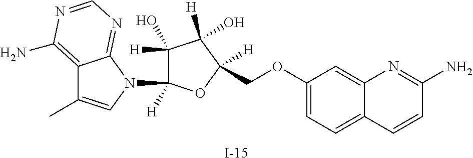 | A | 423.1 | ¹H NMR (400 MHz, CD3OD) δ ppm 8.04 (s, 1H), 7.85 (d, J = 8.8 Hz, 1H), 7.55 (d, J = 8.8 Hz, 1H), 7.28 (d, J = 0.9 Hz, 1H), 7.02 (d, J = 2.3 Hz, 1H), 6.98-6.95 (m, 1H), 6.68 (d, J = 8.8 Hz, 1H), 6.55 (d, J = 4.4 Hz, 1H), 4.60-4.53 (m, 1H), 4.53-4.46 (m, 2H), 4.39-4.31 (m, 1H), 4.28-4.20 (m, 1H), 2.44 (s, 3H). |
| 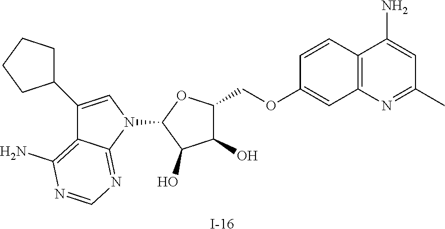 | A | 491.2 | ¹H NMR (400 MHz, DMSO) δ ppm 8.04 (s, 1H), 8.01 (d, J = 8.6 Hz, 1H), 7.13 (d, J = 2.5 Hz, 1H), 7.07 (s, 1H), 6.98 (dd, J = 9.1, 2.4 Hz, 1H), 6.64- 6.49 (m, 4H), 6.30 (s, 1H), 6.16 (d, J = 5.9 Hz, 1H), 5.37 (d, J = 6.3 Hz, 1H), 5.33 (d, J = 4.9 Hz, 1H), 4.50-4.45 (m, 1H), 4.35-4.31 (m, 1H), 4.27-4.17 (m, 3H), 3.31 (s, 1H), 2.36 (s, 3H), 2.03-1.91 (m, 2H), 1.69- 1.56 (m, 4H), 1.48-1.37 (m, 2H). |
| 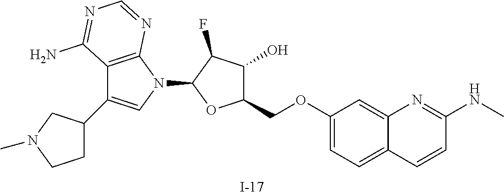 | B | 508.7 | δ ¹H NMR (400 MHz, CD3OD) δ ppm 8.02 (s, 1H), 7.73 (d, J = 8.9 Hz, 1H), 7.50 (d, J = 8.7 Hz, 1H), 7.20 (s, 1H), 6.96-6.95 (m, 1H), 6.93- 6.87 (m, 1H), 6.64-6.57 (m, 2H), 5.14-5.12 (m, 1H), 5.01- 4.99 (m, 1H), 4.67-4.57 (m, 1H), 4.44-4.41 (m, 1H), 4.37- 4.31 (m, 1H), 4.28-4.24 (m, 1H), 3.45-3.37 (m, 1H), 3.06- 2.95 (m, 4H), 2.85-2.75 (m, 1H), 2.55-2.46 (m, 1H), 2.33- |
| | | | 2.32 (m, 3H), 2.27-2.19 (m, |
| | | | 2H), 1.86-1.76 (m, 1H). |
| 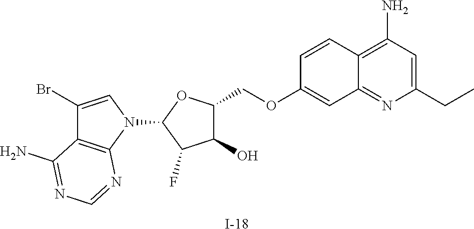 | B | 518.0 | ¹H NMR (400 MHz, CD3OD) δ ppm 8.36 (s, 1H), 8.22 (d, J = 9.3 Hz, 1H), 8.13 (s, 1H), 7.35- 7.32 (m, 1H), 7.25 (d, J = 2.4 Hz, 1H), 7.14 (d, J = 2.4 Hz, 1H), 6.70-6.65 (m, 1H), 6.59 (s, 1H), 5.26-5.07 (m, 1H), 4.74- 4.65 (m, 1H), 4.63-4.59 (m, 1H), 4.50-4.47 (m, 1H), 4.37- 4.27 (m, 1H), 2.88 (q, J = 7.6 Hz, 2H), 1.41 (t, J = 7.6 Hz, 3H). |
| 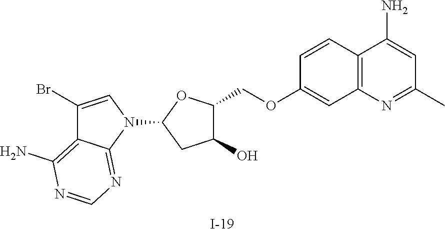 | C | 487.0 | ¹H NMR (400 MHz, DMSO- d6) δ 8.12 (s, 1H), 8.00 (d, J = 9.2 Hz, 1H), 7.67 (s, 1H), 7.14 (d, J = 2.8 Hz, 1H), 6.98 (dd, J = 8.8, 2.4 Hz, 1H), 6.80 (brs, 1H), 6.63 (t, J = 7.0 Hz, 1H), 6.56 (s, 2H), 6.30 (s, 1H), 5.52 (d, J = 4.4 Hz, 1H), 4.51-4.50 (m, 1H), 4.29-4.25 (m, 1H), 4.21-4.15 (m, 2H), 2.68-2.60 (m, 1H), 2.37 (s, 3H), 2.32-2.26 (m, 1H). |
| 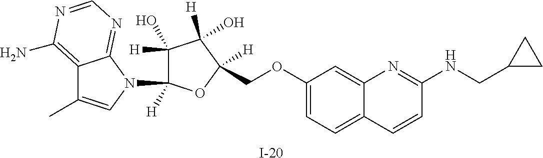 | A | 477.1 | ¹H NMR (400 MHz, CD3OD) δ ppm 8.00 (s, 1H), 7.73 (d, J = 8.9 Hz, 1H), 7.49 (d, J = 8.8 Hz, 1H), 7.22 (s, 1H), 7.04 (t, J = 5.3 Hz, 1H), 6.94 (d, J = 2.1 Hz, 1H), 6.78 (dd, J = 8.7, 2.4 Hz, 1H), 6.69-6.42 (m, 4H), 5.45 (dd, J = 38.3, 4.6 Hz, 2H), 4.48-4.00 (m, 5H), 3.30-3.18 (m, 2H), 2.35 (s, 3H), 1.20-1.01 (m, 1H), 0.54-0.35 (m, 2H), 0.35-0.14 (m, 2H). |
| 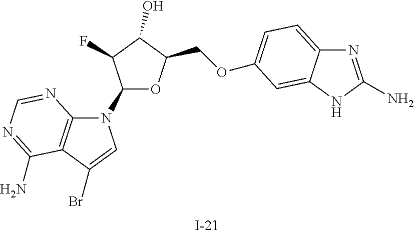 | B | 479 | ¹H NMR (400 MHz, DMSO-d) δ 8.14 (s, 1H), 7.54 (s, 1H), 6.89-6.79 (m, 3H), 6.65 (dd, J = 12.8, 4.4 Hz, 1H), 6.56- 6.54 (m, 1H), 6.16-6.07 (m, 2H), 5.28 (dt, J = 52.8, 4.4 Hz, 1H), 5.55-4.48 (m, 1H), 4.23- 4.10 (m, 3H) |
| 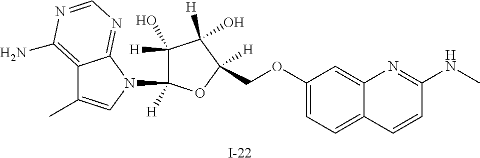 | A | 437.1 | ¹H NMR (400 MHz, CD3OD) δ ppm 8.05 (s, 1H), 7.76 (d, J = 8.9 Hz, 1H), 7.52 (d, J = 8.8 Hz, 1H), 7.29 (d, J = 1.1 Hz, 1H), 7.18 (d, J = 2.4 Hz, 1H), 6.94 (dd, J = 8.7, 2.5 Hz, 1H), 6.60 (d, J = 8.9 Hz, 1H), 6.55 (d, J = 4.5 Hz, 1H), 4.59- 4.55 (m, 1H), 4.51 (dd, J = 9.7, 5.4 Hz, 2H), 4.35 (d, J = 2.7 Hz, 1H), 4.28-4.22 (m, 1H), 3.01 (s, 3H), 2.45 (d, J = 1.1 |
| | | | Hz, 3H). |
| 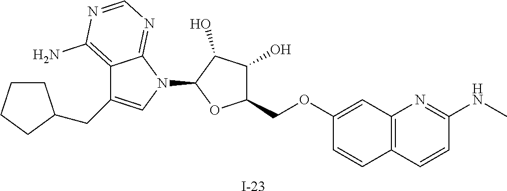 | A | 505.3 | ¹H NMR (400 MHz, DMSO- d6) δ 8.07 (s, 1H), 7.79 (d, J = 9.1 Hz, 1H), 7.56 (d, J = 8.7 Hz, 1H), 7.23 (s, 1H), 7.10 (s, 1H), 6.96 (d, J = 11.1 Hz, 1H), 6.62 (d, J = 8.9 Hz, 1H), 6.31 (d, J = 5.7 Hz, 1H), 4.61-4.58 (m, 1H), 4.51-4.41 (m, 3H), 4.34-4.31 (m, 1H), 3.02 (s, 3H), 2.72-2.69 (m, 1H), 2.12-2.04 (m, 1H), 1.71-1.66 (m, 2H), 1.53-1.50 (m, 2H), 1.43-1.31 (m, 3H), 1.8-1.12 (m, 2H). |
| 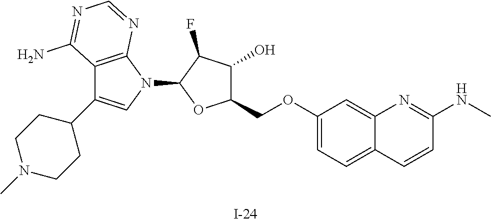 | B | 522.2 | ¹H NMR (400 MHz, DMSO- d6) δ 8.21 (s, 1.64H), 8.08 (s, 1H), 7.74 (d, J = 8.8 Hz, 1H), 7.53 (d, J = 8.7 Hz, 1H), 7.04 (d, J = 2.4 Hz, 1H), 6.98-6.95 (m, 2H), 6.84-6.81 (m, 1H), 6.71-6.53 (m, 4H), 5.30-5.06 (m, 1H), 4.60-4.47 (m, 1H), 4.42-4.39 (m, 1H), 4.31-4.27 (m, 1H), 4.17-4.12 (m, 1H), 3.0-2.88 (m, 6H), 2.36-2.32 (m, 5H), 1.93-1.85 (m, 2H), 1.63- 1.46 (m, 2H). |
| 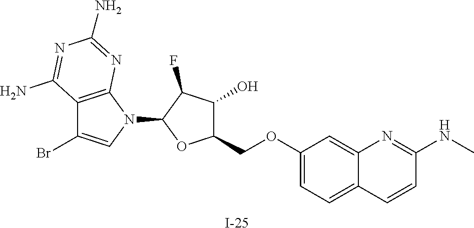 | B | 518.0 | ¹H NMR (400 MHz, DMSO- d6) δ 7.74 (d, J = 8.8 Hz, 1H), 7.52 (d, J = 8.8 Hz, 1H), 7.08 (d, J = 2.0 Hz, 1H), 7.04 (d, J = 2.4 Hz, 1H), 6.94 (brd, J = 4.8 Hz, 1H), 6.80 (dd, J = 8.8, 2.4 Hz, 1H), 6.58 (d, J = 8.8 Hz, 1H), 6.45 (dd, J = 16.0, 4.0 Hz, 1H), 6.30 (brs, 1H), 6.10 (d, J = 4.4 Hz, 1H), 5.95 (s, 2H), 5.13 (dt, J = 52.4, 4.0 Hz, 1H), 4.53- 4.46 (m, 1H), 4.37-4.26 (m, 2H), 4.15-4.11 (m, 1H), 2.89 (d, J = 4.8 Hz, 3H). |
| 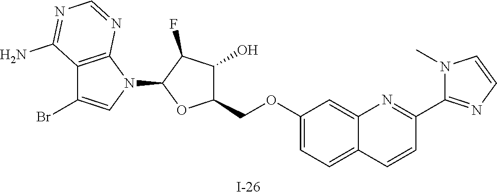 | B | 554.0 | ¹H NMR (400 MHz, CD₃OD) δ 8.28 (d, J = 8.6 Hz, 1H), 8.15 (s, 1H), 7.93-7.72 (m, 4H), 7.54 (s, 1H), 7.48 (s, 1H), 7.26 (d, J = 8.9 Hz, 1H), 7.02-6.78 (m, 1H), 6.71-6.66 (m, 1H), 6.17 (d, J = 4.9 Hz, 1H), 5.25 (d, J = 53.1 Hz, 1H), 4.65-4.55 (m, 1H), 4.52-4.42 (m, 2H), 4.24- 4.20 (m, 1H), 4.16 (s, 3H). |
| 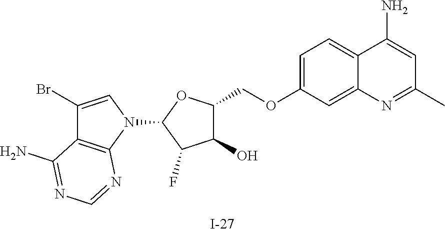 | B | 504.1 | ¹H NMR (400 MHz, DMSO- d6) δ 8.16 (s, 1H), 8.04 (d, J = 9.2 Hz, 1H), 7.57 (d, J = 2.4 Hz 1H), 7.19 (d, J = 2.4 Hz, 1H), 7.02 (dd, J = 6.4 Hz, 1.2 Hz, 1H), 6.57 (s, 2H), 6.32 (s, 1H), 6.18 (d, J = 2.4 Hz 1H), 5.33- 5.18 (m, 1H), 4.42-4.40 (m, 1H), 4.39-4.36 (m, 2H), 4.22- 4.20 (m, 1H), 2.38 (s, 3H). |
| 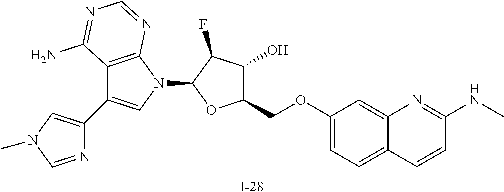 | B | 505.2 | ¹H NMR (400 MHz, DMSO- d6) δ 9.83 (s, 1H), 8.06 (s, 1H), 7.79-7.70 (m, 2H), 7.53 (d, J = 8.7 Hz, 1H), 7.49 (d, J = 2.2 Hz, 1H), 7.43 (s, 1H), 7.18 (s, 1H), 7.06 (d, J = 2.3 Hz, 1H), 6.95 (d, J = 4.5 Hz, 1H), 6.83 (dd, J = 8.7, 2.5 Hz, 1H), 6.71- 6.66 (m, 1H), 6.59 (d, J = 8.8 Hz, 1H), 6.13 (d, J = 4.8 Hz, 1H), 5.30-5.08 (m, 1H), 4.61- 4.48 (m, 1H), 4.45-4.42 (m, 1H), 4.34-4.30 (m, 1H), 4.20 (s, 1H), 3.66 (s, 3H), 2.88 (d, J = |
| | | | 4.7 Hz, 3H). |
| 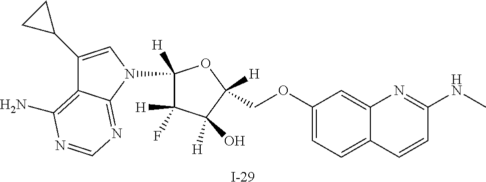 | B | 465.1 | ¹H NMR (400 MHz, DMSO- d6) δ ppm 8.08 (s, 1H), 7.74 (d, J = 8.8 Hz, 1H), 7.53 (d, J = 8.7 Hz, 1H), 7.04 (d, J = 2.2 Hz, 1H), 6.97-6.90 (m, 2H), 6.81 (dd, J = 8.7, 2.4 Hz, 1H), 6.73-6.51 (m, 4H), 6.08 (d, J = 4.9 Hz, 1H), 5.16 (dt, J = 52.9, 4.2 Hz, 1H), 4.55-4.49 (m, 1H), 4.39-4.36 (m, 1H), 4.31-4.26 (m, 1H), 4.16-4.06 (m, 1H), 2.89 (d, J = 4.7 Hz, 3H), 2.10- 1.96 (m, 1H), 0.85 (d, J = 8.2 |
| | | | Hz, 2H), 0.56-0.46 (m, 2H). |
| 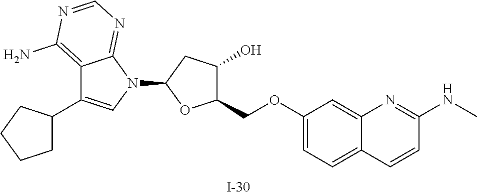 | C | 475.7 | ¹H NMR (400 MHz, DMSO- d6) δ ppm 8.03 (s, 1H), 7.74 (d, J = 8.8 Hz, 1H), 7.51 (d, J = 8.8 Hz, 1H), 7.11 (s, 1H), 7.01 (d, J = 2.4 Hz, 1H), 6.95-6.93 (m, 1H), 6.81 (dd, J = 8.8, 2.0 Hz, 1H), 6.64-6.61 (m, 1H), 6.57 (d, J = 9.0 Hz, 1H), 6.52 (s, 2H), 5.45 (d, J = 4.0 Hz, 1H), 4.49 (s, 1H), 4.29-4.26 (m, 1H), 4.20-4.10 (m, 2H), 3.30- 3.28 (m, 1H), 2.88 (d, J = 4.8 Hz, 3H), 2.68-2.61 (m, 1H), |
| | | | 2.24-2.19 (m, 1H), 1.99-1.89 |
| | | | (m, 2H), 1.63-1.55 (m, 4H), |
| | | | 1.47-1.35 (m, 2H). |
| 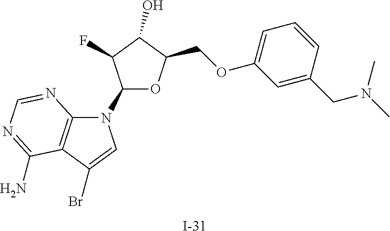 | B | 480.2 | ¹H NMR (400 MHz, DMSO- d6) δ ppm 8.16 (s, 1H), 7.50 (d, J = 1.8 Hz, 1H), 7.29 (t, J = 7.8 Hz, 1H), 7.03-6.90 (m, 3H), 6.67 (dd, J = 13.8, 4.8 Hz, 1H), 5.25 (dt, J = 52.8, 4.4 Hz, 1H), 4.60-4.45 (m, 6H), 4.31-4.23 (m, 2H), 4.17-4.13 (m, 1H), 2.25 (s, 6H). |
| 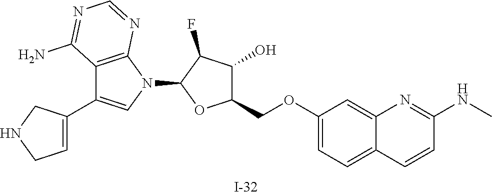 | B | 492.7 | ¹H NMR (400 MHz, DMSO- d6) δ ppm 8.23 (s, 1H), 7.99 (d, J = 8.0 Hz, 1H), 7.68 (d, J = 8.8 Hz, 1H), 7.47 (s, 1H), 7.32 (s, 1H), 7.09-7.06 (m, 1H), 6.78-6.71 (m, 2H), 5.99 (s, 1H), 5.22 (s, 0.5H), 5.09 (s, 0.5H), 4.78-4.62 (m, 2H), 4.48-4.43 (m, 2H), 4.35-4.29 (m, 6H), 3.11 (s, 3H). |
| 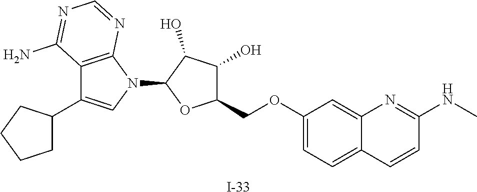 | A | 491.1 | ¹H NMR (400 MHz, DMSO- d6) δ 8.04 (s, 1H), 7.74 (d, J = 8.9 Hz, 1H), 7.52 (d, J = 8.4 Hz, 1H), 7.09 (s, 1H), 7.01 (s, 1H), 6.95 (s, 1H), 6.84-6.78 (m, 1H), 6.65-6.44 (m, 3H), 6.17 (d, J = 5.7 Hz, 1H), 5.37 (d, J = 6.4 Hz, 1H), 5.32 (d, J = 5.32 Hz, 1H), 4.50-4.42 (m, 1H), 4.32-4.28 (m, 1H), 4.23-4.18 (m, 3H), 2.88 (d, J = 4.5 Hz, 3H), 2.12-1.80 (m, 3H), 1.62 (s, 4H), 1.52-1.35 (m, 3H). |
| 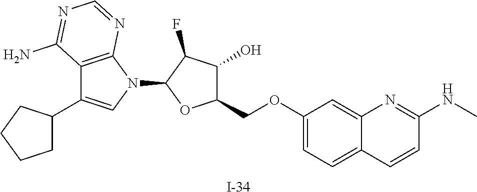 | B | 493.7 | ¹H NMR (400 MHz, CD₃OD) δ ppm 8.08 (s, 1H), 7.78 (d, J = 8.8 Hz, 1H), 7.54 (d, J = 8.8 Hz, 1H), 7.24 (d, J = 2.4 Hz, 1H), 7.02-7.01 (m, 1H), 6.95 (dd, J = 2.4 Hz, 1H), 6.69 (d, J = 4 Hz, 1H), 6.65-6.61 (m, 1H), 5.17-5.02 (m, 1H), 4.68- 4.42 (m, 1H), 4.49-4.46 (m, 1H), 4.40-4.36 (m, 1H), 4.32- 4.28 (m, 1H), 3.28-3.24 (m, 1H), 3.01 (s, 3H), 2.06-1.98 (m, 2H), 1.69-1.64 (m, 4H), |
| | | | 1.52-1.31 (m, 2H). |
| 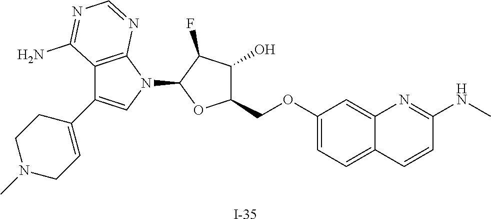 | B | 520.2 | ¹H NMR (400 MHz, DMSO- d6) δ ppm 8.13 (s, 1H), 7.74 (d, J = 8.9 Hz, 1H), 7.52 (d, J = 8.7 Hz, 1H), 7.26 (s, 1H), 7.04 (d, J = 2.1 Hz, 1H), 6.94-6.92 (m, 1H), 6.82-6.79 (m, 1H), 6.71-6.66 (m, 1H), 6.58 (d, J = 8.8 Hz, 1H), 6.37 (s, 1H), 6.11 (d, J = 4.8 Hz, 1H), 5.71 (s, 1H), 5.21 (dt, J = 53.1, 4.1 Hz, 1H), 4.58-4.51 (m, 1H), 4.42- 4.39 (m, 1H), 4.33-4.29 (m, 1H), 4.19-4.16 (m, 1H), 3.01 (s, 2H), 2.88 (d, J = 4.7 Hz, 3H), 2.39 (s, 2H), 2.28 (s, 3H). |
| 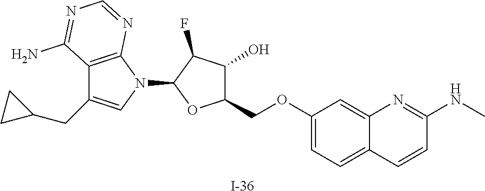 | B | 479.2 | ¹H NMR (400 MHz, DMSO- d6) δ ppm 7.95 (s, 1H), 7.63 (d, J = 8.8 Hz, 1H), 7.41 (d, J = 8.7 Hz, 1H), 6.96-6.92 (m, 2H), 6.85-6.82 (m, 1H), 6.72-6.46 (m, 4H), 6.01 (d, J = 4.9 Hz, 1H), 5.20-4.99 (m, 1H), 4.46- 4.40 (m, 1H), 4..40-3.80 (m, 1H), 4.31-4.27 (m, 1H), 4.20- 4.16 (m, 1H), 4.07-4.03 (m, 1H), 2.77 (d, J = 4.7 Hz, 3H), 2.64-2.53 (m, 2H), 0.87-0.81 (m, 1H), 0.32-0.25 (m, 2H), 0.07-0.12 (m, 2H). |
| 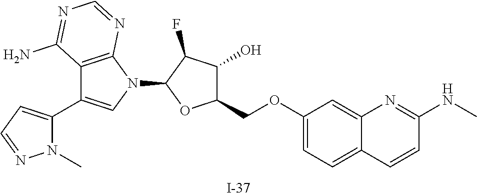 | B | 505.0 | ¹H NMR (400 MHz, DMSO- d₆) δ ppm 8.21 (s, 1H), 7.72 (d, J = 8.8 Hz, 1H), 7.60 (d, J = 2.0 Hz, 1H), 7.55 (d, J = 1.7 Hz, 1H), 7.48 (d, J = 8.7 Hz, 1H), 7.03 (d, J = 2.2 Hz, 1H), 6.93 (d, J = 4.8 Hz, 1H), 6.80- 6.72 (m, 2H), 6.57 (d, J = 8.8 Hz, 1H), 6.39 (d, J = 1.7 Hz, 1H), 6.16 (d, J = 4.8 Hz, 1H), 5.27 (dt, J = 52.8 Hz, J = 4.3 Hz, 1H), 4.62-4.54 (m, 1H), 4.42-4.35 (m, 2H), 4.24-4.20 (m, 1H), 3.72 (s, 3H), 2.87 |
| | | | (d, J = 4.7 Hz, 3H). |
| 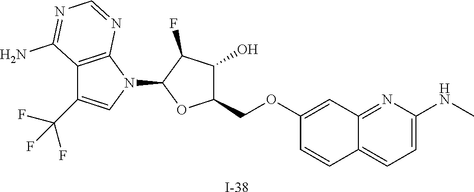 | B | 493.7 | ¹H NMR (400 MHz, DMSO- d6) δ ppm 8.27 (s, 1H), 7.96 (s, 1H), 7.74 (d, J = 9.2 Hz, 1H), 7.53 (d, J = 8.8 Hz, 1H), 7.06 (d, J = 2.4 Hz, 1H), 6.95 (brd, J = 4.8 Hz, 1H), 6.81 (dd, J = 8.8, 2.4 Hz, 1H), 6.73 (dd, J = 13.6, 4.8 Hz, 1H), 6.72 (brs, 1H), 6.58 (d, J = 8.8 Hz, 1H), 6.17 (d, J = 4.8 Hz, 1H), 5.29 (dt, J = 52.8, 4.4 Hz, 1H), 4.63- 4.54 (m, 1H), 4.44-4.35 (m, 2H), 4.24-4.20 (m, 1H), 2.88 (d, J = 4.8 Hz, 3H). |
| 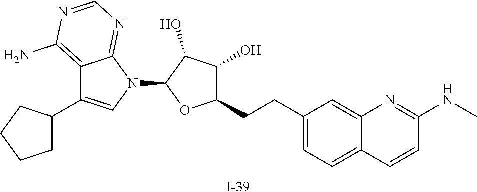 | A | 489.3 | ¹H NMR (400 MHz, DMSO) δ 8.04 (s, 1H), 7.77 (d, J = 8.9 Hz, 1H), 7.51 (d, J = 8.1 Hz, 1H), 7.33 (s, 1H), 7.08 (s, 1H), 7.00 (dd, J = 8.1, 1.5 Hz, 1H), 6.96-6.88 (m, 1H), 6.66 (d, J = 8.9 Hz, 1H), 6.54 (s, 2H), 6.03 (d, J = 5.8 Hz, 1H), 5.23 (d, J = 6.3 Hz, 1H), 5.09 (d, J = 5.2 Hz, 1H), 4.57-4.35 (m, 1H), 4.07-3.90 (m, 1H), 3.87-3.71 (m, 1H), 3.45-3.35 (m, 1H), 2.88 (d, J = 4.7 Hz, 3H), 2.84- 2.62 (m, 2H), 2.15-1.88 (m, 4H), |
| | | | 1.83-1.60 (m, 4H), 1.60-1.45 |
| | | | (m, 2H). |
| 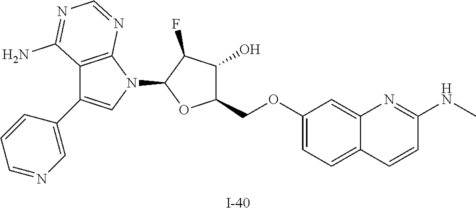 | B | 502.2 | ¹H NMR (400 MHz, DMSO- d6) δ ppm 8.68 (d, J = 2.1 Hz, 1H), 8.56-8.54 (m, 1H), 8.21 (s, 1H), 7.90-7.78 (m, 1H), 7.73 (d, J = 8.9 Hz, 1H), 7.55 (d, J = 1.9 Hz, 1H), 7.48-7.44 (m, 2H), 7.03 (d, J = 2.3 Hz, 1H), 6.96- 6.92 (m, 1H), 6.85-6.71 (m, 2H), 6.57 (d, J = 8.8 Hz, 1H), 6.32 (s, 1H), 6.16-6.15 (m, 1H), 5.28 (dt, J = 52.7, 4.4 Hz, 1H), 4.63-4.57 (m, 1H), 4.43-4.33 (m, 2H), 4.26-4.11 (m, 1H), 2.86 (d, J = 4.7 Hz, 3H). |
| 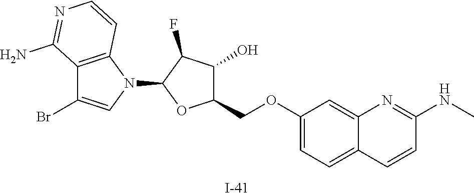 | B | 502.1 | ¹H NMR (400 MHz, DMSO- d6) δ ppm 7.75 (d, J = 8.9 Hz, 1H), 7.65 (d, J = 6.0 Hz, 1H), 7.56-7.54 (m, 2H), 7.07 (d, J = 2.4 Hz, 1H), 7.00-6.93 (m, 2H), 6.85-6.83 (m, 1H), 6.59 (d, J = 8.8 Hz, 1H), 6.43-6.40 (m, 1H), 6.18 (d, J = 4.9 Hz, 1H), 5.97 (s, 2H), 5.24 (dt, J = 53.0, 4.5 Hz, 1H), 4.57-4.48 (m, 1H), 4.44-4.35 (m, 2H), 4.17-4.13 (m, 1H), 2.89 (d, J = 4.7 Hz, 3H). |
| 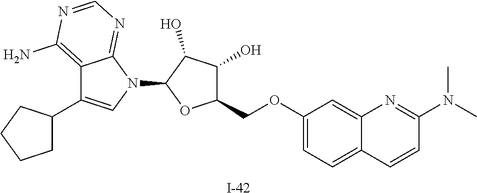 | A | 505.2 | ¹H NMR (400 MHz, DMSO- d6) δ 8.04 (s, 1H), 7.92 (d, J = 9.1 Hz, 1H), 7.59 (d, J = 8.8 Hz, 1H), 7.09 (s, 1H), 7.02 (d, J = 2.4 Hz, 1H), 6.90 (d, J = 9.1 Hz, 1H), 6.86 (dd, J = 8.7, 2.5 Hz, 1H), 6.52 (s, 2H), 6.17 (d, J = 6.0 Hz, 1H), 5.37 (d, J = 6.4 Hz, 1H), 5.31 (d, J = 4.9 Hz, 1H), 4.50-4.45 (m, 1H), 4.34-4.30 (m, 1H), 4.29- 4.14 (m, 3H), 3.35-3.28 (m, 1H), 3.14 (s, 6H), 2.02-1.90 (m, 2H), 1.65-1.54 (m, 4H), |
| | | | 1.50-1.35 (m, 2H). |
| 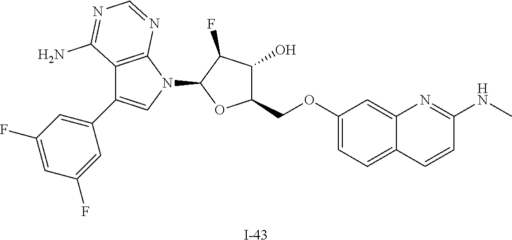 | B | 537.3 | ¹H NMR (400 MHz, DMSO- d6) δ ppm 8.20 (s, 1H), 7.73 (d, J = 8.8 Hz, 1H), 7.58 (s, 1H), 7.46 (d, J = 8.8 Hz, 1H), 7.21- 7.16 (m, 2H), 7.03 (s, 1H), 6.93-6.91 (m, 1H), 6.79-6.72 (m, 2H), 6.56 (d, J = 8.4 Hz, 1H), 6.43 (br, 1H), 6.14 (d, J = 4.8 Hz, 1H), 5.34 (dt, J = 52.8, 4 Hz, 1H), 4.62 (dd, J = 19.6, 4.8 Hz, 1H), 4.43-4.35 (m, 2H), 4.22-4.19 (m, 1H), 2.86 (d, J = 4.8 Hz, 3H) |
| 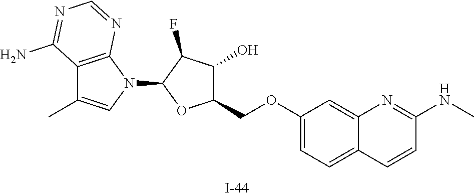 | B | 438.45 | ¹H NMR (400 MHz, DMSO- d6) δ 8.04 (s, 1H), 7.34 (d, J = 8.8 Hz, 1H), 7.54 (d, J = 8.8 Hz, 1H), 7.03 (br, 2H), 6.94-6.93 (m, 1H), 6.81 (dd, J = 8.8, 2.4 Hz, 1H), 6.63-6.57 (m, 3H), 6.09 (d, J = 4.8 Hz, 1H), 5.21 (dt, J = 52.8 Hz, 4 Hz, 1H), 4.54-4.47 (m, 1H), 4.39-4.35 (m, 1H), 4.29-4.25 (m, 1H), 4.16-4.13 (m, 1H), 2.88 (d, J = 4.4 Hz, 3H), 2.33 (s, 3H) |
| 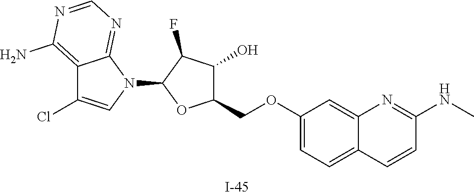 | B | 459.7 | ¹H NMR (400 MHz, DMSO- d6) δ ppm 8.14 (s, 1H), 7.74 (d, J = 9.2 Hz, 1H), 7.53 (d, J = 8.8 Hz, 1H), 7.50 (d, J = 2.0 Hz, 1H), 7.05 (d, J = 2.4 Hz, 1H), 6.95 (brd, J = 4.4 Hz, 2H), 6.81 (dd, J = 8.8, 2.4 Hz, 1H), 6.67 (dd, J = 14.0, 4.8 Hz, 1H), 6.58 (d, J = 9.2 Hz, 1H), 6.14 (d, J = 4.8 Hz, 1H), 5.23 (dt, J = 52.8, 4.4 Hz, 1H), 4.58-4.50 (m, 1H), 4.40-4.31 (m, 2H), 4.19-4.16 (m, 1H), 2.89 (d, J = |
| | | | 5.2 Hz, 3H). |
| 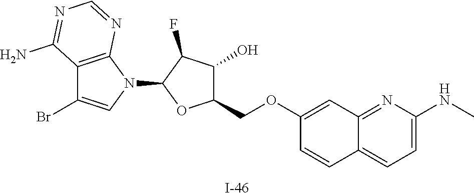 | B | 503.7 | ¹H NMR (400 MHz, DMSO- d6) δ ppm 8.14 (s, 1H), 7.74 ( d, J = 8.8 Hz, 1H), 7.56-7.52 (m, 2H), 7.05 (d, J = 2.0 Hz, 1H), 6.96-6.94 (m, 1H), 6.85- 6.79 (m, 1H), 6.69-6.65 (m, 1H), 6.58 (d, J = 8.8 Hz, 1H), 6.14 (d, J = 4.8 Hz, 1H), 5.31- 5.16 (m, 1H), 4.59-4.50 (m, 1H ), 4.41-4.31 (m, 2H), 4.19- 4.16 (m, 1H), 2.89 (d, J = 4.4 Hz, 3H). |
| 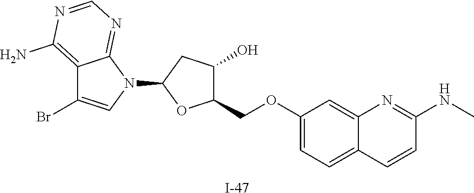 | C | 486.7 | ¹H NMR (400 MHz, DMSO- d6) δ ppm 8.12 (s, 1H), 7.74 (d, J = 8.8 Hz, 1H), 7.67 (s, 1H), 7.52 (d, J = 8.8 Hz, 1H), 7.02 (d, J = 2.4 Hz, 1H), 6.95 (brd, J = 3.6 Hz, 1H), 6.81 (dd, J = 8.8, 2.4 Hz, 1H), 6.81 (brs, 1H), 6.63 (t, J = 7.0 Hz, 1H), 6.58 (d, J = 8.8 Hz, 1H), 5.50 (d, J = 4.0 Hz, 1H), 4.50-4.47 (m, 1H), 4.30-4.26 (m, 1H), 4.21-4.14 (m, 2H), 2.88 (d, J = 4.8 Hz, 3H), 2.65-2.58 (m, 1H), 2.31- 2.25 (m, 1H). |
| 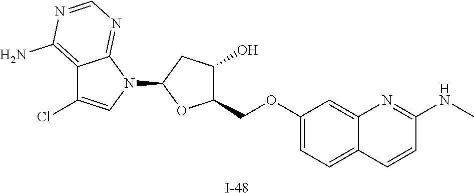 | C | 441.7 | ¹H NMR (400 MHz, DMSO- d6) δ ppm 8.12 (s, 1H), 7.74 (d, J = 8.8 Hz, 1H), 7.61 (s, 1H), 7.52 (d, J = 8.8 Hz, 1H), 7.02 (d, J = 8.8 Hz, 1H), 6.95 (s, 2H), 6.80 (dd, J = 8.8, 2.4 Hz, 1H), 6.64-6.61 (m, 1H), 6.58 (d, J = 9.2 Hz, 1H), 5.50 (d, J = 4.0 Hz, 1H), 4.49-4.48 (m, 1H), 4.29-4.25 (m, 1H), 4.20-4.14 (m, 2H), 2.88 (d, J = 4.8 Hz, 3H), 2.64-2.58 (m, 1H), 2.31- 2.25 (m, 1H). |
| 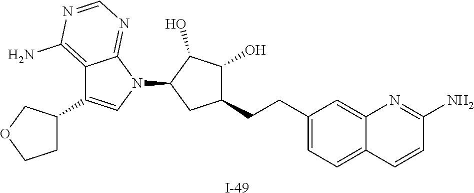 | D | 475.0 | ¹H NMR (400 MHz, CD3OD) δ ppm 8.06 (s, 1H), 7.90 (d, J = 8.8 Hz, 1H), 7.58 (d, J = 8.2 Hz, 1H), 7.40 (s, 1H), 7.22- 7.10 (m, 2H), 6.78 (d, J = 8.8 Hz, 1H), 4.34-4.30 (m, 1H), 4.18-4.09 (m, 2H), 3.96-3.76 (m, 4H), 2.95-2.77 (m, 2H), 2.51-2.35 (m, 2H), 2.16-1.97 (m, 3H), 1.87-1.82 (m, 1H), 1.65-1.62 (m, 1H). |
| 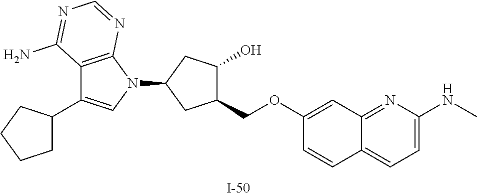 | C | 473.2 | ¹H NMR (400 MHz, DMSO- d6) δ ppm 8.01 (s, 1H), 7.73 (d, J = 9.0 Hz, 1H), 7.50 (d, J = 8.7 Hz, 1H), 7.10 (s, 1H), 7.01 (d, J = 2.3 Hz, 1H), 6.92 (d, J = 5.0 Hz, 1H), 6.81-6.78 (m, 1H), 6.57 (d, J = 8.9 Hz, 1H), 6.43 (s, 2H), 5.29 (d, J = 8.5 Hz, 1H), 4.96 (d, J = 4.1 Hz, 1H), 4.24-4.20 (m, 2H), 4.13-4.03 (m, 1H), 2.88 (d, J = 4.7 Hz, 3H), 2.40-2.34 (m, 2H), 2.22- 2.15 (m, 1H), 2.01-1.94 (m, 3H), 1.76-1.61 (m, 5H), 1.52 |
| | | | (m, 2H). |
| 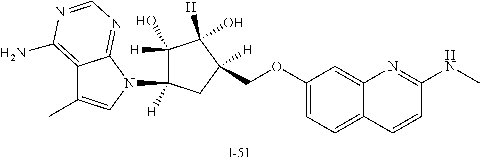 | D | 433.0 | ¹H NMR (400 MHz, DMSO- d6) δ ppm 7.98 (s, 1H), 7.77 (d, J = 8.8 Hz, 1H), 7.51 (d, J = 8.0 Hz, 1H), 7.35 (s, 1H), 7.03- 7.01 (m, 2H), 6.94-6.91 (m, 1H), 6.66 (d, J = 8.8 Hz, 1H), 6.48 (s, 2H), 4.81-4.74 (m, 2H), 4.62 (d, J = 4.4 Hz, 1H), 4.16- 4.11 (m, 1H), 3.74-3.70 (m, 1H), 2.88 (d, J = 4.8 Hz, 3H), 2.78-2.64 (m, 2H), 2.35 (s, 3H), 2.24-2.17 (m, 1H), 1.97-1.90 |
| | | | (m, 1H), 1.87-1.83 (m, 1H), |
| | | | 1.73-1.67 (m, 1H), 1.49-1.41 |
| | | | (m, 1H). |
| 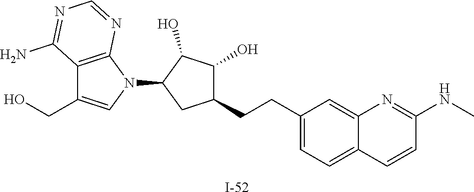 | D | 449.2 | ¹H NMR (400 MHz, DMSO- d6) δ ppm 8.05 (br, 1H), 7.77 (d, J = 5.2 Hz, 1H), 7.51 (d, J = 8.0 Hz, 1H), 7.35 (s, 1H), 7.02 (dd, J = 8.0, 1.2 Hz, 1H), 7.94- 7.92 (m, 1H), 6.85 (br, 1H), 6.66 (d, J = 9.2 Hz, 1H), 4.81- 4.76 (m, 1H), 4.59 (s, 2H), 4.17-4.14 (m, 1H), 3.74-3.71 (m, 1H), 2.88 (d, J = 3.6 Hz, 3H), 2.75-2.67 (m, 2H), 2.23- 2.21 (m, 1H), 1.96-1.85 (m, 2H), 1.70-1.69 (m, 1H), 1.47- 1.44 (m, 1H) |
| 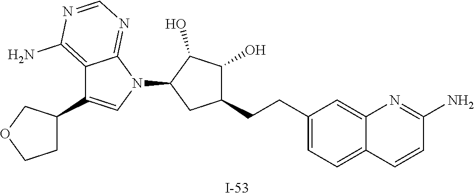 | D | 475.1 | ¹H NMR (400 MHz, CD3OD) δ ppm 8.06 (s, 1H), 7.90 (d, J = 8.9 Hz, 1H), 7.58 (d, J = 8.2 Hz, 1H), 7.39 (s, 1H), 7.21- 7.11 (m, 2H), 6.77 (d, J = 8.9 Hz, 1H), 4.34-4.30 (m, 1H), 4.18-4.09 (m, 2H), 3.97-3.74 (m, 4H), 2.95-2.76 (m, 2H), 2.53-2.33 (m, 2H), 2.16-1.97 (m, 3H), 1.87-1.82 (m, 1H), 1.65-1.62 (m, 1H). |
| 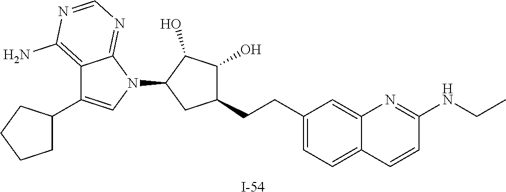 | D | 502.3 | ¹H NMR (400 MHz, DMSO- d6) δ ppm 7.94 (s, 1H), 7.69 (d, J = 9.0 Hz, 1H), 7.47-7.37 (m, 2H), 7.01 (d, J = 6.7 Hz, 1H), 6.94 (s, 1H), 6.59 (d, J = 8.9 Hz, 1H), 4.78-4.72 (m, 1H), 4.26-4.18 (m, 1H), 3.85-3.76 (m, 2H), 3.71 (s, 1H), 3.57 (d, J = 19.4 Hz, 1H), 3.41-3.85 (m, 2H), 3.28-3.25 (m, 1H), 2.84- 2.62 (m, 3H), 2.35-2.23 (m, 1H), 2.08-1.91 (m, 4H), 1.83- 1.46 (m, 9H), 1.19 (t, J = 7.2 |
| | | | Hz, 3H). |
| 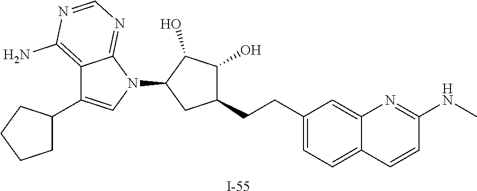 | D | 487.3 | ¹H NMR (400 MHz, CD₃OD) δ ppm 8.05-8.01 (m, 2H), 7.66 (t, J = 8.0 Hz, 2H), 7.30 (d, J = 8.0 Hz, 1H), 7.06 (s, 1H), 6.70 (d, J = 8.0 Hz, 1H), 4.88-4.83 (m, 1H), 4.36-4.33 (m, 1H), 3.91 (t, J = 4.8 Hz, 1H), 3.38- 3.34 (m, 1H), 3.12 (s, 3H), 2.94-2.87(m, 2H), 2.41-2.37 (m, 1H), 2.15-2.03 (m, 4H), 1.88-1.71 (m, 5H), 1.69-1.63 (m, 3H). |
| 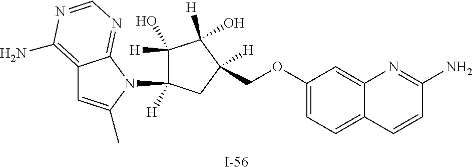 | A | 421.7 | ¹H NMR (400 MHz, CD₃OD) δ ppm 8.16 (d, J = 8.0 Hz, 1H), 8.03 (s, 1H), 7.75 (d, J = 8.0 Hz, 1H), 7.15-7.13 (m, 2H), 6.83 (d, J = 8.8 Hz, 1H), 6.34 (s, 1H), 5.08-4.97 (m, 1H), 4.77-4.71 (m, 1H), 4.44-4.40 (m, 1H), 4.36-4.29 (m, 2H), 2.68-2.59 (m, 1H), 2.49 (s, 3H), 2.42-2.38 (m, 1H), 2.36-2.31 (m, 1H). |
| 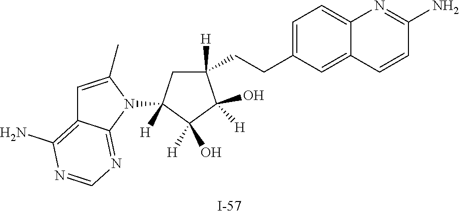 | D | 419.0 | ¹H NMR (400 MHz, CD3OD) δ ppm 8.01 (s, 1H), 7.91-7.88 (m, 1H), 7.50-7.47 (m, 3H), 6.815 (d, J = 8.8 Hz, 1H), 6.27 (s, 1H), 4.90-4.88 (m, 1H), 4.65-4.55 (m, 1H), 4.07 (t, J = 5.6 Hz, 1H), 2.89-2.76 (m, 2H), 2.44 (s, 3H), 2.25-2.20 (m, 2H), 2.15-1.99 (m, 2H), 1.93-1.88 (m, 1H). |
| 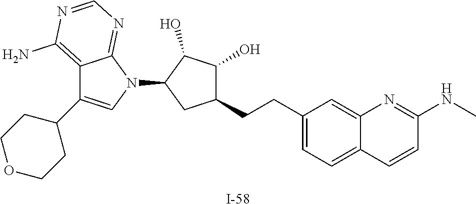 | D | 503.0 | ¹H NMR (400 MHz, CD3OD) δ ppm 8.07 (s, 1H), 7.80 (d, J = 8.8 Hz, 1H), 7.54 (d, J = 7.3 Hz, 2H), 7.20-7.03 (m, 2H), 6.69 (d, J = 8.9 Hz, 1H), 4.65 (s, 1H), 4.41-4.32 (m, 1H), 4.04 (d, J = 7.9 Hz, 2H), 3.98-3.89 (m, 1H), 3.72-3.66 (m, 2H), 3.22-3.13 (m, 1H), 3.02 (s, 3H), 2.93-2.80 (m, 2H), 2.48-2.34 (m, 1H), 2.14-2.08 (m, 1H), 2.02-2.98 (m, 2H), 1.88-1.56 (m, 7H), 1.08-0.95 (m, 1H). |
| 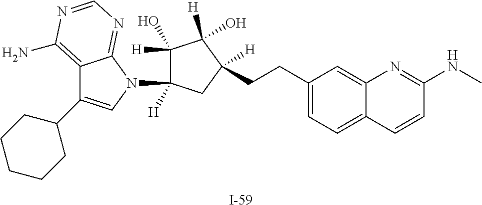 | D | 501.3 | ¹H NMR (400 MHz, CD3OD) δ ppm 8.05 (s, 1H), 7.81 (d, J = 9.0 Hz, 1H), 7.54 (d, J = 7.6 Hz, 2H), 7.13 (d, J = 9.4 Hz, 1H), 7.02 (s, 1H), 6.69 (d, J = 8.9 Hz, 1H), 4.65 (s, 1H), 4.41- 4.29 (m, 1H), 3.93-3.88 (m, 1H), 3.02 (s, 3H), 2.96-2.74 (m, 3H), 2.43-2.39 (m, 1H), 2.13- 2.06 (m, 4H), 1.89-1.79 (m, 4H), 1.69-1.50 (m, 3H), 1.46- 1.30 (m, 3H). |
| 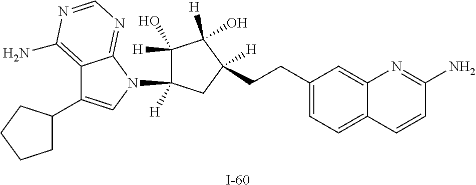 | D | 473.2 | ¹H NMR (400 MHz, CD3OD) δ ppm 8.05 (s, 1H), 7.90 (d, J = 9.2 Hz, 1H), 7.58 (d, J = 8.0 Hz, 1H), 7.19-7.17 (m, 1H), 7.05 (s, 2H), 6.78 (d, J = 9.2 Hz, 1H), 4.65 (s, 2H), 4.35- 4.32 (m, 1H), 4.92-4.90 (m, 1H), 2.91-2.83 (m, 2H), 2.18- 2.05 (m, 4H), 1.88-1.60 (m, 8H), 1.35-1.31 (m, 1H). |
| 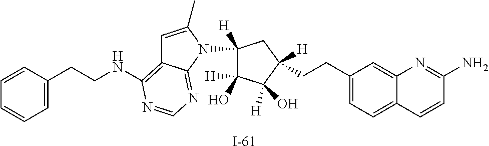 | D | 523.7 | ¹H NMR (400 MHz, CD₃OD) δ ppm 8.08 (s, 1H), 7.91-7.88 (m, 1H), 7.59-7.56 (m, 1H), 7.40 (s, 1H), 7.31-7.27 (m, 4H), 7.21- 7.16 (m, 2H), 6.77 (d, J = 8.8 Hz, 1H), 6.23 (s, 1H), 4.93- 4.89 (m, 1H), 4.61-4.54 (m, 1H), 4.08 (t, J = 6 Hz, 1H), 3.73 (t, J = 7.6 Hz, 2H), 2.96 (t, J = 8 Hz, 2H), 2.91-2.81 (m, 2H), 2.43 (s, 3H), 2.25-2.16 (m, 2H), |
| | | | 2.14-2.09 (m, 1H), 2.05-2.00 |
| | | | (m, 1H), 1.97-1.91 (m, 1H). |
| 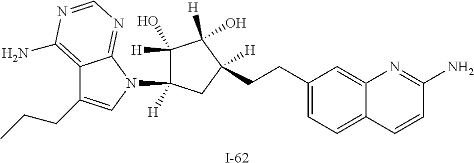 | D | 447.2 | ¹H NMR (400 MHz, DMSO) δ ppm 7.99 (s, 1H), 7.82 (d, J = 8.7 Hz, 1H), 7.53 (d, J = 8.1 Hz, 1H), 7.27 (s, 1H), 7.04 (d, J = 8.2 Hz, 2H), 6.68 (d, J = 8.8 Hz, 1H), 6.41 (s, 2H), 6.34 (s, 2H), 4.87-4.70 (m, 2H), 4.62 (d, J = 5.0 Hz, 1H), 4.19-4.13 (m, 1H), 3.73-3.71 (m, 1H), 2.74-2.61 (m, 3H), 2.21-2.16 (m, 1H), 2.00-1.80 (m, 2H), 1.71-1.69 (m, 1H), 1.61-1.56 |
| | | | (m, 2H), 1.50-1.45 (m, 1H), |
| | | | 1.24 (s, 1H), 0.95 (t, J = 7.3 |
| | | | Hz, 3H). |
| 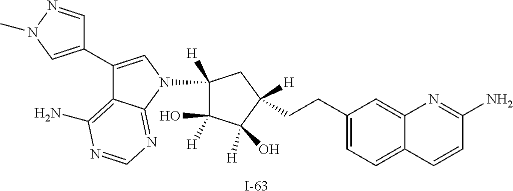 | D | 484.8 | ¹H NMR (400 MHz, CD3OD) δ ppm 8.47 (s, 1H), 8.17-8.13 (m, 2H), 7.77 (s, 1H), 7.74 (d, J = 8 Hz, 1H), 7.63 (s, 1H), 7.47 (s, 1H), 7.36-7.34 (m, 1H), 7.27 (s, 1H), 6.93 (d, J = 9.2 Hz, 1H), 4.89 (s, 1H), 4.65 (s, br, 2H), 4.41-4.37 (m, 1H), 3.98 (s, 3H), 3.95-3.92 (m, 1H), 2.93-2.90 (m, 2H), 2.45-2.42 (m, 1H), 2.09-2.07 (m, 2H), 1.91-1.83 (m, 1H), 1.73-1.63 (m, 1H) |
| 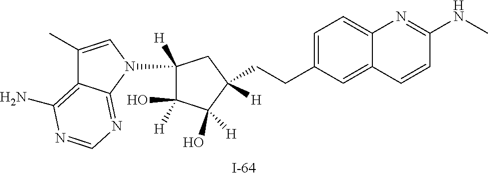 | D | 433.2 | ¹H NMR (400 MHz, CD₃OD) δ ppm 8.03 (s, 1H), 7.80 (d, J = 8.8 Hz, 1H), 7.61 (d, J = 8.4 Hz, 1H), 7.46-7.42 (m, 2H), 7.01 (s, 1H), 6.73 (d, J = 8.8 Hz, 1H), 3.02 (s, 3H), 2.85- 2.78 (m, 2H), 2.45-2.39 (m, 4H), 2.09-2.03 (m, 2H), 1.81- 1.79 (m, 1H), 1.63-1.58 (m, 1H), 1.45-1.30 (m, 2H). |
| 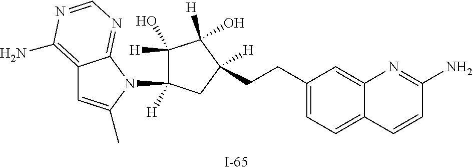 | D | 419.1 | ¹H NMR (400 MHz, DMSO- d6) δ ppm 7.97 (s, 1H), 7.82 (d, J = 8.8 Hz, 1H), 7.53 (d, J = 8.1 Hz, 1H), 7.27 (s, 1H), 7.04 (dd, J = 8.1, 1.4 Hz, 1H), 6.73 (s, 2H), 6.68 (d, J = 8.8 Hz, 1H), 6.34 (s, 2H), 6.23 (s, 1H), 4.69-4.61 (m, 2H), 4.46-4.38 (m, 1H), 3.85-3.77 (m, 2H), 2.83-2.64 (m, 2H), 2.34 (s, 3H), 2.04-1.93 (m, 2H), 2.02-1.69 (m, 3H). |
| 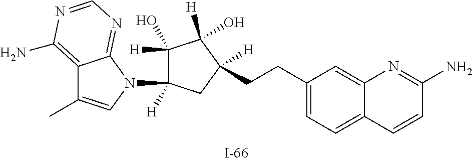 | D | 419.1 | ¹H NMR (400 MHz, DMSO- d6) δ ppm 7.98 (s, 1H), 7.82 (d, J = 8.8 Hz, 1H), 7.52 (d, J = 8.1 Hz, 1H), 7.26 (s, 1H), 7.07- 6.99 (m, 2H), 6.69-6.66 (m, 1H), 6.47 (s, 2H), 6.33 (s, 2H), 4.81-4.71 (m, 2H), 4.60 (d, J = 4.8 Hz, 1H), 4.16-4.11 (m, 1H), 3.74-3.70 (m, 1H), 2.76-2.63 (m, 2H), 2.35 (s, 3H), 2.23-2.16 (m, 1H), 1.97-1.83 (m, 2H), 1.72-1.62 (m, 1H), 1.48-1.40 |
| | | | (m, 1H). |
| 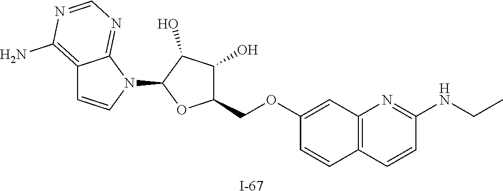 | A | 438.0 | ¹H NMR (400 MHz, DMSO- d₆) δ 8.08 (s, 1H), 7.73 (d, J = 8.8 Hz, 1H), 7.51 (d, J = 8.7 Hz, 1H), 7.38 (d, J = 3.7 Hz, 1H), 7.07-6.96 (m, 3H), 6.92 (t, J = 5.2 Hz, 1H), 6.81 (dd, J = 8.7, 2.5 Hz, 1H), 6.62 (d, J = 3.6 Hz, 1H), 6.57 (d, J = 8.8 Hz, 1H), 6.17 (d, J = 5.6 Hz, 1H), 5.44 (d, J = 6.2 Hz, 1H), 5.37 (d, J = 4.8 Hz, 1H), 4.49- 4.81 (m, 1H), 4.31-4.21 (m, 4H), 3.41-3.39 (m, 2H), 1.20- 1.17 (m, 3H). |
| 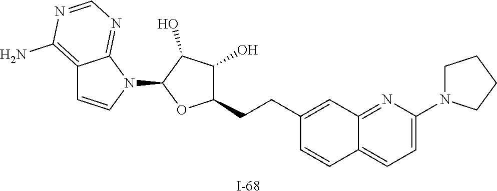 | D | 461.1 | ¹H NMR (400 MHz, CD3OD) δ 8.12 (s, 1H), 7.93 (d, J = 9.0 Hz, 1H), 7.57 (d, J = 8.4 Hz, 2H), 7.29 (d, J = 3.7 Hz, 1H), 7.12 (dd, J = 8.1, 1.6 Hz, 1H), 6.83 (d, J = 9.1 Hz, 1H), 6.68 (d, J = 3.7 Hz, 1H), 6.17 (d, J = 5.1 Hz, 1H), 4.50 (t, J = 5.3 Hz, 1H), 4.12 (t, J = 5.2 Hz, 1H), 4.08-3.95 (m, 1H), 3.63 (t, J = 6.6 Hz, 4H), 3.04-2.77 (m, 2H), 2.30-1.91 (m, 6H). |
| 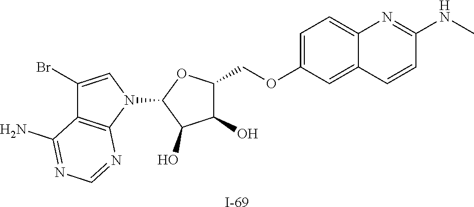 | A | 501.7 | ¹H NMR (400 MHz, DMSO- d6) δ ppm 8.13 (s, 1H), 7.76 (d, J = 8.8 Hz, 1H), 7.66 (s, 1H), 7.47 (d, J = 8.8 Hz, 1H), 7.20- 7.16 (m, 2H), 6.79-6.78 (m, 1H), 6.72 (d, J = 8.8 Hz, 1H), 6.16 (d, J = 5.6 Hz, 1H), 5.50 (d, J = 6.0 Hz, 1H), 5.37 (d, J = 4.8 Hz, 1H), 4.49-4.45 (m, 1H), 4.27-4.17 (m, 1H), 2.86 (d, J = 4.8 Hz, 3H). |
| 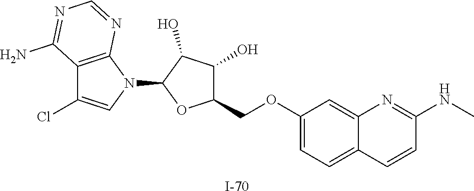 | A | 457 | ¹H NMR (400 MHz, DMSO- d6) δ ppm δ 7.78 (s, 1H), 7.73- 7.72 (d, J = 4.1 Hz, 1H), 7.61 (s, 1H), 7.53-7.51 (d, J = 8.0 Hz, 1H), 7.03 (s, 1H), 7.0 (s, 1H), 6.80-6.78 (d, J = 8.0 Hz 1H), 6.57-6.55 (d, J = 8.1 Hz, 1H), 6.33-6.32 (d, J = 4 Hz, 1H), 4.98-4.95 (t, J = 6.2 Hz, 1H), 4.29-4.27 (d, J = 8.2 Hz, 1H), 4.23 (m, 3H), 2.89- 2.88 (d, J = 4 Hz, 3H). |
| 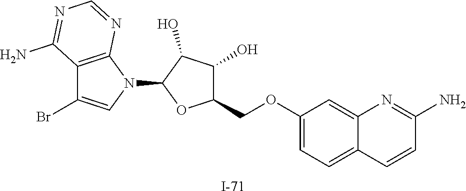 | A | 488.9 | ¹H NMR (400 MHz, DMSO- d₆) δ ppm 8.13 (s, 1H), 7.79 (d, J = 8.8 Hz, 1H), 7.65 (s, 1H), 7.54 (d, J = 8.8 Hz, 1H), 6.93 (d, J = 2.4 Hz, 1H), 6.84 (dd, J = 8.7, 2.5 Hz, 2H), 6.59 (d, J = 8.8 Hz, 1H), 6.34 (s, 2H), 6.17 (d, J = 5.6 Hz, 1H), 5.51 (d, J = 6.0 Hz, 1H), 5.39 (d, J = 4.7 Hz, 1H), 4.48-4.43 (m, 1H), 4.32-4.29 (m, 1H), 4.26-4.21 (m, 3H). |
| 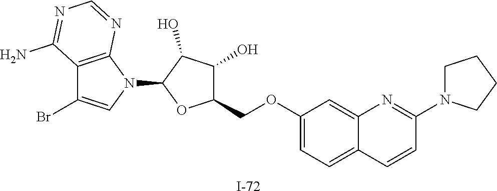 | A | 543.0 | ¹H NMR (400 MHz, DMSO- d₆) δ ppm 8.13 (s, 1H), 7.90 (d, J = 9.0 Hz, 1H), 7.68 (s, 1H), 7.58 (d, J = 8.8 Hz, 1H), 7.02 (d, J = 2.4 Hz, 1H), 6.83 (dd, J = 8.7, 2.5 Hz, 2H), 6.70 (d, J = 4.4 Hz, 1H), 6.18 (d, J = 5.7 Hz, 1H), 5.50 (d, J = 6.1 Hz, 1H), 5.39 (d, J = 4.6 Hz, 1H), 4.48-4.44 (m, 1H), 4.34-4.32 (m, 1H), 4.29-4.19 (m, 3H), 3.52 (s, 4H), 1.99-1.96 (m, 4H). |
| 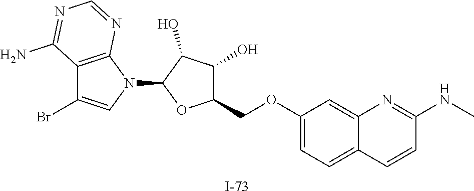 | A | 501.1 | ¹H NMR (400 MHz, DMSO- d6) δ ppm 8.13 (s, 1H), 7.74 (d, J = 8.8 Hz, 1H), 7.67 (s, 1H), 7.52 (d, J = 8.8 Hz, 1H), 7.02 (d, J = 2.4 Hz, 1H), 6.94-6.92 (m, 1H), 6.82 (dd, J = 8.4, 2.4 Hz, 1H), 6.57 (d, J = 8.8 Hz, 1H), 6.17 (d, J = 5.6 Hz, 1H), 5.49 (d, J = 6 Hz, 1H), 5.38 (d, J = 4.4 Hz, 1H), 4.45-4.44 (m, 1H), 4.33-4.31 (m, 1H), 4.26-4.22 (m, 2H), 2.88 (d, J = 4.8 Hz, 3H) |
| 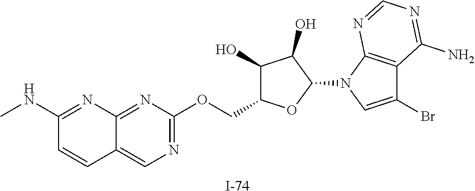 | A | 503.7 | ¹H NMR (400 MHz, DMSO- d6) δ ppm 8.88 (s, 1H), 8.29 (s, 1.5H), 8.12 (s, 1H), 8.01-7.99 (m, 1H), 7.87 (d, J = 8.8 Hz, 1H), 7.69 (s, 1H), 6.69 (d, J = 8.8 Hz, 1H), 6.18 (d, J = 6.0 Hz, 1H), 4.61-4.57 (m, 1H), 4.53-4.49 (m, 2H), 4.26-4.21 (m, 2H), 2.93 (d, J = 4.8 Hz, 3H). |
| 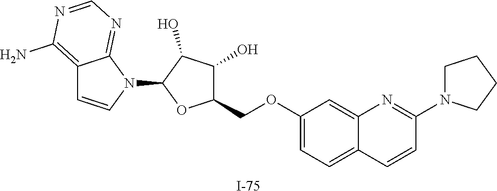 | A | 463.0 | ¹H NMR (400 MHz, DMSO- d6) δ ppm 8.07 (s, 1H), 7.90 (d, J = 9.0 Hz, 1H), 7.57 (d, J = 8.8 Hz, 1H), 7.38 (d, J = 3.7 Hz, 1H), 7.09-6.96 (m, 3H), 6.83 (dd, J = 8.7, 2.5 Hz, 1H), 6.69 (d, J = 9.0 Hz, 1H), 6.61 (d, J = 3.6 Hz, 1H), 5.45 (d, J = 6.2 Hz, 1H), 5.38 (d, J = 5.0 Hz, 1H), 4.52-4.45 (m, 1H), 4.33-4.28 (m, 1H), 4.28-4.19 (m, 3H), 3.52 (s, 4H), 1.97 (t, J = 6.5 Hz, 4H). |
| 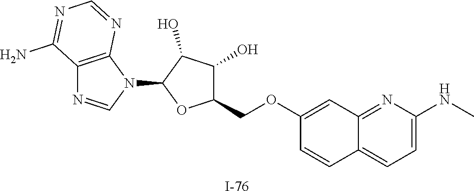 | A | 424.2 | ¹H NMR (400 MHz, CD₃OD) δ 8.32 (s, 1H), 8.24 (s, 1H), 8.14- 8.12 (m, 1H), 7.76 (d, J = 8.8 Hz, 1H), 7.37 (s, 1H), 7.16 (dd, J = 8.8, 2.3 Hz, 1H), 6.86- 6.84 (m, 1H), 6.11 (d, J = 4.0 Hz, 1H), 4.88-4.81 (m, 1H), 4.66 (t, J = 5.1 Hz, 1H), 4.59- 4.56 (m, 1H), 4.52-4.41 (m, 2H), 3.20 (s, 3H). |
| 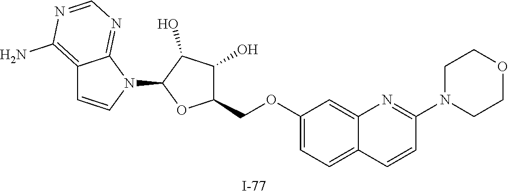 | A | 479.7 | ¹H NMR (400 MHz, CD₃OD) δ ppm 8.12 (s, 1H), 7.96 (d, J = 8.8 Hz, 1H), 7.62 (d, J = 8.8 Hz, 1H), 7.42 (d, J = 3.6 Hz, 1H), 7.19 (d, J = 2.4 Hz, 1H), 7.03-6.99 (m, 2H), 6.64 (d, J = 4.0 Hz, 1H), 6.31 (d, J = 5.2 Hz, 1H), 4.61 (t, J = 5.2 Hz, 1H), 4.48 (t, J = 4.0 Hz, 1H), 4.44-4.40 (m, 2H), 4.36-4.32 (m, 1H), 3.84 (t, J = 4.4 Hz, 4H), 3.69 (t, J = 5.2 Hz, 4H). |
| 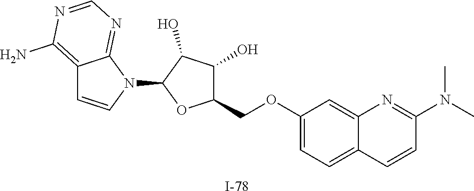 | A | 436.9 | ¹H NMR (400 MHz, CD3OD) δ ppm 8.12 (s, 1H), 7.90 (d, J = 9.1 Hz, 1H), 7.57 (d, J = 8.8 Hz, 1H), 7.43 (d, J = 3.7 Hz, 1H), 7.19 (d, J = 2.3 Hz, 1H), 6.95 (dd, J = 8.7, 2.4 Hz, 1H), 6.89 (d, J = 9.1 Hz, 1H), 6.64 (d, J = 3.7 Hz, 1H), 6.32 (d, J = 5.4 Hz, 1H), 4.61 (t, J = 5.3 Hz, 1H), 4.49-4.47 (m, 1H), 4.45- 4.39 (m, 2H), 4.38-4.29 (m, 1H), 3.22 (s, 6H). |
| 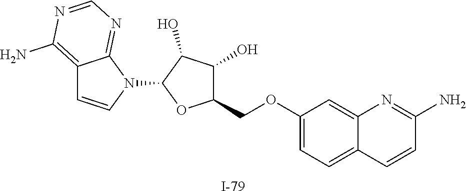 | A | 408.9 | ¹H NMR (400 MHz, CD3OD) δ ppm 8.09 (s, 1H), 7.86 (d, J = 8.8 Hz, 1H), 7.55 (dd, J = 13.0, 6.2 Hz, 2H), 7.03 (d, J = 2.3 Hz, 1H), 6.98 (dd, J = 8.8, 2.4 Hz, 1H), 6.68 (d, J = 8.8 Hz, 1H), 6.61-6.58 (m, 2H), 4.62- 4.56 (m, 1H), 4.54-4.50 (m, 2H), 4.38-4.35 (m, 1H), 4.28- 4.24 (m, 1H). |
| 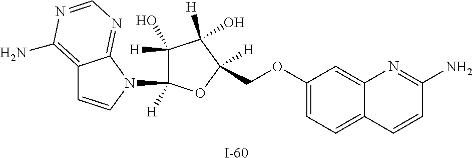 | A | 409.0 | ¹H NMR (400 MHz, CD₃OD) δ ppm 8.12 (s, 1H), 7.87 (d, J = 8.8 Hz, 1H), 7.58 (d, J = 8.8 Hz, 1H), 7.40 (d, J = 3.8 Hz, 1H), 7.05 (d, J = 2.4 Hz, 1H), 6.98 (dd, J = 8.7, 2.5 Hz, 1H), 6.69 (d, J = 8.8 Hz, 1H), 6.64 (d, J = 3.7 Hz, 1H), 6.31 (d, J = 5.3 Hz, 1H), 4.61 (t, J = 5.3 Hz, 1H), 4.50-4.46 (m, 1H), 4.44- 4.38 (m, 2H), 4.35-4.31 (m, 1H). |
| 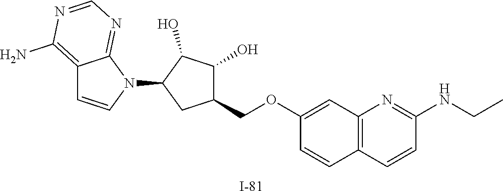 | A | 435.2 | ¹H NMR (400 MHz, DMSO- d₆) δ ppm 8.04 (s, 1H), 7.33 (d, J = 8.4 Hz, 1H), 7.49 (d, J = 8.8 Hz, 1H), 7.32 (d, J = 3.6 Hz, 1H), 6.98 (d, J = 2.0 Hz, 1H), 6.92 (br, 2H), 6.79 (dd, J = 8.8, 2.4 Hz, 1H), 6.57-6.55 (m, 2H), 4.96-4.83 (m, 3H), 4.31-4.29 (m, 1H), 4.21-4.17 (m, 1H), 4.11-4.07 (m, 1H), 3.97-3.93 (m, 1H), 2.40-2.31 (m, 2H), 1.72-1.61 (m, 1H), 1.21-1.17 (t, J = 7.2 Hz, 3H) |
| 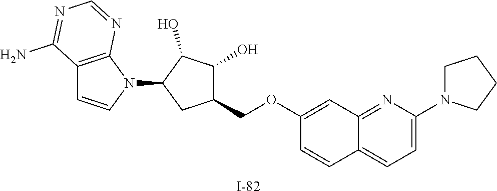 | A | 462.2 | ¹H NMR (400 MHz, DMSO- d6) δ 7.29 (s, 1H), 7.09 (d, J = 9.2 Hz, 1H), 6.76 (d, J = 8.4 Hz, 1H), 6.54 (d, J = 3.6 Hz, 1H), 6.40-6.39 (m, 1H), 6.12 (dd, J = 8.8, 2.4 Hz, 1H), 5.93 (d, J = 8.8 Hz, 1H), 5.85 (d, J = 3.6 Hz, 1H), 4.28- 4.23 (m, 2H), 3.67-3.63 (m, 1H), 3.44 (d, J = 5.2 Hz, 2H), 3.39-3.37 (m, 1H), 2.83-2.79 (m, 4H), 1.78-1.73 (m, 2H), 1.29-1.26 (m, 4H). |
| 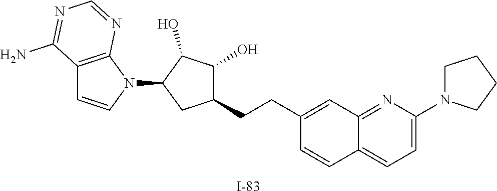 | D | 459.3 | ¹H NMR (400 MHz, DMSO- d₆) δ 8.03 (s, 1H), 7.93 (d, J = 8.0 Hz, 1H),), 7.57 (d, J = 8.1 Hz, 1H), 7.38 (s, 1H), 7.26 (d, J = 3.5 Hz, 1H), 7.04 (dd, J = 8.1, 1.4 Hz, 1H), 6.91 (s, 2H), 6.79 (d, J = 9.0 Hz, 1H), 6.54 (d, J = 3.2 Hz), 4.85-4.74 (m, 2H), 4.64 (d, J = 4.9 Hz, 1H), 4.25-4.17 (m, 1H), 3.77-3.73 (m, 1H), 3.53-3.52 (m, 4H), 2.83-2.64 (m, 2H), 2.28-2.20 (m, 1H), 1.99-1.96 (m, 4H), |
| | | | 1.90-1.83 (m, 1H), 1.76-1.69 |
| | | | (m, 1H), 1.56-1.42 (m, 2H). |
| 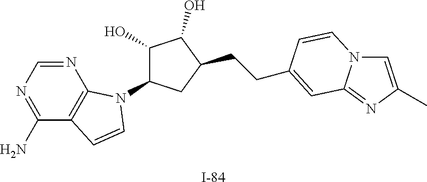 | D | 393.1 | ¹H NMR (400 MHz, DMSO- d6) δ ppm 8.33 (d, J = 6.9 Hz, 1H), 8.03 (s, 1H), 7.56 (s, 1H), 7.28-7.20 (m, 2H), 6.90 (s, 2H), 6.72 (dd, J = 6.9, 1.6 Hz, 1H), 6.54 (d, J = 3.5 Hz, 1H), 4.84- 4.73 (m, 2H), 4.63 (d, J = 4.6 Hz, 1H), 4.21-4.16 (m, 1H), 3.75-3.72 (m, 1H), 2.68-2.60 (m, 2H), 2.29 (s, 3H), 2.26-2.19 (m, 1H), 1.96-1.83 (m, 2H), 1.73-1.64 (m, 1H), 1.54-1.46 (m, 1H). |
| 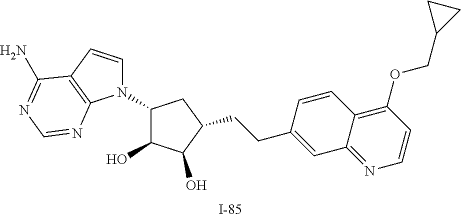 | D | 460.0 | ¹H NMR (400 MHz, DMSO- d6) δ ppm δ 8.68 (d, J = 5.2 Hz, 1H), 8.10-8.07 (m, 2H), 7.76 (s, 1H), 7.49-7.46 (m, 1H), 7.32 (d, J = 3.6 Hz, 1H), 7.17 (s, 1H), 6.95 (d, J = 5.6 Hz, 1H), 6.59 (d, J = 3.6 Hz, 1H), 4.81- 4.79 (m, 2H), 4.68 (s, 1H), 4.20-4.12 (m, 1H), 4.11 (d, J = 6.8 Hz, 2H), 3.76 (s, 1H), 2.84- 2.82 (m, 2H), 2.26-2.23 (m, 1H), 2.01-1.90 (m, 1H), 1.85- 1.83 (m, 1H), 1.72-1.61 (m, 1H), 1.54-1.52 (m, 1H), 1.37-1.23 (m, 1H), 0.66-0.62 |
| | | | (m, 2H), 0.44-0.42 (m, 2H). |
| 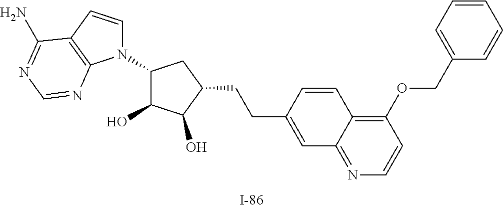 | D | 496 | ¹H NMR (400 MHz, DMSO- d6) δ ppm 8.71 (d, J = 5.2 Hz, 1H), 8.16 (s, 0.5H), 8.12-8.06 (m, 2H), 7.88 (s, 1H), 7.78- 7.75 (m, 1H), 7.58 (d, J = 7.2 Hz, 2H), 7.47 (t, J = 7.2 Hz, 2H), 7.40-7.38 (m, 1H), 7.39- 7.32 (m, 1H), 7.09 (d, J = 5.0 Hz, 1H), 6.95-6.92 (m, 2H), 6.70 (d, J = 2.8 Hz, 1H), 6.57 (d, J = 3.6 Hz, 1H) |
| 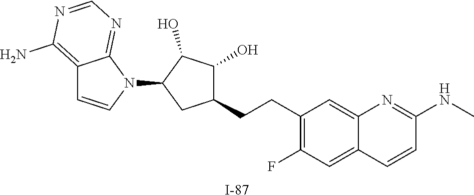 | D | 437.1 | ¹H NMR (400 MHz, CD3OD) δ ppm 8.27 (s, 0.67H), 8.22- 8.10 (m, 2H), 7.77-7.75 (m, 1H), 7.55 (d, J = 9.6 Hz, 1H), 7.39-7.37 (m, 1H), 6.99-6.97 (m, 1H), 6.75-6.73 (m, 1H), 4.97 (s, 1H), 4.40-4.37 (m, 1H), 3.95-3.93 (m, 1H), 3.16 (s, 3H), 2.97-2.93 (m, 2H), 2.51-2.44 (m, 1H), 2.13-2.05 (m, 2H), 1.89-1.85 (m, 1H), 1.76-1.68 (m, 1H) |
| 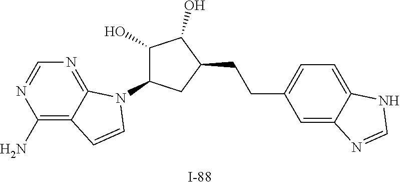 | D | 379.0 | ¹H NMR (400 MHz, CD3OD) δ 8.12 (s, 1H), 8.08 (s, 1H), 7.53-7.48 (m, 2H), 7.26 (d, J = 3.6 Hz, 1H), 7.20 (d, J = 8.3 Hz, 1H), 6.63 (d, J = 3.6 Hz, 1H), 4.40-4.30 (m, 1H), 3.95- 3.85 (m, 1H), 2.89-2.83 (m, 3H), 2.47-2.41 (m, 1H), 2.14- 2.06 (m, 2H), 1.85-1.80 (m, 1H), 1.71-1.62 (m, 1H). |
| 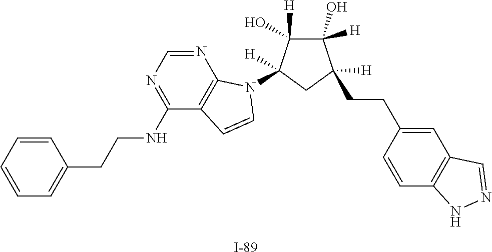 | D | 483.2 | ¹H NMR (400 MHz, DMSO) δ 12.93 (s, 1H), 8.14 (s, 1H), 7.97 (s, 1H), 7.56 (s, 2H), 7.45 (d, J = 8.5 Hz, 1H), 7.34-7.24 (m, 6H), 7.22-7.21 (m, 1H), 6.56 (d, J = 3.4 Hz, 1H), 4.90- 4.72 (m, 2H), 4.63 (d, J = 5.0 Hz, 1H), 4.23-4.19 (m, 1H), 3.81-3.58 (m, 3H), 2.91 (t, J = 7.6 Hz, 2H), 2.67 (s, 1H), 2.26- 2.20 (m, 2H), 1.92-1.83 (m, 2H), 1.70-1.67 (m, 1H), 1.53- 1.50 (m, 1H). |
| 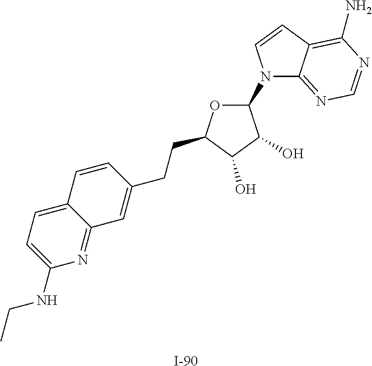 | A | 435.3 | ¹H NMR (400 MHz, CD3OD) δ ppm 8.12 (s, 1H), 7.79 (d, J = 9.0 Hz, 1H), 7.61-7.43 (m, 2H), 7.30 (d, J = 3.7 Hz, 1H), 7.11 (dd, J = 8.1, 1.5 Hz, 1H), 6.82- 6.61 (m, 2H), 6.17 (d, J = 5.0 Hz, 1H), 4.50 (t, J = 5.3 Hz, 1H), 4.12 (t, J = 5.2 Hz, 1H), 4.06-3.95 (m, 1H), 3.49 (q, J = 7.2 Hz, 2H), 3.03-2.91 (m, 1H), 2.91-2.79 (m, 1H), 2.30-1.95 (m, 2H), 1.30 (t, J = 7.2 Hz, 3H). |
| 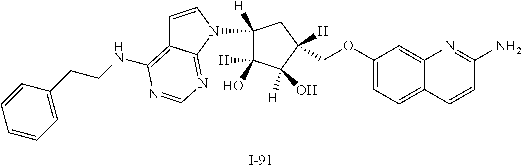 | A | 511.2 | ¹H NMR (400 MHz, DMSO- d6) δ ppm 8.15 (s, 1H), 7.79 (d, J = 8.8 Hz, 1H), 7.56-7.51 (m, 2H), 7.35-7.24 (m, 5H), 7.24- 7.16 (m, 1H), 6.92 (d, J = 2.4 Hz, 1H), 6.83 (dd, J = 8.7, 2.5 Hz, 1H), 6.60-6.56 (m, 2H), 6.31 (s, 2H), 4.99-4.86 (m, 2H), 4.83 (d, J = 3.2 Hz, 1H), 4.35- 4.26 (m, 1H), 4.21-4.15 (m, 1H), 4.13-4.07 (m, 1H), 3.96 (s, 1H), 3.72-3.65 (t, 2H), 2.96- |
| | | | 2.86 (m, 2H), 2.36-2.26 (m, |
| | | | 1H), 1.74-1.64 (m, 1H). |
| 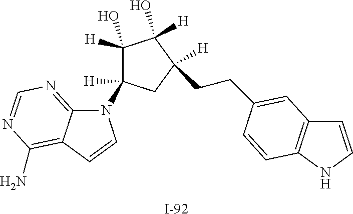 | D | 378.0 | ¹H NMR (400 MHz, DMSO- d6) δ 10.94 (s, 1H), 8.03 (s, 1H), 7.35 (s, 1H), 7.30-7.23 (m, 3H), 7.01-6.82 (m, 3H), 6.54 (d, J = 3.5 Hz, 1H), 6.33 (d, J = 2.7 Hz, 1H), 4.82-4.72 (m, 2H), 4.24-4.15 (m, 1H), 3.74-3.72 (m, 1H), 2.75-2.60 (m, 2H), 2.26-2.20 (m, 1H), 1.93-1.83 (m, 2H), 1.69-1.63 (m, 1H), 1.54-1.47 (m, 1H). |
| 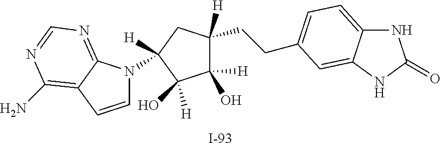 | D | 395.0 | ¹H NMR (400 MHz, DMSO) δ 10.48 (d, J = 16.8 Hz, 2H), 8.03 (s, 1H), 7.25 (d, J = 3.5 Hz, 1H), 6.91 (s, 2H), 6.82- 6.76 (m, 3H), 6.53 (d, J = 3.5 Hz, 1H), 4.82-4.76 (m, 2H), 4.61 (d, J = 4.8 Hz, 1H), 4.22- 4.15 (m, 1H), 3.72-3.69 (m, 1H), 2.70-2.53 (m, 2H), 2.28- 2.14 (m, 1H), 1.87-1.80 (m, 2H), 1.64-1.57 (m, 1H), 1.52- |
| | | | 1.44 (m, 1H). |
| 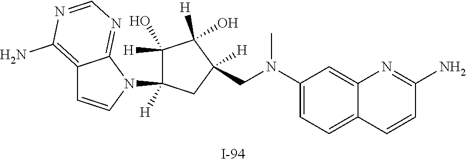 | A | 419.8 | ¹H NMR (400 MHz, CD3OD) δ ppm δ 8.49 (br, 1.6 H), 8.09- 8.07 (m, 2H), 7.65 (d, J = 8.8 Hz, 1H), 7.26 (d, J = 3.6 Hz, 1H), 7.14 (dd, J = 9.2, 2 Hz, 1H), 6.73 (s, 1H), 6.63-6.59 (m, 2H), 4.87-4.85 (m, 1H), 4.48-4.44 (m, 1H), 4.06-4.03 (m, 1H), 3.93-3.91 (m, 1H), 3.64-3.59 (m, 1H), 3.21 (s, 3H), 2.55-2.52 (m, 1H), 2.39-2.35 (m, 1H), |
| | | | 1.84-1.81 (m, 1H) |
| 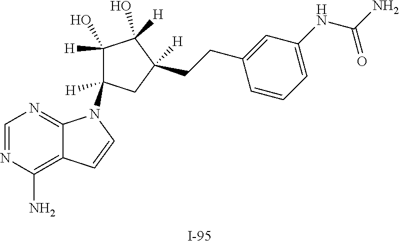 | D | 366.0 | ¹H NMR (400 MHz, DMSO- d6) δ 8.42 (s, 1H), 8.03 (s, 1H), 7.29-7.22 (m, 2H), 7.22-7.17 (m, 1H), 7.11 (t, J = 7.7 Hz, 1H), 6.89 (s, 2H), 6.77-6.75 (m, 1H), 6.53 (d, J = 3.5 Hz, 1H), 5.78 (s, 2H), 4.84-4.71 (m, 2H), 4.60 (d, J = 5.0 Hz, 1H), 4.22- 4.17 (m, 1H), 3.74-3.70 (m, 1H), 2.65-2.51 (m, 2H), 2.22- 2.19 (m, 1H), 1.89-1.83 (m, 2H), 1.69-1.556 (m, 1H), 1.55- 1.43 (m, 1H). |
| 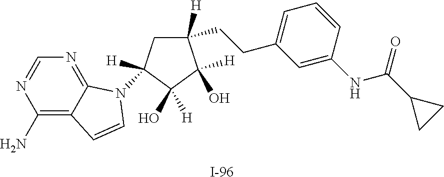 | D | 422.1 | ¹H NMR (400 MHz, DMSO- d6) δ ppm 10.10 (s, 1H), 8.03 (s, 1H), 7.47 (s, 1H), 7.39 (d, J = 7.8 Hz, 1H), 7.25 (d, J = 3.6 Hz, 1H), 7.18 (t, J = 7.8 Hz, 1H), 6.94-6.84 (m, 3H), 6.54 (d, J = 3.5 Hz, 1H), 4.83-4.73 (m, 2H), 4.61 (d, J = 4.9 Hz, 1H), 4.22-4.15 (m, 1H), 3.75- 3.69 (m, 1H), 2.69-2.52 (m, 2H), 2.24-2.19 (m, 1H), 1.89- 1.80 (m, 2H), 1.79-1.73 (m, 1H), 1.64-1.60 (m, 1H), 1.54- |
| | | | 1.43 (m, 1H), 0.78 (t, J = 5.9 |
| | | | Hz, 4H). |
| 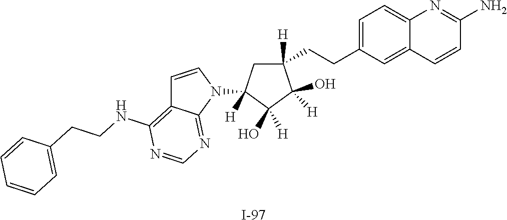 | D | 509.1 | ¹H NMR (400 MHz, CD₃OD) δ ppm 8.15 (s, 1H), 7.90-7.85 (m, 1H), 7.49-7.41 (m, 3H), 7.27 (d, J = 4 Hz, 4H), 7.21 (s, 2H), 6.81-6.78 (m, 1H), 6.58 (d, J = 3.6 Hz, 1H), 4.91-4.86 (m, 1H), 4.35-4.31 (m, 1H), 3.91 (s, 1H), 3.78-3.73 (m, 2H), 3.00-2.95 (m, 2H), 2.82-2.78 (m, 2H), 2.46-2.41 (m, 1H), 2.05 (s, 2H), 1.81-1.79 (m, 1H), 1.66-1.58 (m, 1H). |
| 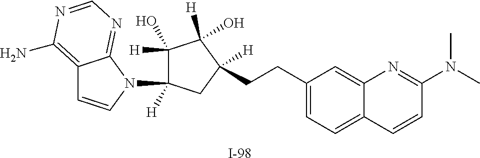 | D | 433.2 | ¹H NMR (400 MHz, CD₃OD) δ ppm 8.03 (s, 1H), 7.95 (d, J = 8.8 Hz, 1H), 7.58 (d, J = 8.4 Hz, 1H), 7.43 (s, 1H), 7.26 (d, J = 3.6 Hz, 1H), 7.08-6.99 (m, 1H), 6.91 (s, 1H), 6.54 (d, J = 3.2 Hz, 1H), 4.91-4.76 (m, 2H), 4.64 (d, J = 4.8 Hz, 1H), 4.21- 4.18 (m, 1H), 3.76-3.74 (m, 1H), 3.14 (s, 6H), 2.77-2.66 (m, 1H), 2.43-2.33 (m, 1H), 2.32- 2.26 (m, 1H), 2.24-2.22 (m, |
| | | | 1H), 1.94-1.53 (m, 1H). |
| 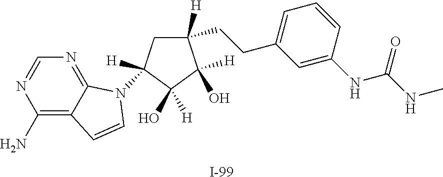 | D | 411.1 | ¹H NMR (400 MHz, DMSO) δ 8.43 (d, J = 10.4 Hz, 1H), 8.12 (s, 0.5H), 8.03 (s, 0.5H), 7.39 (d, J = 4.7 Hz, 0.5H), 7.26- 7.25 (m, 2H), 7.20 (d, J = 8.2 Hz, 1H), 7.13-7.09 (m, 1H), 6.91 (s, 1H), 6.77-6.74 (m, 1H), 6.54-6.52 (m, 1H), 5.97- 5.96 (m, 0.5H), 5.80 (s, 1H), 4.86-4.73 (m, 2H), 4.63 (dd, J = 4.9, 1.8 Hz, 1H), 4.25-4.15 (m, 1H), 3.77-3.64 (m, 1H), 3.31 (s, 1.5H), 2.95 (d, J = 4.6 |
| | | | Hz, 1.5H), 2.63 (d, J = 4.6 Hz, |
| | | | 2H), 2.28-2.17 (m, 1H), 1.93- |
| | | | 1.77 (m, 2H), 1.67-1.56 (m, |
| | | | 1H), 1.55-1.43 (m, 1H). |
| 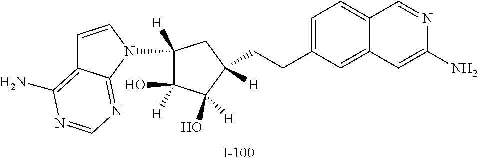 | D | 405.2 | ¹H NMR (400 MHz, CD3OD) δ ppm 8.71 (s, 1H), 8.09 (s, 1H), 7.76 (d, J = 8.5 Hz, 1H), 7.40 (s, 1H), 7.26 (d, J = 3.6 Hz, 1H), 7.18 (dd, J = 8.5, 1.4 Hz, 1H), 6.77 (s, 1H), 6.63 (d, J = 3.6 Hz, 1H), 4.64 (s, 1H), 4.36 (dd, J = 7.8, 6.2 Hz, 1H), 3.96-3.88 (m, 1H), 2.88-2.80 (m, 2H), 2.49-2.42 (m, 1H), 2.12-2.05 (m, 2H), 1.91-1.76 (m, 1H), 1.71-1.63 (m, 1H). |
| 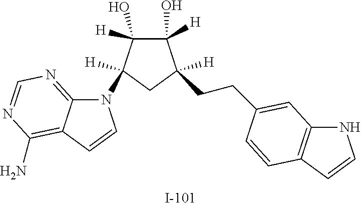 [SMILES: Nc1ncnc2c1ccn2[C@@H]1C[C@H](CCc2ccc3cc[nH]c3c2)[C@@H](O)[C@H]1O] | D | 378.0 | ¹H NMR (400 MHz, DMSO) δ 10.92 (s, 1H), 8.03 (s, 1H), 7.43 (d, J = 8.1 Hz, 1H), 7.27- 7.24 (m, 2H), 7.20 (s, 1H), 6.91 (s, 2H), 6.86 (dd, J = 8.1, 1.1 Hz, 1H), 6.54 (d, J = 3.5 Hz, 1H), 6.37-6.34 (m, 1H), 4.83- 4.76 (m, 2H), 4.62 (s, 1H), 4.26- 4.13 (m, 1H), 3.81-3.68 (m, 1H), 2.82-2.62 (m, 2H), 2.28- 2.21 (m, 1H), 1.97-1.82 (m, 2H), 1.72-1.62 (m, 1H), 1.55- 1.44 (m, 1H). |
| 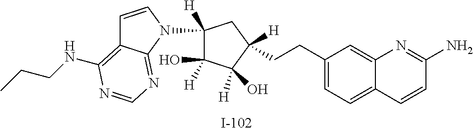 | D | 447.0 | ¹H NMR (400 MHz, DMSO- d6) δ ppm 8.09 (s, 1H), 7.83 (d, J = 8.4 Hz, 1H), 7.53 (d, J = 8.0 Hz, 1H), 7.42 (t, J = 5.2 Hz, 1H), 7.27 (s, 1H), 7.24 (d, J = 3.6 Hz, 1H), 7.04 (d, J = 8.4 Hz, 1H), 6.68 (d, J = 8.8 Hz, 1H), 6.57 (d, J = 3.6 Hz, 1H), 6.39 (s, 2H), 4.82-4.75 (m, 2H), 4.64 (d, J = 4.8 Hz, 1H), 4.23- |
| | | | 4.18 (m, 1H), 3.76-3.72 (m, |
| | | | 1H), 3.44-3.38 (m, 2H), 2.77- |
| | | | 2.67 (m, 2H), 2.27-2.20 (m, |
| | | | 1H), 1.97-1.85 (m, 2H), 1.73- |
| | | | 1.67 (m, 1H), 1.62-1.57 (m, |
| | | | 2H), 1.55-1.47 (m, 1H), 0.92 (t, |
| | | | J = 7.4 Hz, 3H). |
| 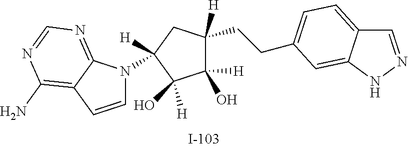 [SMILES: Nc1ncnc2c1ccn2[C@@H]1C[C@H](CCc2ccc3cn[nH]c3c2)[C@@H](O)[C@H]1O] | D | 379.1 | ¹H NMR (400 MHz, DMSO- d6) δ ppm 12.90 (s, 1H), 8.03 (s, 1H), 7.98 (s, 1H), 7.65 (d, J = 8.3 Hz, 1H), 7.33 (s, 1H), 7.26 (d, J = 3.5 Hz, 1H), 7.00 (d, J = 9.0 Hz, 1H), 6.92 (s, 2H), 6.54 (d, J = 3.5 Hz, 1H), 4.85-4.73 (m, 2H), 4.63 (d, J = 4.9 Hz, 1H), 4.22-4.18 (m, 1H), 3.76-3.72 (m, 1H), 2.81-2.67 (m, 2H), 2.26-2.21 (m, 1H), |
| | | | 1.95-1.85 (m, 2H), 1.74-1.68 |
| | | | (m, 1H), 1.55-1.47 (m, 1H). |
| 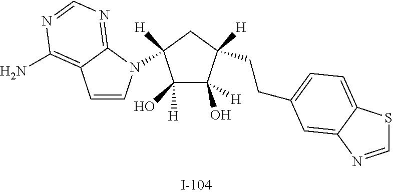 [SMILES: Nc1ncnc2c1ccn2[C@@H]1C[C@H](CCc2ccc3scnc3c2)[C@@H](O)[C@H]1O] | D | 396.0 | ¹H NMR (400 MHz, CD3OD) δ ppm 9.23 (s, 1H), 8.08 (s, 1H), 8.04-7.92 (m, 2H), 7.45- 7.43 (m, 1H), 7.27 (d, J = 3.6 Hz, 1H), 6.63 (d, J = 3.6 Hz, 1H), 4.65 (s, 1H), 4.37-7.34 (m, 1H), 3.98-3.86 (m, 1H), 3.05- 2.83 (m, 2H), 2.49-2.42 (m, 1H), 2.13-2.07 (m, 2H), 1.94- 1.79 (m, 1H), 1.72-1.64 (m, 1H). |
| 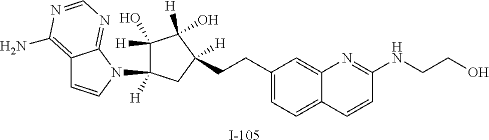 [SMILES: Nc1ncnc2c1ccn2[C@@H]1C[C@H](CCc2ccc3ccc(NCCO)nc3c2)[C@@H](O)[C@H]1O ; "I-105"] | D | 449.2 | ¹H NMR (400 MHz, CD₃OD) δ ppm 8.08 (s, 1H), 7.83 (d, J = 8.8 Hz, 1H), 7.55 (d, J = 8.4 Hz, 1H), 7.46 (s, 1H), 7.26 (d, J = 3.6 Hz, 1H), 7.15-7.13 (m, 1H), 6.76-6.74 (m, 1H), 6.63 (d, J = 3.6 Hz, 1H), 4.65 (s, 1H), 4.37-4.34 (m, 1H), 3.94- 3.91 (m, 1H), 3.79 (t, J = 5.2 Hz, 2H), 3.61 (t, J = 5.6 Hz, 2H), 2.89-2.82 (m, 2H), 2.47- |
| | | | 2.44 (m, 1H), 2.11-2.07 (m, |
| | | | 2H), 1.86-1.84 (m, 1H), 1.69- |
| | | | 1.66 (m, 1H). |
| 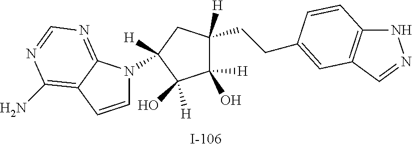 [SMILES: Nc1ncnc2c1ccn2[C@@H]1C[C@H](CCc2ccc3[nH]ncc3c2)[C@@H](O)[C@H]1O] | D | 379.1 | ¹H NMR (400 MHz, DMSO- d6) δ ppm 12.94 (s, 1H), 8.03 (s, 1H), 7.97 (s, 1H), 7.55 (s, 1H), 7.45 (d, J = 8.4 Hz, 1H), 7.26-7.22 (m, 1H), 6.92 (s, 1H), 6.54 (d, J = 3.5 Hz, 1H), 4.82- 4.77 (m, 2H), 4.62 (d, J = 4.9 Hz, 1H), 4.20-4.17 (m, 1H), 3.75-3.71 (m, 1H), 2.77-2.64 (m, 2H), 2.29-2.17 (m, 1H), 1.95-1.84 (m, 2H), 1.70-1.65 |
| | | | (m, 1H), 1.58-1.41 (m, 1H). |
| 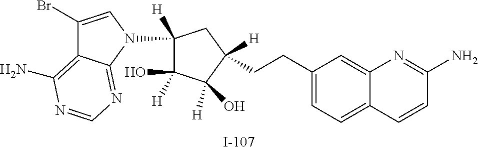 | D | 483.1 | ¹H NMR (400 MHz, DMSO- d6) δ ppm 8.08 (s, 1H), 7.83 (d, J = 8.8 Hz, 1H), 7.61 (s, 1H), 7.54 (d, J = 8.8 Hz, 1H), 7.27 (s, 1H), 7.05 (d, J = 8.0 Hz, 1H), 6.69 (d, J = 8.8 Hz, 2H), 6.33 (s, 2H), 4.86-4.80 (m, 2H), 4.66 (d, J = 4.8 Hz, 1H), 4.21- 4.19 (m, 1H), 3.79-3.70 (m, 1H), 2.74-2.66 (m, 2H), 2.24- 2.21 (m, 1H), 1.93-1.84 (m, |
| | | | 2H), 1.73-1.67 (m, 1H), 1.51- |
| | | | 1.48 (m, 1H) |
| 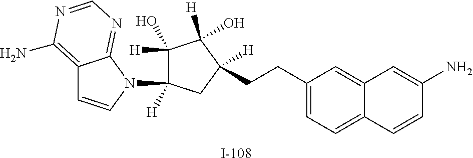 [SMILES: Nc1ccc2ccc(CC[C@H]3C[C@@H](n4ccc5c(N)ncnc54)[C@H](O)[C@@H]3O)cc2c1 ; "I-108"] | D | 404.0 | ¹H NMR (400 MHz, DMSO- d6) δ ppm 8.03 (s, 1H), 7.53 (t, J = 7.9 Hz, 2H), 7.29 (s, 1H), 7.26 (d, J = 3.5 Hz, 1H), 6.98 (d, J = 8.3 Hz, 1H), 6.92 (s, 2H), 6.85 (dd, J = 8.7, 2.1 Hz, 1H), 6.74 (s, 1H), 6.54 (d, J = 3.5 Hz, 1H), 5.31 (s, 2H), 4.85- 4.74 (m, 2H), 4.63 (d, J = 4.9 Hz, 1H), 4.25-4.15 (m, 1H), 3.78-3.69 (m, 1H), 2.76-2.61 (m, 2H), 2.30-2.19 (m, 1H), |
| | | | 2.00-1.83 (m, 2H), 1.69 (s, 1H), |
| | | | 1.58-1.45 (m, 1H). |
| 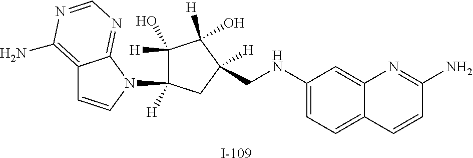 [SMILES: Nc1ccc2ccc(NC[C@H]3C[C@@H](n4ccc5c(N)ncnc54)[C@H](O)[C@@H]3O)cc2n1 ; "I-109"] | D | 406.1 | ¹H NMR (400 MHz, CD3OD) δ 8.47 ppm (s, 4.63H), 8.10 (s, 1H), 8.04 (d, J = 9.1 Hz, 1H), 7.55 (d, J = 8.8 Hz, 1H), 7.29 (d, J = 3.6 Hz, 1H), 6.89 (dd, J = 8.9, 2.0 Hz, 1H), 6.66 (d, J = 3.4 Hz, 2H), 6.58 (d, J = 9.0 Hz, 1H), 4.49-4.41 (m, 1H), 4.10-4.02 (m, 1H), 3.56-3.47 (m, 1H), 3.39 (d, J = 7.1 Hz, 1H), 2.55-2.42 (m, 2H), 1.87- |
| | | | 1.75 (m, 1H). |
| 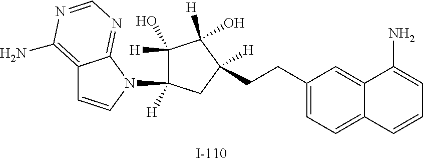 [SMILES: Nc1cccc2ccc(CC[C@H]3C[C@@H](n4ccc5c(N)ncnc54)[C@H](O)[C@@H]3O)cc12 ; "I-110"] | D | 403.9 | ¹H NMR (400 MHz, DMSO- d6) δ ppm 8.03 (s, 1H), 7.86 (s, 1H), 7.65 (d, J = 8.4 Hz, 1H), 7.30 (d, J = 8.3 Hz, 1H), 7.26 (d, J = 3.5 Hz, 1H), 7.12 (t, J = 7.7 Hz, 1H), 7.03 (d, J = 8.0 Hz, 1H), 6.92 (s, 2H), 6.63 (d, J = 7.3 Hz, 1H), 6.54 (d, J = 3.5 Hz, 1H), 5.61 (s, 2H), 4.86- 4.74 (m, 2H), 4.64 (d, J = 5.0 Hz, 1H), 4.25-4.17 (m, 1H), 3.80-3.73 (m, 1H), 2.85-2.68 |
| | | | (m, 2H), 2.41-2.19 (m, 1H), |
| | | | 2.05-2.85 (m, 2H), 1.80-1.68 |
| | | | (m, 1H), 1.59-1.47 (m, 1H). |
| 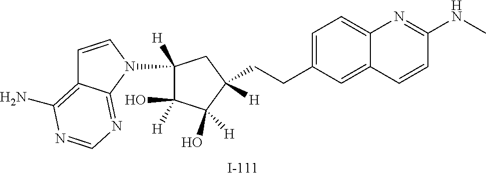 | D | 419.2 | ¹H NMR (400 MHz, CD₃OD) δ ppm 8.08 (s, 1H), 7.79 (d, J = 8.8 Hz, 1H), 7.61 (d, J = 8.8 Hz, 1H), 7.43 (d, J = 8.8 Hz, 2H), 7.26 (d, J = 3.2 Hz, 1H), 6.72 (d, J = 8.8 Hz, 1H), 6.62 (d, J = 3.6 Hz, 1H), 4.37-4.33 (m, 1H), 3.93-3.90 (m, 1H), 3.01 (s, 3H), 2.84-2.75 (m, 2H), 2.48-2.41 (m, 1H), 2.07-2.06 (m, 2H), 1.81-1.80 (m, 1H), 1.69-1.61 (m, 1H). |
| 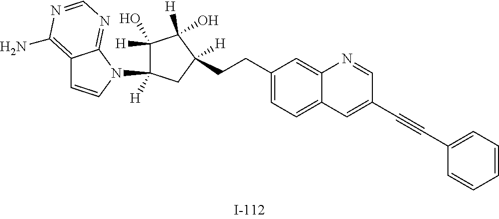 [SMILES: Nc1ncnc2c1ccn2[C@@H]1C[C@H](CCc2ccc3cc(C#Cc4ccccc4)cnc3c2)[C@@H](O)[C@H]1O] | D | 490.0 | ¹H NMR (400 MHz, DMSO- d6) δ ppm 8.97 (d, J = 2.1 Hz, 1H), 8.59 (d, J = 1.8 Hz, 1H), 8.03 (s, 1H), 7.95 (d, J = 8.4 Hz, 1H), 7.89 (s, 1H), 7.66- 7.63 (m, 2H), 7.60 (dd, J = 8.4, 1.3 Hz, 1H), 7.49-7.47 (m, 3H), 7.27 (d, J = 3.5 Hz, 1H), 6.92 (s, 2H), 6.54 (d, J = 3.5 Hz, 1H), 4.85-4.76 (m, 2H), 4.67 (d, J = 5.0 Hz, 1H), 4.24-4.19 (m, 1H), 3.78-3.73 (m, 1H), 2.93-2.84 (m, 2H), 2.30-2.25 (m, 1H), 2.04-1.97 (m, 1H), 1.92-1.86 (m, 1H), 1.81-1.75 |
| | | | (m, 1H), 1.57-1.52 (m, 1H). |
| 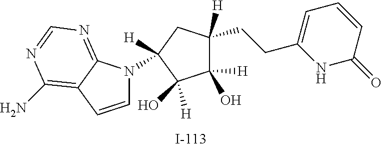 [SMILES: Nc1ncnc2c1ccn2[C@@H]1C[C@H](CCc2cccc(=O)[nH]2)[C@@H](O)[C@H]1O] | D | 356.1 | ¹H NMR (400 MHz, DMSO- d6) δ ppm 8.03 (s, 1H), 7.36- 7.32 (m, 1H), 7.25 (d, J = 3.5 Hz, 1H), 6.93 (s, 2H), 6.55 (d, J = 3.5 Hz, 1H), 6.13 (d, J = 8.8 Hz, 1H), 6.02 (d, J = 6.7 Hz, 1H), 4.83-4.77 (m, 2H), 4.19- 4.16 (m, 1H), 3.74-3.72 (m, 1H), 2.52-2.46 (m, 2H), 2.25- 2.18 (m, 1H), 1.92-1.81 (m, 2H), 2.49-2.37 (m, 1H), 2.34- |
| | | | 2.23 (m, 1H). |
| 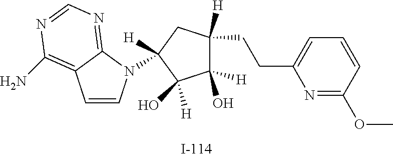 [SMILES: COc1cccc(CC[C@H]2C[C@@H](n3ccc4c(N)ncnc43)[C@H](O)[C@@H]2O)n1] | D | 370.0 | ¹H NMR (400 MHz, DMSO- d6) δ ppm 8.02 (s, 1H), 7.59 (dd, J = 8.1, 7.3 Hz, 1H), 7.25 (d, J = 3.5 Hz, 1H), 6.91 (s, 2H), 6.85 (d, J = 7.2 Hz, 1H), 6.61 (d, J = 8.2 Hz, 1H), 6.54 (d, J = 3.5 Hz, 1H), 4.85-4.73 (m, 2H), 4.63 (d, J = 5.0 Hz, 1H), 4.21-4.12 (m, 1H), 3.75- 3.71 (m, 1H), 2.77-2.61 (m, 2H), 2.26-2.19 (m, 1H), 2.04- 1.95 (m, 1H), 1.88-1.85 (m, |
| | | | 1H), 1.79-1.65 (m, 1H), 1.52- |
| | | | 1.44 (m, 1H). |
| 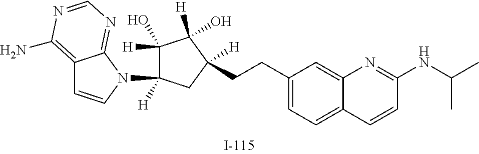 [SMILES: CC(C)Nc1ccc2ccc(CC[C@H]3C[C@@H](n4ccc5c(N)ncnc54)[C@H](O)[C@@H]3O)cc2n1] | D | 433.2 | ¹H NMR (400 MHz, DMSO- d6) δ ppm 8.03 (s, 1H), 7.77 (d, J = 8.9 Hz, 1H), 7.51 (d, J = 8.1 Hz, 1H), 7.34 (s, 1H), 7.27 (d, J = 3.5 Hz, 1H), 7.08-6.85 (m, 4H), 6.67 (d, J = 8.9 Hz, 1H), 6.54 (d, J = 3.5 Hz, 1H), 4.88-4.72 (m, 2H), 4.64 (d, J = 4.9 Hz, 1H), 4.23-4.18 (m, 1H), 3.76-3.73 (m, 1H), 3.43-3.38 (m, 2H), 2.79-2.63 (m, 2H), |
| | | | 2.28-2.21 (m, 1H), 2.01-1.83 |
| | | | (m, 2H), 1.75-1.68 (m, 1H), |
| | | | 1.56-1.47 (m, 1H), 1.19 (t, J = |
| | | | 7.2 Hz, 3H). |
| 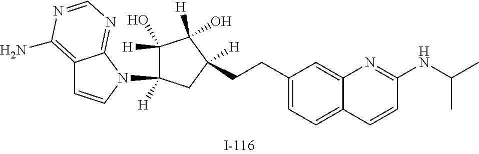 [SMILES: CC(C)Nc1ccc2ccc(CC[C@H]3C[C@@H](n4ccc5c(N)ncnc54)[C@H](O)[C@@H]3O)cc2n1] | D | 447.0 | ¹H NMR (400 MHz, DMSO- d₆) δ ppm 8.03 (s, 1H), 7.75 (d, J = 8.9 Hz, 1H), 7.49 (d, J = 8.1 Hz, 1H), 7.32 (s, 1H), 7.26 (d, J = 3.5 Hz, 1H), 7.01 (dd, J = 8.1, 1.4 Hz, 1H), 6.91 (s, 2H), 6.76 (d, J = 7.5 Hz, 1H), 6.64 (d, J = 8.9 Hz, 1H), 6.54 (d, J = 3.5 Hz, 1H), 4.89-4.72 (m, 2H), 4.63 (d, J = 4.9 Hz, 1H), 4.33-4.08 (m, 2H), 3.78- |
| | | | 3.70 (m, 1H), 2.84-2.59 (m, |
| | | | 2H), 2.35-2.15 (m, 1H), 2.09- |
| | | | 1.80 (m, 2H), 1.80-1.61 (m, |
| | | | 1H), 1.58-1.46 (m, 1H), 1.19 |
| | | | (d, J = 6.5 Hz, 6H). |
| 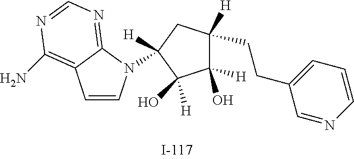 [SMILES: Nc1ncnc2c1ccn2[C@@H]1C[C@H](CCc2cccnc2)[C@@H](O)[C@H]1O] | D | 340.0 | ¹H NMR (400 MHz, DMSO- d6) δ ppm 8.46 (d, J = 1.8 Hz, 1H), 8.40 (dd, J = 4.7, 1.5 Hz, 1H), 8.03 (s, 1H), 7.68-7.63 (m, 1H), 7.32-7.30 (m, 1H), 7.25 (d, J = 3.5 Hz, 1H), 6.92 (s, 2H), 6.54 (d, J = 3.5 Hz, 1H), 4.84-4.74 (m, 2H), 4.66 (d, J = 4.9 Hz, 1H), 4.21-4.15 (m, 1H), 3.75-3.72 (m, 1H), 2.75-2.56 (m, 2H), 2.26-2.19 (m, 1H), |
| | | | 1.90-1.82 (m, 2H), 1.70-1.63 |
| | | | (m, 1H), 1.57-1.36 (m, 1H). |
| 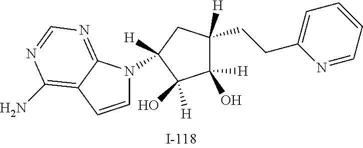 [SMILES: Nc1ncnc2c1ccn2[C@@H]1C[C@H](CCc2ccccn2)[C@@H](O)[C@H]1O] | D | 340.1 | ¹H NMR (400 MHz, CD3OD) δ ppm δ 8.69-8.27 (m, 1H), 8.08 (s, 1H), 7.80-7.76 (m, 1H), 7.38 (d, J = 7.8 Hz, 1H), 7.33- 7.23 (m, 2H), 6.62 (d, J = 3.6 Hz, 1H), 4.95-4.89 (m, 1H), 4.36-4.32 (m, 1H), 3.94-3.91 (m, 1H), 2.98-2.83 (m, 2H), 2.46-2.39 (m, 1H), 2.13-2.03 (m, 2H), 1.93-1.79 (m, 1H), 1.73-1.59 (m, 1H). |
| 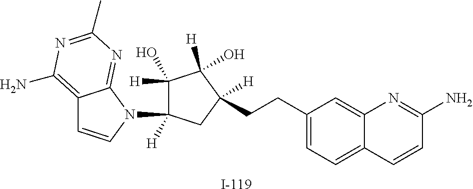 [SMILES: Cc1nc(N)c2ccn([C@@H]3C[C@H](CCc4ccc5ccc(N)nc5c4)[C@@H](O)[C@H]3O)c2n1 ; "I-119"] | D | 419.1 | ¹H NMR (400 MHz, DMSO- d6) δ ppm 7.92 (s, 1H), 7.58 (d, J = 8.1 Hz, 1H), 7.31 (s, 1H), 7.20 (s, 1H), 7.10 (d, J = 8.0 Hz, 1H), 6.91 (s, 2H), 6.73 (d, J = 8.3 Hz, 1H), 6.51 (s, 1H), 4.84-4.78 (m, 2H), 4.63 (d, J = 4.7 Hz, 1H), 4.19-4.16 (m, 1H), 3.82-3.77 (m, 2H), 2.74-2.65 (m, 2H), 2.36 (s, 3H), 2.26-2.19 (m, 1H), 1.97-1.84 (m, 2H), 1.71 (s, 1H), 1.50-1.44 (m, 1H). |
| 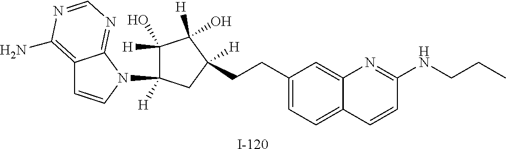 [SMILES: CCCNc1ccc2ccc(CC[C@H]3C[C@@H](n4ccc5c(N)ncnc54)[C@H](O)[C@@H]3O)cc2n1 ; "I-120"] | D | 447.2 | ¹H NMR (400 MHz, DMSO- d6) δ ppm 8.03 (s, 1H), 7.76 (d, J = 8.4 Hz, 1H), 7.50 (d, J = 7.9 Hz, 1H), 7.31-7.28 (m, 2H), 7.02 (d, J = 7.5 Hz, 1H), 6.93 (s, 3H), 6.68 (d, J = 8.8 Hz, 1H), 6.55 (s, 1H), 4.86-4.78 (m, 1H), 4.25-4.20 (m, 1H), 3.77- 3.74 (m, 2H), 3.33-3.30 (m, 2H), 2.82-7.69 (m, 2H), 2.30- 2.22 (m, 1H), 2.03-1.86 (m, |
| | | | 2H), 1.71 (s, 1H), 1.65-1.49 (m, |
| | | | 3H), 1.03-0.90 (m, 3H). |
| 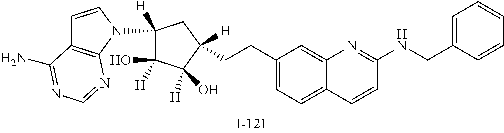 [SMILES: Nc1ncnc2c1ccn2[C@@H]1C[C@H](CCc2ccc3ccc(NCc4ccccc4)nc3c2)[C@@H](O)[C@H]1O] | D | 495.2 | ¹H NMR (400 MHz, DMSO- d6) δ ppm 8.03 (s, 1H), 7.81 (d, J = 8.8 Hz, 1H), 7.53 (d, J = 8.1 Hz, 1H), 7.48-7.43 (m 1H), 7.39 (d, J = 7.2 Hz, 2H), 7.35- 7.28 (m, 3H), 7.28-7.19 (m, 2H), 7.04 (d, J = 8.0 Hz, 1H), 6.91 (s, 2H), 6.76 (d, J = 8.9 Hz, 1H), 6.53 (d, J = 3.6 Hz, 1H), 4.85-4.77 (m, 2H), 4.65- |
| | | | 4.61 (m, 3H), 4.22-4.13 (m, |
| | | | 1H), 3.77-3.71 (m, 1H), 2.77- |
| | | | 2.65 (m, 2H), 2.28-2.21 (m, |
| | | | 1H), 2.01-1.82 (m, 2H), 1.74- |
| | | | 1.65 (m, 1H), 1.60-1.43 (m, |
| | | | 1H). |
| 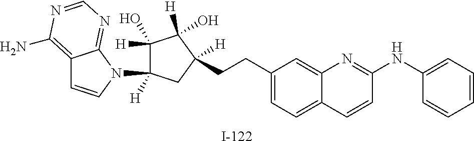 [SMILES: Nc1ncnc2c1ccn2[C@@H]1C[C@H](CCc2ccc3ccc(Nc4ccccc4)nc3c2)[C@@H](O)[C@H]1O ; "I-122"] | D | 481.1 | ¹H NMR (400 MHz, DMSO- d6) δ ppm 9.37 (s, 1H), 8.04- 7.99 (m, 4H), 7.64 (d, J = 8.2 Hz, 1H), 7.53 (s, 1H), 7.34- 7.28 (m, 3H), 7.20-7.18 (m, 1H), 7.00-6.92 (m, 4H), 6.55 (d, J = 3.5 Hz, 1H), 4.85-4.78 (m, 2H), 4.66 (d, J = 4.9 Hz, 1H), 4.24-4.19 (m, 1H), 3.79- 3.75 (m, 1H), 2.82-2.67 (m, |
| | | | 2H), 2.33-2.23 (m, 1H), 2.08- |
| | | | 1.89 (m, 2H), 1.79-1.70 (m, |
| | | | 1H), 1.58-1.50 (m, 1H). |
| 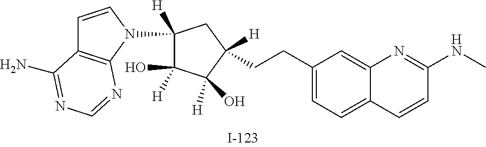 [SMILES: CNc1ccc2ccc(CC[C@H]3C[C@@H](n4ccc5c(N)ncnc54)[C@H](O)[C@@H]3O)cc2n1] | D | 419.2 | ¹H NMR (400 MHz, DMSO- d6) δ ppm 8.03 (s, 1H), 7.77 (d, J = 8.9 Hz, 1H), 7.51 (d, J = 8.1 Hz, 1H), 7.36 (s, 1H), 7.26 (d, J = 3.5 Hz, 1H), 7.03 (dd, J = 8.1, 1.4 Hz, 1H), 6.91 (s, 3H), 6.66 (d, J = 8.9 Hz, 1H), 6.54 (d, J = 3.5 Hz, 1H), 4.83- 4.72 (m, 2H), 4.64 (s, 1H), 4.20 (s, 1H), 3.83-3.76 (m, 1H), 2.88 (d, J = 4.7 Hz, 3H), 2.80-2.63 (m, 2H), 2.29-2.20 (m, 1H), 2.03-1.80 (m, 2H), 1.78-1.66 (m, 1H), 1.53-1.50 (m, 1H). |
| 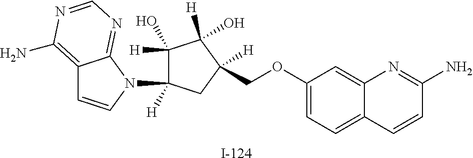 [SMILES: Nc1ccc2ccc(OC[C@H]3C[C@@H](n4ccc5c(N)ncnc54)[C@H](O)[C@@H]3O)cc2n1 ; "I-124"] | A | 407.1 | ¹H NMR (400 MHz, CD₃OD) δ ppm 8.10 (s, 1H), 7.86 (d, J = 8.8 Hz, 1H), 7.57 (d, J = 8.8 Hz, 1H), 7.34 (d, J = 3.6 Hz, 1H), 7.03 (d, J = 2 Hz, 1H), 6.96 (dd, J = 8.8, 2.4 Hz, 1H), 6.69-6.46 (m, 2H), 5.03-5.10 (m, 1H), 4.47-4.44 (m, 1H), 4.24 (d, J = 5.2 Hz, 1H), 4.19- 4.17 (m, 1H), 3.84-3.81 (m, 1H), 2.51-2.63 (m, 2H), 1.89- 1.97 (m, 1H). |
| 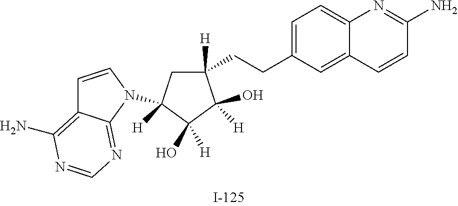 [SMILES: Nc1ccc2cc(CC[C@H]3C[C@@H](n4ccc5c(N)ncnc54)[C@H](O)[C@@H]3O)ccc2n1] | D | 405.2 | ¹H NMR (400 MHz, CD₃OD) δ ppm 8.08 (s, 1H), 7.91 (d, J = 8.8 Hz, 1H), 7.50-7.47 (m, 3H), 7.26 (d, J = 3.6 Hz, 1H), 6.81 (d, J = 8.8 Hz, 1H), 6.62 (d, J = 3.2 Hz, 1H), 4.87 (s, 1H), 4.63 (br, 2H), 4.37-4.33 (m, 1H), 3.93-3.90 (m, 1H), 2.87-2.80 (m, 2H), 2.49-2.42 (m, 1H), 2.10-2.04 (m, 2H), 1.83-1.81 (m, 1H), 1.69-1.64 (m, 1H) |
| 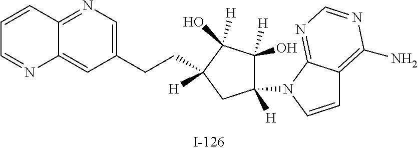 [SMILES: Nc1ncnc2c1ccn2[C@@H]1C[C@H](CCc2cnc3cccnc3c2)[C@@H](O)[C@H]1O] | D | 391.0 | ¹H NMR (400 MHz, DMSO) δ 8.98 (dd, J = 4.1, 1.6 Hz, 1H), 8.95 (d, J = 2.1 Hz, 1H), 8.42- 8.41 (m, 1H), 8.27 (d, J = 1.1 Hz, 1H), 8.03 (s, 1H), 7.75- 7.72 (m, 1H), 7.27 (d, J = 3.6 Hz, 1H), 6.92 (s, 2H), 6.54 (d, J = 3.5 Hz, 1H), 4.86-4.76 (m, 2H), 4.68 (d, J = 4.9 Hz, 1H), 4.24-4.19 (m, 1H), 3.81-3.77 (m, 1H), 2.97-2.87 (m, 2H), |
| | | | 2.30-2.23 (m, 1H), 2.06-1.98 |
| | | | (m, 1H), 1.90-1.77 (m, 2H), |
| | | | 1.60-1.51 (m, 1H). |
| 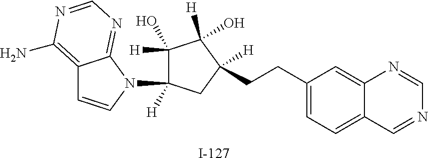 [SMILES: Nc1ncnc2c1ccn2[C@@H]1C[C@H](CCc2ccc3cncnc3c2)[C@@H](O)[C@H]1O ; "I-127"] | D | 390.44 | ¹H NMR (400 MHz, CD₃OD) δ ppm 9.50 (s, 1H), 9.22 (s, 1H), 8.11-8.08 (m, 2H), 7.91 (s, 1H), 7.77-7.75 (m, 1H), 7.27-7.26 (m, 1H), 6.63-6.62 (m, 1H), 4.87 (s, 1H), 4.37-4.36 (m, 1H), 4.97-4.95 (m, 1H), 3.06-3.04 (m, 2H), 2.62-2.52 (m, 1H), 2.08-2.05 (m, 2H), 1.99-1.98 (m, 1H), 1.78-1.62 (m, 1H) |
| 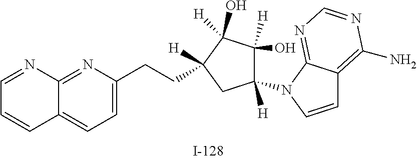 [SMILES: Nc1ncnc2c1ccn2[C@@H]1C[C@H](CCc2ccc3cccnc3n2)[C@@H](O)[C@H]1O] | D | 391.0 | ¹H NMR (400 MHz, DMSO- d6) δ ppm 9.04-9.03 (m, 1H), 8.44-8.37 (m, 2H), 8.04 (s, 1H), 7.64-7.54 (m, 2H), 7.27 (d, J = 3.4 Hz, 1H), 6.94 (s, 2H), 6.55 (d, J = 3.4 Hz, 1H), 4.84-4.78 (m, 2H), 4.68 (d, J = 5.0 Hz, 1H), 4.24-4.19 (m, 1H), 3.81- 3.77 (m, 1H), 3.08-2.97 (m, 2H), 2.30-2.23 (m, 1H), 2.17- 2.06 (m, 1H), 1.96-1.81 (m, 2H), 1.59-1.51 (m, 1H). |
| 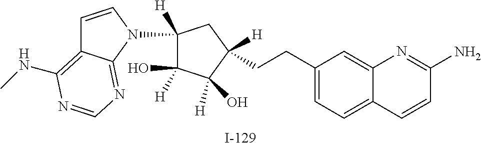 | D | 420.0 | ¹H NMR (400 MHz, DMSO- d6) δ ppm 8.12 (s, 1H), 7.82 (d, J = 8.8 Hz, 1H), 7.53 (d, J = 8.1 Hz, 1H), 7.43-7.36 (m, 1H), 7.30-7.21 (m, 2H), 7.04 (dd, J = 8.2, 1.5 Hz, 1H), 6.68 (d, J = 8.8 Hz, 1H), 6.53 (d, J = 3.5 Hz, 1H), 6.34 (s, 2H), 4.83- 4.78 (m, 2H), 4.64 (d, J = 4.8 Hz, 1H), 4.23-4.17 (m, 1H), 3.74-3.73 (m, 1H), 2.95 (d, J = |
| | | | 4.6 Hz, 3H), 2.78-2.64 (m, 2H), |
| | | | 2.30-2.18 (m, 1H), 1.97-1.82 |
| | | | (m, 2H), 1.76-1.64 (m, 1H), |
| | | | 1.53-1.50 (m, 1H). |
| 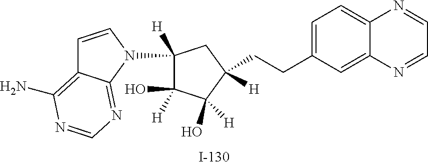 [SMILES: Nc1ncnc2c1ccn2[C@@H]1C[C@H](CCc2ccc3nccnc3c2)[C@@H](O)[C@H]1O] | D | 391.0 | ¹H NMR (400 MHz, DMSO- d6) δ ppm 8.92-8.89 (m, 2H), 8.05-8.03 (m, 2H), 7.94 (d, J = 1.4 Hz, 1H), 7.81-7.78 (m, 1H), 7.27 (d, J = 3.5 Hz, 1H), 6.92 (s, 2H), 6.55 (d, J = 3.5 Hz, 1H), 4.84-4.77 (m, 2H), 4.67 (d, J = 4.6 Hz, 1H), 4.24-4.19 (m, 1H), 3.79-3.74 (m, 1H), 2.98-2.85 (m, 2H), 2.29-2.25 (m, 1H), 2.10-1.96 (m, 1H), 1.93-1.84 (m, 1H), 1.83-1.72 |
| | | | (m, 1H), 1.59-1.51 (m, 1H). |
| 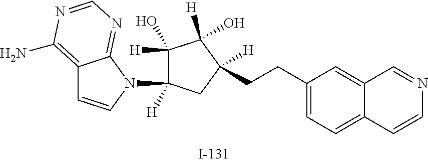 [SMILES: Nc1ncnc2c1ccn2[C@@H]1C[C@H](CCc2ccc3ccncc3c2)[C@@H](O)[C@H]1O] | D | 390.1 | ¹H NMR (400 MHz, CD3OD) δ ppm 9.19 (s, 1H), 9.39 (d, J = 5.6 Hz, 1H), 8.08 (s, 1H), 7.96 (s, 1H), 7.91 (d, J = 8.4 Hz, 1H), 7.81-7.75 (m, 2H), 7.27 (d, J = 3.6 Hz, 1H), 6.63 (d, J = 3.6 Hz, 1H), 4.91 (s, 1H), 4.63 (s, 1H), 4.38-4.35 (m, 1H), 3.96-3.93 (m, 1H), 3.01-2.95 (m, 2H), 2.48-2.45 (m, 1H), 2.12-2.10 (m, 2H), 1.90-1.86 (m, 1H), 1.71-1.68 (m, 1H) |
| 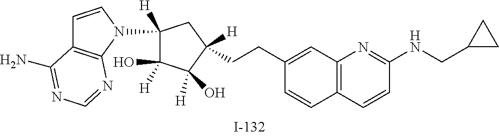 [SMILES: Nc1ncnc2c1ccn2[C@@H]1C[C@H](CCc2ccc3ccc(NCC4CC4)nc3c2)[C@@H](O)[C@H]1O] | D | 459.1 | ¹H NMR (400 MHz, DMSO- d6) δ ppm 8.03 (s, 1H), 7.78- 7.75 (m, 1H), 7.50 (d, J = 8.1 Hz, 1H), 7.32 (s, 1H), 7.26 (d, J = 3.5 Hz, 1H), 7.06-6.99 (m, 2H), 6.91 (s, 2H), 6.71 (d, J = 8.9 Hz, 1H), 6.54 (d, J = 3.5 Hz, 1H), 4.85-4.74 (m, 2H), 4.64 (d, J = 4.8 Hz, 1H), 4.23- 4.18 (m, 1H), 3.77-3.73 (m, |
| | | | 1H), 3.27-3.21 (m, 2H), 2.76- |
| | | | 2.64 (m, 2H), 2.28-2.20 (m, |
| | | | 1H), 1.99-1.85 (m, 2H), 1.75- |
| | | | 1.66 (m, 1H), 1.54-1.46 (m, |
| | | | 1H), 1.13-1.03 (m, 1H), 0.48- |
| | | | 0.43 (m, 2H), 0.27-0.22 (m, |
| | | | 2H). |
| 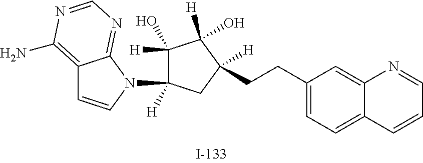 [SMILES: Nc1ncnc2c1ccn2[C@@H]1C[C@H](CCc2ccc3cccnc3c2)[C@@H](O)[C@H]1O] | D | 390.1 | ¹H NMR (400 MHz, DMSO- d6) δ ppm 8.89-8.84 (m, 1H), 8.32 (d, J = 7.9 Hz, 1H), 8.03 (s, 1H), 7.91 (d, J = 8.4 Hz, 1H), 7.85 (s, 1H), 7.53 (d, J = 8.4 Hz, 1H), 7.47 (dd, J = 8.2, 4.2 Hz, 1H), 7.27 (d, J = 3.5 Hz, 1H), 6.92 (s, 2H), 6.54 (d, J = 3.5 Hz, 1H), 4.84-4.78 (m, 2H), 4.70-4.61 (m, 1H), 4.26- 4.17 (m, 1H), 3.81-3.73 (m, 1H), 2.95-2.78 (m, 2H), 2.29- |
| | | | 2.21 (m, 1H), 2.06-1.96 (m, |
| | | | 1H), 1.95-1.87 (m, 1H), 1.81- |
| | | | 1.70 (m, 1H), 1.58-1.52 (m, |
| | | | 1H). |
| 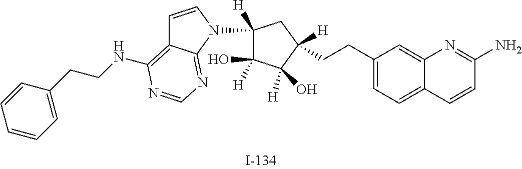 [SMILES: Nc1ccc2ccc(CC[C@H]3C[C@@H](n4ccc5c(NCCc6ccccc6)ncnc54)[C@H](O)[C@@H]3O)cc2n1] | D | 509.1 | ¹H NMR (400 MHz, DMSO- d6) δ ppm 8.14 (s, 1H), 7.82 (d, J = 8.9 Hz, 1H), 7.57-7.51 (m, 2H), 7.34-7.24 (m, 6H), 7.22- 7.20 (m, 1H), 7.07-7.01 (m, 1H), 6.67 (d, J = 8.8 Hz, 1H), 6.56 (d, J = 3.5 Hz, 1H), 6.33 (s, 2H), 4.84-4.72 (m, 2H), 4.64 (d, J = 4.9 Hz, 1H), 4.21-4.19 (m, 1H), 3.79-3.62 (m, 3H), 2.95-2.86 (m, 2H), 2.78-2.62 (m, 2H), 2.30-2.20 (m, 1H), |
| | | | 1.99-1.82 (m, 2H), 1.70-1.62 |
| | | | (m, 1H), 1.56-1.44 (m, 1H). |
| 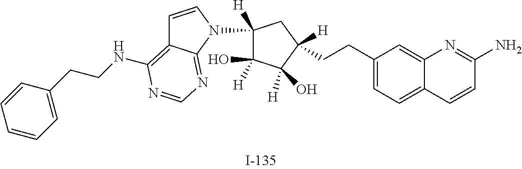 [SMILES: Nc1ccc2ccc(CC[C@H]3C[C@@H](n4ccc5c(NCCc6ccccc6)ncnc54)[C@H](O)[C@@H]3O)cc2n1] | D | 339.1 | ¹H NMR (400 MHz, DMSO- d6) δ 8.02 (s, 1H), 7.33-7.10 (m, 6H), 6.91 (s, 2H), 6.53 (d, J = 3.5 Hz, 1H), 4.83-4.74 (m, 2H), 4.63 (d, J = 4.9 Hz, 1H), 4.22-4.17 (m, 1H), 3.74-37.2 (m, 1H), 2.68-2.57 (m, 2H), 2.25-2.18 (m, 1H), 1.95-1.77 (m, 2H), 1.68-1.61 (m, 1H), 1.52-1.44 (m, 1H). |
| 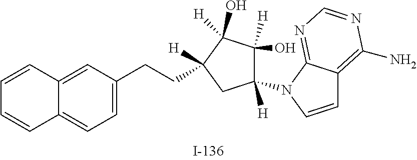 [SMILES: Nc1ncnc2c1ccn2[C@@H]1C[C@H](CCc2ccc3ccccc3c2)[C@@H](O)[C@H]1O] | D | 389.1 | ¹H NMR (400 MHz, CD3OD) δ ppm 8.08 (s, 1H), 7.82-7.79 (m, 3H), 7.70 (s, 1H), 7.45-7.40 (m, 3H), 7.27 (d, J = 3.6 Hz, 1H), 6.63 (d, J = 3.6 Hz, 1H), 4.87 (s, 1H), 4.63 (s, 1H), 4.37- 4.34 (m, 1H), 3.95-3.92 (m, 1H), 2.93-2.90 (m, 2H), 2.46- 2.45 (m, 1H), 2.11-2.09 (m, 2H), 1.90-1.82 (m, 1H), 1.72- 1.62 (m, 1H) |
| 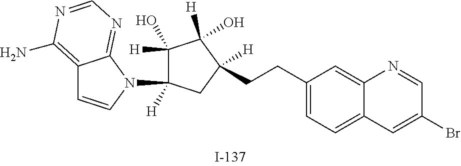 [SMILES: Nc1ncnc2c1ccn2[C@@H]1C[C@H](CCc2ccc3cc(Br)cnc3c2)[C@@H](O)[C@H]1O] | D | 468.0 | ¹H NMR (400 MHz, DMSO) δ ppm 8.91 (d, J = 2.3 Hz, 1H), 8.68 (d, J = 2.2 Hz, 1H), 8.03 (s, 1H), 7.96-7.83 (m, 2H), 7.61-7.58 (m, 1H), 7.26 (d, J = 3.5 Hz, 1H), 6.92 (s, 2H), 6.54 (d, J = 3.5 Hz, 1H), 4.88-4.73 (m, 2H), 4.66 (d, J = 4.9 Hz, 1H), 4.24-4.19 (m, 1H), 3.79- 3.75 (m, 1H), 2.98-2.76 (m, 2H), 2.29-2.22 (m, 1H), 2.09- 1.94 (m, 1H), 1.90-1.86 (m, |
| | | | 1H), 1.83-1.69 (m, 1H), 1.57- |
| | | | 1.52 (m, 1H). |
| 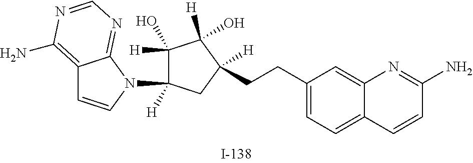 [SMILES: Nc1ccc2ccc(CC[C@H]3C[C@@H](n4ccc5c(N)ncnc54)[C@H](O)[C@@H]3O)cc2n1] | D | 405.2 | ¹H NMR (400 MHz, DMSO- d6) δ ppm 8.03 (s, 1H), 7.82 (d, J = 8.8 Hz, 1H), 7.53 (d, J = 8.1 Hz, 1H), 7.35-7.23 (m, 2H), 7.05-7.02 (m, 1H), 6.91 (s, 2H), 6.67 (d, J = 8.8 Hz, 1H), 6.54 (d, J = 3.5 Hz, 1H), 6.33 (s, 2H), 4.89-4.73 (m, 2H), 4.63 (d, J = 4.9 Hz, 1H), 4.23-4.18 (m, 1H), 3.84-3.68 (m, 1H), 2.84-2.62 (m, 2H), 2.28-2.21 (m, 1H), 2.02-1.81 (m, 2H), |
| | | | 1.71-1.52 (m, 1H), 1.50-1.47 |
| | | | (m, 1H). |
| 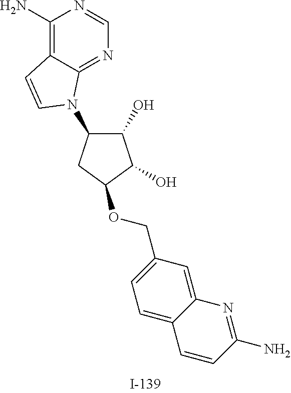 [SMILES: Nc1ccc2ccc(CO[C@H]3C[C@@H](n4ccc5c(N)ncnc54)[C@H](O)[C@@H]3O)cc2n1] | A | 407.0 | ¹H NMR (400 MHz, CD₃OD) δ ppm 8.09 (s, 1H), 7.94 (d, J = 8.8 Hz, 1H), 7.66 (d, J = 8.1 Hz, 1H), 7.58 (s, 1H), 7.32- 7.27 (m, 2H), 6.83 (d, J = 8.9 Hz, 1H), 6.63 (d, J = 3.6 Hz, 1H), 5.12-5.06 (m, 1H), 4.78 (s, 2H), 4.52-4.49 (m, 1H), 4.21- 4.49 (m, 1H), 4.03-4.00 (m, 1H), 2.88-2.77 (m, 1H), 2.03- 1.94 (m, 1H). |
| 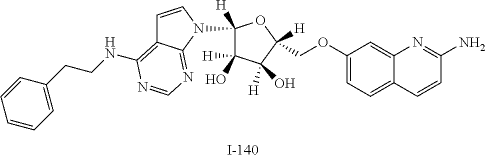 [SMILES: Nc1ccc2ccc(OC[C@H]3O[C@@H](n4ccc5c(NCCc6ccccc6)ncnc54)[C@H](O)[C@@H]3O)cc2n1] | A | 437.1 | ¹H NMR (400 MHz, CD3OD) δ ppm 8.05 (s, 1H), 7.76 (d, J = 8.9 Hz, 1H), 7.52 (d, J = 8.8 Hz, 1H), 7.29 (d, J = 1.1 Hz, 1H), 7.18 (d, J = 2.4 Hz, 1H), 6.94 (dd, J = 8.7, 2.5 Hz, 1H), 6.60 (d, J = 8.9 Hz, 1H), 6.55 (d, J = 4.5 Hz, 1H), 4.59-4.55 (m, 1H), 4.51 (dd, J = 9.7, 5.4 Hz, 2H), 4.35 (d, J = 2.7 Hz, 1H), 4.28-4.22 (m, 1H), 3.01 (s, 3H), 2.45 (d, J = 1.1 Hz, 3H). |
| 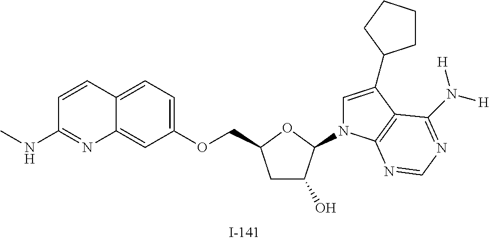 [SMILES: CNc1ccc2ccc(OC[C@@H]3C[C@@H](O)[C@H](n4cc(C5CCCC5)c5c(N)ncnc54)O3)cc2n1] | C | 474.55 | ¹H NMR (400 MHz, DMSO- d6) δ ppm 8.04 (s, 1H), 7.73 (d, J = 8.9 Hz, 1H), 7.51 (d, J = 8.6 Hz, 2H), 7.06 (s, 1H), 6.99 (s, 1H), 6.95-6.91 (m, 1H), 6.80-6.78 (m, 1H), 6.59-6.49 (m, 3H), 6.12 (d, J = 2.9 Hz, 1H), 5.60 (d, J = 4.6 Hz, 1H), 4.55 (d, J = 42.0 Hz, 2H), 4.32 (d, J = 8.3 Hz, 1H), 4.17-4.15 (m, 1H), 2.87 (d, J = 4.8 Hz, 3H), 2.08 (s, 2H), 2.01-1.97 (m, 1H), 1.67-1.64 (m, 2H), 1.48- 1.44 (m, 2H), 1.24 (s, 3H). |
| 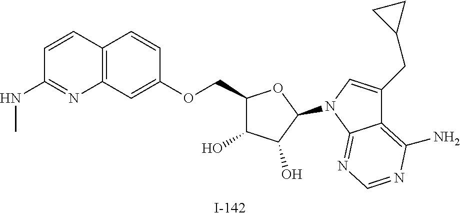 [SMILES: CNc1ccc2ccc(OC[C@H]3O[C@@H](n4cc(CC5CC5)c5c(N)ncnc54)[C@H](O)[C@@H]3O)cc2n1] | A | 477.0 | ¹H NMR (400 MHz, DMSO- d₆) δ ppm 8.04 (s, 1H), 7.75 (d, J = 8.8 Hz, 1H), 7.52 (d, J = 8.7 Hz, 1H), 7.16 (s, 1H), 7.03 (s, 2H), 6.83 (d, J = 8.7 Hz, 1H), 6.58 (d, J = 8.8 Hz, 1H), 6.51 (s, 2H), 6.17 (d, J = 5.9 Hz, 1H), 5.39 (d, J = 6.3 Hz, 1H), 5.32 (d, J = 4.8 Hz, 1H), 4.50-4.46 (m, 1H), 4.32-4.30 (m, 1H), 4.24-4.22 (m, 3H), 2.89 (d, J = 4.7 Hz, 3H), 2.69- 2.63 (m, 2H), 1.01-0.90 (m, 1H), 0.49-0.28 (m, 2H), 0.15- 0.03 (m, 2H). |
| 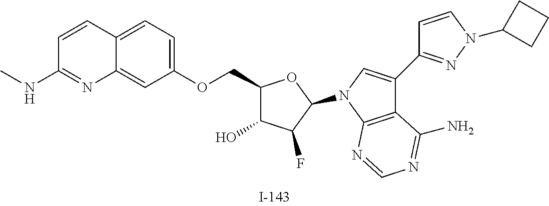 [SMILES: CNc1ccc2ccc(OC[C@H]3O[C@@H](n4cc(-c5ccn(C6CCC6)n5)c5c(N)ncnc54)[C@@H](F)[C@@H]3O)cc2n1] | B | 545.2 | ¹H NMR (400 MHz, DMSO- d₆) δ ppm 9.20 (s, 1H), 8.11 (s, 1H), 7.82 (d, J = 2.4 Hz, 1H), 7.75 (d, J = 8.8 Hz, 1H), 7.67 (d, J = 2.4 Hz, 1H), 7.52 (d, J = 8.8 Hz, 1H), 7.31 (s, 1H), 7.07 (d, J = 2.4 Hz, 1H), 6.96-6.92 (m, 1H), 6.85-6.82 (m, 1H), 6.73-6.68 (m, 1H), 6.63 (d, J = 2.4 Hz, 1H), 6.59 (d, J = 8.8 Hz, 1H), 6.15 (d, J = 4.8 Hz, 1H), 5.31-5.15 (m, 1H), 4.88- 4.84 (m, 1H), 4.62-4.54 (m, 1H), 4.46-4.34 (m, 1H), 4.22- 4.19 (m, 1H), 4.20 (s, 1H), 2.88 |
| | | | (d, J = 4.8 Hz, 3H), 2.46-2.40 |
| | | | (m, 4H), 1.85-1.77 (m, 2H). |
| 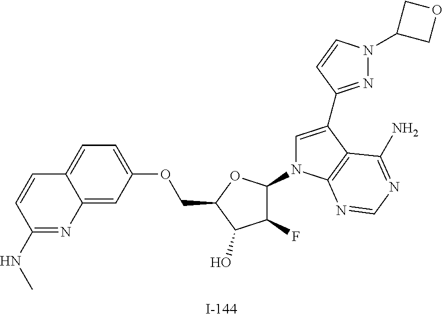 [SMILES: CNc1ccc2ccc(OC[C@H]3O[C@@H](n4cc(-c5ccn(C6COC6)n5)c5c(N)ncnc54)[C@@H](F)[C@@H]3O)cc2n1] | B | 548.3 | ¹H NMR (400 MHz, DMSO- d₆) δ ppm 9.06 (s, 1H), 8.11 (s, 1H), 7.90 (d, J = 2.4 Hz, 1H), 7.74 (d, J = 8.8 Hz, 1H), 7.71 (d, J = 2.4 Hz, 1H), 7.52 (d, J = 8.8 Hz, 1H), 7.06 (d, J = 2.4 Hz, 1H), 6.95-6.91 (m, 1H), 6.84-6.81 (m, 1H), 6.73-6.69 (m, 2H), 6.58 (d, J = 8.8 Hz, 1H), 6.14 (d, J = 4.8 Hz, 1H), 5.64-5.58 (m, 1H), 5.31- 5.15 (m, 1H), 4.97 (t, J = 7.2 Hz, 2H), 4.86 (t, J = 6.4 Hz 2H), 4.62-4.54 (m, 1H), 4.48- 4.34 (m, 2H), 4.21 (s, 1H), 2.88 (d, J = 4.8 Hz, 3H). |
| 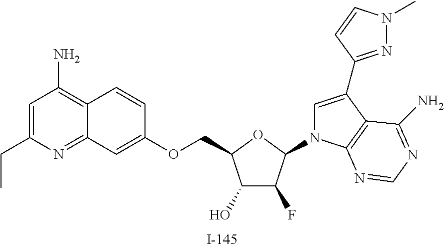 [SMILES: CCc1cc(N)c2ccc(OC[C@H]3O[C@@H](n4cc(-c5ccn(C)n5)c5c(N)ncnc54)[C@@H](F)[C@@H]3O)cc2n1] | B | 519.1 | ¹H NMR (400 MHz, DMSO- d₆) δ ppm 8.22 (s, 1H), 8.17 (d, J = 9.2 Hz, 1H), 7.57-7.51 (m, 2H), 7.35-7.32 (m, 1H), 7.25 (d, J = 2 Hz, 1H), 6.78-6.73 (m, 1H), 6.56 (s, 1H), 6.33 (d, J = 2 Hz, 1H), 5.36-5.28 (m, 1H), 5.17-5.16 (m, 1H), 4.78- 4.63 (m, 2H), 4.55-4.52 (m, 1H), 4.39-4.36 (m, 1H), 3.95 (s, 3H), 2.86-2.81 (m, 2H), 1.37 (t, J = 7.6 Hz, 3H), 1.30 (s, 2H). |
| 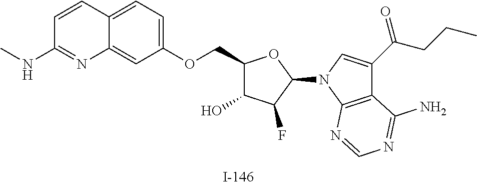 [SMILES: CCCC(=O)c1cn([C@@H]2O[C@H](COc3ccc4ccc(NC)nc4c3)[C@@H](O)[C@@H]2F)c2ncnc(N)c12] | B | 495.1 | ¹H NMR (400 MHz, DMSO- d₆) δ 7.40 (s, 1H), 7.38 (d, J = 8.8 Hz, 2H), 7.35 (s, 1H), 7.32 (s, 1H), 7.31 (s, 1H), 7.28 (s, 1H), 7.19 (s, 1H), 7.17-7.13 (m, 2H), 7.08 (d, J = 10.8 Hz, 1H), 6.85-6.84 (m, 1H), 6.67-6.64 (m, 1H), 6.63-6.61 (m, 2H), 6.20 (s, 1H), 2.96 (s, 3H), 2.85- 2.81 (m, 2H), 1.62-1.60 (m, 2H), 0.88 (t, J = 14.4 Hz, 3H). |
| 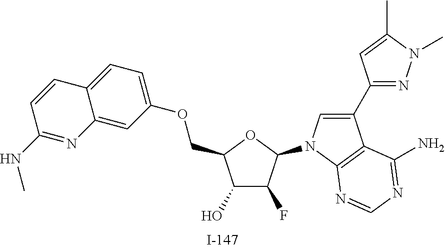 [SMILES: CNc1ccc2ccc(OC[C@H]3O[C@@H](n4cc(-c5cc(C)n(C)n5)c5c(N)ncnc54)[C@@H](F)[C@@H]3O)cc2n1] | B | 519.2 | ¹H NMR (400 MHz, DMSO- d₆) δ ppm 9.07 (br, 1H), 8.08 (s, 1H), 7.75 (d, J = 8.8 Hz, 1H), 7.55-7.52 (m, 2H), 7.21 (br, 1H), 7.07 (d, J = 2.4 Hz, 1H), 6.96-6.94 (m, 1H), 6.85 (dd, J = 8.8, 2.8 Hz, 1H), 6.70 (dd, J = 15.6, 4.4 Hz, 1H), 6.59 (d, J = 8.8 Hz, 1H), 6.32 (s, 1H), 6.14 (d, J = 4.8 Hz, 1H), 5.20 (dt, J = 52.8, 3.6 Hz, 1H), 4.46-4.43 (m, 1H), 4.37-4.35 (m, 2H), 4.22-4.20 (m, 1H), 3.74 (s, 3H), 2.89 (d, J = 4.8 Hz, 3H), 2.24 (s, 3H) |
| 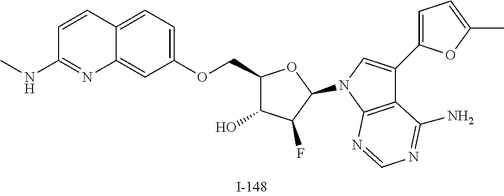 | B | 505.2 | ¹H NMR (400 MHz, DMSO- d₆) δ ppm 8.16 (s, 1H), 8.14 (s, 1H), 7.74 (d, J = 8.8 Hz, 1H), 7.61 (d, J = 2.0 Hz, 1H), 7.52 (d, J = 8.0 Hz, 1H), 7.06 (d, J = 2.4 Hz, 1H), 6.93-6.96 (m, 1H), 6.82-6.86 (m, 1H), 6.71 (dd, J = 4.8, 14.4 Hz, 1H), 6.58 (d, J = 7.2 Hz, 1H), 6.53 (d, J = 3.2 Hz, 1H), 6.18-6.20 (m 1H), 6.14 (d, J = 4 Hz, 1H), 5.18- 5.33 (m, 1H), 4.58 (dd, J = 4.4, 19.2 Hz, 1H), 4.32-4.44 (m, 2H), 4.18-4.21 (m, 1H), 2.88 |
| | | | (d, J = 5.2 Hz, 2H), 2.36 (s, |
| | | | 3H). |
| 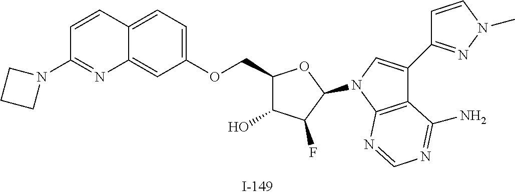 [SMILES: Cn1ccc(-c2cn([C@@H]3O[C@H](COc4ccc5ccc(N6CCC6)nc5c4)[C@@H](O)[C@@H]3F)c3ncnc(N)c23)n1] | B | 531.1 | ¹H NMR (400 MHz, DMSO- d₆) δ ppm 9.05 (br, 1H), 8.09 (s, 1H), 7.92 (d, J = 9.2 Hz, 1H), 7.71 (d, J = 2.0 Hz, 1H), 7.64-7.59 (m, 2H), 7.25 (br, 1H), 7.10 (d, J = 2.4 Hz, 1H), 6.90 (dd, J = 8.8, 2.4 Hz, 1H), 6.71 (dd, J = 16.0, 4.4 Hz, 1H), 6.59 (d, J = 2.4 Hz, 1H), 6.52 (d, J = 8.8 Hz, 1H), 6.16 (d, J = 4.8 Hz, 1H), 5.18 (dt, J = 52.8, 3.6 Hz, 1H), 4.61-4.56 (m, 1H), 4.46-4.34 (m, 2H), 4.22-4.19 (m, 1H), 4.09-4.05 |
| | | | (m, 4H), 3.88 (s, 3H), 2.37-2.33 |
| | | | (m, 2H) |
| 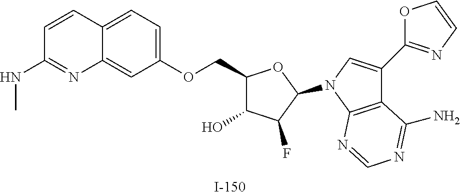 [SMILES: CNc1ccc2ccc(OC[C@H]3O[C@@H](n4cc(-c5ncco5)c5c(N)ncnc54)[C@@H](F)[C@@H]3O)cc2n1] | B | 492.2 | ¹H NMR (400 MHz, DMSO- d₆) δ ppm 9.01 (s, 1H), 8.21- 8.17 (m, 2H), 8.06 (d, J = 1.6 Hz, 1H), 7.75 (d, J = 8.9 Hz, 1H), 7.60-7.57 (m, 2H), 7.41 (d, J = 0.8 Hz, 1H), 7.12 (d, J = 2.4 Hz, 1H), 7.02-6.87 (m, 2H), 6.72 (dd, J = 12.3, 5.0 Hz, 1H), 6.59 (d, J = 8.8 Hz, 1H), 6.18 (s, 1H), 5.33 (dt, J = 52.9, 4.7 Hz, 1H), 4.69-4.56 (m, 1H), 4.49-4.33 (m, 2H), 4.24-4.21 (m, 1H), 2.89 (d, J = 4.7 Hz, 3H). |
| 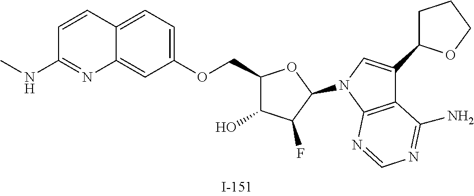 [SMILES: CNc1ccc2ccc(OC[C@H]3O[C@@H](n4cc([C@H]5CCCO5)c5c(N)ncnc54)[C@@H](F)[C@@H]3O)cc2n1] | B | 495.1 | ¹H NMR (400 MHz, DMSO- d₆) δ ppm 8.10 (s, 1H), 7.75 (d, J = 8.8 Hz, 1H), 7.53 (d, J = 8.4 Hz, 1H), 7.15 (d, J = 1.6 Hz, 1H), 7.04 (d, J = 2.4 Hz, 1H), 6.94 (d, J = 5.2 Hz, 1H), 6.84-6.83 (m, 2H), 6.66 (dd, J = 15.6, 4.8 Hz, 1H), 6.59 (d, J = 9.2 Hz, 1H), 6.11 (d, J = 4.8 Hz, 1H), 5.26 (t, J = 1.8 Hz, 1H), 4.90-4.80 (m, 1H), 4.42 (d, J = 2.8 Hz, 1H ), 4.32 (dd, J = 11.2, 6.4 Hz 1H), 4.29- 4.28 (m, 1H), 4.17-4.15 (m, |
| | | | 1H), 3.95 (dd, J = 15.6, 8.0 Hz |
| | | | 1H), 3.82 (dd, J = 15.6, 8.0 |
| | | | Hz, 1H), 2.88 (d, J = 4.8 Hz, |
| | | | 3H), 2.16-2.15 (m, 1H), 1.98- |
| | | | 1.97 (m, 2H), 1.78-1.76 (m, |
| | | | 1H). |
| 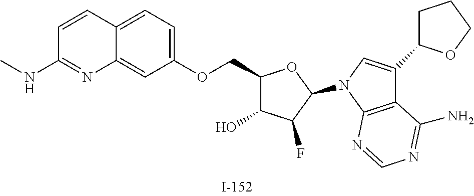 [SMILES: CNc1ccc2ccc(OC[C@H]3O[C@@H](n4cc([C@@H]5CCCO5)c5c(N)ncnc54)[C@@H](F)[C@@H]3O)cc2n1] | B | 495.1 | ¹H NMR (400 MHz, DMSO- d6) δ ppm 8.10 (s, 1H), 7.75 (d, J = 8.8 Hz, 1H), 7.53 (d, J = 8.4 Hz, 1H), 7.15 (d, J = 1.6 Hz, 1H), 7.04 (d, J = 2.4 Hz, 1H), 6.94 (d, J = 5.2 Hz, 1H), 6.84-6.83 (m, 2H), 6.66 (dd, J = 15.6, 4.8 Hz, 1H), 6.59 (d, J = 9.2 Hz, 1H), 6.11 (d, J = 4.8 Hz, 1H), 5.26 (t, J = 1.8 Hz, 1H), 4.90-4.80 (m, 1H), 4.42 (d, J = 2.8 Hz, 1H), 4.32 (dd, J = 11.2, 6.4 Hz 1H), 4.29-4.28 (m, 1H), 4.17-4.15 (m, 1H), |
| | | | 3.95 (dd, J = 15.6, 8.0 Hz, 1H), |
| | | | 3.82 (dd, J = 15.6, 8.0 |
| | | | Hz, 1H), 2.88 (d, J = 4.8 Hz, |
| | | | 3H), 2.16-2.15 (m, 1H), 1.98- |
| | | | 1.97 (m, 2H), 1.78-1.76 (m, |
| | | | 1H). |
| 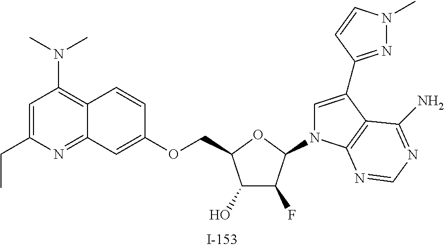 [SMILES: CCc1cc(N(C)C)c2ccc(OC[C@H]3O[C@@H](n4cc(-c5ccn(C)n5)c5c(N)ncnc54)[C@@H](F)[C@@H]3O)cc2n1] | B | 547.1 | ¹H NMR (400 MHz, DMSO- d₆) δ ppm 9.04 (s, 1H), 8.09 (s, 1H), 7.92 (d, J = 9.2 Hz, 1H), 7.71 (d, J = 2.2 Hz, 1H), 7.62 (d, J = 2.2 Hz, 1H), 7.35 (d J = 2.6 Hz, 1H), 7.27 (s, 1H), 7.12 (dd, J = 9.2, 2.6 Hz, 1H), 6.79- 6.63 (m, 2H), 6.58 (d, J = 2.3 Hz, 1H), 6.16 (d, J = 4.9 Hz, 1H), 5.35-5.10 (m, 1H), 4.65- 4.53 (m, 1H), 4.53-4.45 (m, 1H), 4.45-4.35 (m, 1H), 4..30- 4.15 (m, 1H), 3.87 (s, 3H), 2.95 (s, 6H), 2.79 (q, J = 7.6 Hz, 2H), 1.28 (t, J = 7.6 Hz, 3H). |
| 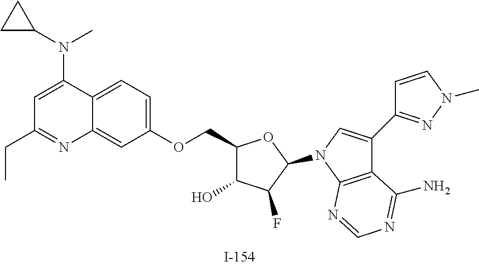 [SMILES: CCc1cc(N(C)C2CC2)c2ccc(OC[C@H]3O[C@@H](n4cc(-c5ccn(C)n5)c5c(N)ncnc54)[C@@H](F)[C@@H]3O)cc2n1] | B | 573.2 | ¹H NMR (400 MHz, DMSO- d₆) δ ppm 9.04 (br, 1H), 8.09 (s, 1H), 7.91 (d, J = 8.8 Hz, 1H), 7.71 (d, J = 2.0 Hz, 1H), 7.62 (d, J = 2.4 Hz, 1H), 7.33 (d, J = 2.4 Hz, 1H), 7.27-7.24 (m, 1H), 7.09 (dd, J = 9.2, 2.8 Hz, 1H), 6.86 (s, 1H), 6.72- 6.67 (m, 1H), 6.58 (d, J = 2.4 Hz, 1H), 6.16 (d, J = 4.8 Hz, 1H), 5.21 (dt, J = 56.8, 4 Hz, 1H), 4.63-4.61 (m, 1H), 4.57- 4.55 (m, 1H), 4.48-4.47 (m, 1H), 4.23-4.21 (m, 1H), 3.87 (s, 3H), 3.07 (s, 3H), 2.83-2.77 (m, 3H), 1.29 (t, J = 7.6 Hz, 3H), 0.79-0.77 (m, 2H), 0.44-0.43 (m, 2H) |
| 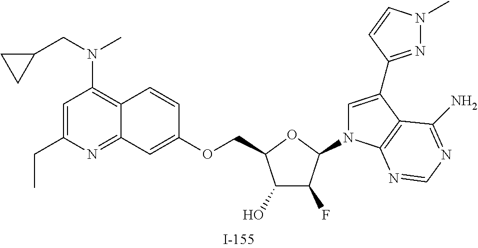 [SMILES: CCc1cc(N(C)CC2CC2)c2ccc(OC[C@H]3O[C@@H](n4cc(-c5ccn(C)n5)c5c(N)ncnc54)[C@@H](F)[C@@H]3O)cc2n1] | B | 587.2 | ¹H NMR (400 MHz, DMSO- d₆) δ ppm 9.04 (s, 1H), 8.08 (s, 1H), 7.94 (d, J = 14.8 Hz, 1H), 7.71 (d, J = 2 Hz, 1H), 7.61 (d, J = 2 Hz, 1H), 7.35 (d, J = 2.4 Hz, 1H), 7.27 (s, 1H), 7.15 (dd, J = 9.2 Hz, 2.4 Hz 1H), 6.75 (s, 1H), 6.72-6.68 (m, 1H), 6.58 (d, J = 2.4 Hz, 1H), 6.17 (d, J = 4.8 Hz, 1H), 5.30-5.15 (m, 1H), 4.62-4.56 (m, 1H), 4.52- 4.38 (m, 2H), 4.25-4.23 (m, 1H), 3.88 (s, 3H), 3.16 (d, J = 6.8 Hz, 2H), 3.04 (s, 3H), 2.83- 2.77 (m, 2H), 1.29 (t, J = 7.6 Hz, 3H), 1.05 (m, 1H), 0.54- 0.48 (m, 2H), 0.17-0.15 (m, |
| | | | 2H). |
| 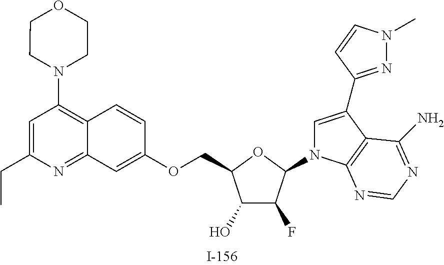 [SMILES: CCc1cc(N2CCOCC2)c2ccc(OC[C@H]3O[C@@H](n4cc(-c5ccn(C)n5)c5c(N)ncnc54)[C@@H](F)[C@@H]3O)cc2n1] | B | 590.0 | ¹H NMR (400 MHz, DMSO- d₆) δ ppm 9.04 (brs, 1H), 8.09 (s, 1H), 7.90 (d, J = 9.2 Hz, 1H), 7.71 (d, J = 2.0 Hz, 1H), 7.61 (d, J = 2.4 Hz, 1H), 7.40 (d, J = 2.8 Hz, 1H), 7.28 (brs, 1H), 7.16 (dd, J = 9.2, 2.4 Hz, 1H), 6.80 (s, 1H), 6.70 (dd, J = 16.0, 4.0 Hz, 1H), 6.57 (d, J = 2.4 Hz, 1H), 6.16 (d, J = 4.8 Hz, 1H), 5.22 (dt, J = 52.4, 4.0 Hz, 1H), 4.62-4.49 (m, 2H), 4.43-4.39 (m, 1H), 4.25-4.21 (m, 1H), 3.87 (s, 3H), 3.87-3.85 (m, 4H), 3.14 (s, 4H), 2.86-2.80 (m, 2H), 1.29 (t, J = 7.6 Hz, 3H). |
| 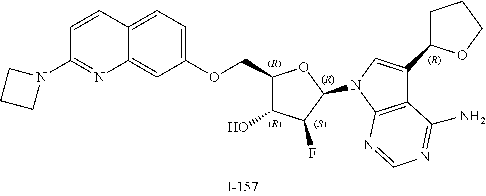 | B | 521.2 | ¹H NMR (400 MHz, DMSO- d₆) δ 8.11 (s, 1H), 7.92 (d, J = 8.8 Hz, 1H), 7.61 (d, J = 8.8 Hz, 1H), 7.17-7.15 (m, 1H), 7.08 (s, 1H), 6.91-6.86 (m, 3H), 6.66-6.61 (m, 1H), 6.52 (d, J = 5.2 Hz, 1H), 5.20 (d, t, J = 50.4, 3.6 Hz, 1H), 4.92-4.90 (m, 1H), 4.56-4.51 (m, 1H), 4.43-4.40 (m, 1H), 4.31-4.28 (m, 1H), 4.16-4.13 (m, 1H), 4.08-4.04 (m, 4H), 3.95-3.90 (m, 1H), 3.82-3.80 (m, 1H), 2.37-2.33 (m, 2H), 2.16-2.08 (m, 1H), |
| | | | 1.97-1.93 (m, 2H), 1.78-1.73 |
| | | | (m, 1H). |
| 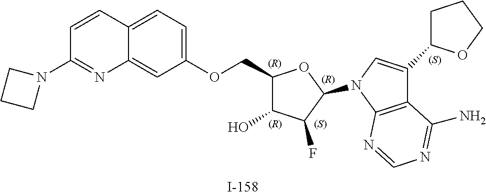 | B | 521.0 | ¹H NMR (400 MHz, DMSO- d₆) δ ppm 8.11 (s, 1H), 7.92 (d, J = 8.8 Hz, 1H), 7.61 (d, J = 8.8 Hz, 1H), 7.17-7.15 (m, 1H), 7.08 (s, 1H), 6.91-6.86 (m, 3H), 6.66-6.61 (m, 1H), 6.52 (d, J = 5.2 Hz, 1H), 5.20 (dt, J = 50.4, 3.6 Hz, 1H), 4.92-4.90 (m, 1H), 4.56-4.51 (m, 1H), 4.43-4.40 (m, 1H), 4.31-4.28 (m, 1H), 4.16-4.13 (m, 1H), 4.08-4.04 (m, 4H), 3.95-3.90 (m, 1H), 3.82-3.80 (m, 1H), 2.37-2.33 (m, 2H), 2.16-2.08 (m, 1H), |
| | | | 1.97-1.93 (m, 2H), 1.78-1.73 |
| | | | (m, 1H). |
| 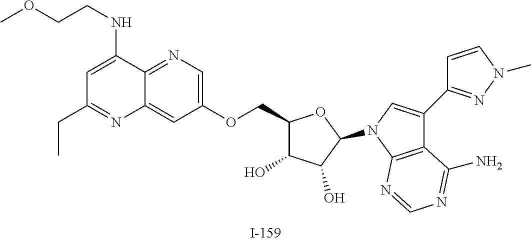 | B | 576.1 | ¹H NMR (400 MHz, DMSO- d₆) δ 8.99 (s, 1H), 8.48 (d, J = 2.7 Hz, 1H), 8.07 (s, 1H), 7.81 (s, 1H), 7.72 (d, J = 2.2 Hz, 1H), 7.59 (d, J = 2.8 Hz, 1H), 7.40-7.15 (m, 1H), 7.15-6.90 (m, 1H), 6.60 (d, J = 2.3 Hz, 1H), 6.50 (s, 1H), 6.22 (d, J = 5.8 Hz, 1H), 5.52 (d, J = 6.3 Hz, 1H), 5.43 (d, J = 5.3 Hz, 1H), 4.60-4.53 (m, 1H), 4.50- 4.41 (m, 1H), 4.39-4.29 (m, 2H), 4.29-4.22 (m, 1H), 3.88 (s, 3H), 3.60 (t, J = 5.6 Hz, 2H), 3.54-3.39 (m, 2H), 3.31 (s, 3H), 2.73 (q, J = 7.6 Hz, 2H), 1.27 (t, J = 7.6 Hz, 3H). |
| 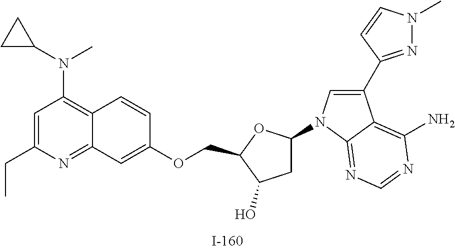 [SMILES: CCc1cc(N(C)C2CC2)c2ccc(OC[C@H]3O[C@@H](n4cc(-c5ccn(C)n5)c5c(N)ncnc54)C[C@@H]3O)cc2n1] | B | 555.1 | ¹H NMR (400 MHz, DMSO- d₆) δ ppm 8.97 (brs, 1H), 8.07 (s, 1H), 7.88 (d, J = 9.2 Hz, 1H), 7.80 (s, 1H), 7.68 (d, J = 2.4 Hz, 1H), 7.30 (d, J = 2.8 Hz, 1H), 7.20 (brs, 1H), 7.09 (dd, J = 9.2, 2.4 Hz, 1H), 6.85 (s, 1H), 6.69-6.65 (m, 1H), 6.51 (d, J = 2.0 Hz, 1H), 5.52 (d, J = 4.0 Hz, 1H), 4.55-4.54 (m, 1H), 4.37-4.34 (m, 1H), 4.27-4.18 (m, 2H), 3.86 (s, 3H), 3.06 (s, 3H), 2.82-2.77 (m, 3H), 2.74- 2.68 (m, 1H), 2.34-2.29 (m, 1H), 1.29 (t, J = 7.6 Hz, 3H), 0.78 (d, J = 6.8 Hz, 2H), 0.43 (s, 2H). |
| 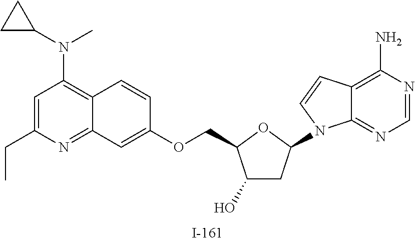 [SMILES: CCc1cc(N(C)C2CC2)c2ccc(OC[C@H]3O[C@@H](n4ccc5c(N)ncnc54)C[C@@H]3O)cc2n1] | B | 475.1 | ¹H NMR (400 MHz, DMSO- d₆) δ ppm 8.08 (s, 1H), 7.91 (d, J = 9.6 Hz, 1H), 7.38 (d, J = 3.6 Hz, 1H), 7.28 (d, J = 2.8 Hz, 1H), 7.06 (dd, J = 9.2, 2.8 Hz, 1H), 7.02 (brs, 2H), 6.86 (s, 1H), 6.64-6.59 (m, 2H), 5.50 (d, J = 3.6 Hz, 1H), 4.51-4.50 (m, 1H), 4.34-4.30 (m, 1H), 4.24-4.16 (m, 2H), 3.08 (s, 3H), 2.83-2.77 (m, 3H), 2.69-2.60 (m, 1H), 2.31-2.25 (m, 1H), 1.29 (t, J = 7.6 Hz, 3H), 0.81- 0.77 (m, 2H), 0.46-0.41 (m, 2H). |
| 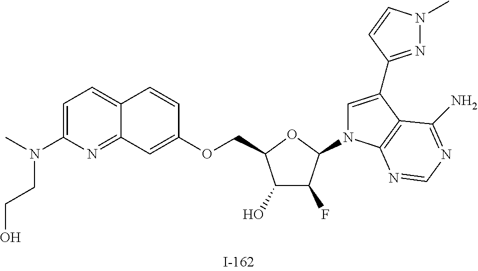 [SMILES: CN(CCO)c1ccc2ccc(OC[C@H]3O[C@@H](n4cc(-c5ccn(C)n5)c5c(N)ncnc54)[C@@H](F)[C@@H]3O)cc2n1] | B | 550.0 | ¹H NMR (400 MHz, DMSO- d₆) δ ppm 9.05 (br, 1H), 8.09 (s, 1H), 7.90 (d, J = 8.8 Hz, 1H), 7.71 (s, 1H), 7.65 (s, 1H), 7.57 (d, J = 8.8 Hz, 1H), 7.23 (br, 1H), 7.04 (s, 1H), 8.91-8.85 (m, 2H), 6.71 (dd, J = 16, 4.8 Hz, 1H), 6.61 (s, 1H), 6.12 (d, J = 4.8 Hz, 1H), 5.18 (dt, J = 50.4, 3.6 Hz, 1H), 4.79-4.75 (m, 1H), 4.62-4.56 (m, 1H), 4.45-4.34 (m, 1H), 4.20 (s, 1H), 3.88 (s, 3H), 3.69-3.63 (m, 4H), 3.17 (s, 3H) |
| 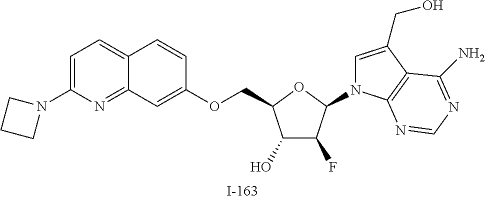 [SMILES: Nc1ncnc2c1c(CO)cn2[C@@H]1O[C@H](COc2ccc3ccc(N4CCC4)nc3c2)[C@@H](O)[C@@H]1F] | B | 481.1 | ¹H NMR (400 MHz, DMSO- d₆) δ ppm 8.10 (s, 1H), 7.92 (d, J = 9.2 Hz, 1H), 7.61 (d, J = 8.8 Hz, 1H), 7.23 (d, J = 2 Hz, 1H), 7.07 (d, J = 2 Hz, 1H), 6.98 (br, 1H), 6.87 (dd, J = 8.4, 2.4 Hz, 1H), 6.65 (dd, J = 15.6, 4.4 Hz, 1H), 6.52 (d, J = 8.4, 2.4 Hz, 1H), 6.14 (d, J = 4.4 Hz, 1H), 5.78 (t, J = 5.2 Hz, 1H), 5.20 (dt, J = 56.8, 4 Hz, 1H), 4.60 (d, J = 4.8 Hz, 2H), 4.55-4.54 (m, 1H), 4.41-4.37 (m, 1H), 4.31-4.26 (m, 1H), |
| | | | 4.18-4.16 (m, 1H), 4.08-4.05 |
| | | | (m, 4H), 2.37-2.33 (m, 2H). |
| 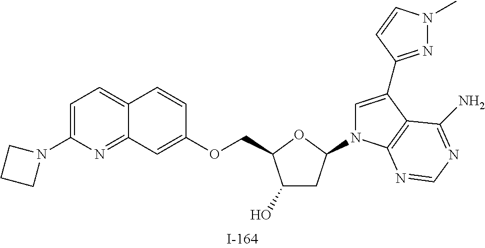 [SMILES: Cn1ccc(-c2cn([C@H]3C[C@H](O)[C@@H](COc4ccc5ccc(N6CCC6)nc5c4)O3)c3ncnc(N)c23)n1] | C | 513.1 | ¹H NMR (400 MHz, DMSO- d₆) δ ppm 8.99 (brs, 1H), 8.06 (s, 1H), 7.92 (d, J = 8.8 Hz, 1H), 7.81 (s, 1H), 7.69 (d, J = 2.4 Hz, 1H), 7.59 (d, J = 8.8 Hz, 1H), 7.22 (brs, 1H), 7.07 (d, J = 2.4 Hz, 1H), 6.91 (dd, J = 8.8, 2.4 Hz, 1H), 6.68-6.64 (m, 1H), 6.53-6.51 (m, 2H), 5.51 (d, J = 4.4 Hz, 1H), 4.54- 4.52 (m, 1H), 4.33-4.30 (m, 1H), 4.24-4.16 (m, 2H), 4.07 (t, J = 7.6 Hz, 4H), 3.87 (s, 3H), 2.73-2.66 (m, 1H), 2.39-2.28 (m, 3H). |
| 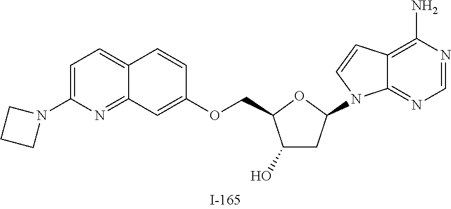 [SMILES: Nc1ncnc2c1ccn2[C@H]1C[C@H](O)[C@@H](COc2ccc3ccc(N4CCC4)nc3c2)O1] | C | 433.0 | ¹H NMR (400 MHz, DMSO- d₆) δ ppm 8.18 (s, 1H), 8.07 (s, 1H), 7.91 (d, J = 8.8 Hz, 1H), 7.59 (d, J = 8.8 Hz, 1H), 7.39 (d, J = 3.6 Hz, 1H), 7.04-7.02 (m, 3H), 6.86 (dd, J = 8.8, 2.4 Hz, 1H), 6.64-6.59 (m, 2H), 6.52 (d, J = 8.8 Hz, 1H), 4.50- 4.48 (m, 1H), 4.28-4.25 (m, 1H), 4.20-4.15 (m, 2H), 4.07 (t, J = 7.6 Hz, 4H), 2.67-2.60 (m, 1H), 2.39-2.32 (m, 2H), 2.30- 2.24 (m, 1H). |
| 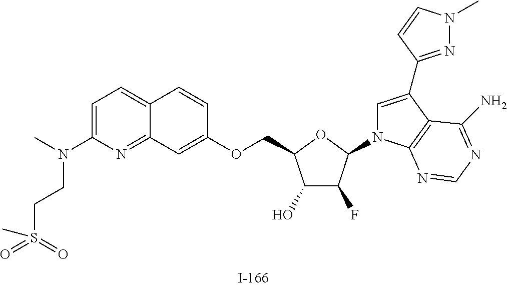 [SMILES: CN(CCS(C)(=O)=O)c1ccc2ccc(OC[C@H]3O[C@@H](n4cc(-c5ccn(C)n5)c5c(N)ncnc54)[C@@H](F)[C@@H]3O)cc2n1] | B | 610.9 | ¹H NMR (400 MHz, DMSO- d₆) δ ppm 9.41 (s, 1H), 8.17 (s, 1H), 8.00 (d, J = 9.1 Hz, 1H), 7.78-7.60 (m, 2H), 7.64 (d, J = 8.8 Hz, 1H), 7.62-7.46 (m, 1H), 7.10 (s, 1H), 7.02-6.84 (m, 2H), 6.71 (dd, J = 15.0, 4.7 Hz, 1H), 6.65 (d, J = 2.2 Hz, 1H), 6.16 (d, J = 4.8 Hz, 1H), 5.35-5.15 (m, 1H), 4.70-4.55 (m, 1H), 4.50-4.35 (m, 2H), 4.27-4.16 (m, 1H), 4.14-4.00 (m, 2H), 3.89 (s, 3H), 3.60-3.44 (m, 2H), 3.16 (s, 3H), 3.07 (s, 3H). |
| 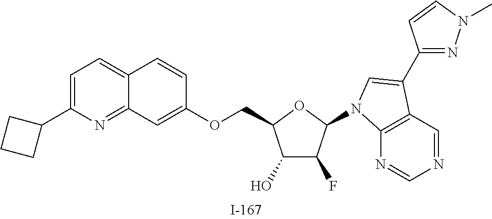 [SMILES: Cn1ccc(-c2cn([C@@H]3O[C@H](COc4ccc5ccc(C6CCC6)nc5c4)[C@@H](O)[C@@H]3F)c3ncncc23)n1] | B | 530.2 | ¹H NMR (400 MHz, DMSO- d₆) δ ppm 8.18 (d, J = 8.4 Hz, 1H), 8.09 (s, 1H), 7.83 (d, J = 5.2 Hz, 1H), 7.71 (d, J = 2.0 Hz, 1H), 7.65 (s, 1H), 7.46 (d, J = 2.4 Hz, 1H), 7.27 (d, J = 8.4 Hz, 1H), 7.22 (dd, J = 2.8, 8.8 Hz, 1H), 6.70 (dd, J = 4.4, 16.0 Hz, 1H), 6.60 (d, J = 2.0 Hz, 1H), 6.16 (d, J = 4.8 Hz, 1H), 5.30-5.15 (m, 1 H), 4.57- 4.51 (m, 2 H), 4.46-4.42 (m, 1H), 3.87 (s, 3H), 3.80 (t, J = 8.4 Hz, 1H), 2.42-2.29 (m, 4H), 2.09-1.86 (m, 2H). |
| 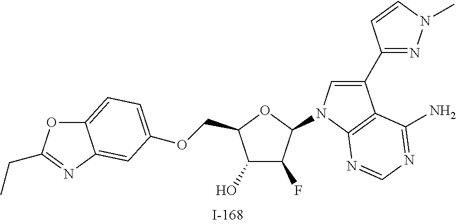 [SMILES: CCc1nc2cc(OC[C@H]3O[C@@H](n4cc(-c5ccn(C)n5)c5c(N)ncnc54)[C@@H](F)[C@@H]3O)ccc2o1] | B | 494.0 | ¹H NMR (400 MHz, DMSO- d₆) δ ppm 9.03 (s, 1H), 8.09 (s, 1H), 7.73 (s, 1H), 7.63-7.53 (m, 2H), 7.33 (s, 1H), 7.00 (dd, J = 8.7, 1.7 Hz, 1H), 6.68 (dd, J = 16.3, 4.3 Hz, 1H), 6.60 (d, J = 1.1 Hz, 1H), 6.13 (d, J = 4.8 Hz, 1H), 5.28 (s, 1H), 5.15 (s, 1H), 4.58-4.50 (m, 1H), 4.40- 4.26 (m, 2H), 4.17 (s, 1H), 3.88 (s, 3H), 2.96-2.81 (m, 3H), 1.33 (t, J = 7.5 Hz, 3H). |
| 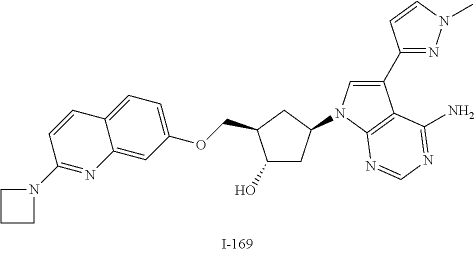 [SMILES: Cn1ccc(-c2cn([C@@H]3C[C@H](COc4ccc5ccc(N6CCC6)nc5c4)[C@@H](O)C3)c3ncnc(N)c23)n1] | E | 511.0 | ¹H NMR (400 MHz, DMSO- d₆) δ ppm 8.95 (s, 1H), 8.04 (s, 1H), 7.91 (d, J = 8.8 Hz, 1H), 7.82 (s, 1H), 7.71 (d, J = 2.2 Hz, 1H), 7.58 (d, J = 8.8 Hz, 1H), 7.13-7.06 (m, 2H), 6.87 (dd, J = 8.7 Hz 2.5 Hz, 1H), 6.63 (d, J = 2.3 Hz, 1H), 6.51 (d, J = 8.8 Hz, 1H), 5.32 (d, J = 9.6 Hz, 1H), 5.01 (s, 1H), 4.27- 4.21 (m, 2H), 4.12-4.02 (m, 5H), 3.87 (s, 3H), 3.32 (s, 1H), 2.39-2.30 (m, 3H), 2.26-2.20 (m, 1H), 2.08-2.03 (m, 1H), 1.80-1.74 (m, 1H). |
| 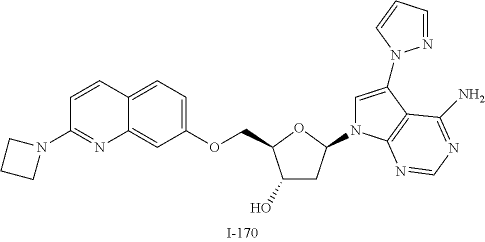 [SMILES: Nc1ncnc2c1c(-n1cccn1)cn2[C@H]1C[C@H](O)[C@@H](COc2ccc3ccc(N4CCC4)nc3c2)O1] | C | 499.1 | ¹H NMR (400 MHz, DMSO- d₆) δ ppm 8.30 (d, J = 2.4 Hz, 1H), 8.14 (s, 1H), 7.90 (d, J = 9.2 Hz, 1H), 7.87 (s, 1H), 7.83 (d, J = 1.6 Hz, 1H), 7.56 (d, J = 8.8 Hz, 1H), 7.06 (d, J = 2.4 Hz, 1H), 6.87 (dd, J = 9.2 2.4 Hz, 1H), 6.72 (t, J = 7.2 Hz, 1H), 6.54 (t, J = 2.4 Hz, 1H), 6.51 (d, J = 8.8 Hz, 1H), 5.54 (d, J = 4.0 Hz, 1H), 4.55-4.54 (m, 1H), 4.33-4.28 (m, 1H), 4.24-4.17 (m, 2H), 4.06 (t, J = 1.2 Hz, 4H), 2.70-2.63 (m, 1H), 2.38-2.31 (m, 3H). |
| 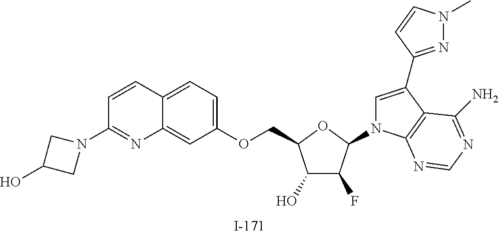 [SMILES: Cn1ccc(-c2cn([C@@H]3O[C@H](COc4ccc5ccc(N6CC(O)C6)nc5c4)[C@@H](O)[C@@H]3F)c3ncnc(N)c23)n1] | B | 547.8 | ¹H NMR (400 MHz, DMSO- d₆) δ ppm 9.04 (s, 1H), 8.09 (s, 1H), 7.93 (d, J = 8.8 Hz, 1H), 7.72 (d, J = 2.4 Hz, 1H), 7.64 (d, J = 2.4 Hz, 1H), 7.60 (d, J = 8.8, 1H), 7.25 (s, 1H), 7.10 (d, J = 2.4 Hz, 1H), 6.89 (dd, J = 8.8 Hz, 2.4 Hz, 1H), 6.70 (dd, J = 8 Hz, J = 4 Hz, 1H), 6.62 (d, J = 2.4 Hz, 1H), 6.55 (d, J = 8.8 Hz, 1H), 6.14 (s, J = 4.8 Hz, 1H), 5.71 (d, J = 6.8 Hz, 1H), 5.29-5.14 (m, 1H), 4.62- 4.52 (m, 2H), 4.46-4.43 (m, 1H), 4.38-4.29 (m, 1H), 4.28 (t, J = 2.4 Hz, 2H), 4.26-4.28 (m, 1H), 3.88 (s, 3H), 3.82-3.77 (m, |
| | | | 2H). |
| 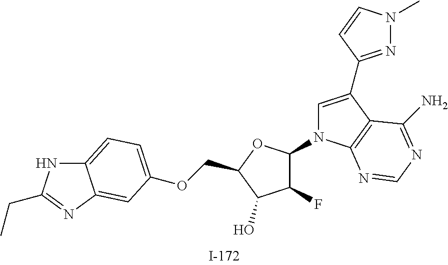 [SMILES: CCc1nc2cc(OC[C@H]3O[C@@H](n4cc(-c5ccn(C)n5)c5c(N)ncnc54)[C@@H](F)[C@@H]3O)ccc2[nH]1] | B | 493.5 | ¹H NMR (400 MHz, DMSO- d₆) δ ppm 9.10 (s, 1H), 8.10 (s, 1H), 7.73 (d, J = 2 Hz, 1H), 7.64 (d, J = 2 Hz 1H), 6.84 (s, 1H), 6.83 (s, 1H), 6.82 (d, J = 4 Hz, 1H), 6.72 (d, J = 4.4 Hz, 1H), 6.67 (s, 1H), 6.14 (s, 2H), 5.29-5.16 (m, 1 H), 4.34- 4.33 (m, 1H), 4.30-4.28 (m, 3H), 4.19 (s, 3H), 2.83-2.81 (m, 2H), 1.33-1.31 (m, 4H). |
| 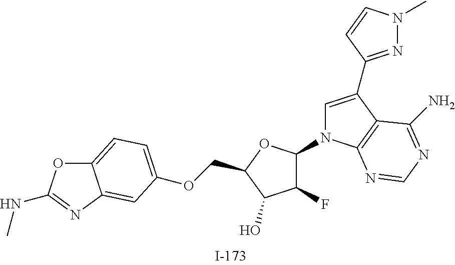 [SMILES: CNc1nc2cc(OC[C@H]3O[C@@H](n4cc(-c5ccn(C)n5)c5c(N)ncnc54)[C@@H](F)[C@@H]3O)ccc2o1] | B | 495.0 | ¹H NMR (400 MHz, DMSO- d₆) δ ppm 9.14 (brs, 1H), 8.11 (s, 1H), 7.76 (d, J = 4.8 Hz 1H), 7.73 (d, J = 2.4 Hz, 1H), 7.63 (d, J = 1.6 Hz, 1H), 7.22 (d, J = 8.8 Hz, 1H), 6.92 (d, J = 2.4 Hz, 1H), 6.67 (dd, J = 2.4 Hz 12.0 Hz, 1H), 6.62-6.58 (m, 2H), 6.11 (d, J = 2.4 Hz 1H), 5.28-5.13 (m, 1H), 4.56-4.49 (m, 1H), 4.32-4.22 (m, 2H), 4.16-4.12 (m, 1H), 3.88 (s, 3H), 2.87 (d, J = 4.8 Hz, 3H). |
| 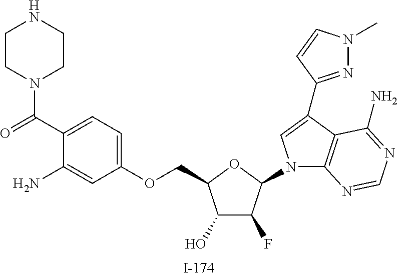 [SMILES: Cn1ccc(-c2cn([C@@H]3O[C@H](COc4ccc(C(=O)N5CCNCC5)c(N)c4)[C@@H](O)[C@@H]3F)c3ncnc(N)c23)n1] | B | 552.0 | ¹H NMR (400 MHz, DMSO- d₆) δ ppm 9.10-9.00 (br, 1H), 8.09 (s, 1H), 7.73 (d, J = 2.3 Hz, 1H), 7.61 (d, J = 2.3 Hz, 1H), 7.32-7.23 (br, 1H), 6.93 (d, J = 8.4 Hz, 1H), 6.67 (dd, J = 16.5 4.4 Hz, 1H), 6.63 (d, J = 2.3 Hz, 1H), 6.33 (d, J = 2.4 Hz, 1H), 6.23 (dd, J = 8.5 2.4 Hz, 1H), 6.11 (d, J = 4.9 Hz, 1H), 5.29 (s, 2H), 5.27- 5.12 (m, 1H), 4.54-4.47 (m, 1H), 4.27-4.11 (m, 3H), 3.88 (s, 3H), 3.40-3.36 (m, 4H), 2.68-2.62 (m, 4H). |
| 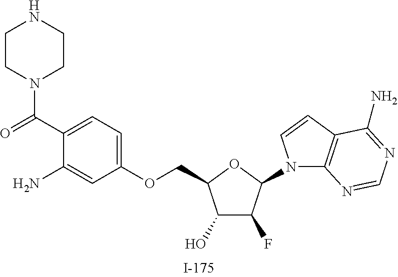 [SMILES: Nc1cc(OC[C@H]2O[C@@H](n3ccc4c(N)ncnc43)[C@@H](F)[C@@H]2O)ccc1C(=O)N1CCNCC1] | B | 472.0 | ¹H NMR (400 MHz, DMSO- d₆) δ ppm 8.09 (s, 1H), 7.23- 7.21 (m, 1H), 7.11-7.06 (br, 2H), 6.93 (d, J = 8.5 Hz, 1H), 6.65-6.57 (m, 2H), 6.32 (d, J = 2.4 Hz, 1H), 6.21 (dd, J = 8.5 2.4 Hz, 1H), 6.10-6.09 (m, 1H), 5.29 (s, 2H), 5.24-5.09 (m, 1H), 4.50-4.42 (m, 1H), 4.22-4.09 (m, 3H), 3.39-3.35 (m, 5H), 2.69-2.63 (m, 4H). |
| 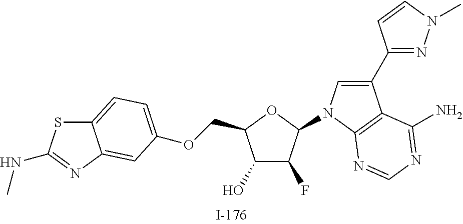 | B | 510.0 | ¹H NMR (400 MHz, DMSO- d₆) δ ppm 9.06 (s, 1H), 8.09 (s, 1H), 7.94-9.91 (m, 1H), 7.72 (d, J = 2 Hz, 1H), 7.62 (s, 1H), 7.52 (d, J = 8.8, 1H), 7.25 (s, 1H), 6.72-6.64 (m, 2H), 6.61 (d, J = 2 Hz, 1H), 6.11 (d, J = 4.8 Hz, 1H), 5.18-5.23 (m, 1H), 4.57-4.49 (m, 1H), 4.36-4.23 (m, 2H), 4.48-4.34 (m, 1H), 3.88 (s, 3H), 2.92 (d, J = 4.8 Hz, 3H). |
| 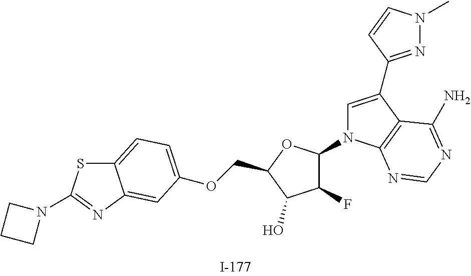 [SMILES: Cn1ccc(-c2cn([C@@H]3O[C@H](COc4ccc5sc(N6CCC6)nc5c4)[C@@H](O)[C@@H]3F)c3ncnc(N)c23)n1] | B | 537.0 | ¹H NMR (400 MHz, DMSO- d₆) δ ppm 9.09 (s, 1H), 8.09 (s, 1H), 7.73 (d, J = 5.6 Hz, 1H), 7.63 (d, J = 8.8 Hz, 1H), 7.61 (d, J = 2 Hz, 1H), 7.33 (s, 1H), 7.16 (d, J = 2.4 Hz, 1H), 6.76 (dd, J = 8 Hz 2.4 Hz, 1H), 6.67 (dd, J = 16 Hz 4.4 Hz, 1H), 6.60 (dd, J = 2.4 Hz, 1H), 6.12 (d, J = 4.4 Hz, 1H), 5.28-5.13 (m, 1H), 4.47-4.51 (m, 1H), 4.39-4.25 (m, 2H), 4.18-4.06 (m, 1H), 3.97 (t, J = 7.6 Hz, 3H), 3.33 (s, 1H), 0.54-0.48 (m, 2H), 2.47-2.41 (m, 2H). |
| 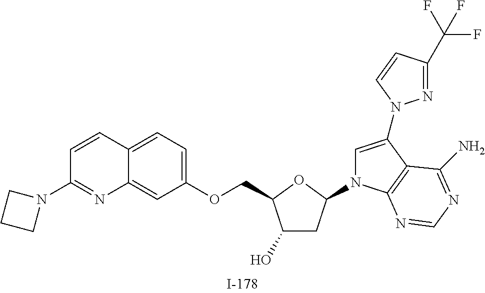 [SMILES: Nc1ncnc2c1c(-n1ccc(C(F)(F)F)n1)cn2[C@H]1C[C@H](O)[C@@H](COc2ccc3ccc(N4CCC4)nc3c2)O1] | C | 567.0 | ¹H NMR (400 MHz, DMSO- d₆) δ ppm 8.55 (d, J = 1.2 Hz, 1H), 8.18 (s, 1H), 8.04 (s, 1H), 7.89 (d, J = 8.8 Hz, 1H), 7.56- 7.53 (m, 3H), 7.08 (d, J = 2.4 Hz, 1H), 7.05 (d, J = 2.0 Hz, 1H), 6.86 (dd, J = 8.8 Hz 2.4 Hz, 1H), 6.72 (t, J = 6.4 Hz, 1H), 6.50 (d, J = 8.8 Hz, 1H), 5.52 (d, J = 4.0 Hz, 1H), 4.58- 4.55 (m, 1H), 4.34-4.30 (m, 1H), 4.24-4.17 (m, 2H), 4.05 (t, J = 7.2 Hz, 4H), 2.69-2.62 (m, 1H), 2.41-2.31 (m, 3H). |
| 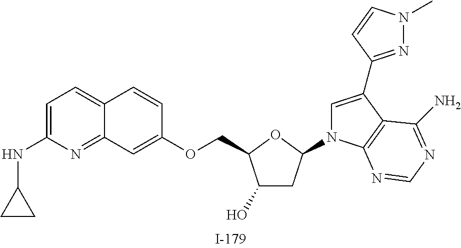 [SMILES: Cn1ccc(-c2cn([C@H]3C[C@H](O)[C@@H](COc4ccc5ccc(NC6CC6)nc5c4)O3)c3ncnc(N)c23)n1] | C | 513.0 | ¹H NMR (400 MHz, DMSO- d₆) δ ppm 8.98 (brs, 1H), 8.09 (s, 1H), 7.82-7.79 (m, 2H), 7.68 (d, J = 2.4 Hz, 1H), 7.53 (d, J = 8.8 Hz, 1H), 7.18-7.15 (m, 2H), 7.02 (d, J = 2.4 Hz, 1H), 6.86 (dd, J = 8.8 Hz 2.4 Hz, 1H), 6.68-6.65 (m, 2H), 6.52 (d, J = 2.0 Hz, 1H), 5.50 (d, J = 4.0 Hz, 1H), 4.54-4.53 (m, 1H), 4.33-4.30 (m, 1H), 4.23-4.17 (m, 2H), 3.86 (s, 3H), 2.78-2.67 (m, 2H), 2.33-2.28 (m, 1H), 0.76-0.71 (m, 2H), 0.48-0.45 (m, 2H). |
| 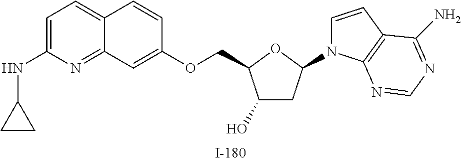 [SMILES: Nc1ncnc2c1ccn2[C@H]1C[C@H](O)[C@@H](COc2ccc3ccc(NC4CC4)nc3c2)O1] | C | 433.0 | ¹H NMR (400 MHz, DMSO- d₆) δ ppm 8.07 (s, 1H), 7.82 (d, J = 7.2 Hz, 1H), 7.55 (d, J = 8.8 Hz, 1H), 7.38 (d, J = 3.6 Hz, 1H), 7.23 (brs, 1H), 7.02 (s, 3H), 6.85 (d, J = 8.4 Hz, 1H), 6.68-6.58 (m, 3H), 5.48 (d, J = 4.4 Hz, 1H), 4.50-4.49 (m, 1H), 4.29-4.26 (m, 1H), 4.20-4.16 (m, 2H), 2.77-2.76 (m, 1H), 2.69-2.62 (m, 1H), 2.30-2.24 (m, 1H), 0.78-0.74 |
| | | | (m, 2H), 0.48-0.44 (m, 2H). |
| 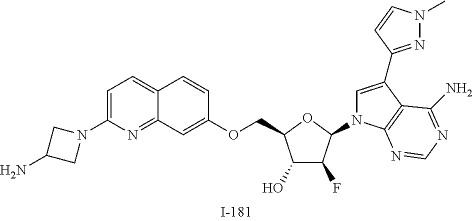 [SMILES: Cn1ccc(-c2cn([C@@H]3O[C@H](COc4ccc5ccc(N6CC(N)C6)nc5c4)[C@@H](O)[C@@H]3F)c3ncnc(N)c23)n1] | B | 546.0 | ¹H NMR (400 MHz, DMSO- d₆) δ ppm 9.03 (brs, 1H), 8.08 (s, 1H), 7.94 (d, J = 8.8 Hz 1H), 7.71 (d, J = 2.4 Hz, 1H), 7.64-7.60 (m, 2H), 7.26 (s, 1H), 7.10 (d, J = 2.4 Hz, 1H), 6.90 (dd, J = 2.0 Hz 8.4 Hz, 1H), 6.69 (dd, J = 4.8 Hz 16.0 Hz, 1H), 6.61 (d, J = 2.4 Hz 1H), 6.57 (d, J = 8.8 Hz 1H), 6.13 (d, J = 4.8 Hz, 1H), 5.29-5.14 (m, 1H), 4.60-4.53 (m, 1H), 4.62-4.42 (m, 1H), 4.37-4.18 (m, 5H), 3.96-3.91 (m, 1H), 3.88 (s, 3H), 3.79 (s, 2H). |
| 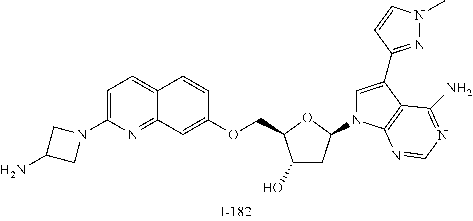 [SMILES: Cn1ccc(-c2cn([C@H]3C[C@H](O)[C@@H](COc4ccc5ccc(N6CC(N)C6)nc5c4)O3)c3ncnc(N)c23)n1] | C | 528.0 | ¹H NMR (400 MHz, DMSO- d₆) δ ppm 8.98 (brs, 1H), 8.07 (s, 1H), 7.89 (d, J = 8.8 Hz, 1H), 7.81 (s, 1H), 7.69 (d, J = 2.4 Hz, 1H), 7.58 (d, J = 8.8 Hz, 1H), 7.19 (brs, 1H), 7.06 (d, J = 2.4 Hz, 1H), 6.89 (dd, J = 8.8 Hz 2.4 Hz, 1H), 6.68- 6.65 (m, 1H), 6.53-6.51 (m, 2H), 5.50 (d, J = 3.6 Hz, 1H), 4.53 (s, 1H), 4.33-4.30 (m, 1H), 4.24-4.16 (m, 4H), 3.87 (s, 3H), 3.86-3.82 (m, 1H), 3.68-3.64 (m, 2H), 2.73-2.66 (m, 1H), 2.33-2.28 (m, 1H). |
| 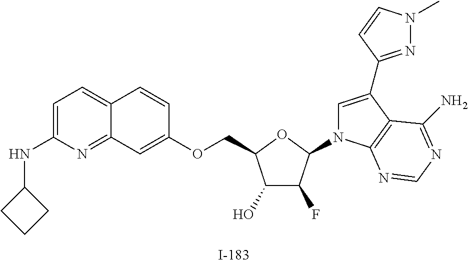 [SMILES: Cn1ccc(-c2cn([C@@H]3O[C@H](COc4ccc5ccc(NC6CCC6)nc5c4)[C@@H](O)[C@@H]3F)c3ncnc(N)c23)n1] | B | 545.0 | ¹H NMR (400 MHz, DMSO- d₆) δ ppm 9.05 (s, 1H), 8.10 (s, 1H), 7.75 (d, J = 8.9 Hz, 1H), 7.71 (d, J = 2.1 Hz, 1H), 7.66 (d, J = 1.8 Hz, 1H), 7.51 (d, J = 8.7 Hz, 1H), 7.35-7.15 (m, 2H), 7.02 (d, J = 1.9 Hz, 1H), 6.82 (dd, J = 8.7 Hz 2.2 Hz, 1H), 6.70 (dd, J = 15.8, 4.4 Hz, 1H), 6.62 (d, J = 2.1 Hz, 1H), 6.55 (d, J = 8.8 Hz, 1H), 6.15 (d, J = 4.7 Hz, 1H), 5.33-5.13 (m, 1H), 4.65-4.48 (m, 2H), 4.47-4.31 (m, 2H), 4.25-4.15 (m, 1H), 3.88 (s, 3H), 2.38-2.26 (m, 2H), 1.99-1.82 (m, 2H), 1.80-1.59 (m, 2H). |
| 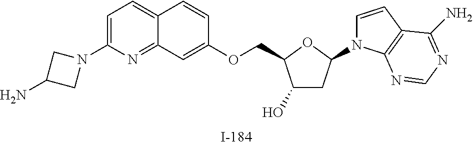 [SMILES: Nc1ncnc2c1ccn2[C@H]1C[C@H](O)[C@@H](COc2ccc3ccc(N4CC(N)C4)nc3c2)O1] | C | 224.6 | ¹H NMR (400 MHz, DMSO- d₆) δ ppm 8.08 (s, 1H), 7.92 (d, J = 8.8 Hz, 1H), 7.60 (d, J = 8.8 Hz, 1H), 7.38 (d, J = 3.6 Hz, 1H), 7.04 (d, J = 2.4 Hz, 1H), 7.02 (s, 2H), 6.87 (dd, J = 8.8 Hz 2.4 Hz, 1H), 6.64-6.59 (m, 2H), 6.55 (d, J = 8.8 Hz, 1H), 5.49 (d, J = 4.0 Hz, 1H), 4.50-4.49 (m, 1H), 4.31-4.23 (m, 3H), 4.20-4.14 (m, 2H), |
| | | | 3.94-3.88 (m, 1H), 3.77-3.73 |
| | | | (m, 2H), 2.67-2.62 (m, 1H), |
| | | | 2.30-2.24 (m, 1H). |
| 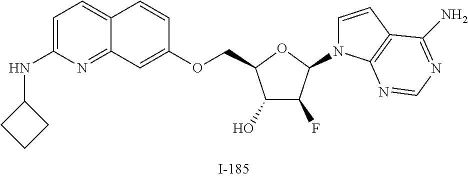 [SMILES: Nc1ncnc2c1ccn2[C@@H]1O[C@H](COc2ccc3ccc(NC4CCC4)nc3c2)[C@@H](O)[C@@H]1F] | B | 465.0 | ¹H NMR (400 MHz, DMSO- d₆) δ ppm 8.09 (s, 1H), 7.75 (d, J = 8.6 Hz, 1H), 7.51 (d, J = 8.7 Hz, 1H), 7.32-7.27 (m, 1H), 7.21 (s, 1H), 7.07 (s, 2H), 6.99 (d, J = 2.2 Hz, 1H), 6.81 (dd, J = 8.8 Hz 2.3 Hz, 1H), 6.69- 6.59 (m, 2H), 6.59-6.49 (m, 1H), 6.11 (d, J = 4.9 Hz, 1H), 5.34-5.01 (m, 1H), 4.65-4.45 (m, 2H), 4.44-4.24 (m, 2H), 4.24-4.09 (m, 1H), 2.40-2.24 (m, 2H), 2.05-1.80 (m, 2H), |
| | | | 1.70 (s, 2H). |
| 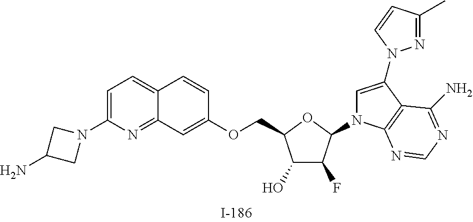 [SMILES: Cc1ccn(-c2cn([C@@H]3O[C@H](COc4ccc5ccc(N6CC(N)C6)nc5c4)[C@@H](O)[C@@H]3F)c3ncnc(N)c23)n1] | B | 546.0 | ¹H NMR (400 MHz, DMSO- d₆) δ ppm 8.24 (d, J = 2.0 Hz, 1H), 8.14 (s, 1H), 7.90 (d, J = 9.2 Hz, 1H), 7.67 (d, J = 2.0 Hz, 1H), 7.58 (d, J = 9.2 Hz, 1H), 7.08 (d, J = 2.4 Hz, 1H), 6.86 (dd, J = 8.8, 2.8 Hz, 1H), 6.75 (dd, J = 14.8 Hz 4.4 Hz, 1H), 6.53 (d, J = 8.8 Hz, 1H), 6.33 (d, J = 2.4 Hz, 1H), 6.16- 6.13 (m, 1H), 5.25 (dt, J = 52.4 Hz 4.0 Hz, 1H), 4.60-4.55 (m, 1H), 4.46-4.42 (m, 1H), 4.38- 4.34 (m, 1H), 4.24-4.19 (m, 3H), 3.87-3.81 (m, 1H), 3.68- |
| | | | 3.65 (m, 2H), 2.30 (s, 3H). |
| 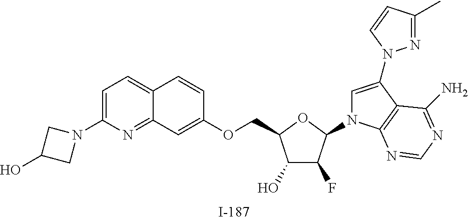 [SMILES: Cc1ccn(-c2cn([C@@H]3O[C@H](COc4ccc5ccc(N6CC(O)C6)nc5c4)[C@@H](O)[C@@H]3F)c3ncnc(N)c23)n1] | B | 547.1 | ¹H NMR (400 MHz, MeOD-d₄) δ ppm 8.23 (d, J = 2.4 Hz, 1H), 8.14 (s, 1H), 7.92 (d, J = 6.8 Hz, 1H), 7.66 (d, J = 1.6 Hz, 1H), 7.60 (d, J = 8.8 Hz, 1H), 7.09 (d, J = 2.4 Hz, 1H), 6.88 (dd, J = 2 Hz 8.8 Hz, 1H), 6.75 (dd, J = 4.8 Hz 15.2 Hz, 1H), 6.54 (d, J = 8.8 Hz, 1H), 6.33 (d, J = 2 Hz, 1H), 6.15 (d, J = 4.8 Hz, 1H), 5.70 (d, J = 2.4 Hz, 1H), 5.31-5.17 (m, 1H), 4.82-4.56 (m, 2H), 4.43-4.42 (m, 1H), 4.43-4.34 (m, 1H), 4.29-4.26 (m, 2H), 4.21-4.20 |
| | | | (m, 1H), 3.82-3.76 (m, 2H), |
| | | | 2.33-2.30 (s, 3H). |
| 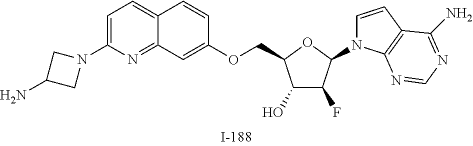 [SMILES: Nc1ncnc2c1ccn2[C@@H]1O[C@H](COc2ccc3ccc(N4CC(N)C4)nc3c2)[C@@H](O)[C@@H]1F] | B | 465.9 | ¹H NMR (400 MHz, DMSO- d₆) δ ppm 8.09 (s, 1H), 7.90 (d, J = 8.4 Hz, 1H), 7.59 (d, J = 8.8 Hz, 1H), 7.30-7.28 (m, 1H), 7.07 (s, 2H), 7.06 (s, 1H), 6.87 (dd, J = 8.8 Hz 2.4 Hz, 1H), 6.68-6.62 (m, 2H), 6.53 (d, J = 8.8 Hz, 1H), 6.12 (d, J = 4.4 Hz, 1H), 5.19 (dt, J = 52.4 Hz 4.0 Hz, 1H), 4.55-4.50 (m, 1H), 4.40-4.37 (m, 1H), 4.32-4.16 |
| | | | (m, 4H), 3.87-3.82 (m, 1H), |
| | | | 3.68-3.63 (m, 2H). |
| 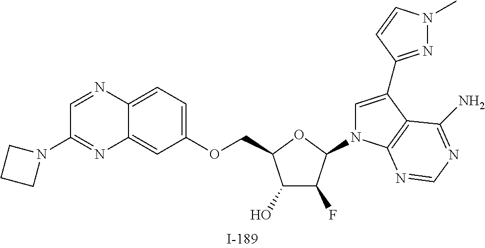 [SMILES: Cn1ccc(-c2cn([C@@H]3O[C@H](COc4ccc5ncc(N6CCC6)nc5c4)[C@@H](O)[C@@H]3F)c3ncnc(N)c23)n1] | B | 532.0 | ¹H NMR (400 MHz, DMSO- d₆) δ ppm 9.05-9.00 (br, 1H), 8.10 (d, J = 11.2 Hz, 2H), 7.73- 7.70 (m, 2H), 7.62 (d, J = 2.3 Hz, 1H), 7.34-7.25 (br, 1H), 7.13 (d, J = 2.7 Hz, 1H), 7.06 (dd, J = 9.0 Hz 2.8 Hz, 1H), 6.69 (dd, J = 16.3 Hz 4.4 Hz, 1H), 6.59 (d, J = 2.3 Hz, 1H), 6.13 (d, J = 4.9 Hz, 1H), 5.29- 5.14 (m, 1H), 4.61-4.53 (m, 1H), 4.50-4.44 (m, 1H), 4.44- 4.35 (m, 1H), 4.21-4.17 (m, 4H), 3.88 (s, 3H), 3.57 (s, 1H), 2.44-2.40 (m, 2H). |
| 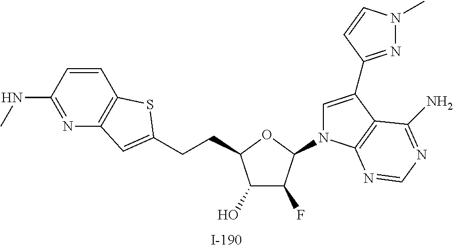 [SMILES: CNc1ccc2sc(CC[C@H]3O[C@@H](n4cc(-c5ccn(C)n5)c5c(N)ncnc54)[C@@H](F)[C@@H]3O)cc2n1] | H | 508.9 | ¹H NMR (400 MHz, MeOD-d₄) δ ppm 8.10 (s, 1H), 7.80 (d, J = 8.8 Hz, 1H), 7.62-7.53 (m, 2H), 7.08 (s, 1H), 6.65-6.57 (m, 1H), 6.56 (d, J = 2.3 Hz, 1H), 6.50 (d, J = 8.9 Hz, 1H), 5.12-4.94 (m, 1H), 4.36-4.24 (m, 1H), 4.02-3.96 (m, 1H), 3.95 (s, 3H), 3.25-3.05 (m, 2H), 2.93 (s, 3H), 2.35-2.20 (m, 2H). |
| 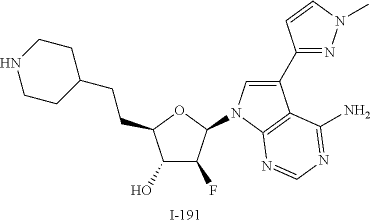 [SMILES: Cn1ccc(-c2cn([C@@H]3O[C@H](CCC4CCNCC4)[C@@H](O)[C@@H]3F)c3ncnc(N)c23)n1] | H | 430.0 | ¹H NMR (400 MHz, DMSO- d₆) δ ppm 9.04 (s, 1H), 8.08 (s, 1H), 7.74 (d, J = 2.3 Hz, 1H), 7.55 (d, J = 2.5 Hz, 1H), 7.23 (s, 1H), 6.69 (d, J = 2.3 Hz, 1H), 6.54 (dd, J = 17.9 Hz 4.2 Hz, 1H), 5.88 (d, J = 4.2 Hz, 1H), 5.16-4.95 (m, 1H), 4.19 (d, J = 19.9 Hz, 1H), 3.88 (s, 3H), 3.70 (dt, J = 10.6 Hz 5.2 Hz, 1H), 2.94 (d, J = 11.1 Hz, 2H), 1.86-1.49 (m, 5H), 1.45- 1.19 (m, 4H), 1.01 (m, 3H). |
| 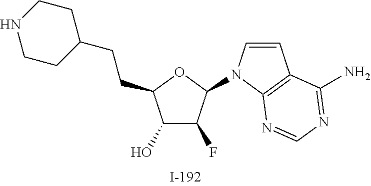 [SMILES: Nc1ncnc2c1ccn2[C@@H]1O[C@H](CCC2CCNCC2)[C@@H](O)[C@@H]1F] | H | 350.0 | ¹H NMR (400 MHz, DMSO- d₆) δ ppm 8.07 (s, 1H), 7.18- 7.11 (m, 1H), 7.07 (s, 2H), 6.61 (d, J = 3.7 Hz, 1H), 6.50 (dd, J = 18.7, 4.1 Hz, 1H), 5.87 (s, 1H), 5.10-4.91 (m, 1H), 4.14 (d, J = 20.3 Hz, 1H), 3.70 (dt, J = 9.6, 4.9 Hz, 1H), 2.91 (d, J = 11.6 Hz, 2H), 2.42 (s, 2H), 1.63 (dd, J = 37.5 Hz 25.2 Hz, 5H), 1.30 (m, 3H), 0.98 (d, J = 11.0 Hz, 2H). |
| 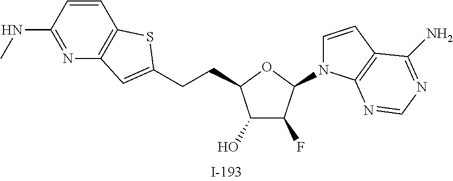 [SMILES: CNc1ccc2sc(CC[C@H]3O[C@@H](n4ccc5c(N)ncnc54)[C@@H](F)[C@@H]3O)cc2n1] | H | 429.6 | ¹H NMR (400 MHz, DMSO- d₆) δ ppm 8.08 (s, 1H), 7.81 (d, J = 8.9 Hz, 1H), 7.28-7.16 (m, 1H), 7.07 (s, 2H), 7.01 (s, 1H), 6.63 (d, J = 3.7 Hz, 1H), 6.60- 6.48 (m, 2H), 6.44 (d, J = 8.8 Hz, 1H), 5.92 (d, J = 4.9 Hz, 1H), 5.20-4.95 (m, 1H), 4.34- 4.22 (m, 1H), 3.94-3.69 (m, 1H), 3.15-2.85 (m, 2H), 2.79 (d, J = 4.8 Hz, 3H), 2.23-1.89 (m, 2H). |
| 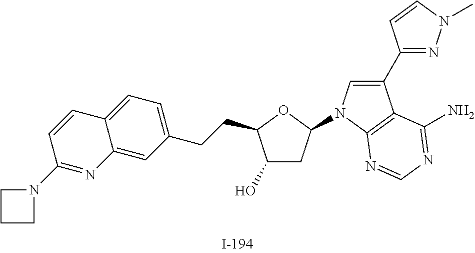 [SMILES: Cn1ccc(-c2cn([C@H]3C[C@H](O)[C@@H](CCc4ccc5ccc(N6CCC6)nc5c4)O3)c3ncnc(N)c23)n1] | H | 511.1 | ¹H NMR (400 MHz, DMSO- d₆) δ ppm 9.18 (s, 1H), 8.10 (s, 1H), 7.81 (s, 1H), 7.73 (d, J = 2.4 Hz, 1H), 7.65 (d, J = 8 Hz, 1H), 7.44 (s, 1H), 7.27 (s, 1H), 7.14 (s, 1H), 6.70 (d, J = 2.4 Hz, 1H), 6.57-6.53 (m, 1H), 5.24 (d, J = 4 Hz, 1H), 4.26 (s, 1H), 4.11 (s, 1H), 3.88 (s, 3H), 3.74 (s, 1H), 2.80-2.60 (m, 2H), 2.4- 2.33 (m, 2H), 2.30-2.20 (m, 2H), 2.02-1.90 (m, 2H). |
| 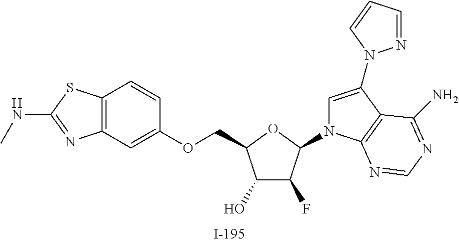 [SMILES: CNc1nc2cc(OC[C@H]3O[C@@H](n4cc(-n5cccn5)c5c(N)ncnc54)[C@@H](F)[C@@H]3O)ccc2s1] | B | 496.9 | ¹H NMR (400 MHz, DMSO- d₆) δ ppm 8.41 (d, J = 2.0 Hz, 1H), 8.18 (s, 1H), 7.96 (d, J = 4.8 Hz, 1H), 7.85 (d, J = 1.6 Hz, 1H), 7.75 (d, J = 2.0 Hz, 1H), 7.53 (d, J = 8.4 Hz, 1H), 7.09 (d, J = 2.4 Hz, 1H), 6.75 (dd, J = 15.2, 4.4 Hz, 1H), 6.70 (dd, J = 8.4 Hz 2.4 Hz, 1H), 6.57 (t, J = 2.0 Hz, 1H), 6.15 (d, J = 4.4 Hz, 1H), 5.25 (dt, J = 52.8, 4.0 Hz, 1H), 4.59-4.52 (m, 1H), 4.37-4.34 (m, 1H), 4.30-4.26 (m, 1H), 4.20-4.16 (m, 1H), 2.92 (d, J = 4.4 Hz, 3H). |
| 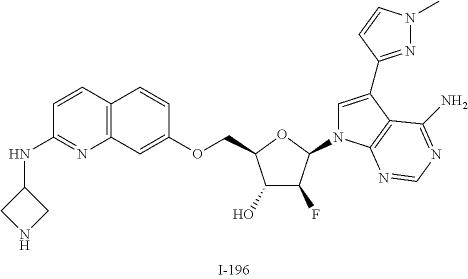 [SMILES: Cn1ccc(-c2cn([C@@H]3O[C@H](COc4ccc5ccc(NC6CNC6)nc5c4)[C@@H](O)[C@@H]3F)c3ncnc(N)c23)n1] | B | 546.0 | ¹H NMR (400 MHz, MeOD-d₄) δ ppm 8.11-8.09 (m, 1H), 8.04 (s, 1H), 7.55 (s, 1H), 7.50 (d, J = 2.4 Hz, 1H), 7.48 (d, J = 2 Hz, 1H), 7.23 (d, J = 2.4 Hz, 1H), 6.60 (d, J = 4 Hz, 1H), 6.43 (d, J = 2.4 Hz, 1H), 6.22 (d, J = 2 Hz, 1H), 5.08-5.06 (m, 1H), 4.95-4.93 (m, 1H), 4.76- 4.71 (m, 2H), 4.55-4.54 (m, 1H), 4.51-4.50 (m, 1H), 4.46- 4.44 (m, 1H), 4.42-4.40 (m, 1H), 4.31-4.28 (m, 2H), 3.94 (s, 3H). |
| 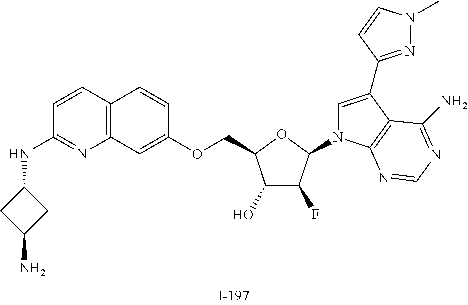 [SMILES: Cn1ccc(-c2cn([C@@H]3O[C@H](COc4ccc5ccc(N[C@H]6C[C@H](N)C6)nc5c4)[C@@H](O)[C@@H]3F)c3ncnc(N)c23)n1] | B | 560.0 | ¹H NMR (400 MHz, DMSO- d₆) δ ppm 9.03 (s, 1H), 8.09 (s, 1H), 7.73 (dd, J = 9.5 Hz 5.5 Hz, 2H), 7.65 (d, J = 2.2 Hz, 1H), 7.51 (d, J = 8.8 Hz, 1H), 7.35-7.17 (m, 2H), 7.01 (d, J = 2.2 Hz, 1H), 6.82 (dd, J = 8.7, 2.4 Hz, 1H), 6.70 (dd, J = 15.7 Hz 4.6 Hz, 1H), 6.59 (dd, J = 19.5 Hz 5.6 Hz, 2H), 6.13 (s, 1H), 5.33-5.11 (m, 1H), 4.58 (d, J = 20.1 Hz, 2H), 4.39 (ddd, J = 17.1 Hz 11.0 Hz 5.0 Hz, 2H), 4.19 (d, J = 3.5 Hz, 1H), 3.88 (s, 3H), 3.56 (s, 1H), 2.12 (dd, J = 13.8 Hz 7.7 Hz, 4H). |
| 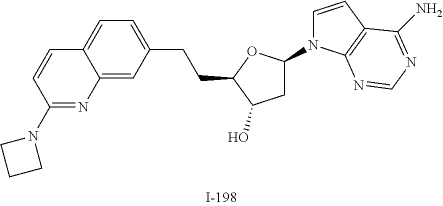 [SMILES: Nc1ncnc2c1ccn2[C@H]1C[C@H](O)[C@@H](CCc2ccc3ccc(N4CCC4)nc3c2)O1] | F | 431.5 | ¹H NMR (400 MHz, DMSO- d₆) δ ppm 8.07 (s, 1H), 7.96 (d, J = 8.8 Hz, 1H), 7.60 (d, J = 8.8 Hz, 1H), 7.39 (s, 1H), 7.32 (d, J = 4.4 Hz, 1H), 7.07 (d, J = 8 Hz, 1H), 7.02 (s, 1H), 6.63 (d, J = 8.4 Hz, 1H), 6.62 (d, J = 4.4 Hz, 1H), 6.54-6.50 (m, 1H), 5.27 (d, J = 4.4 Hz, 1H), 4.24 (s, 1H), 4.08 (t, J = 7.2 Hz, 4H), 3.74 (s, 1H), 2.67-2.52 (m, 2H), 2.40-2.32 (m, 2H), 2.21- 2.26 (m, 2H), 2.20-2.90 (m, 2H). |
| 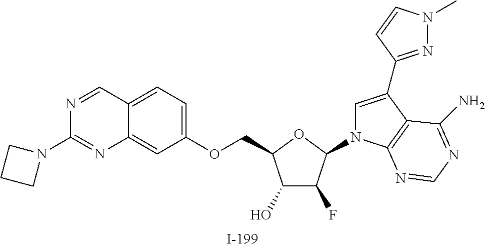 [SMILES: Cn1ccc(-c2cn([C@@H]3O[C@H](COc4ccc5cnc(N6CCC6)nc5c4)[C@@H](O)[C@@H]3F)c3ncnc(N)c23)n1] | B | 532.0 | ¹H NMR (400 MHz, DMSO- d₆) δ ppm 9.11-9.05 (br, 1H), 8.99 (s, 1H), 8.10 (s, 1H), 7.75- 7.72 (m, 2H), 7.62 (d, J = 2.3 Hz, 1H), 7.35-7.27 (br, 1H), 7.03 (d, J = 2.3 Hz, 1H), 6.92 (dd, J = 8.8 Hz 2.4 Hz, 1H), 6.70 (dd, J = 16.3 Hz 4.4 Hz, 1H), 6.61 (d, J = 2.3 Hz, 1H), 6.14 (d, J = 4.9 Hz, 1H), 5.29- 5.14 (m, 1H), 4.61-4.48 (m, 2H), 4.42-4.38 (m, 1H), 4.22- 4.19 (m, 1H), 4.11 (t, J = 7.5 Hz, 4H), 3.88 (s, 3H), 2.37- 2.29 (m, 2H). |
| 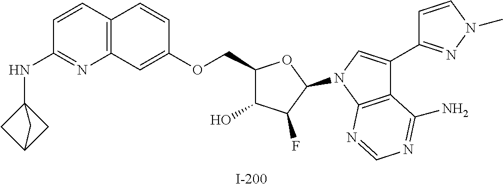 [SMILES: Cn1ccc(-c2cn([C@@H]3O[C@H](COc4ccc5ccc(NC67CC(C6)C7)nc5c4)[C@@H](O)[C@@H]3F)c3ncnc(N)c23)n1] | B | 557.2 | ¹H NMR (400 MHz, DMSO- d₆) δ ppm 9.05 (s, 1H), 8.09 (s, 1H), 7.78 (d, J = 8.8 Hz, 1H), 7.75 (d, J = 2.4 Hz, 1H), 7.66 (d, J = 2.4 Hz, 1H), 7.56 (s, 1H), 7.53 (d, J = 8.8 Hz, 1H), 7.28 (s, 1H), 7.03 (d, J = 2.4 Hz, 1H), 6.86 (dd, J = 8.8 Hz 2.4 Hz, 1H), 6.70 (dd, J = 15.6 Hz, 4.8 Hz, 1H), 6.61 (d, J = 2 Hz, 1H), 6.58 (d, J = 8.8 Hz, 1H), 6.12 (d, J = 4.8 Hz, 1H), 5.31-5.16 (m, 1H), 4.64-4.56 |
| | | | (m, 1H), 4.45-4.35 (m, 2H), |
| | | | 4.21-4.17 (m, 1H), 3.87 (s, 3H), |
| | | | 2.49 (s, 1H), 2.13 (s, 6H). |
| 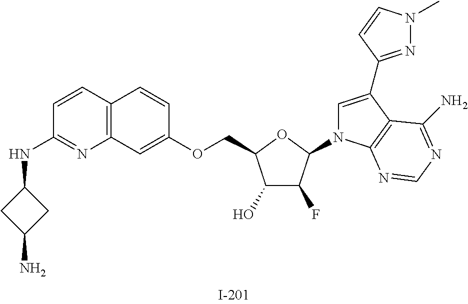 [SMILES: Cn1ccc(-c2cn([C@@H]3O[C@H](COc4ccc5ccc(N[C@H]6C[C@@H](N)C6)nc5c4)[C@@H](O)[C@@H]3F)c3ncnc(N)c23)n1] | B | 560.7 | ¹H NMR (400 MHz, DMSO- d₆) δ ppm 9.03 (s, 1H), 8.09 (s, 1H), 7.74-7.71 (m, 2H), 7.65 (d, J = 2.1 Hz, 1H), 7.49 (d, J = 8.7 Hz, 1H), 7.08 (d, J = 7.1 Hz, 1H), 7.00 (d, J = 2.3 Hz, 1H), 6.81 (dd, J = 8.7 Hz 2.4 Hz, 1H), 6.70 (dd, J = 15.9 Hz 4.6 Hz, 1H), 6.62 (d, J = 2.2 Hz, 1H), 6.55 (d, J = 8.9 Hz, 1H), 6.13 (s, 1H), 5.29-5.15 (m, 1H), 4.60-4.55 (m, 1H), 4.43- 4.40 (m, 2H), 4.20 (s, 1H), 4.06 (s, 1H), 3.88 (s, 3H), 3.11-2.92 (m, 1H), 2.63-2.61 (m, 2H), 1.84-1.80 (m, 1H), 1.57-1.55 (m, 2H). |
| 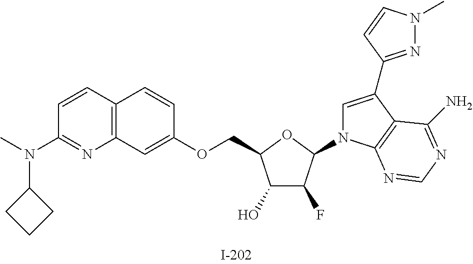 [SMILES: CN(c1ccc2ccc(OC[C@H]3O[C@@H](n4cc(-c5ccn(C)n5)c5c(N)ncnc54)[C@@H](F)[C@@H]3O)cc2n1)C1CCC1] | B | 559.0 | ¹H NMR (400 MHz, DMSO- d₆) δ ppm 9.03 (s, 1H), 8.09 (s, 1H), 7.92 (d, J = 9.1 Hz, 1H), 7.71 (d, J = 2.3 Hz, 1H), 7.65 (d, J = 2.3 Hz, 1H), 7.58 (d, J = 8.8 Hz, 1H), 7.25 (s, 1H), 7.07 (d, J = 2.4 Hz, 1H), 6.97-6.80 (m, 2H), 6.70 (dd, J = 15.9 Hz 4.5 Hz, 1H), 6.60 (d, J = 2.3 Hz, 1H), 6.12 (d, J = 4.9 Hz, 1H), 5.35-5.11 (m, 1H), 5.08- 4.89 (m, 1H), 4.65-4.50 (m, 1H), 4.50-4.40 (m, 1H), 4.39- 4.30 (m, 1H), 4.25-4.15 (m, 1H), 3.88 (s, 3H), 3.07 (s, 3H), 2.25-2.10 (m, 4H), 1.79-1.43 (m, 2H). |
| 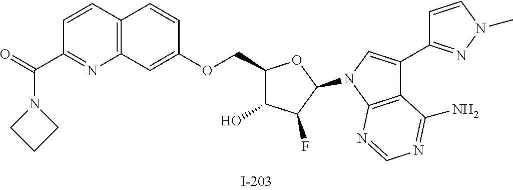 [SMILES: Cn1ccc(-c2cn([C@@H]3O[C@H](COc4ccc5ccc(C(=O)N6CCC6)nc5c4)[C@@H](O)[C@@H]3F)c3ncnc(N)c23)n1] | B | 559.0 | ¹H NMR (400 MHz, DMSO- d₆) δ ppm 9.03 (s, 1H), 8.41 (d, J = 8.8 Hz, 1H), 8.09 (s, 1H), 7.96 (d, J = 8.8 Hz, 1H), 7.87 (d, J = 8.8 Hz, 1H), 7.70 (s, 1H), 7.63 (s, 1H), 7.59 (s, 1H), 7.40 (d, J = 8.8 Hz, 1H), 7.25 (s, 1H), 6.71 (dd, J = 16 Hz 4.4 Hz, 1H), 6.57 (dd, J = 1.2 Hz, 1H), 6.16 (d, J = 4.4 Hz, 1H), 5.32-5.18 (m, 1H), 4.73 (t, J = 8 Hz, 2H), 4.69-4.57 (m, 1H), 4.57-4.47 (m, 2H), 4.25 (s, 1H), 4.12 (t, J = 8.8 Hz, 1H), |
| | | | 3.88 (s, 3H), 2.34-2.27 (m, 2H). |
| 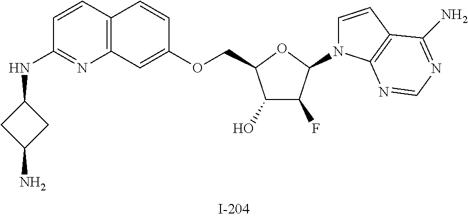 [SMILES: Nc1ncnc2c1ccn2[C@@H]1O[C@H](COc2ccc3ccc(N[C@H]4C[C@@H](N)C4)nc3c2)[C@@H](O)[C@@H]1F] | B | 480.0 | ¹H NMR (400 MHz, DMSO- d₆) δ ppm 8.09 (s, 1H), 7.78- 7.69 (m, 1H), 7.53-7.45 (m, 1H), 7.30 (s, 1H), 7.07 (s, 3H), 7.00-6.94 (m, 1H), 6.80 (dd, J = 8.7 Hz 2.4 Hz, 1H), 6.68- 6.62 (m, 2H), 6.58-6.54 (m, 1H), 6.12 (s, 1H), 5.30-5.09 (m, 1H), 4.55-4.51 (m, 1H), 4.38-4.36 (m, 1H), 4.31-4.24 (m, 1H), 4.17 (s, 1H), 3.04 (s, 1H), 2.63-2.61 (m, 2H), 1.82- 1.80 (m, 1H), 1.57-1.55 (m, 2H). |
| 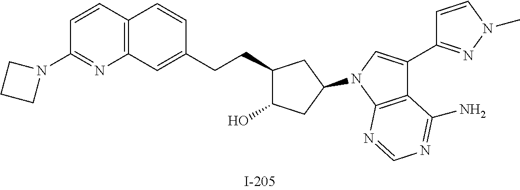 [SMILES: Cn1ccc(-c2cn([C@@H]3C[C@H](CCc4ccc5ccc(N6CCC6)nc5c4)[C@@H](O)C3)c3ncnc(N)c23)n1] | F | 255.1 | ¹H NMR (400 MHz, DMSO- d₆) δ ppm 8.96 (s, 1H), 8.03 (s, 1H), 7.95 (d, J = 8.8 Hz, 1H), 7.80 (s, 1H), 7.71 (d, J = 2.2 Hz, 1H), 7.60 (d, J = 8.2 Hz, 1H), 7.42 (s, 1H), 7.10 (dd, J = 8.1 Hz 1.6 Hz, 2H), 6.64 (dd, J = 16.3 Hz 5.5 Hz, 2H), 5.21 (dd, J = 17.3 Hz 8.6 Hz, 1H), 4.86 (d, J = 4.7 Hz, 1H), 4.04 (dd, J = 11.9 Hz 7.2 Hz, 5H), 3.87 (s, 3H), 2.86-2.65 (m, 2H), 2.35 (dd, J = 14.9 Hz 7.3 Hz, |
| | | | 3H), 2.25-2.12 (m, 1H), 2.10- |
| | | | 1.92 (m, 2H), 1.83 (d, J = 5.1 |
| | | | Hz, 1H), 1.75-1.55 (m, 2H). |
| 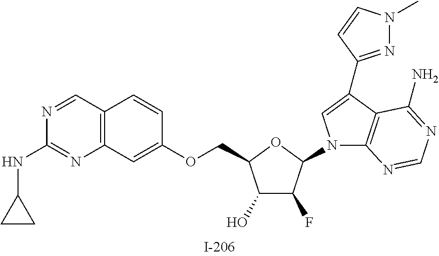 [SMILES: Cn1ccc(-c2cn([C@@H]3O[C@H](COc4ccc5cnc(NC6CC6)nc5c4)[C@@H](O)[C@@H]3F)c3ncnc(N)c23)n1] | B | 532.0 | ¹H NMR (400 MHz, DMSO- d₆) δ ppm 9.11-8.98 (br, 1H), 8.92 (s, 1H), 8.09 (s, 1H), 7.72- 7.69 (m, 2H), 7.64 (d, J = 2.2 Hz, 1H), 7.50-7.46 (m, 1H), 7.31-7.20 (br, 1H), 7.03-6.97 (m, 1H), 6.90 (dd, J = 8.8 Hz 2.4 Hz, 1H), 6.70 (dd, J = 16.2 Hz 4.5 Hz, 1H), 6.62 (d, J = 2.3 Hz, 1H), 6.14 (d, J = 4.9 Hz, 1H), 5.30-5.14 (m, 1H), 4.63- 4.46 (m, 2H), 4.43-4.37 (m, 1H), 4.24-4.17 (m, 1H), 3.88 (s, 3H), 2.85-2.79 (m, 1H), 0.72- 0.66 (m, 2H), 0.54-0.49 (m, 2H). |
| 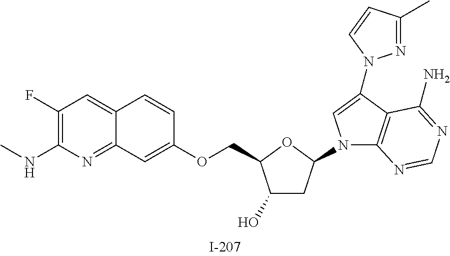 [SMILES: CNc1nc2cc(OC[C@H]3O[C@@H](n4cc(-n5ccc(C)n5)c5c(N)ncnc54)C[C@@H]3O)ccc2cc1F] | C | 504.9 | ¹H NMR (400 MHz, DMSO- d₆) δ ppm 8.17 (s, 1H), 7.85 (d, J = 11.9 Hz, 1H), 7.76 (d, J = 8.2 Hz, 1H), 7.58 (m, 1H), 7.44-7.37 (m, 1H), 7.21 (m, 1H), 7.16-7.14 (m, 1H), 6.06 (d, J = 2.3 Hz, 1H), 5.99-5.95 (m, 1H), 5.64-5.61 (m, 1H), 5.11 (d, J = 3.9 Hz, 1H), 4.31- 4.26 (m, 1H), 3.81-3.76 (m, 1H), 3.67-3.62 (m, 1H), 3.55- 3.49 (m, 1H), 3.30 (m, 2H), 3.25-3.15 (m, 1H), 2.95 (d, J = 4.6 Hz, 3H), 2.25 (s, 3H), 2.03- 1.96 (m, 1H). |
| 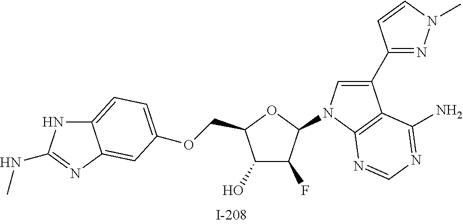 | B | 494.0 | ¹H NMR (400 MHz, DMSO- d₆) δ ppm 9.02 (d, 1H), 8.08 (s, 1H), 7.71 (d, 1H), 7.63 (d, 1H), 7.24 (d, 1H), 6.99 (d, 1H), 6.81 (d, 1H), 6.67 (dd, 1H), 6.61- 6.53 (m, 2H), 6.39 (m, 1H), 6.09 (d, J = 5.4 Hz, 1H), 4.56- 4.49 (m, 1H), 4.28-4.11 (m, 3H), 3.89 (d, 3H), 2.81 (dd, 3H). |
| 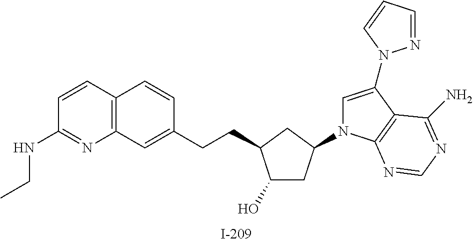 [SMILES: CCNc1ccc2ccc(CC[C@H]3C[C@@H](n4cc(-n5cccn5)c5c(N)ncnc54)C[C@@H]3O)cc2n1] | F | 483.1 | ¹H NMR (400 MHz, DMSO- d₆) δ ppm 8.39 (d, J = 2.1 Hz, 1H), 8.10 (s, 1H), 7.88 (s, 1H), 7.81 (d, J = 1.6 Hz, 1H), 7.76 (d, J = 8.9 Hz, 1H), 7.51 (d, J = 8.1 Hz, 1H), 7.35 (s, 1H), 7.03 (d, J = 8.0 Hz, 1H), 6.89 (s, 1H), 6.66 (d, J = 8.9 Hz, 1H), 6.55 (t, J = 4 Hz, 1H), 5.51- 5.01 (m, 1H), 4.86 (d, J = 4.7 Hz, 1H), 4.10-3.95 (m, 1H), 3.46-3.35 (m, 2H), 2.87-2.64 (m, 2H), 2.45-2.35 (m, 1H), 2.26-2.12 (m, 1H), 2.10-1.95 (m, 2H), 1.90-1.80 (m, 1H), 1.76-1.47 (m, 2H), 1.18 (t, J = |
| | | | 7.2 Hz, 3H). |
| 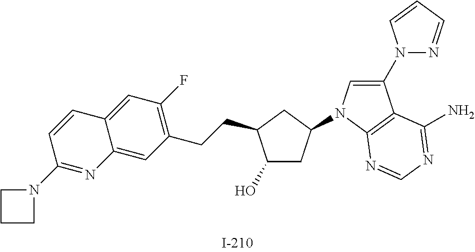 [SMILES: Nc1ncnc2c1c(-n1cccn1)cn2[C@@H]1C[C@H](CCc2cc3nc(N4CCC4)ccc3cc2F)[C@@H](O)C1] | F | 513.0 | ¹H NMR (400 MHz, DMSO- d₆) δ ppm 8.39 (d, J = 2.2 Hz, 1H), 8.10 (s, 1H), 7.96 (d, J = 8.9 Hz, 1H), 7.88 (s, 1H), 7.81 (d, J = 1.7 Hz, 1H), 7.51 (d, J = 7.4 Hz, 1H), 7.46 (d, J = 10.6 Hz, 1H), 6.69 (d, J = 8.9 Hz, 1H), 6.55 (t, J = 4 Hz, 1H), 5.38-5.16 (m, 1H), 4.90 (d, J = 4.8 Hz, 1H), 4.15-3.90 (m, 5H), 2.87-2.71 (m, 2H), 2.46-2.40 (m, 1H), 2.37-2.31 (m, 2H), 2.25-2.12 (m, 1H), 2.07-1.96 (m, 2H), 1.93-1.76 (m, 1H), 1.75-1.52 (m, 2H). |
| 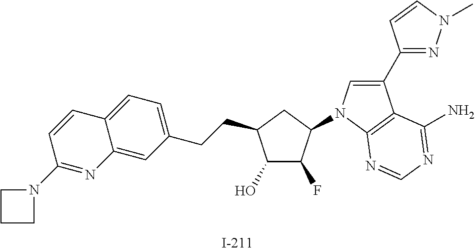 [SMILES: Cn1ccc(-c2cn([C@@H]3C[C@H](CCc4ccc5ccc(N6CCC6)nc5c4)[C@@H](O)[C@@H]3F)c3ncnc(N)c23)n1] | G | 527.3 | ¹H NMR (400 MHz, DMSO- d₆) δ ppm 1.73-1.94 (m, 2 H), 1.99-2.21 (m, 2 H), 2.30- 2.43 (m, 3 H), 2.71-2.92 (m, 2 H), 3.84-3.95 (m, 4 H), 4.08 (t, J = 7.3 Hz, 4 H), 4.68-4.92 (m, 1 H), 5.05-5.29 (m, 1 H), 5.45 (d, J = 5.4 Hz, 1 H), 6.63 (d, J = 8.8 Hz, 1 H), 6.69 (d, J = 2.2 Hz, 1 H), 7.14 (d, J = 7.1 Hz, 2 H), 7.45 (s, 1 H), 7.62 (d, J = 8.3 Hz, 1 H), 7.73 (dd, J = 14.2, 1.7 Hz, 2 H), 7.97 (d, J = 9.0 Hz, 1 H), 8.06 (s, 1 H), 9.01 (br. s, 1 H) |
| 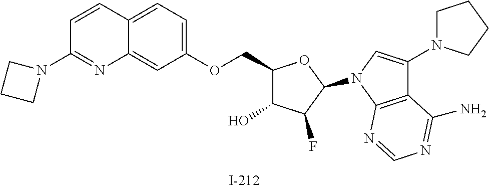 [SMILES: Nc1ncnc2c1c(N1CCCC1)cn2[C@@H]1O[C@H](COc2ccc3ccc(N4CCC4)nc3c2)[C@@H](O)[C@@H]1F] | B | 520.2 | ¹H NMR (400 MHz, DMSO- d₆) δ ppm 1.85 (br s, 4 H), 2.29- 2.40 (m, 2 H), 2.79-2.97 (m, 4 H), 4.00-4.17 (m, 5 H), 4.25- 4.34 (m, 1 H), 4.38-4.58 (m, 2 H), 4.94-5.32 (m, 1 H), 6.07 (br d, J = 4.6 Hz, 1 H), 6.36- 6.55 (m, 2 H), 6.62 (br dd, J = 15.9, 4.2 Hz, 1 H), 6.71-6.82 (m, 1 H), 6.88 (br d, J = 8.3 Hz, 1 H), 7.08 (br s, 1 H), 7.60 (br d, J = 8.6 Hz, 1 H), 7.92 (br d, J = 8.8 Hz, 1 H), 8.05 (s, 1 H). |
| 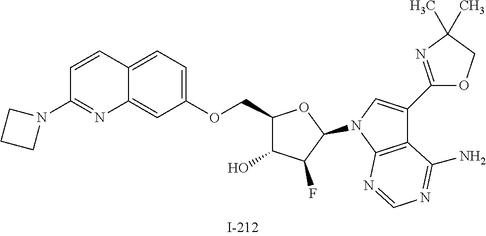 [SMILES: CC1(C)COC(c2cn([C@@H]3O[C@H](COc4ccc5ccc(N6CCC6)nc5c4)[C@@H](O)[C@@H]3F)c3ncnc(N)c23)=N1] | B | 548.2 | ¹H NMR (400 MHz, DMSO- d₆) δ 9.34-9.23 (m, 1H), 8.14 (s, 1H), 7.93 (d, J = 8.8 Hz, 1H), 7.89 (d, J = 1.7 Hz, 1H), 7.63 (d, J = 8.8 Hz, 1H), 7.46- 7.36 (m, 1H), 7.13 (d, J = 2.4 Hz, 1H), 6.98 (dd, J = 8.8, 2.7 Hz, 1H), 6.67 (dd, J = 11.5, 5.1 Hz, 1H), 6.53 (d, J = 8.8 Hz, 1H), 6.15 (d, J = 4.9 Hz, 1H), 5.41-5.22 (m, 1H), 4.65-4.53 (m, 1H), 4.45-4.31 (m, 2H), 4.24-4.13 (m, 3H), 4.07 (t, J = 7.5 Hz, 4H), 2.41-2.30 (m, 2H), 1.32 (d, J = 6.8 Hz, 6H). |
| 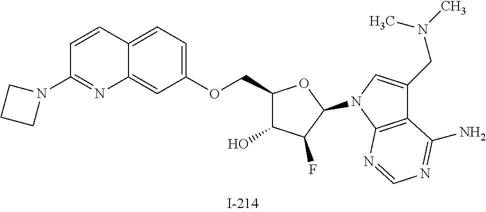 [SMILES: CN(C)Cc1cn([C@@H]2O[C@H](COc3ccc4ccc(N5CCC5)nc4c3)[C@@H](O)[C@@H]2F)c2ncnc(N)c12] | B | 508.3 | ¹H NMR (400 MHz, DMSO- d₆) δ 8.06 (s, 1H), 7.92 (d, J = 8.8 Hz, 1H), 7.61 (d, J = 8.8 Hz, 1H), 7.17 (d, J = 2.2 Hz, 1H), 7.08 (d, J = 2.4 Hz, 1H), 6.88 (dd, J = 8.6, 2.4 Hz, 1H), 6.61 (dd, J = 16.3, 4.5 Hz, 1H), 6.52 (d, J = 8.8 Hz, 1H), 6.11 (s, 1H), 5.26-5.08 (m, 1H), 4.57-4.46 (m, 1H), 4.44-4.36 (m, 1H), 4.34-4.26 (m, 1H), 4.19-4.12 (m, 1H), 4.07 (t, J = 7.3 Hz, 4H), 3.50-3.38 (m, 2H), 2.35 (quin, J = 7.5 Hz, 2H), 2.19 (s, 6H). |
| 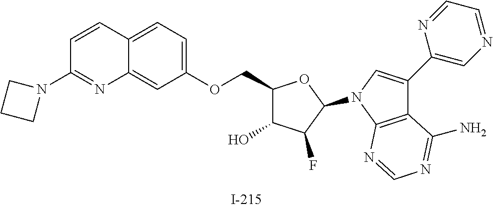 [SMILES: Nc1ncnc2c1c(-c1cnccn1)cn2[C@@H]1O[C@H](COc2ccc3ccc(N4CCC4)nc3c2)[C@@H](O)[C@@H]1F] | B | 529.2 | ¹H NMR (400 MHz, DMSO- d₆) δ 2.32-2.40 (m, 2H), 4.06 (t, J = 7.5 Hz, 4 H), 4.22-4.30 (m, 1 H), 4.47 (d, J = 5.4 Hz, 2 H), 4.59-4.71 (m, 1 H), 5.19- 5.39 (m, 1 H), 6.18 (d, J = 3.9 Hz, 1 H), 6.52 (d, J = 8.6 Hz, 1 H), 6.77 (dd, J = 14.5, 4.8 Hz, 1 H), 6.90 (d, J = 8.8 Hz, 1 H), 7.13 (s, 1 H), 7.60 (d, J = 9.0 Hz, 1H), 7.92 (d, J = 8.8 Hz, 1 H), 8.16 (s, 1H), 8.28 (s, 1 H), 8.48 (d, J = 2.7 Hz, 1 H), 8.58- 8.65 (m, 1 H), 9.37 (s, 1 H). |
| 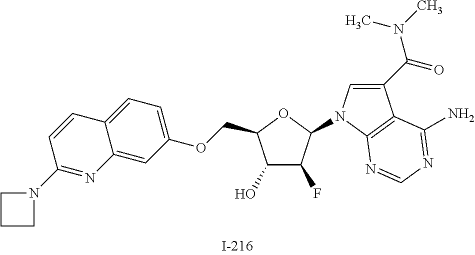 [SMILES: CN(C)C(=O)c1cn([C@@H]2O[C@H](COc3ccc4ccc(N5CCC5)nc4c3)[C@@H](O)[C@@H]2F)c2ncnc(N)c12] | B | 522.2 | ¹H NMR (400 MHz, DMSO- d₆) δ 8.16 (s, 1H), 7.92 (d, J = 8.8 Hz, 1H), 7.66 (d, J = 2.0 Hz, 1H), 7.61 (d, J = 8.8 Hz, 1H), 7.36 (br s, 2H), 7.08 (d, J = 2.4 Hz, 1H), 6.87 (dd, J = 8.8, 2.4 Hz, 1H), 6.70 (dd, J = 14.9, 4.6 Hz, 1H), 6.52 (d, J = 8.8 Hz, 1H), 6.14 (d, J = 4.9 Hz, 1H), 5.33-5.17 (m, 1H), 4.62-4.51 (m, 1H), 4.47-4.40 (m, 1H), 4.39-4.31 (m, 1H), 4.26-4.19 (m, 1H), 4.06 (t, J = 7.5 Hz, 4H), 3.08 (br s, 6H), 2.35 (quin, J = 7.4 Hz, 2H). |
| 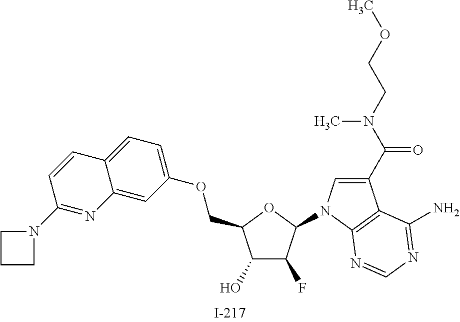 [SMILES: COCCN(C)C(=O)c1cn([C@@H]2O[C@H](COc3ccc4ccc(N5CCC5)nc4c3)[C@@H](O)[C@@H]2F)c2ncnc(N)c12] | B | 566.3 | ¹H NMR (400 MHz, DMSO- d₆) δ 2.35 (quin, J = 7.3 Hz, 2 H), 3.01-3.14 (m, 3 H), 3.22 (s, 3 H), 3.48-3.57 (m, 2 H), 3.62-3.72 (m, 2 H), 4.06 (t, J = 7.3 Hz, 4 H), 4.19-4.27 (m, 1 H), 4.27-4.36 (m, 1 H), 4.38- 4.47 (m, 1 H), 4.48-4.60 (m, 1 H), 5.15-5.33 (m, 1 H), 6.16 (d, J = 4.4 Hz, 1 H), 6.52 (d, J = 8.8 Hz, 1 H), 6.71 (dd, J = 15.9, 4.4 Hz, 1 H), 6.86 (dd, J = 8.8, 2.2 Hz, 1 H), 7.07 (d, J = 1.2 Hz, 1 H), 7.28 (br s, 2 H), 7.60 (d, J = 8.8 Hz, 1 H), 7.69-7.85 (m, 1 H), 7.92 (d, J = 8.8 Hz, 1 H), 8.16 (s, 1 H). |
| 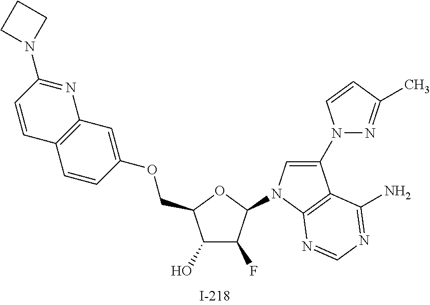 [SMILES: Cc1ccn(-c2cn([C@@H]3O[C@H](COc4ccc5ccc(N6CCC6)nc5c4)[C@@H](O)[C@@H]3F)c3ncnc(N)c23)n1] | B | 531.3 | ¹H NMR (400 MHz, DMSO- d₆) δ 2.30 (s, 3 H), 2.32-2.40 (m, 2 H), 4.06 (t, J = 7.5 Hz, 4 H), 4.17-4.25 (m, 1 H), 4.32- 4.40 (m, 1 H), 4.41-4.48 (m, 1 H), 4.57 (dq, J = 18.9, 4.5 Hz, 1 H), 5.14-5.35 (m, 1 H), 6.14 (d, J = 4.6 Hz, 1 H), 6.33 (d, J = 2.2 Hz, 1 H), 6.52 (d, J = 8.8 Hz, 1 H), 6.75 (dd, J = 15.2, 4.4 Hz, 1 H), 6.87 (dd, J = 8.8, 2.4 Hz, 1 H), 7.09 (d, J = 2.2 Hz, 1 H), 7.59 (d, J = 8.8 Hz, 1 H), 7.66 (d, J = 2.0 Hz, 1 H), 7.91 (d, J = 8.8 Hz, 1 H), 8.13 (s, 1 H), 8.23 (d, J = 2.4 Hz, 1 H). |
| 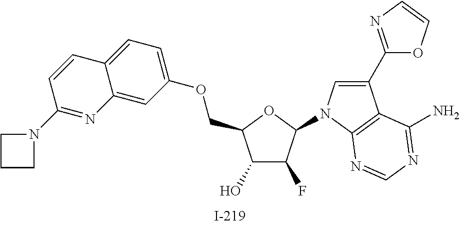 [SMILES: Nc1ncnc2c1c(-c1ncco1)cn2[C@@H]1O[C@H](COc2ccc3ccc(N4CCC4)nc3c2)[C@@H](O)[C@@H]1F] | B | 518.2 | ¹H NMR (400 MHz, DMSO- d₆) δ 2.27-2.42 (m, 2 H), 4.07 (br t, J = 7.3 Hz, 4 H), 4.18- 4.27 (m, 1 H), 4.34-4.49 (m, 2 H), 4.55-4.71 (m, 1H), 5.16- 5.45 (m, 1 H), 6.17 (br d, J = 4.6 Hz, 1 H), 6.53 (d, J = 8.8 Hz, 1 H), 6.71 (dd, J = 12.5, 4.6 Hz, 1 H), 6.98 (dd, J = 8.8, 2.0 Hz, 1 H), 7.15 (s, 1H), 7.41 (s, 1 H), 7.58 (br s, 1 H), 7.65 (d, J = 8.8 Hz, 1 H), 7.92 (d, J = 8.8 Hz, 1 H), 8.04 (s, 1 H), 8.18 (s, 2 H), 9.00 (br s, 1 H). |
| 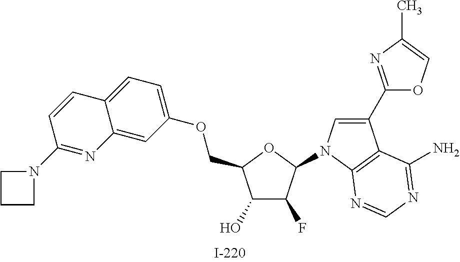 [SMILES: Cc1coc(-c2cn([C@@H]3O[C@H](COc4ccc5ccc(N6CCC6)nc5c4)[C@@H](O)[C@@H]3F)c3ncnc(N)c23)n1] | B | 532.2 | ¹H NMR (400 MHz, DMSO- d₆) δ 2.17 (s, 3 H), 2.35 (quin, J = 7.4 Hz, 2 H), 4.07 (t, J = 7.5 Hz, 4 H), 4.17-4.26 (m, 1 H), 4.32-4.48 (m, 2 H), 4.61 (dq, J = 19.4, 4.9 Hz, 1 H), 5.20- 5.42 (m, 1 H), 6.16 (d, J = 4.9 Hz, 1 H), 6.52 (d, J = 8.8 Hz, 1 H), 6.70 (dd, J = 12.6, 4.8 Hz, 1 H), 6.97 (dd, J = 8.8, 2.4 Hz, 1 H), 7.15 (d, J = 2.2 Hz, 1 H), 7.55 (br s, 1 H), 7.64 (d, J = 8.8 Hz, 1 H), 7.83-7.89 (m, 1 H), 7.92 (d, J = 8.8 Hz, 1 H), 7.97- 8.03 (m, 1 H), 8.16 (s, 1 H), 9.01 (br s, 1 H). |
| 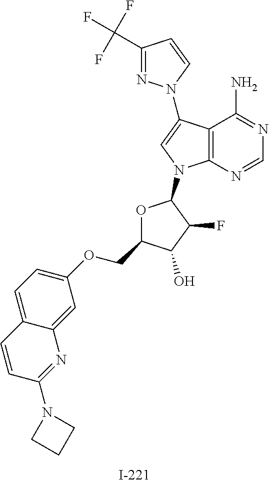 [SMILES: Nc1ncnc2c1c(-n1ccc(C(F)(F)F)n1)cn2[C@@H]1O[C@H](COc2ccc3ccc(N4CCC4)nc3c2)[C@@H](O)[C@@H]1F] | B | 585.2 | ¹H NMR (400 MHz, DMSO- d₆) δ 2.35 (quin, J = 7.4 Hz, 2 H), 4.05 (t, J = 7.3 Hz, 4 H), 4.17-4.28 (m, 1 H), 4.33- 4.41 (m, 1 H), 4.41-4.48 (m, 1 H), 4.59 (dq, J = 18.8, 4.6 Hz, 1 H), 5.16-5.39 (m, 1 H), 6.16 (d, J = 4.9 Hz, 1 H), 6.51 (d, J = 8.8 Hz, 1 H), 6.78 (dd, J = 15.0, 4.5 Hz, 1 H), 6.86 (dd, J = 8.7, 2.6 Hz, 1 H), 7.09 (dd, J = 6.6, 2.4 Hz, 2 H), 7.54-7.63 (m, 3 H), 7.91 (d, J = 9.0 Hz, 1 H), 7.94 (d, J = 1.7 Hz, 1 H), 8.19 (s, 1 H), 8.65 (d, J = 1.5 Hz, 1 H). |
| 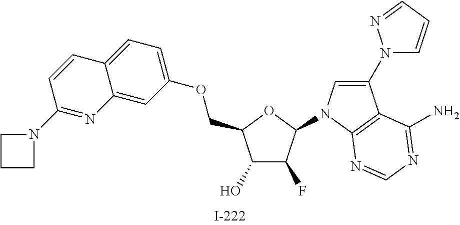 [SMILES: Nc1ncnc2c1c(-n1cccn1)cn2[C@@H]1O[C@H](COc2ccc3ccc(N4CCC4)nc3c2)[C@@H](O)[C@@H]1F] | B | 517.2 | ¹H NMR (400 MHz, DMSO- d₆) δ ppm 2.35 (quin, J = 7.4 Hz, 2 H), 4.06 (t, J = 7.3 Hz, 4 H), 4.17-4.26 (m, 1 H), 4.32- 4.40 (m, 1 H), 4.41-4.48 (m, 1 H), 4.52-4.65 (m, 1 H), 5.14- 5.35 (m, 1 H), 6.15 (d, J = 4.6 Hz, 1 H), 6.52 (d, J = 8.8 Hz, 1 H), 6.54-6.58 (m, 1 H), 6.76 (dd, J = 15.4, 4.6 Hz, 1 H), 6.87 (dd, J = 8.8, 2.2 Hz, 1 H), 7.09 (d, J = 1.7 Hz, 1 H), 7.59 (d, J = 8.8 Hz, 1 H), 7.74 (d, J = 1.5 Hz, 1 H), 7.84 (d, J = 1.2 Hz, 1 H), 7.91 (d, J = 8.8 Hz, 1 H), |
| | | | 8.13-8.17 (m, 1H), 8.39 (d, J = |
| | | | 2.0 Hz, 1 H). |
| 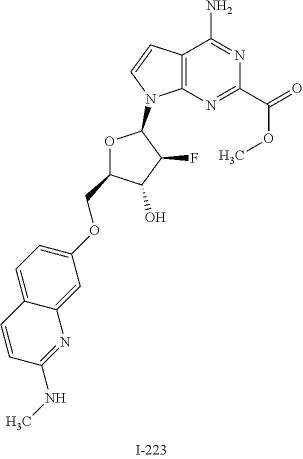 [SMILES: CNc1ccc2ccc(OC[C@H]3O[C@@H](n4ccc5c(N)nc(C(=O)OC)nc54)[C@@H](F)[C@@H]3O)cc2n1] | B | 483.2 | ¹H NMR (400 MHz, DMSO- d₆) δ ppm 2.89 (d, J = 4.2 Hz, 3 H), 3.85 (s, 3 H), 4.16-4.25 (m, 1 H), 4.31-4.45 (m, 2 H), 4.56-4.68 (m, 1 H), 5.17- 5.37 (m, 1 H), 6.14 (d, J = 4.9 Hz, 1 H), 6.58 (d, J = 8.8 Hz, 1 H), 6.66 (dd, J = 15.3, 4.3 Hz, 1 H), 6.72 (d, J = 3.4 Hz, 1 H), 6.81 (d, J = 8.3 Hz, 1 H), 6.93 (br. s, 1 H), 7.03 (br. s, 1 H), 7.37-7.59 (m, 4 H), 7.74 (d, J = 8.6 Hz, 1 H). |
| 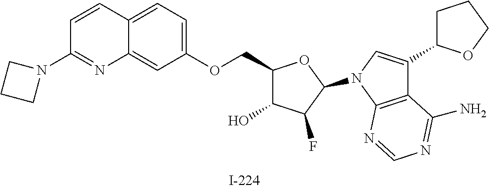 [SMILES: Nc1ncnc2c1c([C@@H]1CCCO1)cn2[C@@H]1O[C@H](COc2ccc3ccc(N4CCC4)nc3c2)[C@@H](O)[C@@H]1F] | B | 521.0 | ¹H NMR (400 MHz, DMSO- d₆) δ ppm 8.11 (s, 1H), 7.92 (d, J = 8.8 Hz, 1H), 7.61 (d, J = 8.8 Hz, 1H), 7.17-7.15 (m, 1H), 7.08 (s, 1H), 6.91-6.86 (m, 3H), 6.66-6.61 (m, 1H), 6.52 (d, J = 5.2 Hz, 1H), 5.20 (dt, J = 50.4, 3.6 Hz, 1H), 4.92-4.90 (m, 1H), 4.56-4.51 (m, 1H), 4.43-4.40 (m, 1H), 4.31-4.28 (m, 1H), 4.16-4.13 (m, 1H), 4.08-4.04 (m, 4H), 3.95-3.90 (m, 1H), 3.82-3.80 (m, 1H), 2.37-2.33 |
| | | | (m, 2H), 2.16-2.08 (m, 1H), |
| | | | 1.97-1.93 (m, 2H), 1.78-1.73 |
| | | | (m, 1H). |
| 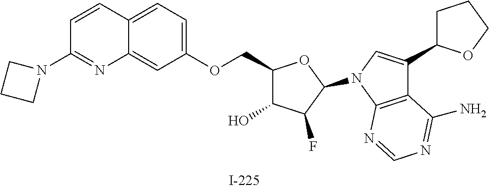 [SMILES: Nc1ncnc2c1c([C@H]1CCCO1)cn2[C@@H]1O[C@H](COc2ccc3ccc(N4CCC4)nc3c2)[C@@H](O)[C@@H]1F] | B | 521.2 | ¹H NMR (400 MHz, DMSO- d₆) δ 8.11 (s, 1H), 7.92 (d, J = 8.8 Hz, 1H), 7.61 (d, J = 8.8 Hz, 1H), 7.17-7.15 (m, 1H), 7.08 (s, 1H), 6.91-6.86 (m, 3H), 6.66-6.61 (m, 1H), 6.52 (d, J = 5.2 Hz, 1H), 5.20 (d, t, J = 50.4. 3.6 Hz, 1H), 4.92-4.90 (m, 1H), 4.56-4.51 (m, 1H), 4.43-4.40 (m, 1H), 4.31-4.28 (m, 1H), 4.16-4.13 (m, 1H), 4.08-4.04 (m, 4H), 3.95-3.90 (m, 1H), 3.82-3.80 (m, 1H), 2.37-2.33 |
| | | | (m, 2H), 2.16-2.08 (m, 1H), |
| | | | 1.97-1.93 (m, 2H), 1.78-1.73 |
| | | | (m, 1H). |
| 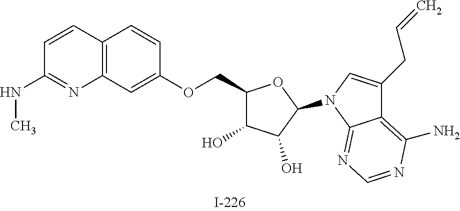 [SMILES: C=CCc1cn([C@@H]2O[C@H](COc3ccc4ccc(NC)nc4c3)[C@@H](O)[C@H]2O)c2ncnc(N)c12] | A | 463.0 | ¹H NMR (400 MHz, DMSO- d6) δ ppm 8.06 (s, 1H), 7.74 (d, J = 8.8 Hz, 1H), 7.53 (d, J = 8.7 Hz, 1H), 7.12 (s, 1H), 7.02 (d, J = 2.0 Hz, 1H), 6.95 (s, 1H), 6.82 (dd, J = 8.7, 2.3 Hz, 1H), 6.58 (d, J = 8.8 Hz, 1H), 6.50 (s, 2H), 6.18 (d, J = 5.7 Hz, 1H), 6.03-5.96 (m, 1H), 5.40 (d, J = 6.2 Hz, 1H), 5.33 (d, J = 4.9 Hz, 1H), 5.12-5.06 (m, 2H), 4.44-4.40 (m, 1H), 4.34-4.17 (m, 4H), 3.53 (d, J = 6.1 Hz, 2H), 2.89 (d, J = 4.7 Hz, 3H). |
| 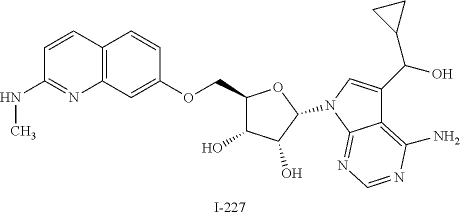 [SMILES: CNc1ccc2ccc(OC[C@H]3O[C@H](n4cc(C(O)C5CC5)c5c(N)ncnc54)[C@H](O)[C@@H]3O)cc2n1] | A | 493.2 | ¹H NMR (400 MHz, DMSO- d6) δ ppm 8.07 (s, 1H), 7.94 (s, 1H), 7.67 (s, 1H), 7.47 (s, 1H), 7.21 (s, 1H), 6.98 (s, 1H), 6.83- 6.65 (m, 4H), 6.18 (d, J = 5.6 Hz, 1H), 6.01-5.87 (m, 1H), 5.47 (d, J = 6.0 Hz, 1H), 5.36 (d, J = 5.0 Hz, 1H), 4.57 (s, 1H), 4.51-4.42 (m, 1H), 4.40- 4.32 (m, 1H), 4.30-4.16 (m, 3H), 3.49 (t, J = 6.8 Hz, 2H), 2.97 (s, 3H), 2.36-2.31 (m, 2H). |
| 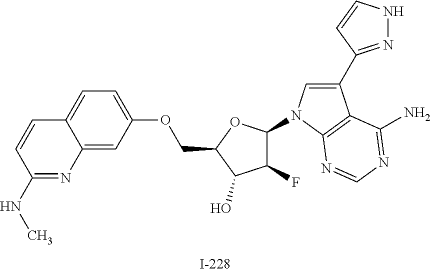 [SMILES: CNc1ccc2ccc(OC[C@H]3O[C@@H](n4cc(-c5cc[nH]n5)c5c(N)ncnc54)[C@@H](F)[C@@H]3O)cc2n1] | B | 492.1 | ¹H NMR (400 MHz, DMSO- d₆) δ ppm 12.90 (s, 1H), 8.10 (s, 1H), 7.88 (t, J = 1.6 Hz, 1H), 7.75 (d, J = 8.8 Hz, 1H), 7.67 (d, J = 2.4 Hz, 1H), 7.52 (d, J = 8.4 Hz, 1H), 7.304 (s, 1H), 7.07 (d, J = 2.4 Hz, 1H), 6.96-6.92 (m, 1H), 6.85- 6.82 (m, 1H), 6.63-6.68 (m, 1H), 6.67-6.66 (m, 1H), 6.59 (d, J = 8.8 Hz, 1H), 6.15 (d, J = 4.8 Hz 1H), 5.31-5.16 (m, 1H), 4.63-4.55 (m, 1H), 4.46-4.34 (m, 2H), 4.22-4.18 (m, 1H), 2.89 (d, J = 4.8 Hz, 3H). |
| 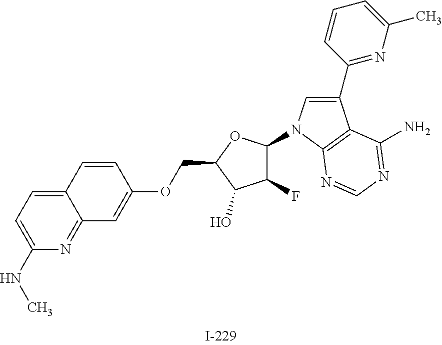 [SMILES: CNc1ccc2ccc(OC[C@H]3O[C@@H](n4cc(-c5cccc(C)n5)c5c(N)ncnc54)[C@@H](F)[C@@H]3O)cc2n1] | B | 516.1 | ¹H NMR (400 MHz, DMSO- d₆) δ ppm 10.17 (br, 1H), 8.11 (s, 1H), 7.96 (s, 1H), 7.76-7.64 (m, 3H), 7.55 (d, J = 8.8 Hz, 1H), 7.25 (br, 1H), 7.12-7.09 (m, 2H), 6.95-6.94 (m, 1H), 6.85 (dd, J = 8.8, 2.4 Hz, 1H), 6.75 (dd, J = 15.2, 4.8 Hz, 1H), 6.59 (d, J = 8.8 Hz, 1H), 6.16 (d, J = 4.8 Hz, 1H), 5.19 (dt, J = 52.8, 3.6 Hz, 1H), 4.65-4.59 (m, 1H), 4.49-4.38 (m, 2H), 4.26-4.23 (m, 1H), 2.88 (d, J = 4.8 Hz, 3H), 2.51 (s, 3H). |
| 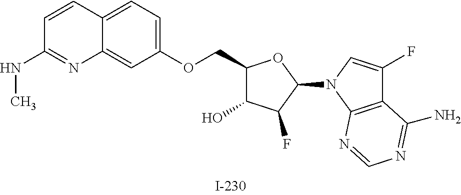 [SMILES: CNc1ccc2ccc(OC[C@H]3O[C@@H](n4cc(F)c5c(N)ncnc54)[C@@H](F)[C@@H]3O)cc2n1] | B | 443.2 | 1H NMR (400 MHz, DMSO- d6) δ 8.11 (s, 1H), 7.90 (s, 1H), 7.72-7.52 (m, 1H), 7.24 (s, 2H), 7.08 (s, 2H), 6.94 (s, 1H), 6.79- 6.62 (m, 2H), 6.23-6.08 (m, 1H), 5.43-5.08 (m, 1H), 4.60- 4.45 (m, 1H), 4.44-4.30 (m, 2H), 4.23-4.13 (m, 1H), 2.97 (s, 3H). |
| 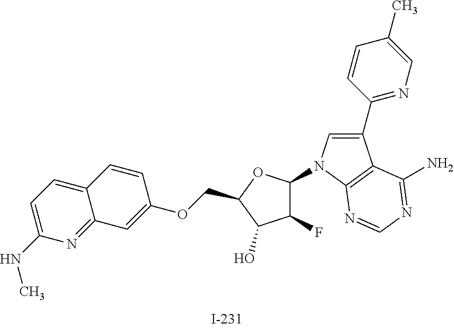 [SMILES: CNc1ccc2ccc(OC[C@H]3O[C@@H](n4cc(-c5ccc(C)cn5)c5c(N)ncnc54)[C@@H](F)[C@@H]3O)cc2n1] | B | 517.0 | ¹H NMR (400 MHz, DMSO- d₆) δ ppm 9.87 (s, 1H), 8.39 (s, 1H), 8.10 (s, 1H), 7.93 (d, J = 1.9 Hz, 1H), 7.82 (d, J = 8.3 Hz, 1H), 7.75 (d, J = 8.9 Hz, 1H), 7.61 (dd, J = 8.3, 2.0 Hz, 1H), 7.53 (d, J = 8.7 Hz, 1H), 7.31 (s, 1H), 7.08 (d, J = 2.4 Hz, 1H), 6.96-9.62 (m, 1H), 6.84 (dd, J = 8.7, 2.5 Hz, 1H), 6.73 (dd, J = 15.1, 4.6 Hz, 1H), 6.59 (d, J = 8.8 Hz, 1H), 6.15 (d, J = 4.8 Hz, 1H), 5.33-5.29 (m, 1H), 5.20-5.16 (m, 1H), 4.66-4.57 (m, 1H), 4.49- 4.46 (m, 1H), 4.42-4.37 (m, 1H), 4.25-4.21 (m, 1H), 2.88 (d, J = 4.7 Hz, 3H), 2.30 (s, 3H). |
| 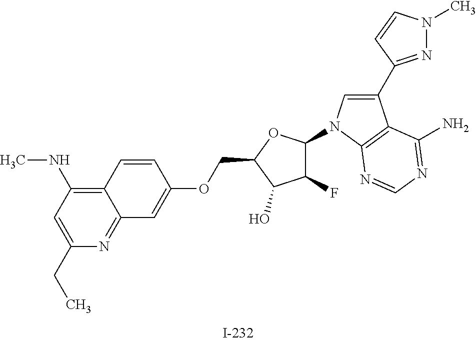 | B | 534.1 | ¹H NMR (400 MHz, DMSO- d₆) δ ppm 9.04 (s, 1H), 8.09 (s, 1H), 8.01 (d, J = 5.2 Hz, 1H), 7.71 (d, J = 2.4 Hz, 1H), 7.22 (s, 1H), 7.06-7.03 (m, 1H), 8.72-8.67 (m, 1H), 8.59 (d, J = 2.4 Hz, 1H), 8.22 (s, 1H), 8.17 (d, J = 4 Hz, 1H), 5.30-5.15 (m, 1H), 4.62-4.59 (m, 1H), 4.48- 4.36 (m, 2H), 4.23-4.20 (m, 1H), 3.88 (s, 1H), 2.88 (d, J = 4.4 Hz, 3H), 2.75-2.67 (m, 2H), 1.29-1.25 (m, 3H). |
| 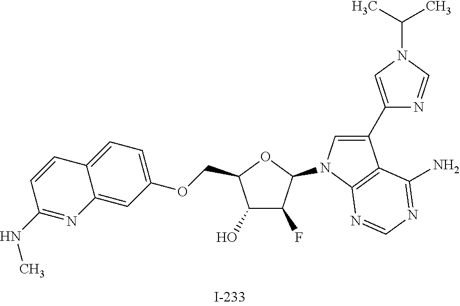 [SMILES: CNc1ccc2ccc(OC[C@H]3O[C@@H](n4cc(-c5cn(C(C)C)cn5)c5c(N)ncnc54)[C@@H](F)[C@@H]3O)cc2n1] | B | 533.0 | 1H NMR (400 MHz, DMSO- d6) δ ppm 9.94 (s, 1H), 8.07 (s, 1H), 7.85 (d, J = 1.2 Hz 1H), 7.73 (d, J = 1.2 Hz, 1H), 7.55 (d, J = 2.4 Hz, 2H), 7.532 (s, 1H), 7.09 (s, 1H), 6.87 (d, J = 8.4 Hz, 1H), 6.73-6.68 (m, 1H), 6.61 (d, J = 9.6 Hz, 1H), 6.15 (d, J = 5.2 Hz, 1H), 5.30-5.16 (m, 1H), 4.62-4.54 (m, 1H), 4.45-4.38 (m, 2H), 4.35-4.31 (m, 1H), 4.22-4.19 (m, 1H), 2.90 (d, J = 4.4 Hz, 3H), 1.44-1.42 (m, 6H). |
| 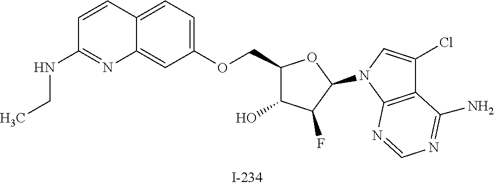 [SMILES: CCNc1ccc2ccc(OC[C@H]3O[C@@H](n4cc(Cl)c5c(N)ncnc54)[C@@H](F)[C@@H]3O)cc2n1] | B | 473.1 | 1H NMR (400 MHz, DMSO- d₆) δ ppm 8.13 (s, 1H), 7.73 (d, J = 8.8 Hz, 1H), 7.49-7.53 (m, 2H), 7.02 (d, J = 2.0 Hz, 1H), 6.92 (t, J = 5.2 Hz, 1H), 6.80 (dd, J = 8.8, 2.8 Hz, 1H), 6.67 (dd, J = 14.4, 4.8 Hz, 1H), 6.57 (d, J = 8.8 Hz, 1H), 6.13 (d, J = 5.2 Hz, 1H), 5.15-5.30 (m, 1H), 4.51-4.57 (m, 1H), 4.31-4.40 (m, 2H), 4.17-4.19 (m, 1H), 3.35-3.43 (m, 2H), 1.17-1.23 (m, 3H), |
| 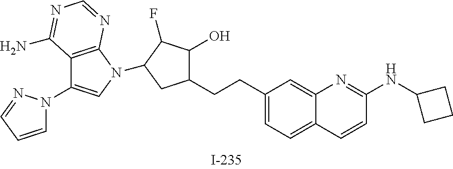 [SMILES: Nc1ncnc2c1c(-n1cccn1)cn2C1CC(CCc2ccc3ccc(NC4CCC4)nc3c2)C(O)C1F] | G | 526.7 | ¹H NMR (400 MHz, DMSO- d6) δ ppm 8.43 (d, J = 2.4 Hz, 1H), 8.12 (s, 1H), 7.87-7.77 (m, 3H), 7.52 (d, J = 8.0 Hz, 1H), 7.36 (s, 1H), 7.19 (d, J = 8.4 Hz, 1H), 7.05 (d, J = 8.4 Hz, 1H), 6.64 (d, J = 8.8 Hz, 1H), 6.56-6.55 (m, 1H), 6.64 (d, J = 4.8 Hz, 1H), 6.56-6.55 (m, 1H), 5.48 (d, J = 4.8 Hz, 1H), 5.36- 5.19 (m, 1H), 4.91-4.77 (m, 1H), 4.59-4.46 (m, 1H), 3.95- |
| | | | 3.87 (m, 1H), 2.78-2.75 (m, |
| | | | 2H), 2.44-2.41 (m, 2H), 2.33- |
| | | | 2.31 (m, 2H), 2.13-2.07 (m, |
| | | | 2H), 1.93-1.88 (m, 3H), 1.73- |
| | | | 1.67 (m, 2H). |

### Biological Assays

### METTL3-14 Standard Enzyme Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 250 μM MgCl2, 1 mM DTT, 0.01% Tween, 0.01% BSG, 0.004 U/μl RNAseOUT (cat. No. 10777019, ThermoFisher Scientific, Waltham, Mass.). For compound screening METTL3/METTL14 (final concentration, f.c.=2.5 nM) was added using a Multidrop Combi (ThermoFisher Scientific, Waltham, Mass.) and preincubated for 5 min. Reactions were started by adding 3′ biotinylated RNA (UCUGGACUAAA-biotin) (f.c.=100 nM) and 3H-SAM (f.c.=100 nM) substrates. Reactions proceeded for 30 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

### METTL1 Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 250 μM MgCl2, 1 mM DTT, 0.01% Tween, 0.01% BSG, 0.004 U/μl RNAseOUT (cat. No. 10777019, ThermoFisher Scientific, Waltham, Mass.). For compound screening METTL1/WDR4 (final concentration, f.c.=6.25 nM) was added using a Multidrop Combi (ThermoFisher Scientific, Waltham, Mass.) and preincubated for 5 min. Reactions were started by adding 3′ biotinylated RNA (GCCGAGAUAGCUCAGUUGGGAGAGCGUUAGACUGAAGAUCUAAAGGUCCCUG GUUCAAUCCCGGGUUUCGGCA-biotin) (f.c.=25 nM) and 3H-SAM (f.c.=60 nM) substrates. Reactions proceeded for 20 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

### METTL16 Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 1 mM DTT, 0.01% Tween, 0.01% BSG, 0.004 U/μl RNAseOUT (cat. No. 10777019, ThermoFisher Scientific, Waltham, Mass.). For compound screening METTL16 (final concentration, f.c.=100 nM) was added using a Multidrop Combi (ThermoFisher Scientific, Waltham, Mass.) and preincubated for 5 min. Reactions were started by adding 3′ biotinylated RNA (CGAUACAGAGAAGAUUAGCAUACGCAAAUUCGUGAAGCG-biotin) (f.c.=50 nM), 3H-SAM (f.c.=200 nM) and non-radiolabeled SAM (f.c.=800 nM) substrates. Reactions proceeded for 20 minutes and were quenched with excess non-radioactive SAM (f.c.=100 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

### PRMT5 Assay

Assays were performed in a 25 μl-volume in 384-well V-bottom polypropylene microplates (Greiner Bio-One, cat. No. 781280) at ambient temperature. Optimized 1× assay buffer was 20 mM Tris-HCl pH 8.0, 1 mM DTT, 0.01% Tween, 0.01%. For compound screening PRMT5-MEP50 (final concentration, f.c.=2.5 nM) was added using a Multidrop Combi (ThermoFisher Scientific, Waltham, Mass.) and preincubated for 5 min. Reactions were started by adding 3′ biotinylated histone H4 peptide acetylated on serine 1 (Ac-SGRGKGGKGLGKGGAKRHRKVGGK-Biotin) (f.c.=100 nM) and 3H-SAM (f.c.=250 nM) substrates. Reactions proceeded for 60 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

### m⁶A-mRNA LC-MS/MS Assay

5×10⁶ MOLM-13 (DSMZ) cells were seeded into 10 cm dishes in RPMI 1640 media containing 10% fetal bovine serum and placed in a humidified tissue culture incubator at 37° C. overnight. Compounds were resuspended in 100% DMSO and dosed into each dish at a fixed concentration to comprise an 8-point dose response with a 4-fold serial dilution ranging from 25 μM to 1.5 nM in 0.25% DMSO final and allowed to incubate for 24 hours in a humidified tissue culture incubator at 37° C. Cells were harvested by centrifugation followed by mRNA extraction using DIRECT Dynabeads mRNA DIRECT kit (Life Technologies). mRNA was quantified on NanoDrop spectrophotometer (Thermo Fisher Scientific) and digested into single nucleosides using Nucleoside Digestion Mix (New England Biolabs). Nucleosides are quantified with retention time on a BEH C₁₈ column (Waters) and the nucleoside-to-base ion mass transition of 282.1-150.1 (m⁶A) and 268-136 (A) on an API 6500+ triple quadrupole mass spectrometer. Quantification is performed in comparison with the standard curve, obtained from pure nucleoside standards (Selleck Chemicals) running with the same batch of samples. Percentage m⁶A in cellular mRNA is calculated as 100*(m⁶A/A).

### MOLM-13 48 Hour Cell Proliferation Assay

MOLM-13 (DSMZ) cells were seeded at 1000 cells per well in a volume of 44 μL in a Falcon 384-well tissue culture treated clear bottom microplate in RPMI 1640 media containing 10% fetal bovine serum using a Multidrop Combi (ThermoFisher Scientific). Cells were incubated overnight at 37° C. in a humidified tissue culture incubator. The Mosquito® HTS Liquid Handler was used to make a compound/media intermediate plate by aliquoting 1 μL of compound from the initial compound dilution plate (concentrations ranging from 10.0 mM to 38.0 nM in 100% DMSO) into a V bottom 384-well screen matrix plate containing 49 μL of media containing the appropriate serum (50-fold dilution, 2% DMSO). The Apricot liquid handling system was used to transfer 6.2 μL compounds from the intermediate plate into the Falcon 384-well tissue culture plate containing 44 μL cells (10-point, 4-fold dilution spanning concentrations 25.0 μM to 95.1 μM, 0.25% DMSO final), and placed in a humidified tissue culture incubator at 37° C. After 48 hours, 25 μL of Cell Titer-Glo reagent (Promega) was added to each well using a Multidrop Combi. The plate was protected from light and placed on an IKA plate shaker for at 300 rpm for 10 minutes at room temperature. The plate was read on an EnVision plate reader (Perkin Elmer) using the Ultra Sensitive Luminescence protocol. Data analysis was performed by normalizing the raw luminescence units to an average of the positive control values for staurosporine (100% cell death) and the negative control values for DMSO (0% cell death). An IC₅₀ was calculated using a 4-parameter logistic nonlinear regression model in GraphPad Prism.

### MOLM-13 96 Hour Cell Proliferation Assay

MOLM-13 (DSMZ) cells were seeded at 600 cells per well in a volume of 44 μL in a Greiner Bio-One CELLSTAR™ 384 Well Polystyrene Cell Culture clear bottom microplate in RPMI 1640 media containing 10% fetal bovine serum using a Multidrop Combi (ThermoFisher Scientific). Cells were incubated overnight at 37° C. in a humidified tissue culture incubator. To prevent evaporation or to reduce edge effect, add 50 μL or more H₂O to an empty plate, cover, and place on top of the cell plates. The Mosquito® HTS Liquid Handler was used to make a compound/media intermediate plate by aliquoting 1 μL of compound from the initial compound dilution plate (concentrations ranging from 10.0 mM to 38.0 nM in 100% DMSO) into Bio-One 384-well polypropylene conical bottom microplate containing 49 μL of media containing the appropriate serum (50-fold dilution, 2% DMSO, compound concentrations ranging from 200.0 μM and 760.8 μM). The Apricot liquid handling system was used to transfer 6.2 μL compounds from the intermediate plate into the seeding plate containing 44 μL cells (8-fold dilution spanning concentrations 25.0 μM to 95.1 μM, 0.25% DMSO final), and placed in a humidified tissue culture incubator at 37° C. After 96 hours, 25 μL of Cell Titer-Glo reagent (Promega) was added to each well using an Integra liquid handling system. The plate was protected from light using TopSeal-A (Black) film over the entire plate, added White Bottom seal film on the bottom of the entire plate for a better reading on the Envision, and placed on an IKA plate shaker for at 300 rpm for 10 minutes at room temperature. The plate was read on an EnVision plate reader (Perkin Elmer) using the Ultra Sensitive Luminescence protocol. Data analysis was performed by normalizing the raw luminescence units to an average of the positive control values for staurosporine (100% cell death) and the negative control values for DMSO (0% cell death). An IC₅₀ was calculated using a 4-parameter logistic nonlinear regression model in GraphPad Prism.

Table 2 shows IC₅₀ values for selected compounds of this invention measured in the METTL3 biochemical assay, PRMT5 biochemical assay, METTL1 biochemical assay, METTL16 biochemical assay, m⁶A cellular assay and MOLM-13 cell proliferation assay, wherein each compound number corresponds to the compound numbering set forth in Examples 1-235 of Table 1 disclosed above. For METTL3, PRMT5, METTL1 and METTL16 biochemical assays, “A” represents an IC₅₀ of less than 10 nM (i.e., IC₅₀<10 nM); “B” represents an IC₅₀ of equal to or greater than 10 nM and lesser than 100 nM (i.e., 10 nM≤IC₅₀<100 nM); “C” represents an IC₅₀ of equal to or greater than 100 nM and less than 1000 nM (i.e., 100 nM≤IC₅₀<1000 nM); and “D” represents an IC₅₀ of equal to or greater than 1000 nM (i.e., IC₅₀≥1000 nM). For m⁶A cellular assay and MOLM-13 48 Hour and 96 Hour cell proliferation assay, “*” represents an IC₅₀ of equal to or greater than 10 μM (i.e., IC₅₀≥10 μM); “**” represents an IC₅₀ value of equal to or greater than 1 μM and less than 10 μM (i. e., 1 μM≤IC₅₀<10 μM); and “***” represents an IC₅₀ of less than 1 μM (i. e., IC₅₀<1 μM).

TABLE 2

| | | | | | | MOLM-13 | MOLM-13 |
|---|---|---|---|---|---|---|---|
| Compound | | | | | | 48 Hour cell | 96 Hour cell |
| No. | METTL3 | PRMT5 | METTL1 | METTL16 | m⁶A | proliferation | proliferation |
| I-1 | A | A | D | | ** | | |
| I-2 | A | C | | | | | *** |
| I-3 | A | B | | | | | *** |
| I-4 | A | B | D | | | | |
| I-5 | A | B | D | | ** | | |
| I-6 | A | A | D | | *** | | |
| I-7 | A | B | D | | | | *** |
| I-8 | A | A | D | | *** | | |
| I-9 | A | B | D | | ** | | |
| I-10 | A | C | | D | ** | ** | *** |
| I-11 | B | C | | D | | | |
| I-12 | A | B | | D | *** | *** | *** |
| I-13 | C | B | | D | | | * |
| I-14 | B | C | | D | | | |
| I-15 | D | C | D | | | | |
| I-16 | B | B | | D | | | |
| I-17 | B | C | | D | | | |
| I-18 | B | C | | D | | | |
| I-19 | C | C | D | D | | | * |
| I-20 | D | C | D | | | | |
| I-21 | C | B | D | D | | | |
| I-22 | D | C | D | | | | |
| I-23 | A | B | D | D | ** | * | |
| I-24 | B | C | D | D | | | |
| I-25 | C | B | D | D | | | |
| I-26 | D | C | D | D | | | |
| I-27 | B | C | | D | | | |
| I-28 | A | C | | D | ** | ** | |
| I-29 | A | C | D | D | ** | *** | *** |
| I-30 | A | D | D | | ** | ** | *** |
| I-31 | D | C | D | D | | | |
| I-32 | B | C | D | D | | | |
| I-33 | A | A | D | D | *** | ** | *** |
| I-34 | A | C | D | D | ** | ** | ** |
| I-35 | B | C | D | D | | | |
| I-36 | A | C | D | D | ** | ** | *** |
| I-37 | B | C | D | D | | | |
| I-38 | B | C | D | D | | | |
| I-39 | A | A | D | D | ** | ** | *** |
| I-40 | B | C | D | D | | | |
| I-41 | C | C | D | D | | | |
| I-42 | A | B | D | | ** | ** | ** |
| I-43 | C | C | D | D | | | |
| I-44 | B | B | D | D | | | |
| I-45 | A | B | D | | ** | ** | ** |
| I-46 | A | B | D | | ** | ** | *** |
| I-47 | A | C | D | | | | |
| I-48 | A | C | D | | ** | * | ** |
| I-49 | C | A | D | | | | |
| I-50 | B | C | D | D | | | |
| I-51 | A | A | D | | ** | ** | *** |
| I-52 | A | A | D | D | ** | | |
| I-53 | C | A | D | | | | |
| I-54 | A | A | D | | ** | ** | ** |
| I-55 | A | A | D | | ** | ** | *** |
| I-56 | D | A | D | | | | |
| I-57 | D | B | D | | | | |
| I-58 | B | A | D | | | | |
| I-59 | A | A | D | | ** | ** | *** |
| I-60 | B | A | D | | | ** | *** |
| I-61 | D | A | D | | | | |
| I-62 | C | A | D | | | | |
| I-63 | C | A | D | | | | |
| I-64 | C | B | D | | | | |
| I-65 | D | A | D | | | | |
| I-66 | | A | D | | | | |
| I-67 | A | A | D | D | | | |
| I-68 | B | C | D | D | | | ** |
| I-69 | B | B | D | D | | | |
| I-70 | A | A | D | D | ** | ** | *** |
| I-71 | B | A | D | D | | | |
| I-72 | B | B | D | D | | | |
| I-73 | A | A | D | D | ** | ** | *** |
| I-74 | D | C | D | D | | | * |
| I-75 | B | B | D | | ** | * | |
| I-76 | B | A | D | | | | |
| I-77 | B | C | D | | | | |
| I-78 | B | B | D | | * | * | ** |
| I-79 | D | C | D | | | | |
| I-80 | B | A | D | | | | |
| I-81 | B | A | | D | | | |
| I-82 | C | C | D | D | | | |
| I-83 | C | B | D | D | | | |
| I-84 | D | A | D | D | | | |
| I-85 | D | A | D | | | | |
| I-86 | D | A | D | | | | |
| I-87 | B | A | D | | | | |
| I-88 | D | A | D | | | | |
| I-89 | D | C | D | | | | |
| I-90 | B | A | | D | | | |
| I-91 | D | A | D | | | | |
| I-92 | D | C | D | | | | |
| I-93 | D | B | D | | | | |
| I-94 | D | A | D | | | | |
| I-95 | D | C | D | | | | |
| I-96 | D | C | D | | | | |
| I-97 | D | C | D | | | | |
| I-98 | B | B | D | | * | * | ** |
| I-99 | D | C | D | | | | |
| I-100 | D | B | D | | | | |
| I-101 | D | C | D | | | | |
| I-102 | D | A | D | | | | |
| I-103 | D | B | D | | | | |
| I-104 | D | A | D | | | | |
| I-105 | C | A | D | | | | |
| I-106 | D | C | D | | | | |
| I-107 | B | A | D | | | | |
| I-108 | D | C | D | | | | |
| I-109 | D | B | D | | | | |
| I-110 | D | C | D | | | | |
| I-111 | C | A | D | | | | ** |
| I-112 | D | B | D | | | | |
| I-113 | D | C | D | | | | |
| I-114 | D | D | D | | | | |
| I-115 | B | A | D | | | | |
| I-116 | B | A | D | | | | |
| I-117 | D | B | D | | | | |
| I-118 | D | C | D | | * | * | * |
| I-119 | D | A | D | | | | |
| I-120 | | A | D | | | | |
| I-121 | C | A | D | | | | |
| I-122 | D | A | D | | | | |
| I-123 | A | A | D | | | * | *** |
| I-124 | C | A | D | | | | |
| I-125 | C | A | D | | | | ** |
| I-126 | D | A | D | | | | |
| I-127 | D | A | D | | | | |
| I-128 | D | A | D | | | | |
| I-129 | D | A | D | | | | |
| I-130 | | A | D | | | | *** |
| I-131 | | B | D | | | | |
| I-132 | | A | D | | | | |
| I-133 | | A | D | | | | |
| I-134 | | A | D | | | | |
| I-135 | | D | D | | | | |
| I-136 | | D | D | | | | |
| I-137 | | A | D | | | | |
| I-138 | C | A | D | | | | |
| I-139 | A | A | D | | | | |
| I-140 | C | A | D | | | | |
| I-141 | C | C | D | D | | | |
| I-142 | A | B | D | | | ** | |
| I-143 | A | C | D | | | ** | |
| I-144 | A | C | D | | | ** | |
| I-145 | A | C | D | | | | |
| I-146 | B | D | D | | | | |
| I-147 | B | D | D | | | | |
| I-148 | A | C | D | | *** | *** | *** |
| I-149 | A | D | D | D | *** | *** | *** |
| I-150 | A | B | | | *** | *** | *** |
| I-151 | A | C | | | ** | ** | *** |
| I-152 | A | D | | | *** | ** | *** |
| I-153 | A | | | | | ** | *** |
| I-154 | A | | | | | ** | ** |
| I-155 | A | | | | | ** | *** |
| I-156 | A | | | | | ** | *** |
| I-157 | A | | | | *** | ** | *** |
| I-158 | A | | | | *** | ** | *** |
| I-159 | A | | | | | * | ** |
| I-160 | A | | | | | ** | ** |
| I-161 | B | | | | | | |
| I-162 | A | D | | | | ** | *** |
| I-163 | A | D | | | | ** | ** |
| I-164 | A | D | | | *** | ** | *** |
| I-165 | A | D | | | | * | ** |
| I-166 | C | D | | | | | |
| I-167 | A | D | | | | ** | ** |
| I-168 | D | D | | | | | |
| I-169 | A | D | | | ** | | ** |
| I-170 | A | D | | | *** | ** | ** |
| I-171 | A | D | | | ** | ** | ** |
| I-172 | C | D | | | | | |
| I-173 | C | D | | | | | |
| I-174 | C | D | | | | | |
| I-175 | D | D | | | | | |
| I-176 | C | D | | | | | |
| I-177 | B | D | | | | | |
| I-178 | A | D | | | | ** | ** |
| I-179 | A | C | | | ** | ** | *** |
| I-180 | B | C | | | | * | * |
| I-181 | A | D | | | | *** | *** |
| I-182 | A | D | | | | | |
| I-183 | A | D | | | *** | *** | *** |
| I-184 | C | B | | | | | |
| I-185 | A | D | | | | | |
| I-186 | B | C | | | | | |
| I-187 | A | D | | | | | |
| I-188 | B | D | | | | | |
| I-189 | B | D | | | | | |
| I-190 | A | B | | | | | *** |
| I-191 | B | C | | | | | |
| I-192 | C | D | | | | | |
| I-193 | B | B | | | | | |
| I-194 | A | D | | | | | |
| I-195 | C | D | | | | | |
| I-196 | C | D | | | | | |
| I-197 | B | C | | | | | |
| I-198 | B | D | | | | | |
| I-199 | A | D | | | | | ** |
| I-200 | A | C | | | | | |
| I-201 | B | C | | | | | |
| I-202 | A | D | | | | | |
| I-203 | D | D | | | | | |
| I-204 | C | C | | | | | |
| I-205 | A | D | | | ** | | *** |
| I-206 | B | D | | | | | |
| I-207 | D | D | | | | | |
| I-208 | A | C | | | | | |
| I-209 | A | | | | | | |
| I-210 | A | D | | | | | |
| I-211 | A | D | | | | | *** |
| I-212 | A | D | | | | | ** |
| I-213 | A | D | | | | | |
| I-214 | A | D | | | | | |
| I-215 | A | D | | | | | |
| I-216 | B | C | | | | | |
| I-217 | B | D | | | | | * |
| I-218 | A | D | | | ** | | ** |
| I-219 | A | D | | | | | *** |
| I-220 | A | D | | | *** | ** | *** |
| I-221 | A | D | | | | ** | ** |
| I-222 | A | D | | | *** | *** | *** |
| I-223 | D | D | D | D | | | |
| I-224 | A | C | | | *** | ** | *** |
| I-225 | A | D | | | | ** | ** |
| I-226 | A | A | D | | | *** | |
| I-227 | A | A | D | | | * | |
| I-228 | A | C | D | | ** | *** | *** |
| I-229 | A | B | D | | | ** | |
| I-230 | A | B | D | | | ** | |
| I-231 | A | C | D | | | *** | |
| I-232 | A | C | D | | * | * | |
| I-233 | B | D | D | | | | |
| I-234 | A | B | D | | ** | ** | ** |
| I-235 | C | B | | | | | |

In Vivo Studies

The following are various models of AML that will be used to assess the PK/PD relationship and efficacy of compounds in vivo.

### A. Subcutaneous Xenograft* Model:

Several human-derived AML cell lines will be tested in immunocompromised mice to elucidate the PK/PD relationship as well as the efficacy of compounds to inhibit tumor growth. Compounds will be administered to mice using an appropriate route of administration and dosing regimen at various concentrations and samples taken at various timepoints after dosing to evaluate plasma and tumoral exposure (pharmacokinetic measurements) as well as the effect on the m⁶A-mRNA pharmacodynamic biomarker extracted from tumors at varying timepoints. Body weight will be measured daily to assess tolerability.

### B. Disseminated Xenograft* Model:

Studies analogous to the ones described above will be conducted but rather as a disseminated model of disease achieved by tail-vein injection of various human AML cell lines. Cell lines may be luciferized for whole body imaging to assess disease burden at various doses and timepoints following drug administration. A Kaplan-Meier estimate will be used to assess survival over time. Other measurements of disease burden will be taken such as effects on composition of the bone marrow and spleen size.

*note: xenograft includes both cell-line derived (CDX) and patient-derived (PDX) models

Various genetically engineered mouse models (GEMMs) of AML in immunocompotent mice will also be used for the in vivo PK/PD/efficacy studies described above.

1. A compound of formula (I′) or (II′): 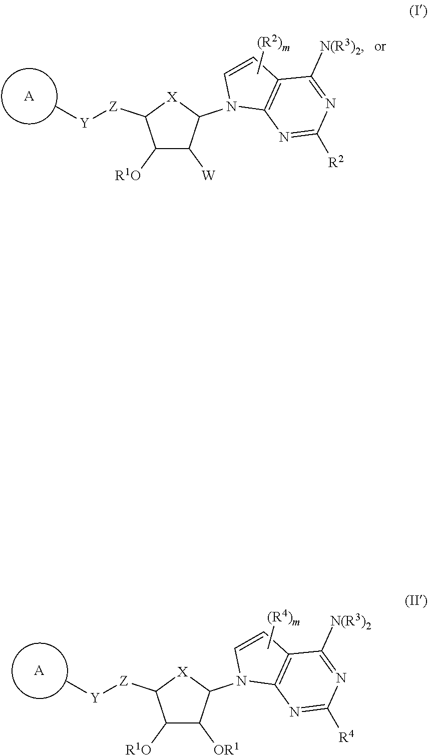
or a pharmaceutically acceptable salt thereof, wherein:
X is selected from O and CH₂;
R¹ is selected from H, C₁₋₆alkyl and —C(═O)—C₁₋₆alkyl;
W is selected from H, halo, C₁₋₆alkyl and —NH₂;
Y is selected from O, S, C(R^{a})₂ and NR^{b};
R^{a}, for each occurrence, is independently selected from H, C₁₋₆alkyl and halo;
R^{b} is H or C₁₋₆alkyl;
Z is selected from C₁₋₆alkyl, C₂₋₆alkenyl and C₂₋₆ alkynyl, each of which is optionally substituted with 1 to 3 halo;
Ring A is selected from benzene, naphthalene, 4 to 7-membered monocyclic heterocycloalkyl, 5 to 6-membered monocyclic heteroaromatic ring, and 8- to 10-membered bicyclic heteroaromatic ring, each of which is optionally substituted with 1 to 4 independently selected R₅;
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —C(═O)OR^{2a}, —C(═O)R^{2a}, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo,—CN, —OR^{2a}, —C(═O)N(R^{2a})₂, and —N(R^{2a})₂;
R^{2a}, for each occurrence, is independently selected from H, C₁₋₆alkyl, C₃₋₈cycloalkyl, and 4 to 6-membered heterocycloalkyl, wherein the C₁₋₆alkyl is optionally substituted with C₁₋₆alkoxy;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl,5 to 6-membered heteroaryl, halo, —CN, —OR^{2a}, —N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{4a}, —C(═O)N(R^{4a})₂, and —N(R^{4a})₂;
R^{4a}, for each occurrence, is independently selected from H, C₁₋₆alkyl, C₃₋₈cycloalkyl, and 4 to 6-membered heterocycloalkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂, —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{5a}—N(R^{5a})₂, —C(O)N(R^{5a})₂, —C(O)R^{5a}, and —C(O)OR^{5a};
R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, —NH₂, —SO₂C₁₋₆alkyl, —OC₁₋₆alkyl, C₁₋₆alkyl, C₁₋₆ haloalkyl, C₃₋₆cycloalkyl, phenyl, and 4 to 7-membered heterocycloalkyl;
or two R^{5a} together with the N atom from which they are attached form a 4 to 6-membered heterocycloalkyl optionally containing an additional heteroatom selected from O N and S, wherein the 4 to 6-membered heterocycloalkyl is optionally substituted with 1 to 3 substituents independently selected from halo, —OH, —NH₂, C₁₋₄alkyl and C₁₋₄haloalkyl; and
m is 1 or 2,
provided that the compound is not any one of the following, or a pharmaceutically acceptable salt thereof:

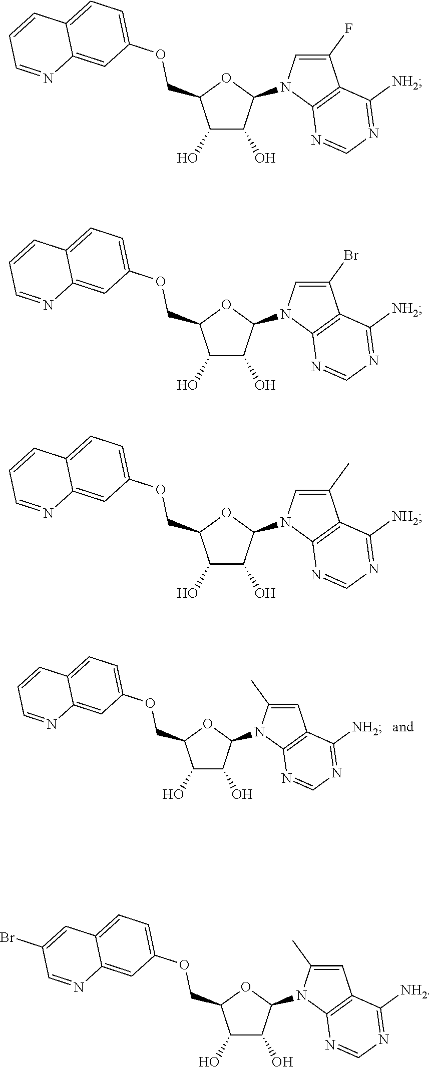 [SMILES: Nc1ncnc2c1c(F)cn2[C@@H]1O[C@H](COc2ccc3cccnc3c2)[C@@H](O)[C@H]1O ; Nc1ncnc2c1c(Br)cn2[C@@H]1O[C@H](COc2ccc3cccnc3c2)[C@@H](O)[C@H]1O ; Cc1cn([C@@H]2O[C@H](COc3ccc4cccnc4c3)[C@@H](O)[C@H]2O)c2ncnc(N)c12 ; Cc1cc2c(N)ncnc2n1[C@@H]1O[C@H](COc2ccc3cccnc3c2)[C@@H](O)[C@H]1O ; "and" ; Cc1cc2c(N)ncnc2n1[C@@H]1O[C@H](COc2ccc3cc(Br)cnc3c2)[C@@H](O)[C@H]1O]

2. The compound of claim 1, wherein the compound is represented by formula (I) or (II): 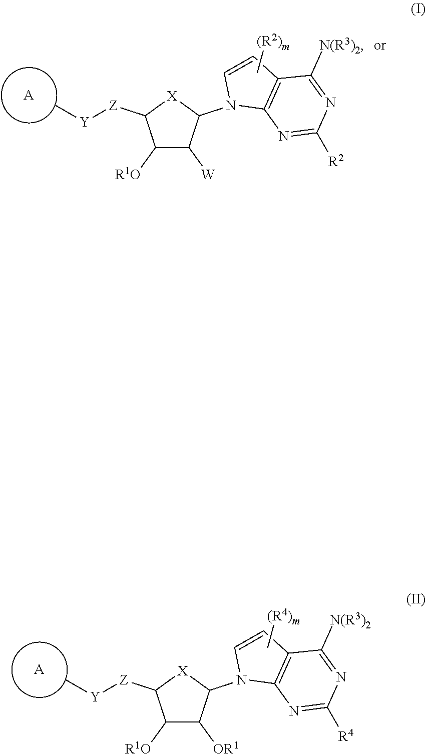
or a pharmaceutically acceptable salt thereof, wherein:
X is selected from O and CH₂;
R¹ is selected from H, C₁₋₆alkyl and —C(═O)—C₁₋₆alkyl;
W is selected from H, halo, C₁₋₆alkyl and —NH₂;
Y is selected from O, S, C(R^{a})₂ and NR^{b};
R^{a}, for each occurrence, is independently selected from H, C₁₋₆alkyl and halo;
R^{b} is H or C₁₋₆alkyl;
Z is selected from C₁₋₆alkyl, C₂₋₆alkenyl and C₂₋₆ alkynyl, each of which is optionally substituted with 1 to 3 halo;
Ring A is selected from benzene, naphthalene, 5 to 6-membered monocyclic heteroaromatic ring, and 8- to 10-membered bicyclic heteroaromatic ring, each of which is optionally substituted with 1 to 4 independently selected R⁵;
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl,5 to 6-membered heteroaryl, halo, —CN, —OR^{2a}, N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂;
R^{2a}, for each occurrence, is independently selected from H, C₁₋₆alkyl, C₃₋₈cycloalkyl, and 4 to 6-membered heterocycloalkyl;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl,5 to 6-membered heteroaryl, halo, —CN, —OR^{2a}, N(R^{2a})₂, and —C(═O)N(R^{2a})₂, wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{4a}, —C(═O)N(R^{4a})₂, and —N(R^{4a})₂,
R^{4a}, for each occurrence, is independently selected from H, C₁₋₆alkyl, C₃₋₈cycloalkyl, and 4 to 6-membered heterocycloalkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂, —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{5a}—N(R^{5a})₂, —C(O)N(R^{5a})₂, —C(O)R^{5a}, and —C(O)OR^{5a};
R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl, and 4 to 7-membered heterocycloalkyl; or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl; and
m is 1 or 2,
provided that the compound is not any one of the following, or a pharmaceutically acceptable salt thereof:

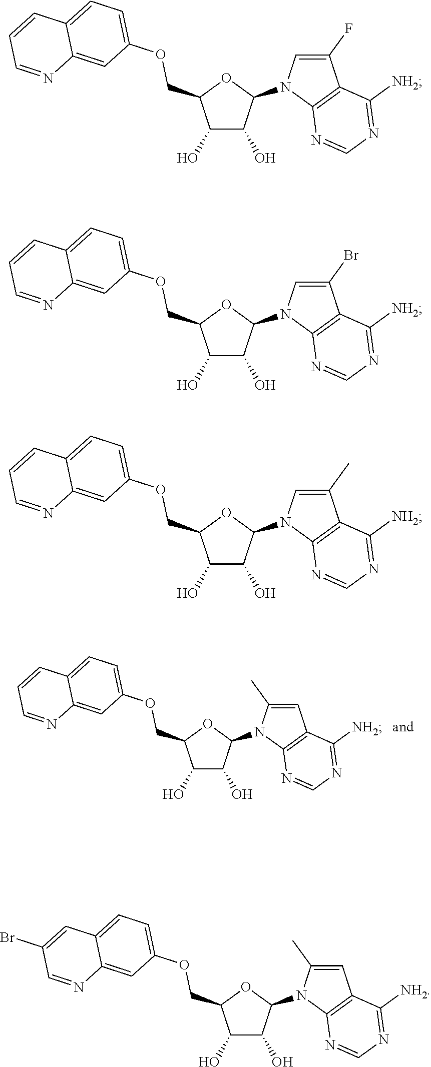 [SMILES: Nc1ncnc2c1c(F)cn2[C@@H]1O[C@H](COc2ccc3cccnc3c2)[C@@H](O)[C@H]1O ; Nc1ncnc2c1c(Br)cn2[C@@H]1O[C@H](COc2ccc3cccnc3c2)[C@@H](O)[C@H]1O ; Cc1cn([C@@H]2O[C@H](COc3ccc4cccnc4c3)[C@@H](O)[C@H]2O)c2ncnc(N)c12 ; Cc1cc2c(N)ncnc2n1[C@@H]1O[C@H](COc2ccc3cccnc3c2)[C@@H](O)[C@H]1O ; "and" ; Cc1cc2c(N)ncnc2n1[C@@H]1O[C@H](COc2ccc3cc(Br)cnc3c2)[C@@H](O)[C@H]1O]

3. The compound of claim 1 or 2, wherein the compound is represented by the following formula: 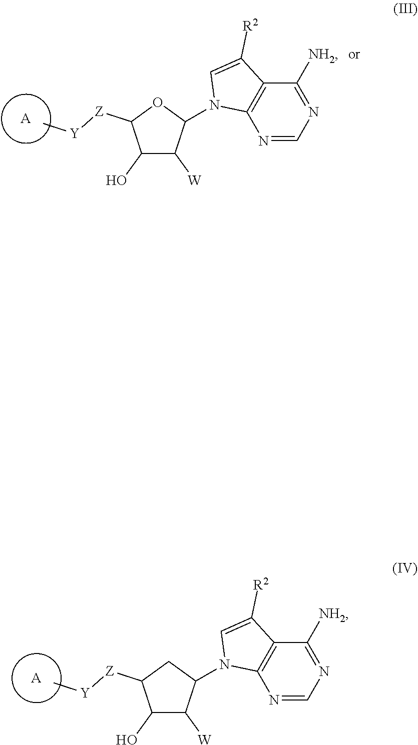
or a pharmaceutically acceptable salt thereof.

4. The compound of claim 1 or 2, wherein the compound is represented by the following formula: 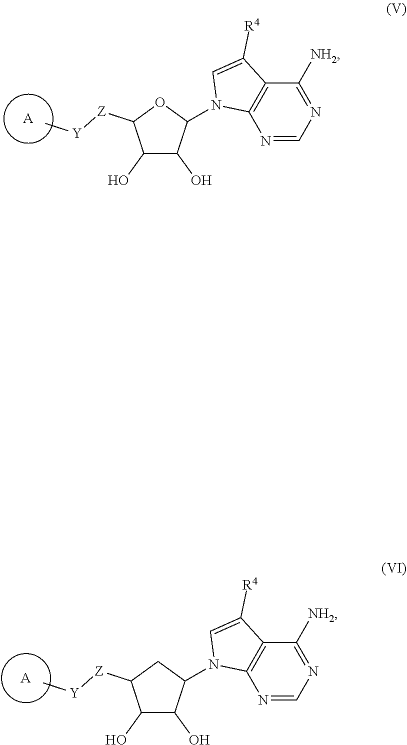
or a pharmaceutically acceptable salt thereof.

5. The compound of any one of claims 1-4, or a pharmaceutically acceptable salt thereof, wherein Y is O or C(R^{a})₂ and R^{a}, for each occurrence, is independently H or halo.

6. The compound of any one of claims 1-5, or a pharmaceutically acceptable salt thereof, wherein Y is O.

7. The compound of any one of claims 1-5, or a pharmaceutically acceptable salt thereof, wherein Y is CH₂.

8. The compound of any one of claims 1-7, or a pharmaceutically acceptable salt thereof, wherein Z is selected from C₁₋₄alkyl and C₂₋₄ alkenyl, each of which is optionally substituted with 1 to 3 halo.

9. The compound of any one of claims 1-8, or a pharmaceutically acceptable salt thereof, wherein Z is CH₂.

10. The compound of claim 1 or 2, wherein the compound is represented by the following formula: 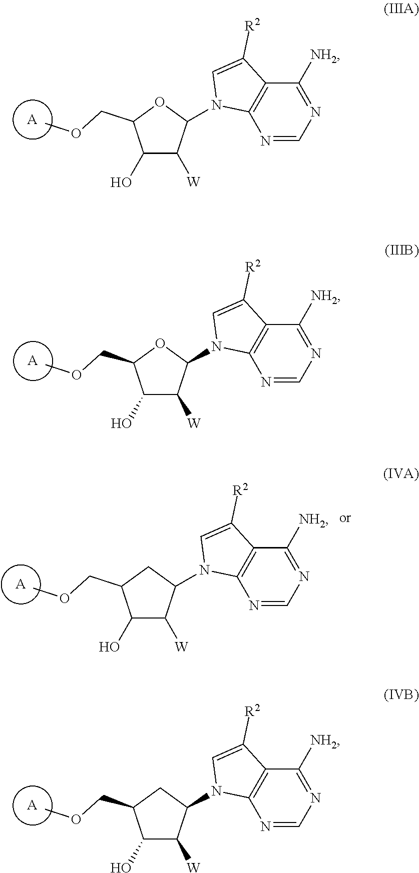
or a pharmaceutically acceptable salt thereof.

11. The compound of claim 1 or 2, wherein the compound is represented by the following formula: 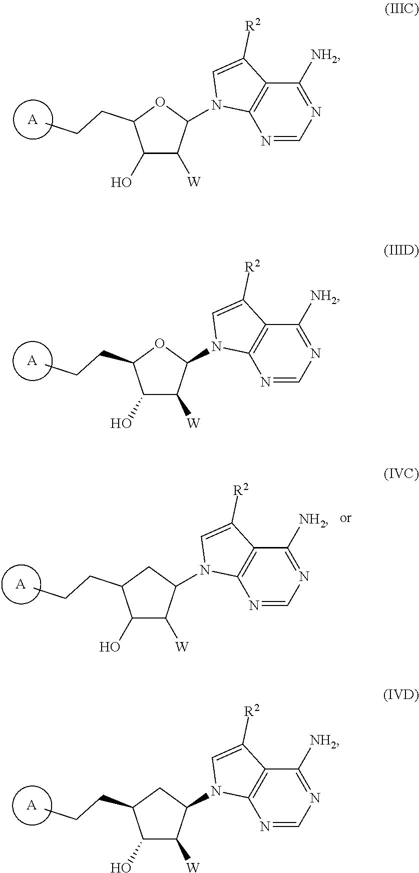
or a pharmaceutically acceptable salt thereof.

12. The compound of claim 1 or 2, wherein the compound is represented by the following formula: 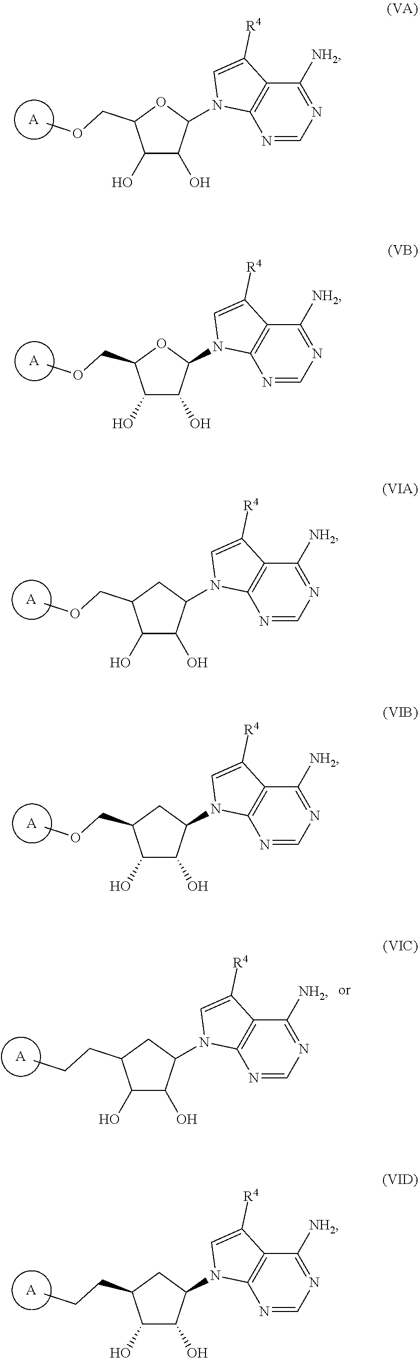
or a pharmaceutically acceptable salt thereof.

13. The compound of claim 1 or 2, wherein the compound is represented by the following formula: 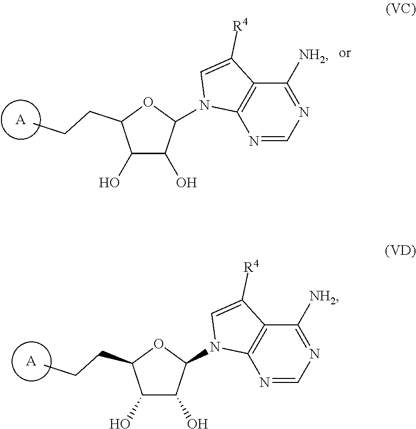
or a pharmaceutically acceptable salt thereof.

14. The compound of any one of claims 1-13, or a pharmaceutically acceptable salt thereof, wherein ring A is a 9- to 10-membered bicyclic heteroaromatic ring optionally substituted with 1-4 R⁵ groups.

15. The compound of any one of claims 1-13, or a pharmaceutically acceptable salt thereof, wherein ring A is selected from quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, isothiazolopyridine, thienopyridine, pyridine, piperidine, and benzene, each of which is optionally substituted with 1 to 3 independently selected R⁵.

16. The compound of any one of claims 1-13, or a pharmaceutically acceptable salt thereof, wherein ring A is selected from quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, and isothiazolopyridine, each of which is optionally substituted with 1 to 3 independently selected R⁵.

17. The compound of claim 16, or a pharmaceutically acceptable salt thereof, wherein ring A is selected from quinoline, quinazoline, quinoxaline, benzoimidiazole, benzothiazole, napththyridine, indole, pyrrolopyrimidine and indazole, each of which is optionally substituted with 1 to 3 independently selected R⁵.

18. The compound of any one of claims 1-13, or a pharmaceutically acceptable salt thereof, wherein ring A is selected from benzene, naphthalene and pyridine.

19. The compound of claim 17, or a pharmaceutically acceptable salt thereof, wherein ring A is quinoline optionally substituted with 1 to 3 independently selected R⁵.

20. The compound of any one of claims 1-13, or a pharmaceutically acceptable salt thereof, wherein ring A is represented by the following formula: 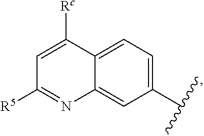
wherein R^{c} is selected from H, halo, C₁₋₄alkyl, 4 to 6-membered heterocycloalkyl, —OR^{c1} and —N(R^{c1})₂, wherein R^{c1}, for each occurrence, is independently H, C₁₋₄alkyl, or C₃₋₆ cycloalkyl, wherein the C₁₋₄alkyl is optionally substituted with C₃₋₆cycloalkyl or phenyl.

21. The compound of any one of claims 1-13, or a pharmaceutically acceptable salt thereof, wherein ring A is represented by the following formula: 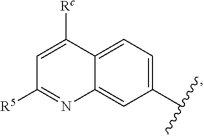
wherein R^{c} is selected from H, halo, C₁₋₄alkyl, —OR^{c1} and —N(R^{c1})₂, and R^{c1}, for each occurrence, is independently H or C₁₋₄alkyl optionally substituted with C₃₋₆cycloalkyl or phenyl.

22. The compound of claim 20 or 21, or a pharmaceutically acceptable salt thereof, wherein R^{c} is H.

23. The compound of any one of claims 1-22, or a pharmaceutically acceptable salt thereof, wherein R⁵, for each occurrence, is independently selected from H, C₁₋₆alkyl, C₂₋₆alkynyl, C₃₋₆cycloalkyl, 4 to 6-membered heterocycloalkyl, 5 to 6-membered heteroaryl, halo, —OR^{5a}, —C(═O)N(R^{5a})₂, —N(R^{5a})₂, —NR^{5a}C(═O)R^{5a}, and —NR^{5a}C(═O)N(R^{5a})₂, wherein the C₁₋₆alkyl, C₂₋₆alkynyl, C₃₋₆cycloalkyl, 4 to 6-membered heterocycloalkyl, and 5 to 6-membered heteroaryl are each optionally substituted with 1 to 3 substituents independently selected from C₁₋₆alkyl, C₁₋₆ haloalkyl, C₃₋₆cycloalkyl, —N(R^{5a})₂, phenyl, halo, —OH, —NH₂, and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OH, —NH₂, phenyl, —SO₂C₁₋₃alkyl, —OC₁₋₃alkyl, C₁₋₃alkyl and C₃₋₈cycloalkyl, or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, —OH, —NH₂, C₁₋₄alkyl and C₁₋₄ haloalkyl.

24. The compound of any one of claims 1-22, or a pharmaceutically acceptable salt thereof, wherein R⁵, for each occurrence, is independently selected from H, C₁₋₆alkyl, C₂₋₆alkynyl, 4 to 6-membered heterocycloalkyl, 5 to 6-membered heteroaryl, halo, —ORS^{a}—N(R^{5a})₂, —NR^{5a}C(═O)R^{5a}, and —NR^{5a}C(═O)N(R^{5a})₂, wherein the C₁₋₆alkyl, C₂₋₆alkynyl, 4 to 6-membered heterocycloalkyl, 5 to 6-membered heteroaryl are each optionally substituted with 1 to 3 substituents independently selected from C₁₋₆alkyl, C₁₋₆ haloalkyl, C₃₋₆cycloalkyl, phenyl, halo and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OH, C₁₋₃alkyl and C₃₋₈cycloalkyl, or two R^{5a} together with the N atom from which they are attached form a 4 to 6-membered heterocycloalkyl optionally containing an additional heteroatom selected from O, N and S.

25. The compound of any one of claims 1-24, or a pharmaceutically acceptable salt thereof, wherein R⁵, for each occurrence, is independently selected from H, C₁₋₆alkyl and —N(R^{5a})₂; and R^{5a}, for each occurrence, is independently H or C₁₋₆alkyl, or two R^{5a} together with the N atom from which they are attached form a 4 to 6-membered heterocycloalkyl optionally containing an additional heteroatom selected from O, N and S.

26. The compound ofany one of claims 1-23, or a pharmaceutically acceptable salt thereof, wherein R⁵, for each occurrence, is independently selected from H, Br, F, —CH₃, —CH₂CH₃, —CH₂N(CH₃)₂, —OH, —OCH₃, —NH₂, —NHCH₃, —NHCH₂CH₃, —N(CH₃)₂, —NHCH(CH₃)₂, —NHCH₂CH₂CH₃, —NHCH₂CH₂OH, —NHCH₂-cyclopropyl, —NH-cyclobutyl, —NHCH₂Ph, —N(CH₃)CH₂Ph, —NHPh, —NHC(O)NH₂, —NH—C(═O)-cyclopropyl, —NHC(═O)NHCH₃, —C≡C-Ph, imidazolyl, pyrrolidinyl, morpholinyl and azetidinyl.

27. The compound of any one of claims 1-23, or a pharmaceutically acceptable salt thereof, wherein R⁵, for each occurrence, is independently selected from H, Br, F, —CH₃, —CH₂CH₃, —CH₂N(CH₃)₂, —OH, —OCH₃, —NH₂, —NHCH₃, —NHCH₂CH₃, —N(CH₃)₂, —NHCH(CH₃)₂, —NHCH₂CH₂CH₃, —NHCH₂CH₂OH, —NHCH₂-cyclopropyl, —NHCH₂Ph, —N(CH₃)CH₂Ph, —NHPh, —NHC(O)NH₂, —NH—C(═O)-cyclopropyl, —NHC(═O)NHCH₃, —C≡C-Ph, imidazolyl, pyrrolidinyl and morpholinyl.

28. The compound of any one of claims 1-23, or a pharmaceutically acceptable salt thereof, wherein R⁵, for each occurrence, is independently selected from —NHCH₃, —NHCH₂CH₃, —NH-cyclobutyl, and azetidinyl.

29. The compound of any one of claims 1-3, 5-11, and 14-28, or a pharmaceutically acceptable salt thereof, wherein W is selected from H and halo.

30. The compound of claim 29, or a pharmaceutically acceptable salt thereof, wherein W is selected from H and F.

31. The compound of any one of claims 1-3, 5-11, and 14-30, or a pharmaceutically acceptable salt thereof, wherein R² is H, halo, C₁₋₆alkyl, C₃₋₈cycloalkyl, C₅₋₈cycloalkenyl, 4 to 6-membered heterocycloalkyl, 4 to 6-membered heterocycloalkenyl, phenyl, 5 to 6-membered heteroaryl, wherein the C₁₋₆alkyl, C₃₋₈cycloalkyl, C₅₋₈cycloalkenyl, 4 to 6-membered heterocycloalkyl, 4 to 6-membered heterocycloalkenyl and 5 to 6-membered heteroaryl are each optionally substituted with 1 to 3 substituents independently selected from halo, C₁₋₄alkyl, C₁₋₄haloalkyl and C₃₋₆cycloalkyl.

32. The compound of claim 31, or a pharmaceutically acceptable salt thereof, wherein R² is selected from halo, C₃₋₆cycloalkyl, 4 to 6-membered heterocycloalkyl, and 5 to 6-membered heteroaryl, wherein the C₃₋₆cycloalkyl, 4 to 6-membered heterocycloalkyl, and 5 to 6-membered heteroaryl are each optionally substituted with halo, C₁₋₄alkyl and C₁₋₄haloalkyl.

33. The compound of claim 31, or a pharmaceutically acceptable salt thereof, wherein R² is selected from halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

34. The compound of claim 31, or a pharmaceutically acceptable salt thereof, wherein R² is selected from H, Br, Cl, —CH₃, —CF3, —CH₂-cyclopropyl, cyclopropyl, cyclopentyl, 1-methylimidazolyl, dihydropyrrolyl, 1-methyl-1,2,3, 6-tetrahydropyridinyl, tetrahydrofuranyl, tetrahydro-2H-pyranyl, 5-methylfuranyl, 1-methylpyrazolyl, 1-ethylpyrazolyl, 1-isopropylpyrazolyl, methyltetrahydropyridinyl, pyridinyl, 1-methylpyrrolidinyl, 1-methylpiperidinyl, and diflurophenyl.

35. The compound of claim 31, or a pharmaceutically acceptable salt thereof, wherein R² is selected from H, Br, Cl, —CH₃, —CF3, —CH₂-cyclopropyl, cyclopropyl, cyclopentyl, 1-methylimidazolyl, dihydropyrrolyl, 1-methyl-1,2,3, 6-tetrahydropyridinyl, tetrahydro-2H-pyranyl, 1-methylpyrazolyl, 1-ethylpyrazolyl, 1-isopropylpyrazolyl, methyltetrahydropyridinyl, pyridinyl, 1-methylpyrrolidinyl, 1-methylpiperidinyl, and diflurophenyl.

36. The compound of claim 31, or a pharmaceutically acceptable salt thereof, wherein R² is selected from cyclopentyl, tetrahydrofuranyl, 5-methylfuranyl, and 1-methylpyrazolyl.

37. The compound of any one of claims 1, 2, 4-9, and 12-30, or a pharmaceutically acceptable salt thereof, wherein R⁴ is selected from C₃₋₈cycloalkyl, C₅₋₈cycloalkenyl, 4 to 6-membered heterocycloalkyl, 4 to 6-membered heterocycloalkenyl, phenyl, 5 to 6-membered heteroaryl, halo, —CN, —OR^{2a}, —N(R^{2a})₂, and —C(O)N(R^{2a})₂, wherein the C₃₋₈cycloalkyl, C₅₋₈cycloalkenyl, 4 to 6-membered heterocycloalkyl, 4 to 6-membered heterocycloalkenyl, phenyl, 5 to 6-membered heteroaryl are each optionally substituted with 1 to 3 substituents independently selected from C₁₋₆alkyl, C₁₋₆ haloalkyl, C₃₋₈cycloalkyl, 4 to 7-membered heterocycloalkyl, phenyl, 5- to 6-membered heteroaryl, halo, —CN, —OR^{4a}, —C(O)N(R^{4a})₂, and —N(R^{4a})₂; and
R^{4a}, for each occurrence, is independently selected from H, C₁₋₆alkyl, C₃₋₈cycloalkyl, and 4 to 6-membered heterocycloalkyl.

38. The compound of claim 37, or a pharmaceutically acceptable salt thereof, wherein R⁴ is selected from halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

39. The compound of claim 37, wherein R⁴ is selected from C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

40. The compound of claim 37, or a pharmaceutically acceptable salt thereof, wherein R⁴ is selected from H, C₁, Br, —CH₃, —CH₂CH₂CH₃, propyl, —CH₂-cyclopentyl, —CH₂—OH, cyclopentyl, cyclohexyl, difluorocyclohexyl, tetrahydrofuranyl, tetrahydropyranyl, and methylpyrazolyl.

41. The compound of claim 37, or a pharmaceutically acceptable salt thereof, wherein R⁴ is selected from cyclopentyl and 1-methylpyrazolyl.

42. The compound of claim 1 or 2, wherein the compound is represented by the following formula: 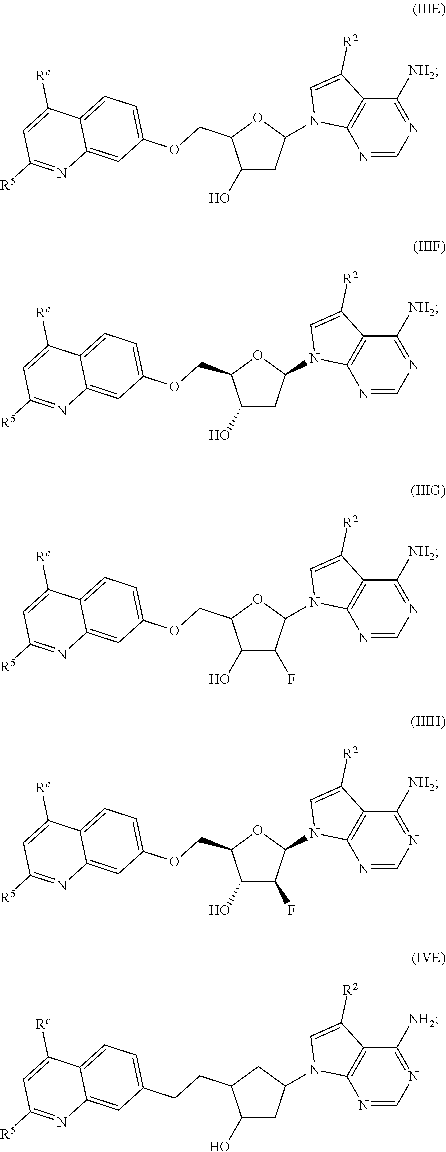 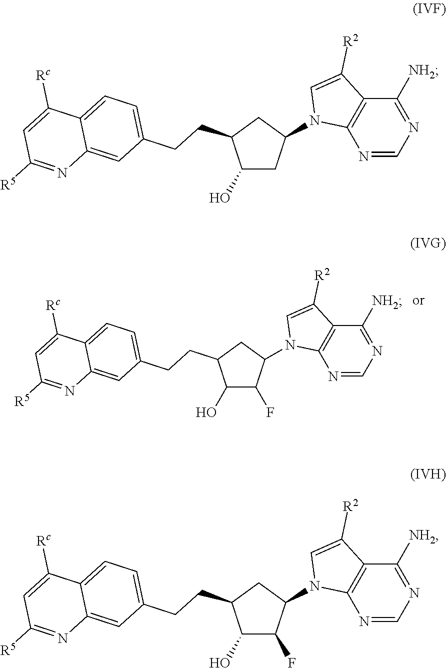
or a pharmaceutically acceptable salt thereof, wherein:
W is H or F;
R² is selected from H, halo, C₃₋₆cycloalkyl, 4 to 6-membered heteroccloalkyl, and 5 to 6-membered heteroaryl, wherein the C₃₋₆cycloalkyl, 4 to 6-membered heteroccloalkyl, and 5 to 6-membered heteroaryl are each optionally substituted with halo, C₁₋₄alkyl and C₁₋₄haloalkyl; and
R⁵ is —N(R^{5a})₂;
R^{5a}, for each occurrence, is independently selected from H, C₁₋₆alkyl, and C₃₋₆cycloalkyl, or two R^{5a} together with the N atom from which they are attached form a 4 to 6-membered heterocycloalkyl optionally containing an additional heteroatom selected from O and N;
R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂, —NR^{c1}(═O)R^{c1}, —NR^{c1}(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and
R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl and 4 to 7-membered heterocycloalkyl, or two R^{c1} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl.

43. The compound of claim 1 or 2, wherein the compound is represented by the following formula: 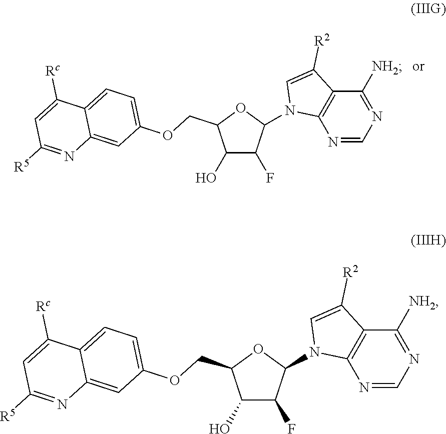
or a pharmaceutically acceptable salt thereof, wherein:
R² is selected from H, halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl; and
R⁵ is —N(R^{5a})₂;
R^{5a}, for each occurrence, is independently selected from H and C₁₋₆alkyl, or two R^{5a} together with the N atom from which they are attached form a 4 to 6-membered heterocycloalkyl optionally containing an additional heteroatom selected from O and N;
R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂, —NR^{c1}(═O)R^{c1}, —NR^{c1}(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and
R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl and 4 to 7-membered heterocycloalkyl, or two R^{c} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl.

44. The compound of claim 1 or 2, wherein the compound is represented by the following formula: 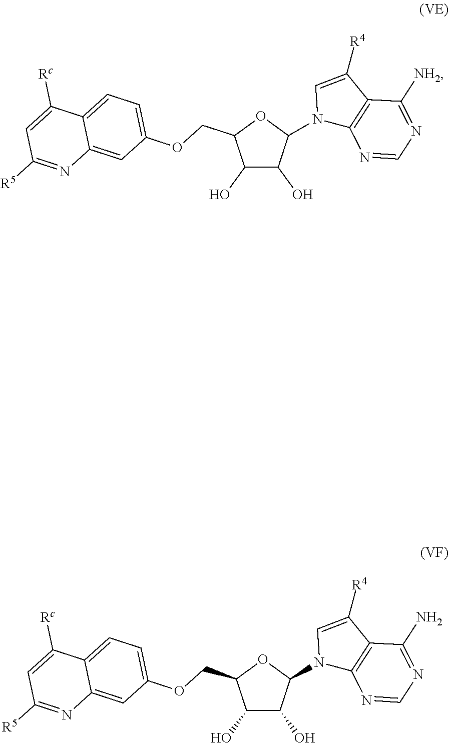
or a pharmaceutically acceptable salt thereof, wherein:
R⁴ is selected from halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl;
R⁵ is —N(R^{5a})₂;
R^{5a}, for each occurrence, is independently selected from H and C₁₋₆alkyl, or two R^{5a} together with the N atom from which they are attached form a 4 to 6-membered heterocycloalkyl optionally containing an additional heteroatom selected from O and N;
R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂, —NR^{c1}(═O)R^{c1}, —NR^{c1}(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆ haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and
R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆ haloalkyl, C₃₋₆cycloalkyl, phenyl and 4 to 7-membered heterocycloalkyl, or two R^{c1} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl.

45. The compound of any one of claims 42-44, or a pharmaceutically acceptable salt thereof, wherein R^{c} is selected from H, halo, C₁₋₄alkyl and —N(R^{c1})₂, and R^{c1}, for each occurrence, is independently H or C₁₋₄alkyl.

46. The compound of claim any of claims 42-44, or a pharmaceutically acceptable salt thereof, wherein R^{c} is H.

47. The compound of any one of claims 42-46, or a pharmaceutically acceptable salt thereof, wherein for formula (IIIE), (IIIF), (IIIG), (IIIH), (IVE), (IVF), (IVG) or (IVH), R² is cyclopentyl, 5-membered heterocycloalkyl or 5-membered heteroaryl, each of which is optionally substituted with C₁₋₄alkyl; and for formula (VE) or (VF), R⁴ is cyclopentyl, 5-membered heterocycloalkyl or 5-membered heteroaryl, each of which is optionally substituted with 1 to 2 substituents independently selected from C₁₋₄alkyl.

48. The compound of claim 47, or a pharmaceutically acceptable salt thereof, wherein R² is cyclopentyl, tetrahydrofuranyl, furanyl or pyrazolyl, each of which is optionally substituted with 1 or 2 independently selected from C₁₋₄alkyl; and R⁴ is cyclopentyl, tetrahydrofuranyl, furanyl or pyrazolyl, each of which is optionally substituted with 1 or 2 independently selected from C₁₋₄alkyl.

49. A pharmaceutical composition comprising a compound of any one of claims 1-48, or a pharmaceutically acceptable salt thereof, and a pharmaceutically acceptable excipient.

50. A method of treating a disease or disorder responsive to inhibition of METTL3 activity in a subject comprising administering to the subject an effective amount of the compound according any one of claims 1-48, or a pharmaceutically acceptable salt thereof.

51. The method of claim 50, wherein the disease or disorder is an infection.

52. The method of claim 51, wherein the infection is a viral infection.

53. The method of claim 50, wherein the disease or disorder is cancer.

54. The method of claim 53, wherein the cancer is selected from glioblastoma, leukemia, stomach cancer, prostate cancer, colorectal cancer, endometrial cancer, breast cancer, pancreatic cancer, kidney cancer, lung cancer, bladder cancer, ovarian cancer, esophageal/upper aerodigestive cancer, liver cancer, bone cancer, acute lymphocytic leukemia, non-Hodgkin's lymphoma (NHL), multiple myeloma, mesothelioma and sarcoma.

55. The method of claim 54, wherein the cancer is acute myeloid leukemia


SEQUENCE LISTING (4 sequences)

SEQ ID NO: 1 - RNA, 11 nt, Artificial Sequence
  source: /note=“Description of Artificial Sequence Synthetic oligonucleotide”
ucuggacuaaa

SEQ ID NO: 2 - RNA, 73 nt, Artificial Sequence
  source: /note=“Description of Artificial Sequence Synthetic oligonucleotide”
gccgagauagcucaguugggagagcguuagacugaagaucuaaaggucccugguucaaucccggguuucggca

SEQ ID NO: 3 - RNA, 39 nt, Artificial Sequence
  source: /note=“Description of Artificial Sequence Synthetic oligonucleotide”
cgauacagagaagauuagcauacgcaaauucgugaagcg

SEQ ID NO: 4 - protein, 24 aa, Artificial Sequence
  source: /note=“Description of Artificial Sequence Synthetic peptide”
SGRGKGGKGLGKGGAKRHRKVGGK